SEQUENCE LISTING (72 sequences)

SEQ ID NO: 1 - protein, 8 aa, Gallus gallus
SIINFEKL

SEQ ID NO: 2 - protein, 8 aa, synthetic construct
  REGION 1..8: note = Made in Lab - Altered ovalbumin peptide
SAINFEKL

SEQ ID NO: 3 - protein, 8 aa, synthetic construct
  REGION 1..8: note = Made in Lab - Altered ovalbumin peptide
SIITFEKL

SEQ ID NO: 4 - protein, 9 aa, Lymphocytic choreomeningitis virus
KAVYNFATM

SEQ ID NO: 5 - protein, 5 aa, unidentified
  REGION 1..5: note = Sortase recognition sequence motif
  VARIANT 4: note = X is any amino acid residue
LPXTG

SEQ ID NO: 6 - protein, 5 aa, unidentified
  REGION 1..5: note = Modified sortase recognition sequence motif
  VARIANT 3: note = X is any amino acid residue
LAXTG

SEQ ID NO: 7 - protein, 5 aa, unidentified
  REGION 1..5: note = Modified sortase recognition sequence motif
  VARIANT 3: note = X is any amino acid residue
LAXSG

SEQ ID NO: 8 - skipped

SEQ ID NO: 9 - protein, 5 aa, unidentified
  REGION 1..5: note = Sortase recognition sequence
LPKTG

SEQ ID NO: 10 - protein, 5 aa, unidentified
  REGION 1..5: note = Sortase recognition sequence
LPATG

SEQ ID NO: 11 - protein, 5 aa, unidentified
  REGION 1..5: note = Sortase recognition sequence
LPNTG

SEQ ID NO: 12 - protein, 5 aa, unidentified
  REGION 1..5: note = Sortase recognition sequence
LPETG

SEQ ID NO: 13 - protein, 5 aa, unidentified
  REGION 1..5: note = Sortase recognition sequence
  VARIANT 3: note = X is any amino acid residue
LPXAG

SEQ ID NO: 14 - protein, 5 aa, unidentified
  REGION 1..5: note = Sortase recognition sequence
LPNAG

SEQ ID NO: 15 - protein, 5 aa, unidentified
  REGION 1..5: note = Sortase recognition sequence
  VARIANT 3: note = X is any amino acid residue
LPXTA

SEQ ID NO: 16 - protein, 5 aa, unidentified
  REGION 1..5: note = Sortase recognition sequence
LPNTA

SEQ ID NO: 17 - protein, 5 aa, unidentified
  REGION 1..5: note = Sortase recognition sequence
  VARIANT 3: note = X is any amino acid residue
LGXTG

SEQ ID NO: 18 - protein, 5 aa, unidentified
  REGION 1..5: note = Sortase recognition sequence
LGATG

SEQ ID NO: 19 - protein, 5 aa, unidentified
  REGION 1..5: note = Sortase recognition sequence
  VARIANT 3: note = X is amino acid residue
IPXTG

SEQ ID NO: 20 - protein, 5 aa, unidentified
  REGION 1..5: note = Sortase recognition sequence
IPNTG

SEQ ID NO: 21 - protein, 5 aa, unidentified
  REGION 1..5: note = Sortase recognition sequence
IPETG

SEQ ID NO: 22 - protein, 5 aa, unidentified
  REGION 1..5: note = Sortase recognition sequence
LAETG

SEQ ID NO: 23 - skipped

SEQ ID NO: 24 - protein, 5 aa, unidentified
  REGION 1..5: note = Sortase recognition sequence
NPQTN

SEQ ID NO: 25 - protein, 5 aa, unidentified
  REGION 1..5: note = Sortase recognition sequence
NPKTG

SEQ ID NO: 26 - protein, 5 aa, unidentified
  REGION 1..5: note = Sortase recognition sequence
NSKTA

SEQ ID NO: 27 - protein, 5 aa, unidentified
  REGION 1..5: note = Sortase recognition sequence
NPQTG

SEQ ID NO: 28 - protein, 5 aa, unidentified
  REGION 1..5: note = Sortase recognition sequence
NAKTN

SEQ ID NO: 29 - protein, 5 aa, unidentified
  REGION 1..5: note = Sortase recognition sequence
NPQSS

SEQ ID NO: 30 - protein, 416 aa, synthetic construct
  REGION 1..416: note = Made in Lab - FUT6 lentivirus construct
MAKGFYISKSLGILGILLGVAAVCTIIALSVVYSQEKNKNANSSPVASTTPSASATTNPASATTLGGYPYDVPDYAEFASTSLYKKAGSENLYFQGDPTVYPNGSRFPDSTGTPAHSIPLILLWTWPFNKPIALPRCSEMVPGTADCNITADRKVYPQADAVIVHHREVMYNPSAQLPRSPRRQGQRWIWFSMESPSHCWQLKAMDGYFNLTMSYRSDSDIFTPYGWLEPWSGQPAHPPLNLSAKTELVAWAVSNWGPNSARVRYYQSLQAHLKVDVYGRSHKPLPQGTMMETLSRYKFYLAFENSLHPDYITEKLWRNALEAWAVPVVLGPSRSNYERFLPPDAFIHVDDFQSPKDLARYLQELDKDHARYLSYFRWRETLRPRSFSWALAFCKACWKLQEESRYQTRGIAAWFT

SEQ ID NO: 31 - DNA, 1251 bp, synthetic construct
  misc_feature 1..1251: note = Made in Lab - FUT6 lentivirus construct
  source 1..1251: mol_type = other DNA
atggccaagggcttctatatttccaagtccctgggcatcctggggatcctcctgggcgtggcagccgtgtgcacaatcatcgcactgtcagtggtgtactcccaggagaagaacaagaacgccaacagctcccccgtggcctccaccaccccgtccgcctcagccaccaccaaccccgcctcggccaccaccttgggcggctacccatacgatgttccagattacgctgagttcgccagcaccagcctgtacaagaaggccggcagcgagaacctgtacttccagggcgatcccactgtgtaccctaatgggtcccgcttcccagacagcacagggacccccgcccactccatccccctgatcctgctgtggacgtggccttttaacaaacccatagctctgccccgctgctcagagatggtgcctggcacggctgactgcaacatcactgccgaccgcaaggtgtatccacaggcagacgcggtcatcgtgcaccaccgagaggtcatgtacaaccccagtgcccagctcccacgctccccgaggcggcaggggcagcgatggatctggttcagcatggagtccccaagccactgctggcagctgaaagccatggacggatacttcaatctcaccatgtcctaccgcagcgactccgacatcttcacgccctacggctggctggagccgtggtccggccagcctgcccacccaccgctcaacctctcggccaagaccgagctggtggcctgggcagtgtccaactgggggccaaactccgccagggtgcgctactaccagagcctgcaggcccatctcaaggtggacgtgtacggacgctcccacaagcccctgccccagggaaccatgatggagacgctgtcccggtacaagttctatctggccttcgagaactccttgcaccccgactacatcaccgagaagctgtggaggaacgccctggaggcctgggccgtgcccgtggtgctgggccccagcagaagcaactacgagaggttcctgccacccgacgccttcatccacgtggacgacttccagagccccaaggacctggcccggtacctgcaggagctggacaaggaccacgcccgctacctgagctactttcgctggcgggagacgctgcggcctcgctccttcagctgggcactcgctttctgcaaggcctgctggaaactgcaggaggaatccaggtaccagacacgcggcatagcggcttggttcacctga

SEQ ID NO: 32 - DNA, 8622 bp, synthetic construct
  misc_feature 1..8622: note = Made in Lab - lentivirus expression vector
  source 1..8622: mol_type = other DNA
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggttaacttttaaaagaaaaggggggattggggggtacagtgcaggggaaagaatagtagacataatagcaacagacatacaaactaaagaattacaaaaacaaattacaaaaattcaaaattttatcgagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcggtaccgcggccgcccggggatccatggccaagggcttctatatttccaagtccctgggcatcctggggatcctcctgggcgtggcagccgtgtgcacaatcatcgcactgtcagtggtgtactcccaggagaagaacaagaacgccaacagctcccccgtggcctccaccaccccgtccgcctcagccaccaccaaccccgcctcggccaccaccttgggcggctacccatacgatgttccagattacgctgagttcgccagcaccagcctgtacaagaaggccggcagcgagaacctgtacttccagggcgatcccactgtgtaccctaatgggtcccgcttcccagacagcacagggacccccgcccactccatccccctgatcctgctgtggacgtggccttttaacaaacccatagctctgccccgctgctcagagatggtgcctggcacggctgactgcaacatcactgccgaccgcaaggtgtatccacaggcagacgcggtcatcgtgcaccaccgagaggtcatgtacaaccccagtgcccagctcccacgctccccgaggcggcaggggcagcgatggatctggttcagcatggagtccccaagccactgctggcagctgaaagccatggacggatacttcaatctcaccatgtcctaccgcagcgactccgacatcttcacgccctacggctggctggagccgtggtccggccagcctgcccacccaccgctcaacctctcggccaagaccgagctggtggcctgggcagtgtccaactgggggccaaactccgccagggtgcgctactaccagagcctgcaggcccatctcaaggtggacgtgtacggacgctcccacaagcccctgccccagggaaccatgatggagacgctgtcccggtacaagttctatctggccttcgagaactccttgcaccccgactacatcaccgagaagctgtggaggaacgccctggaggcctgggccgtgcccgtggtgctgggccccagcagaagcaactacgagaggttcctgccacccgacgccttcatccacgtggacgacttccagagccccaaggacctggcccggtacctgcaggagctggacaaggaccacgcccgctacctgagctactttcgctggcgggagacgctgcggcctcgctccttcagctgggcactcgctttctgcaaggcctgctggaaactgcaggaggaatccaggtaccagacacgcggcatagcggcttggttcacctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 33 - protein, 10 aa, synthetic construct
  REGION 1..10: note = Made in Lab - complementarity determining region sequence
ASGTDYAEQF

SEQ ID NO: 34 - protein, 13 aa, synthetic construct
  REGION 1..13: note = Made in Lab - complementarity determining region sequence
ASSPQLGGRREQY

SEQ ID NO: 35 - protein, 12 aa, synthetic construct
  REGION 1..12: note = Made in Lab - complementarity determining region sequence
ASSIGTANTEVF

SEQ ID NO: 36 - protein, 9 aa, synthetic construct
  REGION 1..9: note = Made in Lab - complementarity determining region sequence
AWSGNTEVF

SEQ ID NO: 37 - protein, 12 aa, synthetic construct
  REGION 1..12: note = Made in Lab - complementarity determining region sequence
ASRSGGSAETLY

SEQ ID NO: 38 - protein, 12 aa, synthetic construct
  REGION 1..12: note = Made in Lab - complementarity determining region sequence
ASSFVSSAETLY

SEQ ID NO: 39 - protein, 13 aa, synthetic construct
  REGION 1..13: note = Made in Lab - complementarity determining region sequence
ASSSDRGSAETLY

SEQ ID NO: 40 - protein, 12 aa, synthetic construct
  REGION 1..12: note = Made in Lab - complementarity determining region sequence
ASSDRGGQDTQY

SEQ ID NO: 41 - protein, 11 aa, synthetic construct
  REGION 1..11: note = Made in Lab - complementarity determining region sequence
ASSSGTDTEVF

SEQ ID NO: 42 - protein, 11 aa, synthetic construct
  REGION 1..11: note = Made in Lab - complementarity determining region sequence
AWRDWGGAEQF

SEQ ID NO: 43 - protein, 10 aa, synthetic construct
  REGION 1..10: note = Made in Lab - complementarity determining region sequence
ASSGLGETLY

SEQ ID NO: 44 - protein, 12 aa, synthetic construct
  REGION 1..12: note = Made in Lab - complementarity determining region sequence
ASSLDNSGNTLY

SEQ ID NO: 45 - protein, 12 aa, synthetic construct
  REGION 1..12: note = Made in Lab - complementarity determining region sequence
ASSLDRVQDTQY

SEQ ID NO: 46 - protein, 6 aa, synthetic construct
  REGION 1..6: note = Made in Lab - complementarity determining region sequence
AWTEVF

SEQ ID NO: 47 - protein, 12 aa, synthetic construct
  REGION 1..12: note = Made in Lab - complementarity determining region sequence
ASSFGQNYAEQF

SEQ ID NO: 48 - protein, 12 aa, synthetic construct
  REGION 1..12: note = Made in Lab - complementarity determining region sequence
ASSDGTSAETLY

SEQ ID NO: 49 - protein, 11 aa, synthetic construct
  REGION 1..11: note = Made in Lab - complementarity determining region sequence
ASRPGSAETLY

SEQ ID NO: 50 - protein, 10 aa, synthetic construct
  REGION 1..10: note = Made in Lab - complementarity determining region sequence
ASSPQLYEQY

SEQ ID NO: 51 - protein, 14 aa, synthetic construct
  REGION 1..14: note = Made in Lab - complementarity determining region sequence
ASSDGLGVNQDTQY

SEQ ID NO: 52 - protein, 12 aa, synthetic construct
  REGION 1..12: note = Made in Lab - complementarity determining region sequence
ASSDGGGGTEVF

SEQ ID NO: 53 - protein, 13 aa, synthetic construct
  REGION 1..13: note = Made in Lab - complementarity determining region sequence
AWSLRLGGTYEQY

SEQ ID NO: 54 - protein, 12 aa, synthetic construct
  REGION 1..12: note = Made in Lab - complementarity determining region sequence
ASSLTISNERLF

SEQ ID NO: 55 - protein, 12 aa, synthetic construct
  REGION 1..12: note = Made in Lab - complementarity determining region sequence
ASSFWGRQDTQY

SEQ ID NO: 56 - protein, 12 aa, synthetic construct
  REGION 1..12: note = Made in Lab - complementarity determining region sequence
ASSFWGRGNTLY

SEQ ID NO: 57 - protein, 13 aa, synthetic construct
  REGION 1..13: note = Made in Lab - complementarity determining region sequence
ASGGPGQGFAEQF

SEQ ID NO: 58 - protein, 11 aa, synthetic construct
  REGION 1..11: note = Made in Lab - complementarity determining region sequence
ASSPTGAIMNS

SEQ ID NO: 59 - protein, 14 aa, synthetic construct
  REGION 1..14: note = Made in Lab - complementarity determining region sequence
ASSLYRDRGYAEQF

SEQ ID NO: 60 - protein, 13 aa, synthetic construct
  REGION 1..13: note = Made in Lab - complementarity determining region sequence
AWSLPLGQSYEQY

SEQ ID NO: 61 - protein, 10 aa, synthetic construct
  REGION 1..10: note = Made in Lab - complementarity determining region sequence
ASSFRGYEQY

SEQ ID NO: 62 - protein, 10 aa, synthetic construct
  REGION 1..10: note = Made in Lab - complementarity determining region sequence
ASSDDTYEQY

SEQ ID NO: 63 - protein, 11 aa, synthetic construct
  REGION 1..11: note = Made in Lab - complementarity determining region sequence
ASSDGDRYEQY

SEQ ID NO: 64 - protein, 11 aa, synthetic construct
  REGION 1..11: note = Made in Lab - complementarity determining region sequence
ASSDNYNSPLY

SEQ ID NO: 65 - protein, 13 aa, synthetic construct
  REGION 1..13: note = Made in Lab - complementarity determining region sequence
ASRDWGGRAETLY

SEQ ID NO: 66 - protein, 12 aa, synthetic construct
  REGION 1..12: note = Made in Lab - complementarity determining region sequence
ASSLELGGREQY

SEQ ID NO: 67 - protein, 13 aa, synthetic construct
  REGION 1..13: note = Made in Lab - complementarity determining region sequence
ASSDPGAANTEVF

SEQ ID NO: 68 - protein, 12 aa, synthetic construct
  REGION 1..12: note = Made in Lab - complementarity determining region sequence
ASSLDGADSDYT

SEQ ID NO: 69 - protein, 10 aa, synthetic construct
  REGION 1..10: note = Made in Lab - complementarity determining region sequence
ASSMNNERLF

SEQ ID NO: 70 - protein, 13 aa, synthetic construct
  REGION 1..13: note = Made in Lab - complementarity determining region sequence
ASSQVGGASETLY

SEQ ID NO: 71 - protein, 14 aa, synthetic construct
  REGION 1..14: note = Made in Lab - complementarity determining region sequence
ASGDATDYSGNTLY

SEQ ID NO: 72 - protein, 12 aa, synthetic construct
  REGION 1..12: note = Made in Lab - complementarity determining region sequence
ASGEGPANTEVF

(19) United States
(12) Patent Application Publication
(10) Pub. No.: US 2025/0027930 A1
(43) Pub. Date: Jan. 23, 2025

(54) SMALL MOLECULES FOR REPROGRAMING ANTI-TUMOR IMMUNITY OF T CELLS

(71) Applicant: THE SCRIPPS RESEARCH INSTITUTE, La Jolla, CA (US)

(72) Inventors: Peng Wu, San Diego, CA (US); Yujie Shi, San Diego, CA (US); Yingqin Hou, San Diego, CA (US)

(21) Appl. No.: 18/350,798

(22) Filed: Jul. 12, 2023

Related U.S. Application Data
(63) Continuation-in-part of application No. PCT/US22/12029, filed on Jan. 11, 2022.
(60) Provisional application No. 63/136,585, filed on Jan. 12, 2021.

(51) Int. Cl. G01N 33/50; C12N 5/0783

(52) U.S. Cl. CPC G01N 33/505; C12N 5/0636; G01N 33/5023; C12N 2501/73; C12N 2501/90

(57) ABSTRACT

Provided herein are methods for expanding activated T cells to produce T cells with less differentiated status and enhanced in vivo persistence after adoptive transfer. Activated T cells suitable for the methods include, e.g., progenitor exhausted (Tim3−TCF-1+), stem memory, and central memory T cells. The invention also provides methods for identifying progenitor exhausted T cells specific to an antigen. Further, the invention provides progenitor exhausted T cells, pharmaceutical compositions, and kits as well as methods for the treatment of cancer or infection using the same.

### CROSS-REFERENCE TO RELATED APPLICATIONS

This application is a continuation-in-part of PCT Patent Application No. 2022/012029 (filed Jan. 11, 2022; now pending), which claims the benefit of priority to U.S. Provisional Patent Application No. 63/136,585 (filed Jan. 12, 2021; now expired). The full disclosures of the priority applications are incorporated herein by reference in their entirety and for all purposes.

### STATEMENT OF GOVERNMENT INTEREST

This invention was made with government support under GM139643 and AI143884 awarded by the National Institutes of Health (NIH). The government has certain rights in the invention.

### SEQUENCE LISTING

This application includes by incorporation of reference a sequence listing in the XML format, 2051_1US_Replacement Sequence Listing, which was created on Apr. 27, 2024 and contains 101 KB in content.

### TECHNICAL FIELD

This disclosure relates generally to T cell culture and adoptive T cell transplant.

### BACKGROUND

Novel therapies are constantly developed for the treatment of cancer and infections. One such approach includes adoptive cell therapies (ACT). ACT involve the passive transfer of ex vivo grown cells, most commonly immune cells, into a host with the goal of transferring the immunologic functionality and characteristics of the transplant. However, such therapies are not without limitation. The efficacy of such treatments is hindered by these expanded cells' inadequate ability to persist in vivo, resulting in the absence of sustained clinical response. In several clinical trials, persistent T cells are absent in the majority of patients receiving ex vivo expanded tumor infiltrating lymphocytes (TILs). This observation suggests that the infused cells, which had been extensively expanded from a single reactive cell, may have been driven to terminal differentiation ex vivo and consequently possess very limited replication potential after transfer.

Thus, there remains a need in the art for more effective cells and adoptive cell therapies. In particular, how to increase in vivo persistence and replication potential of tumor antigen-reactive T cells is a major challenge in the field. This invention addresses this and other unmet needs in the art.

### SUMMARY

In one aspect, the invention provides methods for expanding activated T cells to produce T cells with enhanced in vivo persistence after adoptive transfer. The method entail contacting, a cell population that contain activated T cells, under appropriate culturing conditions, with an effective amount of a compound that uncouples T cell expansion from differentiation. In various embodiments, the activated T cells to be expanded in the methods can be, e.g., stem memory T cells (Tscm), central memory T cells (Tcm), effector memory T cells, effector T cells, progenitor exhausted T cells (Tpex), or terminally exhausted T cells (Ttex). In some preferred embodiments, the produced T cells are less differentiated relative to the activated T cells not treated with the compound. In some methods, the employed compound is a sugar or a sugar derivative. In some embodiments, the employed sugar is trehalose, sucrose, lactose, fructose or neuraminic acid. In some embodiments, the employed sugar derivative is N-Acetylglucosamine (GlcNAc) or N-acetylneuraminic acid (Neu5Ac). In some other methods, the employed compound is an inhibitor of Enhancer of zeste homolog 2 (EZH2). In some of these embodiments, the EZH2 inhibitor used for expanding activated T cells is Tazemetostat. In still some other methods, the employed compound is a small molecule inhibitor of KRAS (G12C) (also termed K-Ras(g12c)) mutant. In some of these embodiments, the employed inhibitor compound is KRAS (G12C) inhibitor 9 or 12.

In some embodiments of the invention, the activated T cells to be expanded are tumor infiltrating lymphocytes (TIL) or genetically engineered T cells. In some of these methods, the TILs to be expanded are CD8⁺ T cells or CD4⁺ T cells. In some other methods, the genetically engineered T cell to be expanded are TCR-modified T cells or CAR-T cells.

In a related aspect, the invention provides methods for converting TCF-1 negative T cells into TCF-1 positive or partially positive T cells. These methods involve contacting a population of TCF-1 negative T cells under suitable growth conditions with an effective amount of a compound that uncouples T cell expansion from differentiation. In some embodiments, the employ, the employed compound is N-acetylneuraminic acid (Neu5Ac) or N-Acetylglucosamine (GlcNAc).

In another aspect, the invention provides kits or therapeutic combinations to be used for in vitro expansion of an activated T cell for adoptive cell therapy. The kits or therapeutic combinations contain (1) an effective amount of a compound that uncouples T cell expansion from differentiation, and (2) an instruction of co-culturing the compound with a population of cells comprising the activated T cell. In various embodiments, the kits or therapeutic combinations of the invention can be used for expanding stem memory T cells (Tscm), central memory T cells (Tcm), effector memory T cells, effector T cells, progenitor exhausted T cells (Tpex), and terminally exhausted T cells (Ttex). In some other embodiments, the kits or therapeutic combinations of the invention are intended for expanding tumor infiltrating T cells or genetically engineered T cells. Some of these kits or therapeutic combinations can be used for expanding progenitor-like CD8⁺ T cells or terminally exhausted CD8⁺ T cells. Some other kits or therapeutic combinations can be used for expanding engineered T cells are TCR-modified T cells or CAR-T cells. In various embodiments, the compound in the kits or therapeutic combinations that uncouples T cell expansion from differentiation can be a sugar or a sugar derivative. For example, the compound can be a sugar such as trehalose, sucrose, lactose, glucose, galactose, fructose or neuraminic acid. In some specific embodiments, the compound is sugar derivative N-Acetylglucosamine (GlcNAc) or N-acetylneuraminic acid (Neu5Ac). In some other embodiments, the compound in the kits or therapeutic combinations that uncouples T cell expansion from differentiation can be an inhibitor of Enhancer of zeste homolog 2 (EZH2), e.g., Tazemetostat. In still some other embodiments, the compound in the kits or therapeutic combinations that uncouples T cell expansion from differentiation can be a small molecule inhibitor of KRAS (G12C) mutant, e.g., KRAS (G12C) inhibitor 9 or 12.

In still another aspect, the invention provides methods screening for compounds that promote proliferation of activated T cells and/or maintain expanded T cells in a less differentiated state. These methods entail (a) providing T cells from a T cell-containing tissue from a subject; (b) culturing activated T cells from the T cell-containing tissue in the presence of a compound; maintaining said culturing for a time period sufficient to permit proliferation of T cells, wherein T cells from the T cell-containing tissue have undergone stimulating by an activating agent, wherein stimulating occurs prior to or concurrently with culturing; and (c) measuring: (i) an amount of T cells expressing TCF-1; and/or (ii) an amount of TCF-1 expression, wherein an increase in both (i) and (ii) relative to T cells after expansion not treated with the compound identifies the compound as promoting proliferation of progenitor exhausted T cells and maintaining expanded T cells in a less differentiated state, and wherein an increase in (ii) and not (i) relative to T cells after expansion not treated with the compound identifies the compound as promoting maintaining expanded T cells in a less differentiated state. Some screening methods of the invention can further include re-stimulating and expanding the T cells in the culture following (c), and maintaining said culturing for a time period sufficient to permit expansion of T cells. In these methods, the T cells are assessed by measuring: (i) an amount of T cells expressing TCF-1; and/or (ii) an amount of TCF-1 expression. An increase in both (i) and (ii) relative to T cells after expansion not treated with the compound identifies the compound as promoting proliferation of progenitor exhausted T cells and maintaining expanded T cells in a less differentiated state. An increase in (ii) and not (i) relative to T cells after expansion not treated with the compound identifies the compound as promoting maintaining expanded T cells in a less differentiated state.

In another related aspect, the invention provides methods of screening for compounds that promote proliferation of progenitor exhausted T cells. These methods involve (a) providing splenocytes from a transgenic subject comprising a polynucleotide encoding a labeled Tcf7 gene and a polynucleotide encoding a T cell receptor specific to a peptide antigen in complex with a major histocompatibility complex (MHC) molecule; (b) culturing activated CD8+ T cells from the splenocytes in the presence of a compound; maintaining said culturing for a time period sufficient to permit proliferation of T cells, wherein T cells from the splenocytes have undergone stimulating by an activating agent, wherein stimulating occurs prior to or concurrently with culturing; (c) measuring: (i) an amount of labeled Tcf 7 T cells; and/or (ii) an amount of TCF-1 expression. An increase in (i) and (ii) relative to splenocytes not treated with the compound identifies the compound as promoting proliferation of progenitor exhausted T cells.

Other aspects and advantages of the invention will be readily apparent from the following detailed description of the invention.

### DESCRIPTION OF FIGURES

For the purpose of illustrating the disclosure, there are depicted in the drawings of certain embodiments of the disclosure. However, the disclosure is not limited to the precise arrangements and instrumentalities of the embodiments depicted in the drawings.

FIGS. 1A-1C show screening for small molecule modulators that can increase TCF-1+ cells during in vitro activation and expansion of antigen-specific P14 CD8+ T cells. FIG. 1A depicts compound screening assay for enhanced proliferation of progenitor exhausted T cells (Tim3−TCF-1+ T cells). FIG. 1B depicts flow cytometry analysis of expanded CD8+ T cells (percentage of Tim3−TCF-1+) exposed to various compounds (control, K+, sucrose, lactose, trehalose, galactose, fructose, and T5224) at different concentration on day 6 of culturing. FIG. 1C depicts flow cytometry analysis of expanded CD8+ T cells (percentage of Tim3−TCF-1+) exposed to various compounds (control, K+, sucrose, lactose, trehalose, galactose, fructose, and T5224) at different concentration on day 9 of culturing.

FIGS. 2A-2C show sugar-treated virus-specific CD8+ T cells expanded better in vivo than the untreated T cells. FIG. 2A depicts a schematic for evaluating in vivo expansion of P14 CD8+ T cells after LCMV-C113 infection. FIG. 2B depicts analysis of T cells expanded by various compounds (control and sugars) and flow cytometry analysis of expanded T cells from particular regions (blood, spleen, and liver) at varying time points after transferring cells at day 6. FIG. 2C depicts analysis of T cells expanded by various compounds (control and sugars) and flow cytometry analysis of expanded T cells from particular regions (blood, spleen, and liver) at varying time points after transferring cells at day 9.

FIGS. 3A-3E show sugar-treated antigen-specific OT-1 CD8+ T cells exhibit significantly better capabilities to suppress tumor growth in vivo than the untreated T cells. FIG. 3A depicts a schematic of in vitro activation of OT-1 expanded splenocytes for flow cytometry analysis and in vivo transfer for the treatment of B16-OVA tumor. FIG. 3B depicts cell growth curve of expanded cells under various conditions (sucrose, lactose, glucose, galactose, and fructose) on day 7. FIG. 3C depicts flow cytometry analysis of expanded cells under various conditions (sucrose, lactose, glucose, galactose, and fructose) on day 7. FIG. 3D depicts tumor volume over time post treatment of cells expanded with and without sugars. FIG. 3E depicts apoptosis analysis of naïve OT-splenocytes stimulated by OVA peptide and without IL-2 in the presence and absence of sugars for 5 days.

FIGS. 4A-4G show Neu5Ac and GlcNAc supplements enable enhanced functional characteristics of TCF1+ CD8+ Tpex cells. FIG. 4A depicts a schematic of naïve OT-1 splenocytes stimulated by OVA peptide and without IL-2 under varying conditions (control, GlcNAc, and Neu5Ac) for 5 days and analysis of Annexin V for apoptosis analysis via flow cytometry. FIG. 4B depicts percentage of apoptotic cells under the varying conditions. FIG. 4C depicts self-production of IL2 released by cells in culture medium. FIG. 4D depicts a schematic of naïve OT-1 splenocytes stimulated by OVA peptide and with IL-2 under varying conditions (control, GlcNAc, and Neu5Ac) for 7 or 8 days and analysis of various measurements (ROS MFI, NADPH/NADP+, and GSH/GSSG). FIG. 4E depicts measurement of ROS MFI of the cells under varying conditions. FIG. 4F depicts measurement of NADPH/NADP+ ratio of the cells under varying conditions. FIG. 4G depicts measurement of GHS/GSSG of the cells under varying conditions.

FIG. 5 shows Neu5Ac and GlcNAc supplements enable expansion of TCF1+ CD8+ Tpex cells in vivo and exhibit tumor suppression in vivo. Panel A depicts a schematic of in vitro activation and expansion of OT-1 splenocytes under varying conditions and in vivo transfer for the treatment of B16-OVA tumor treatment supplemented with anti-PD1. Panel B depicts measurement of tumor volume by days post treatment under varying conditions (control, without sugar, without sugar and with anti-PD1, with Neu5Ac, and with Neu5Ac and with anti-PD1). Panel C depicts measurement of percent survival by days post treatment under varying conditions (control, without sugar, without sugar and with anti-PD1, with Neu5Ac, and with Neu5Ac and with anti-PD1). Panel D depicts a schematic for analyzing in vivo tumor infiltrating OT-1 cells in B16-OVA tumor mice from cells activated and expanded under varying conditions (control, GlcNAc, and Neu5Ac) in vitro. Panel E depicts percentage of OT-1 T cells of total CD8+ T cells from isolated tumor. Panel F depicts the number of OT-1 T cells per gram of tumor tissue.

FIGS. 6A-6G show sugars increase TCF-1+ cells during in vitro expansion of CD8+ TSA-reactive CD8+ TILs. FIG. 6A depicts a schematic for FucoID labeling for TSA-reactive TIL isolation. FIG. 6B depicts Tim3−TCF-1+ population percentage from T cells under various conditions (without sugar, with sucrose, with GlcNAc, and with Neu5Ac). FIGS. 6C and 6D depict phenotypic comparison of MC38 TSA-reactive TILs expanded under different conditions (without sugar, with sucrose, with GlcNAc, and with Neu5Ac) by flow cytometry. FIG. 6E depicts a method of using screened cells and various expansion compounds for adoptive transfer T cell therapy for the treatment of tumors. FIG. 6F depicts tumor control efficacy of OVA-specific TILs from B16-OVA tumors expanded with or without sucrose. Tumor size was measured on day 16 after adoptive transfer. FIG. 6G depicts in vivo expansion after LM-OVA challenge of OVA-specific TTLs expanded with or without sucrose.

FIGS. 7A-7E show that sugars increase the proliferation and decrease exhaustion phenotypes of GD2-CarT cells. Human PBMCs were activated with Human T-Expander CD3/cd38 Dynabeads beads at 1:1 ratio for 24 h in T cells medium and then transduced with retroviral vector encoding the disialoganglioside (GD2)-specific14g2a scFv, CD3ζ and CD28 signaling domains (HA-28z). On day 5 after transfection, CD8⁺ CAR T cells or CD4⁺ CAR T cells were sorted for in vitro expansion with 150 IU/mL TL-2 in the presence or absence of sugars (FIG. 7A). Cell numbers of CD8⁺ CAR T cells (FIG. 7B) and CD4⁺ CAR T cells (FIG. 7C) were recorded on day 7 and day 14. Phenotype of CD8⁺ CAR T cells (FIG. 7D) and CD4⁺ CAR T cells (FIG. 7E) were analyzed on day 14 after expansion. 3 repeats for each group (n=3), ^{ns}SP>0.05; *P<0.05; **P<0.01; ***P<0.001; ****P<0.0001; analyzed by one-way ANOVA followed by Tukey's multiple comparisons test.

FIGS. 8A-8C show that K-Ras(G12C) inhibitor 12 facilitates the production of progenitor-like (Tₚₑₓ, Tim-3⁻, TCF-1⁺) antigen-specific CD8⁺ TILs during in vitro rapid expansion. Post-expansion cell growth and phenotypes compared to pre-expansion. (FIG. 8A) Untreated cells on day 8 post-expansion expand to 1.18×10⁶ TILs, while K-Ras(G12C) inhibitor 12 (5 μM)-treated cells expand to 0.397×10⁶ TILs (pre-expansion cell number is 0.1×10⁶ TILs). (FIG. 8B) M8 tetramer* CD8⁺ TILs were analyzed for the expression of inhibitory receptors Tim-3 and PD-1. (FIG. 8C) the expression of Tim-3 and TCF-1 via fluorochrome-labelled antibody staining and flow cytometry. For all graphs and figures, pre-expansion n=1; untreated control n=1; K-Ras(G12C) inhibitor 12 (5 μM) n=1.

FIGS. 9A-9E show that K-Ras(G12C) inhibitor 12 (2 μM) upregulates progenitor-like and less-differentiated (T_{SCM}) phenotypes in CD8+ T cells from PBMCs of healthy human donors. (FIG. 9A) Donor hPBMCs were activated twice with irradiated (50 Gy) feeder cell mixture, initially on day 0 and then similarly on day 6. (FIG. 9B) hPBMCs treated with K-Ras(G12C) inhibitor 12 (2 μM) from both donors experience no statistically significant compromise in cell growth. hPBMCs from both donors were stained with fluorochrome-labelled antibodies and immunophenotyped utilizing flow cytometry. (FIG. 9C) Gated CD8⁺ hPBMCs were analyzed for the expression of inhibitory receptors Tim-3 and PD-1 (terminally exhausted phenotype downregulation not statistically significant in either donor). (FIG. 9D) the expression of Tim-3 and TCF-1. (FIG. 9E) the expression of CCR7 and CD45RA. All bar graphs represent mean±SD; untreated control n=3, K-Ras(G12C) inhibitor 12 (2 μM) n=3; ns P>0.05, * P<0.05, ** P<0.01, *** P<0.001, **** P<0.0001; either two-way ANOVA followed by Sidak multiple comparisons test (B, E) or unpaired t-test (C, D).

FIGS. 10A-10D show transient inhibition of EZH2 by tazemetostat maintain progenitor phenotypes of OT-I cells. (FIGS. 10A and 10B) Phenotype analysis of OT-I/TCF-7-GFP splenocytes were activated by OVA₂₅₇₋₂₆₄ (500 nM) with 60 IU/mL IL-2 for 3 days and expanded in presence or absence of Taz (400 nM) for 4 days (day3-7). Cell proliferation of OT-I cells expanded w/ or w/o Taz (400 nM, Day3-7). (FIG. 10C) Western blot of histone proteins from OT-I cells activated by OVA₂₅₇₋₂₆₄ (500 nM) with 60 IU/mL IL-2 for 3 days and expanded in presence or absence of Taz (400 nM) for 12, 24, 48 h. (FIG. 10D) Histone 3 (H3) was used as a reference protein. 3 repeats for each group (n=3), ^{ns}SP>0.05; *P<0.05; **P<0.01; ***P<0.001; ****P<0.0001; analyzed by student T-test.

FIGS. 11A-11D show EZH2 inhibition by tazemetostat in OT-I cells during in vitro expansion improves in vivo homeostasis and response of OT-I cells for LM-OVA infection.

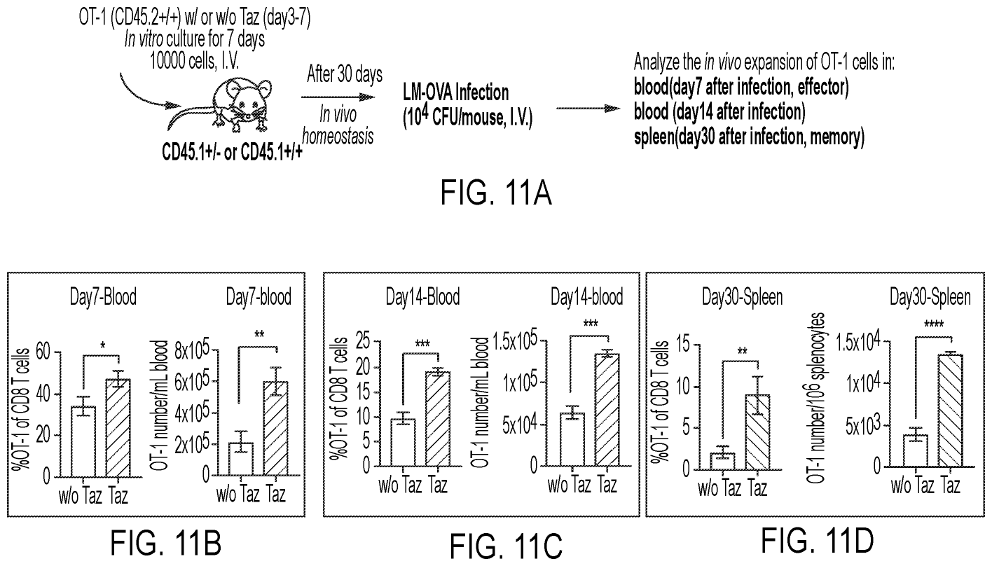

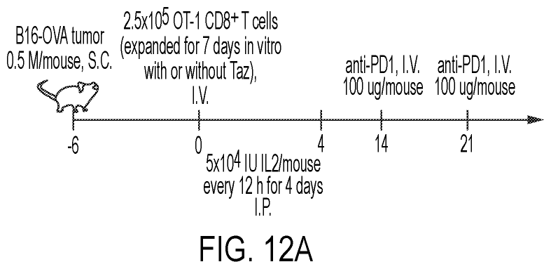

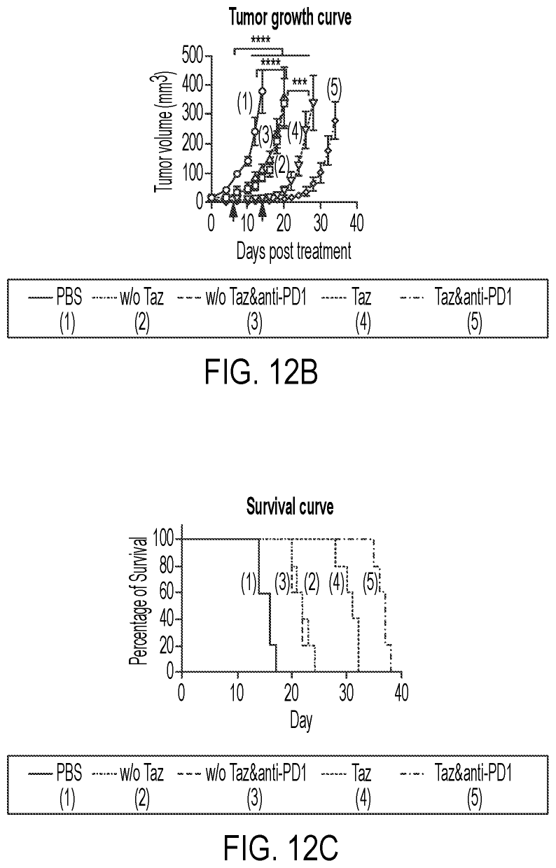

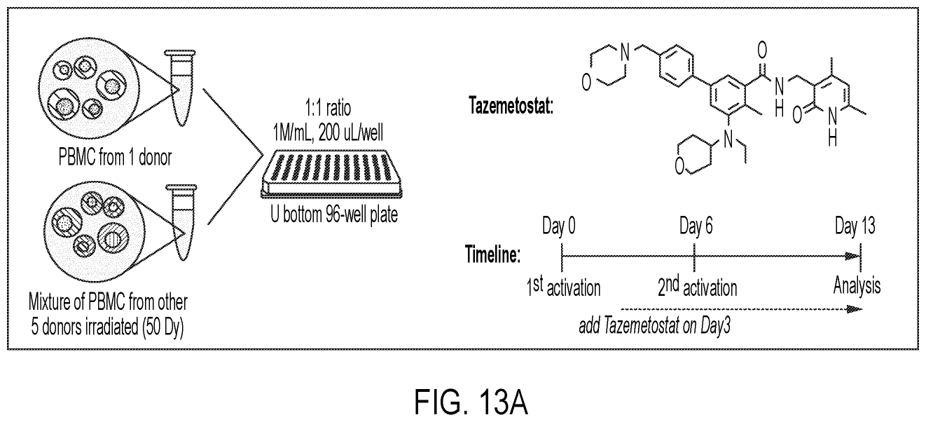

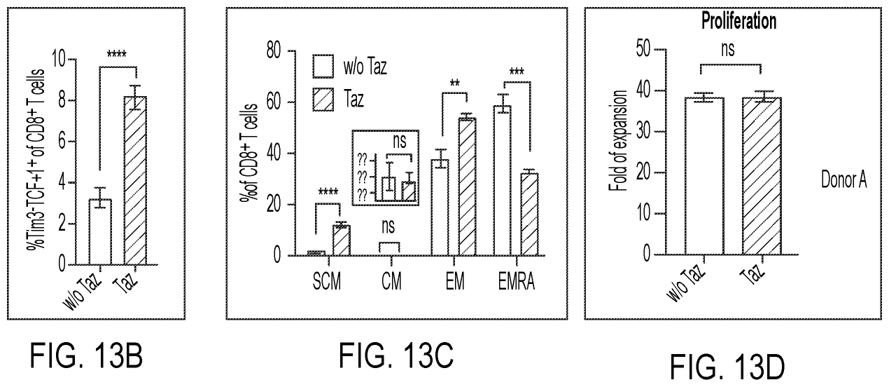

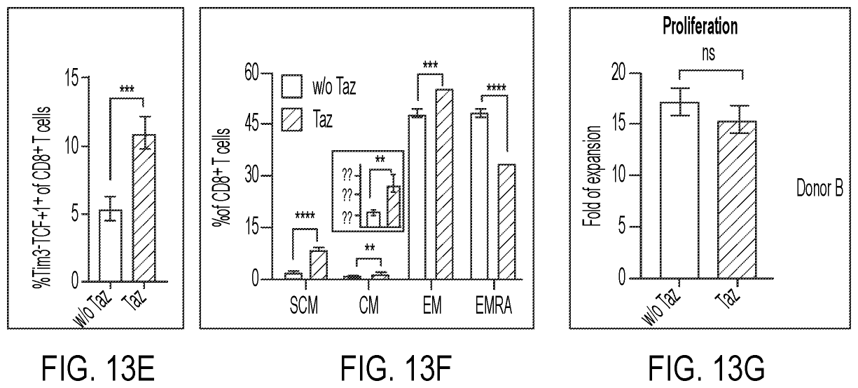

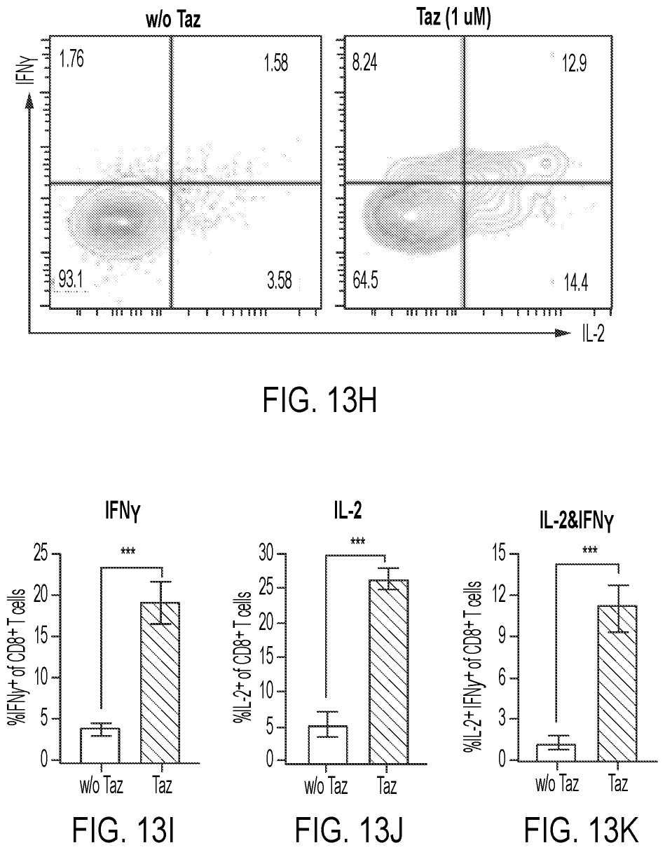

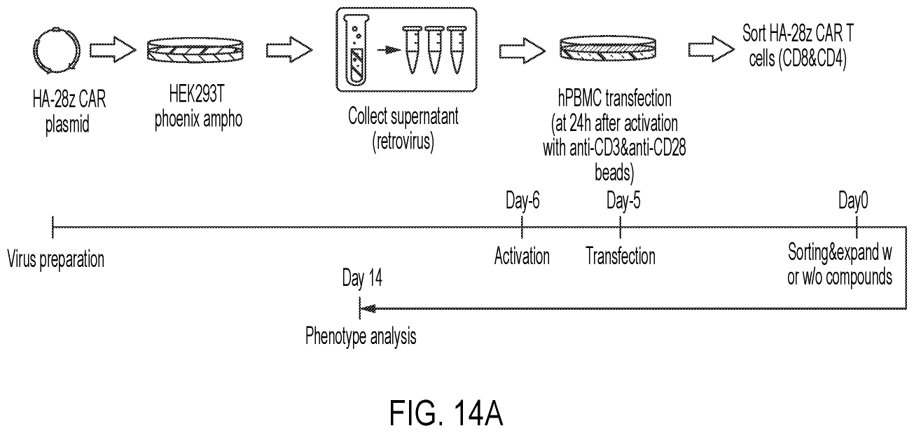

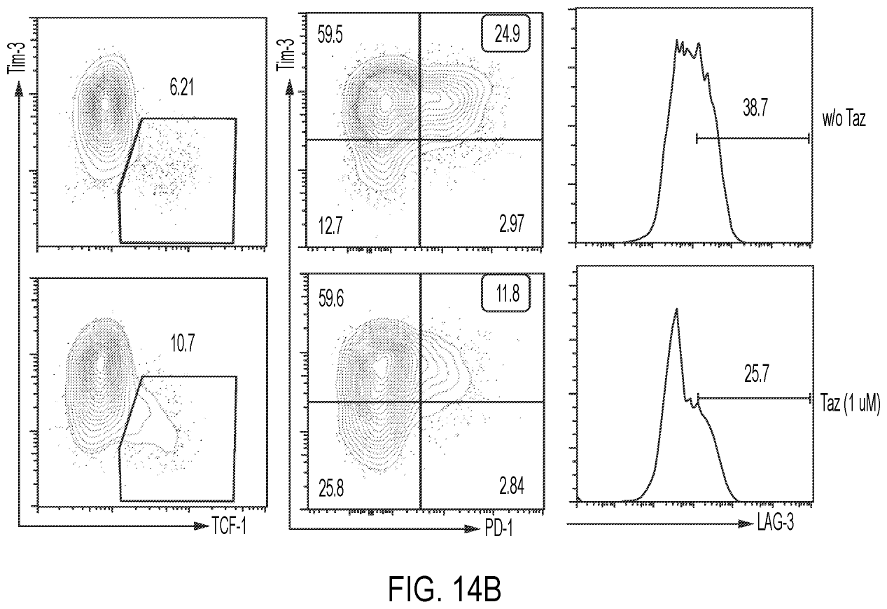

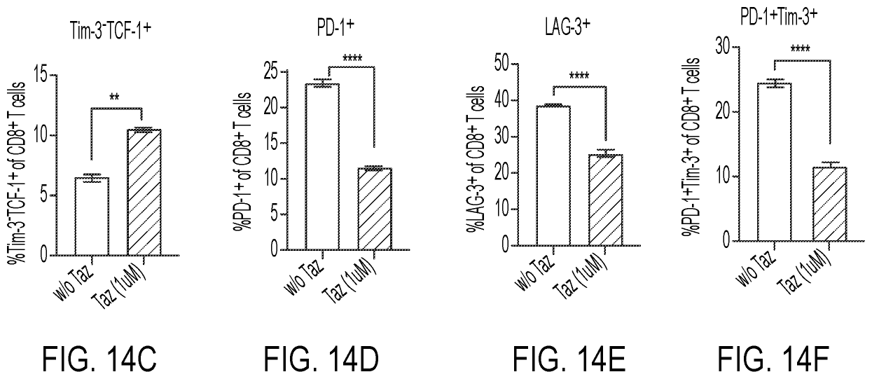

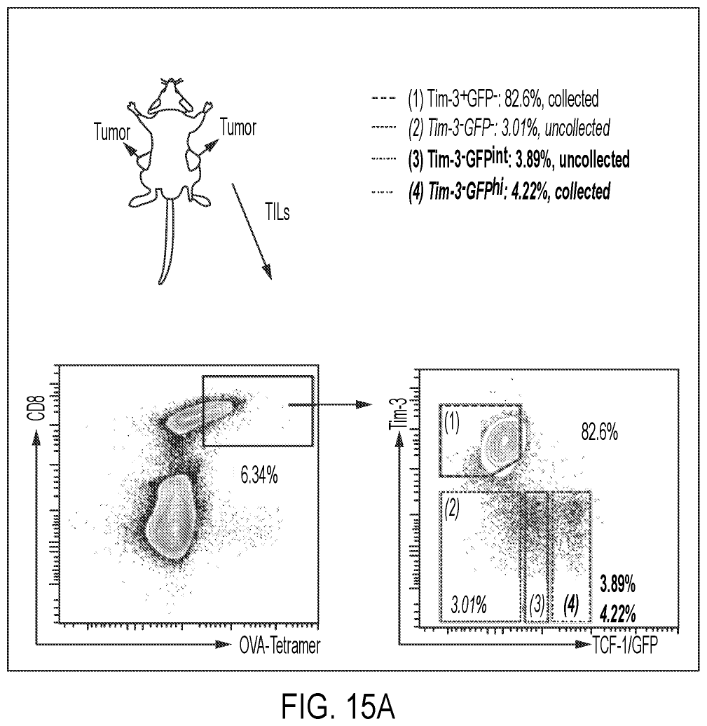

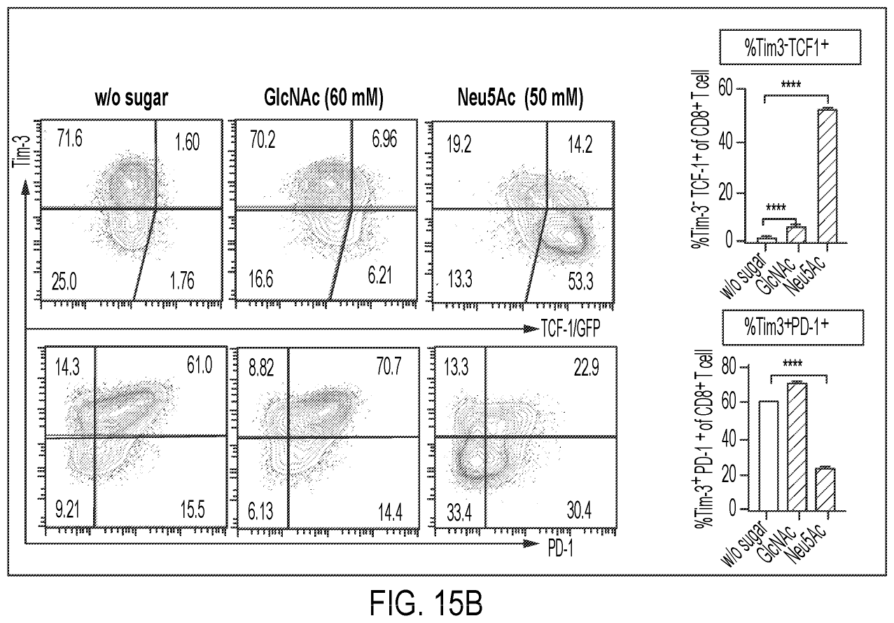

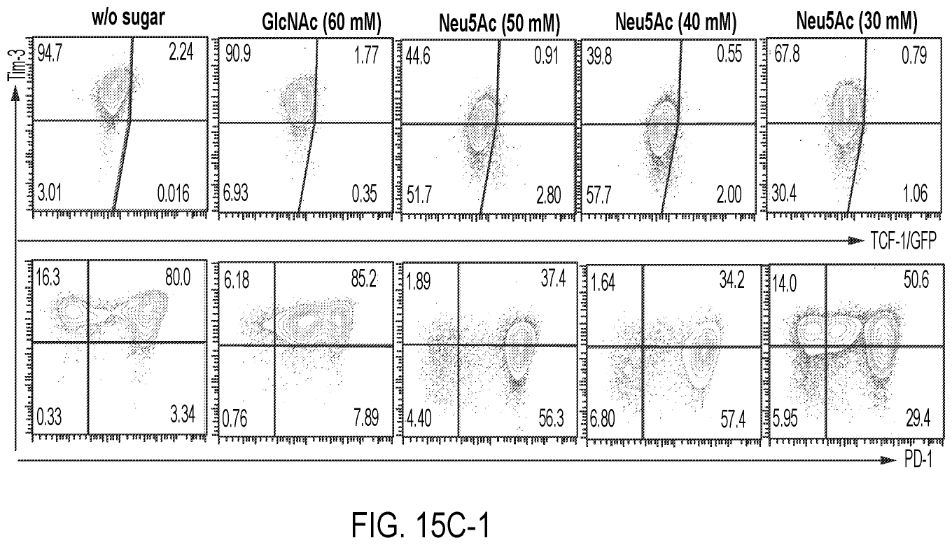

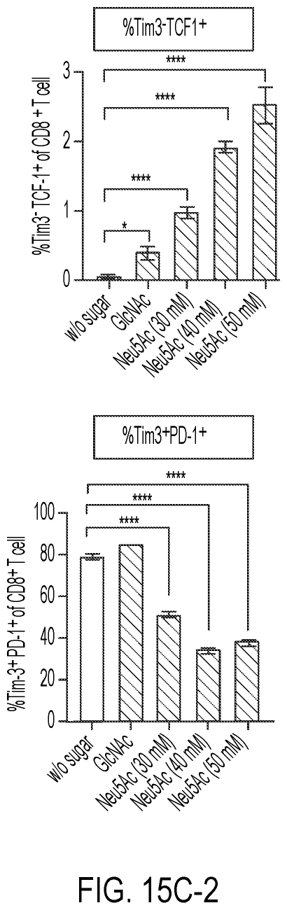

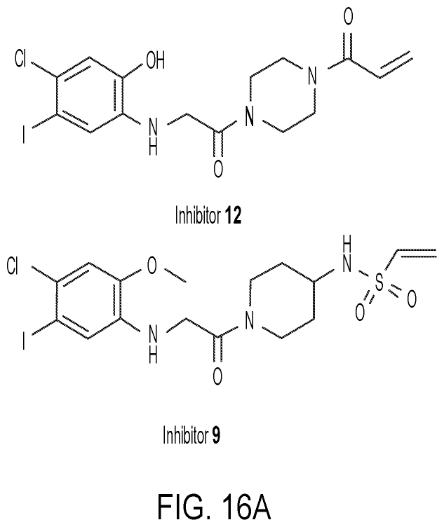

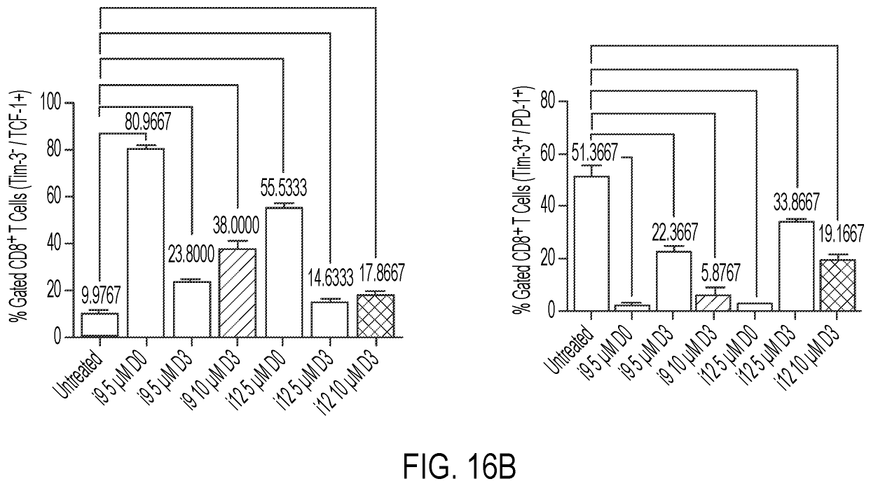

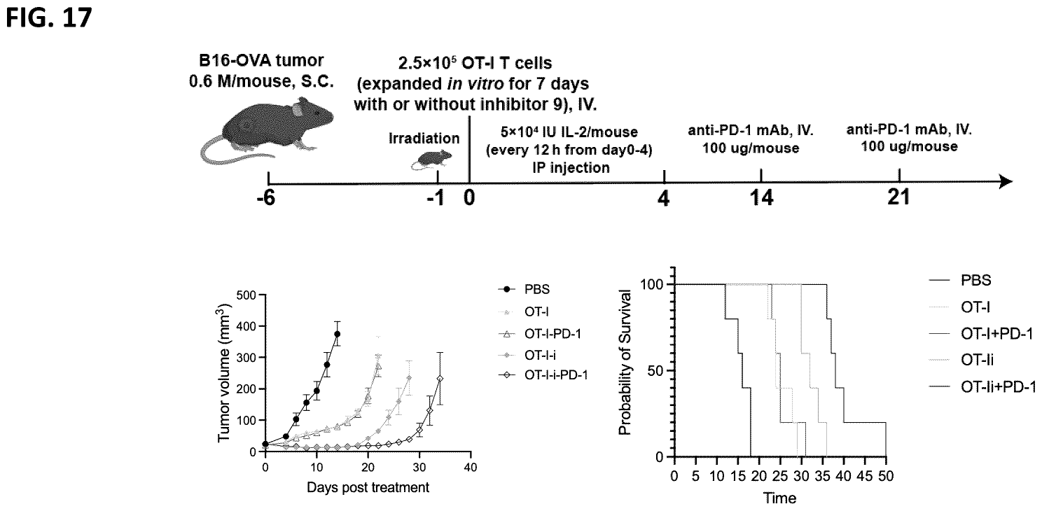

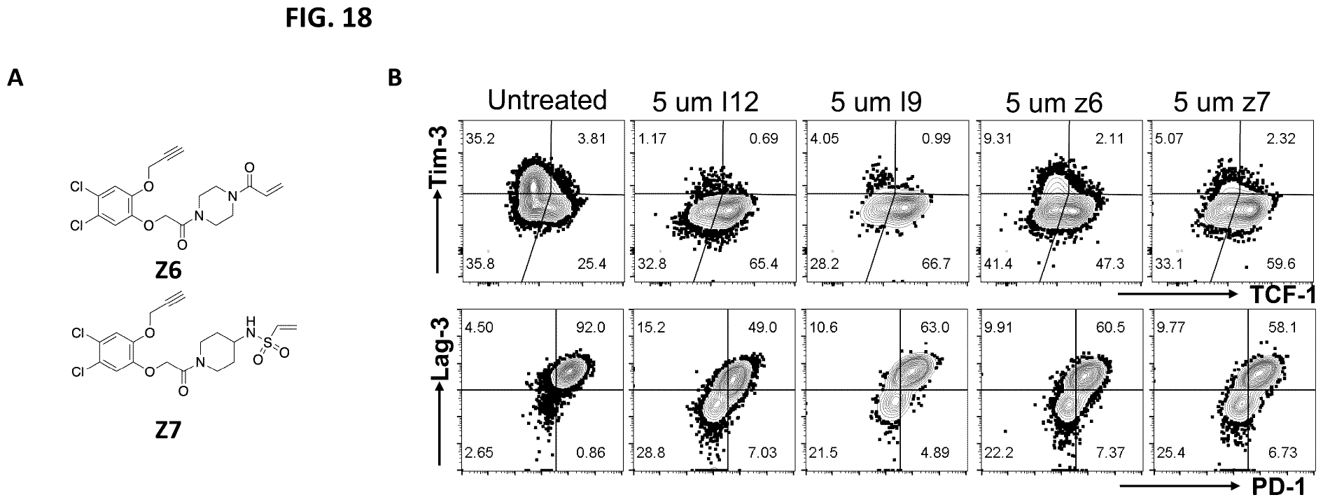

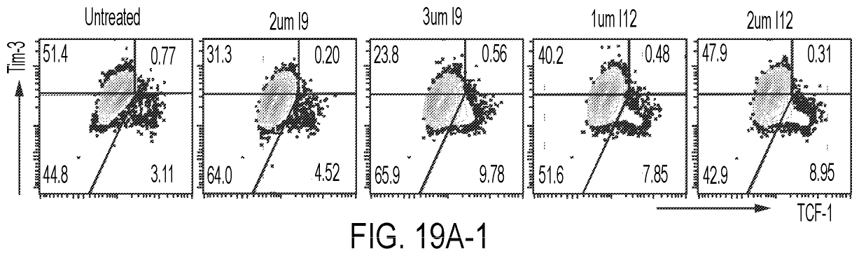

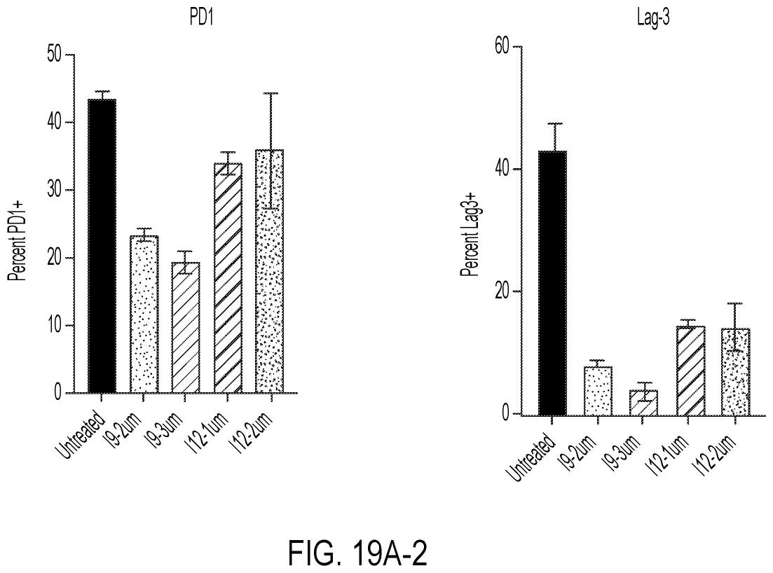

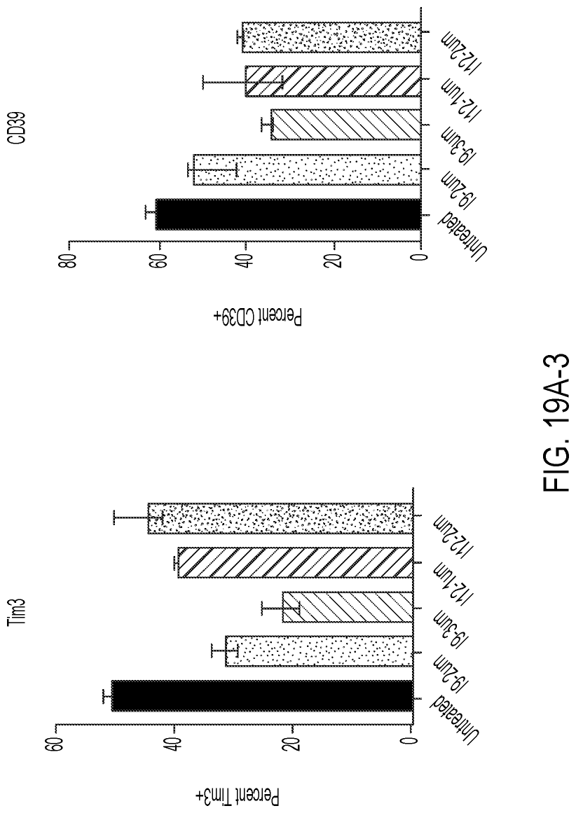

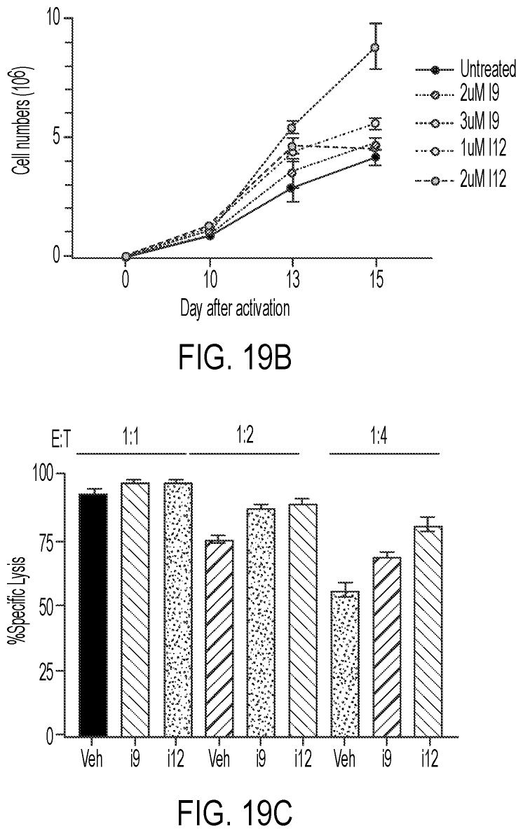

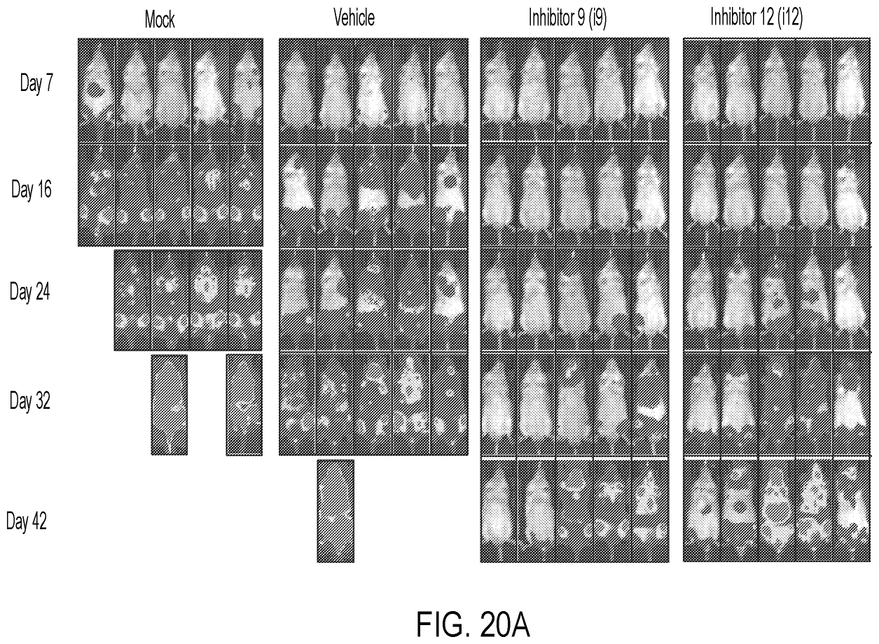

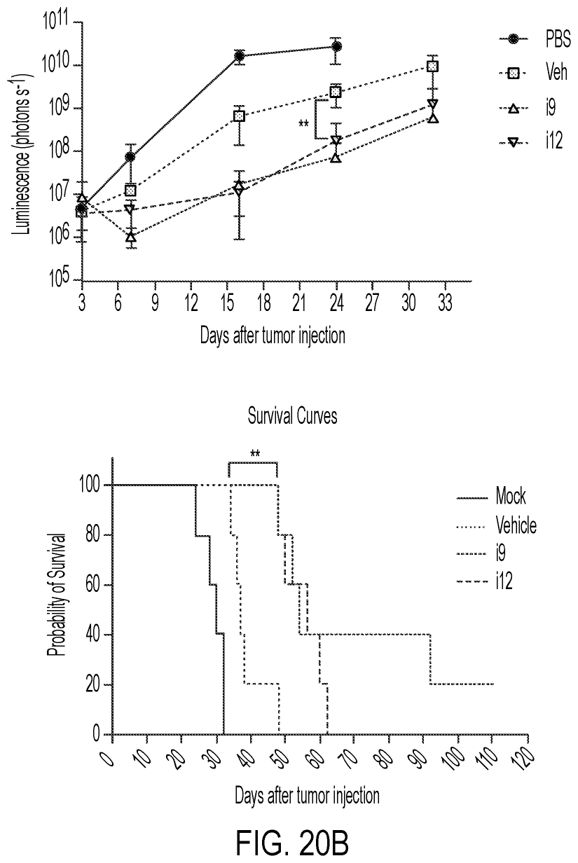

OT-I splenocytes (CD45.2+/+) were activated with 500 nM OVA₂₅₇₋₂₆₄ for 3 days and expanded in presence of absence of Taz (400 nM) for 4 days (day3-day7). 30 days after 10⁴ OT-I cells (CD8⁺) were intravenously transferred into C57BL/6 mice (CD45.1+/+ or CD45.1+/−), mice were infected with 10⁴ CFU LM-OVA (FIG. 11A). Ratio of OT-I cells in CD8+ T cells or exact number of OT-I cells in blood were analyzed on day 7 (FIG. 11B) and day 14 (FIG. 11C), in spleen were analyzed on day 30 (FIG. 11D). 3 repeats for each group (n=3), ^{ns}SP>0.05; *P<0.05; **P<0.01; ***P<0.001; ****P<0.0001; analyzed by student T-test.

FIGS. 12A-12C show that EZH2 inhibition by tazemetostat in OT-I cells during in vitro expansion improves in vivo efficacy and immune checkpoint response of OT-I cells against B16-OVA tumors. C57BL/6 mice were subcutaneously inoculated with B16-ova tumor cells (6×10⁵). After 6 days (Day 0), CD8⁺ OT-I T cells (2×10⁵), in vitro expanded with and without Taz (400 nM) for 4 days (day3-day7), respectively, were transferred, followed by IL-2 administration and anti-PD-1 treatment on day 14 and day21 (FIG. 12A). In control groups, mice were administered with PBS buffer only. Tumor sizes (FIG. 12B, anti-PD-1 injections were indicated by black arrows) and survival (FIG. 12C) were recorded every 2/3 days. 5 mice were included in each group (n=5), ^{ns}P>0.05; *P<0.05; **P<0.01; ***P<0.001; ****P<0.0001; one-way ANOVA followed by Tukey's multiple comparisons test.

FIGS. 13A-13K show that tazemetostat enhances desired properties in human PBMCs. Human PBMCs were activated with an irradiated (50 Gy)mixture hPBMCs from another five different donors on day0 and day6, and expanded with 300 IU/mL IL-2 in presence or absence of Taz (1 uM, day3-day14) (FIG. 13A). Phenotype (FIGS. 13B and 13C for donor A, FIGS. 13E and 13F for donor B) and proliferation (FIG. 13D for donor A, FIG. 13G for donor B) analysis of CD8⁺ T cells on day 14 for 2 donors. SCM: CD45RA⁺CCR7⁺, CM: CD45RA⁻ CCR7⁺, EMRA: CD45RA⁺CCR7⁻, EM: CD45RA-CCR7−. Cytokine production of hPBMCs were cultured w/ or w/o Taz (1 μM, day3-day14) and activated with coated anti-CD3 antibody (1 ug/mL) and suspended anti-CD28 antibody (1 ug/mL) for 5 h (FIGS. 13H-13K). 3 repeats for each group (n=3), ^{ns}SP>0.05; *P<0.05; **P<0.01; ***P<0.001; ****P<0.0001; analyzed by student T-test.

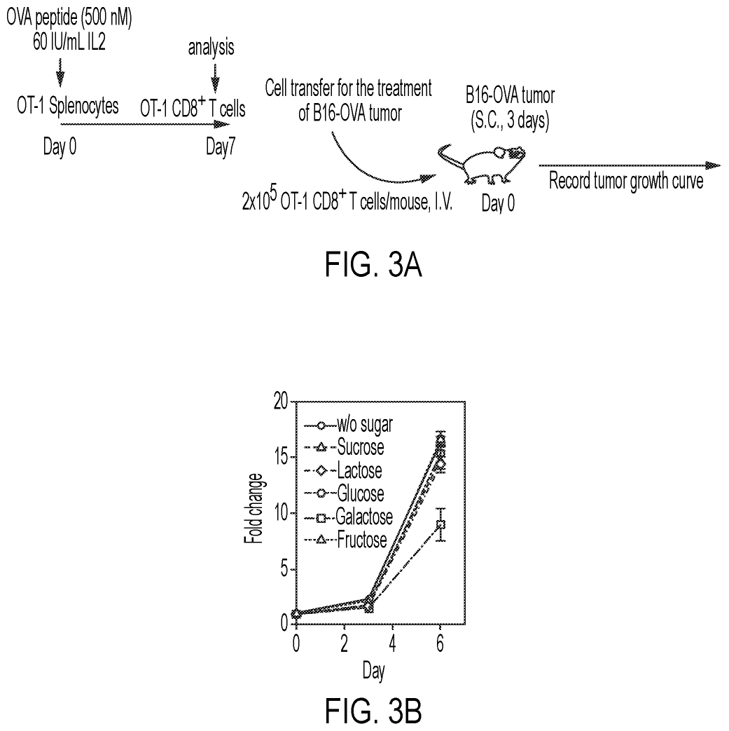

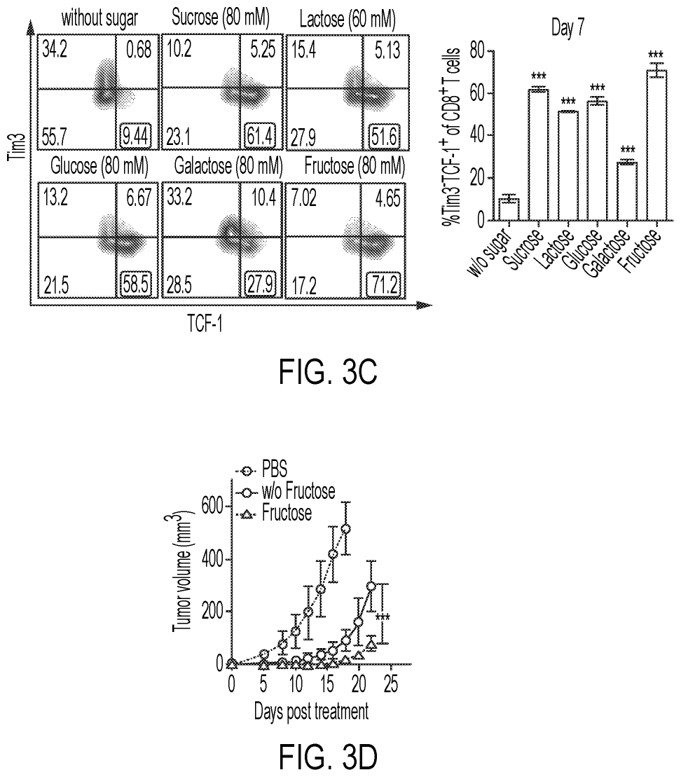

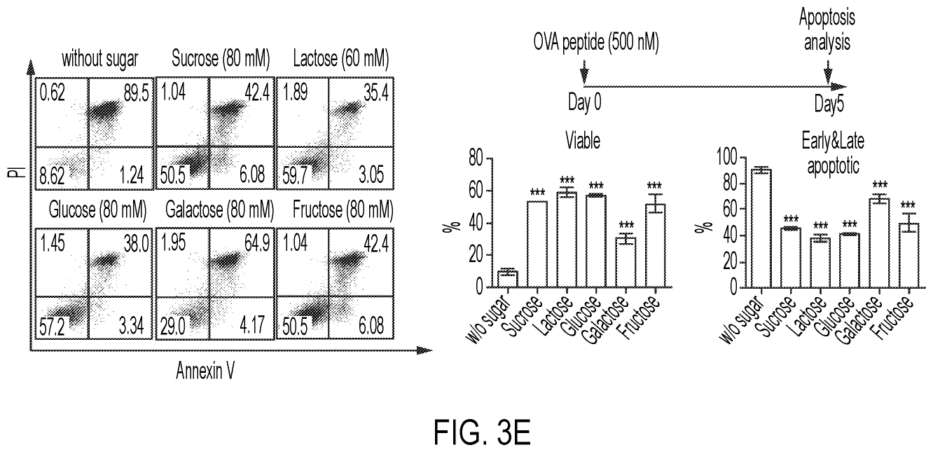

FIGS. 14A-14F show that tazemetostat decreases exhaustion phenotypes of GD2-CarT cells. Human PBMCs were transinfected with ha-28z and CD8+GD2+ T cells were sorted for in vitro expansion with 150 IU/mL IL-2 in the presence or absence of Taz (1 μM) (FIG. 14A), phenotype analysis on day 14 after expansion (FIGS. 14B-14F), 3 repeats for each group (n=3), ^{ns}SP>0.05; *P<0.05; **P<0.01; ***P<0.001; ****P<0.0001; analyzed by student T-test.

FIGS. 15A-15C show that sugars maintain progenitor exhaustion features and reverse terminal exhausted T cells to progenitor stage of TILs from B16-OVA tumor. TILs from B16-OVA tumor were isolated on day10 after inoculation, and Tim-3⁻TCF/GFP⁺ and Tim-3⁺TCF/GFP⁻ in tetramer positive population were collected by live cell sorting (FIG. 15A). Phenotype features of Tim-3⁻TCF/GFP⁺ (FIG. 15B) and Tim-3⁺TCF/GFP⁻ (FIG. 15C) were analyzed on day 7 after rapid expansion with feeder cells (TILs/feeder cell: 1/200) in the presence or absence of sugars. 3 repeats for each group (n=3), ^{ns}P>0.05; *P<0.05; **P<0.01; ***P<0.001; ****P<0.0001; analyzed by one-way ANOVA followed by Tukey's multiple comparisons test.

FIGS. 16A and 16B show that K-Ras(G12C) Inhibitors 9 and 12 maintained OT-I cell stemness.

FIG. 17 shows that OT-I cells treated with K-Ras(G12C) Inhibitor 9 provided improved tumor control and enhanced response to PD-1 blockade.

FIG. 18 shows that alkyne analogues of K-Ras(G12C) Inhibitors 9 and 12, Z7 and Z6, maintained OT-I cell stemness. (A). Structures of Z7 and Z6, which are derived from inhibitor 9 and inhibitor 12; (B) Activities of the analogues on T cells.

FIGS. 19A-19C show activities of K-Ras(G12C) Inhibitors 9 and 12 on CAR-T cells. (FIG. 19A) Inhibitor 9/12 treatment increased progenitor features and decreased the expression of inhibitory receptors (PD-1, LAG3, Tim-3 and CD39) of anti-GD2 CAR-T cells. (FIG. 19B) Inhibitor 9 also facilitated the proliferation of CAR-T cells. (FIG. 19C) Compared to vehicle treated CAR-T cells, anti-GD2 CAR-T cells treated with i9 (3 uM) or i12 (1 uM) exhibited higher anti-tumor activity against the target cell.

FIGS. 20A and 20B show that, compared to vehicle treated CAR-T cells, administration of anti-GD2 CAR-T cells treated with i9 (3 uM) or i12 (1 uM) resulted in better in vivo tumor control and improved survival time of tumor bearing mice. Statistical analyses were performed using GraphPad Prism software (version 9.0). Comparisons over groups were analyzed using one-way ANOVA followed by Tukey's multiple comparisons test. Survival data were analyzed by Log-rank (Mantel-Cox) test. n=3-5, ns, p>0.05; *p<0.05; **p<0.01; ***p<0.001; ****p<0.0001.

### DETAILED DESCRIPTION

### Overview

T cell-based immunotherapies may be used to treat patients experiencing malignancies or infection. For the treatment of malignancies, tumor-specific antigen (TSA)-reactive T cells may be isolated from a patient's tumor and then cultured in vitro to generate large numbers of cells for adoptive transfer. Alternatively, T cells from a patient's peripheral blood mononuclear cells (PBMCs) may be engineered to express a TSA-reactive T-cell receptor (TCR) or chimeric antigen receptor (CAR), and expanded in vitro to generate large numbers of cells for adoptive transfer. Additionally, current methods of identifying antigen candidates using reverse immunology and predictive analysis are limited by available computational tools that cannot accurately predict T-cell reactivity. The efficacy of these T cell-based immunotherapies can be limited, in part, by the capacity of the cells to persist in vivo after transfer, wherein absences of sustained clinical responses are observed. Differentiation of these cells is coupled to (e.g., connected to) expansion. Thus, replication potential, and in vivo persistence after adoptive transfer, of T cells negatively correlates with terminal differentiation.

Naïve T cells, upon antigen encounter, undergo activation and differentiation. Cellular differentiation is accompanied by loss of ‘stemness’—the capability of cells to be multipotent and self-renewing. Along the pathway of T cell differentiation (naïve T cell→stem cell memory T cell (Tscm)→central memory (Tcm)→effector memory T cells (Tem)→effector T cells (Teff), there is a graduate decrease of the expression of the transcription factor TCF-1. A unique feature of these TCF-1⁺ T cells is their self-renewal potential. Adoptive transfer experiments demonstrated that only TCF-1⁺ T cells, but not their TCF-1-counterparts, have the capacity to both self-renew and give rise to a progeny of TCF-1⁻ cells endowed with effector potentials and high levels of checkpoint receptor expression. In addition, less-differentiated T cell subsets such as stem cell memory (Tscm) and central memory (Tcm) have lower levels of reactive oxygen species (ROS) whereas terminally differentiated effector memory and effector T cells (Tem and Teff) have higher ROS levels that are required for their effector function such as cytotoxicity. Increased oxidative stress and DNA damage as a result of ROS accumulation may directly drive CD8+ T cells towards terminal differentiation with characteristics of loss of T cell proliferation, T cell effector function, and impaired self-renewal (https://pubmed.ncbi.nlm.nih.gov/28412583/)

Studies have revealed that the adoptive transfer of TCR-T and CAR-T cells with fully-differentiated Teff phenotype are less effective in controlling tumor growth than utilizing less-differentiated Tscm or Tcm subsets. Likewise, progenitor exhausted T cells (Tpex), a sub-population of dysfunctional CD8+ T cells expressing the transcription factor TCF-1 that were recently identified in mouse persistent Lymphocytic choriomeningitis virus (LCMV) clone 13 (C113), patients infected with hepatitis C virus (HCV) and HIV, cancer models and cancer patients, were found to be required to sustain antiviral T cell responses to chronic infections and antitumor responses in the tumor microenvironment. These Tpex cells (Tim3−TCF-1+) provide the proliferative burst and effector function by forming more differentiated terminally exhausted T cells (Ttex, Tim3+TCF-1−) following anti-PD-1/PD-L1 therapy. Furthermore, meta-analysis of The Cancer Genome Atlas (TCGA) database for correlations with patient survival in a primary melanomas cohort revealed that a Tcf7 (encoding TCF-1)/pdcd1 (encoding PD-1) signature in tumor infiltrating lymphocytes (TILs) correlates with improved patient survival.

Given that positive response to anti-PD-1 therapy in humans correlates with the accumulation of Tpex in tumors coupled to the fact that only a finite number of PD-1-responsive CD8+ T cells exist in patients, producing the Tpex rather than the Ttex phenotype during in vitro expansion of the isolated TSA-reactive TILs is hypothesized to be important for improving the efficacy of immunotherapy in both chronic infections and cancer. Likewise, producing TCR-T and CAR-T cells with less-differentiated Tscm or Tcm phenotypes and high TCF-1 expression during in vitro expansion will likely improve the efficacy of immunotherapy treatments.

The present invention is derived in part from studies undertaken by the inventors to identify small molecules and other compounds that are capable of uncoupling expansion or proliferation of activated T cells (e.g., progenitor exhausted T cells (Tpex)) from their differentiation. As detailed herein, several compounds identified from the studies are capable of expanding activated T cells to produce T cells with less differentiated status and high TCF-1 expression. These T cells with less differentiated status possess the capability of enhanced in vivo persistence after adoptive transfer. In some of the studies, the inventors' discovered that contacting various sugars, outside physiological levels, with T cells improves expansion while maintaining stemness. Moreover, the cells expanded with said sugars exhibit enhanced expansion and proliferation in vivo (also referred to as enhanced in vivo persistence) and enhanced tumor suppression capabilities compared to untreated control T cells.

Specifically, as detailed below, results from these studies revealed that treatment of a virus-specific TCR-modified CD8+ T cell (P14) with sugar or sugar derivative compounds expanded better in vivo than the untreated T cells. It was also observed that an antigen-specific TCR-modified CD8+ T cell (OT-1) treated with the sugar compounds during in vitro expansion exhibit significantly better capabilities to suppress tumor growth in vivo than the untreated T cells. In particular, it was shown that treatment of TCF1+ CD8+ Tpex cells with two sugar derivative compounds, Neu5Ac and GlcNAc, resulted in enhanced functional characteristics of the T cells. These sugar derivatives promote expansion of TCF1+ CD8+ Tpex cells in vivo, exhibit tumor suppression activities in vivo, and increase TCF-1+ cells during in vitro expansion of CD8+ TSA-reactive CD8+ TILs. Further, these identified sugar compounds were further found to be able to increase the proliferation and decrease exhaustion phenotypes of human PBMC-derived CAT-T cells. In a related study, it was found that the sugar derivative compounds can maintain progenitor exhausted features and reverse terminal exhausted TILs isolated from tumors back to progenitor exhausted T cells.

In addition to sugars or sugar derivatives, the inventors' studies also identified other compounds capable of uncoupling T cell expansion from differentiation. For example, they discovered that a well-known known inhibitor compound of K-Ras(G12C) mutant, Inhibitor 12, facilitates the generation of progenitor-like CD8⁺ T cells without compromising cell growth during in vitro expansion. It was also observed that this compound upregulates progenitor-like and less-differentiated (T_{SCM}) phenotypes in CD8⁺ T cells from PBMCs of healthy human donors. Similarly, another compound, EZH2 inhibitor tazemetostat, was found to be able to maintain the progenitor phenotypes of a TCR-modified T cell (OT-1). It was also shown that EZH2 inhibition by tazemetostat in T cells during in vitro expansion improves in vivo homeostasis, in vivo efficacy, and immune checkpoint response of the adoptively transferred T cells. Tazemetostat was further found to enhances desired properties in human PBMCs, and decrease exhaustion phenotypes of human PBMC-derived CAR-T cells. Additional studies revealed that purine and pyrimidine biosynthesis intermediates and end products, as well as an inhibitor compound of glucose-6-phosphate dehydrogenase (G6PDi-1), can also maintain the less-differentiated phenotype of progenitor exhausted T cells.

In accordance with these studies, the invention provides methods for expanding activated T cells to produce T cells with less differentiated status. In related embodiments, the invention provides methods for maintaining or keeping the less-differentiated status of activated T cells, e.g., maintaining the phenotype of progenitor exhausted T cells. In general, the methods of the invention utilize small molecule compounds described herein that demonstrated the ability to uncouple T cell expansion from differentiation. Such compounds include, e.g., specific sugars or sugar derivatives, KRAS (G12C) inhibitors, EZH2 inhibitor, and purine or pyrimidine biosynthesis related compounds. Due to their enhanced in vivo persistence, these treated T cells are therefore better suitable for adoptive T cell transfer therapies.

In another aspect, the disclosure provides methods for screening for compounds that uncouple expansion from differentiation during the in vitro expansion of T cells to enhance the persistence of T cells in vivo after transfer during adoptive cell transfer therapies. In a related aspect, the disclosure provides screening methods useful, in part, to identify modulators (e.g., small molecules) that can be employed during in vitro expansion to improve the quality of TILs and TCR/CAR-engineered T cells by boosting the numbers of anti-PD-1 responsive CD8+ T cells, in which said cells exhibit less differentiated phenotypes. In another related aspect, the disclosure provides methods for screening for modulators (e.g., compounds) that promote proliferation of T cells, wherein the proliferation of the T cells promoted by the compound uncouples T cell expansion from differentiation. Additionally, the disclosure provides methods for identifying, isolating, and expanding non-terminally differentiated T cells specific to an antigen. The disclosure further provides T cells, pharmaceutical compositions, kits and methods of treatment using the same.

### Definitions

To facilitate understanding of the invention, a number of terms and abbreviations as used herein are defined below as follows:

As used herein the singular forms “a”, “an” and “the” include plural aspects unless the context clearly dictates otherwise. For example, reference to “a vector” includes a single vector, as well as two or more vectors; reference to “a cell” includes one cell, as well as two or more cells; and so forth.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

By “consisting of” is meant including, and limited to, whatever follows the phrase “consisting of”. Thus, the phrase “consisting of” indicates that the listed elements are required or mandatory and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference to the term “e.g.” is intended to mean “e.g., but not limited to” and thus it should be understood that whatever follows is merely an example of a particular embodiment, but should in no way be construed as being a limiting example. Unless otherwise indicated, use of “e.g.” is intended to explicitly indicate that other embodiments have been contemplated and are encompassed by the present invention.

Reference throughout this specification to “embodiment” or “one embodiment” or “an embodiment” or “some embodiments” or “certain embodiments” means that a particular feature, structure or characteristic described in connection with the embodiment is included in at least one embodiment of the present invention. Thus, the appearances of the phrases “in one embodiment” or “in an embodiment” or “in certain embodiments” or “in some embodiments” in various places throughout this specification are not necessarily all referring to the same embodiment. Furthermore, the particular features, structures, or characteristics may be combined in any suitable manner in one or more embodiments.

An “increased” or “enhanced” amount is typically a “statistically significant” amount, and may include an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. Similarly, a “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

As used herein, “optional” or “optionally” means that the subsequently described event, or circumstances, may or may not occur, and that the description includes instances where said event or circumstance occurs and instances in which it does not.

As used herein, “substantially” or “essentially” means of ample or considerable amount, quantity, size; nearly totally or completely; for instance, 95% or greater of some given quantity.

The terminology used herein is for the purpose of describing particular embodiments only and is not intended to be limiting. Unless defined otherwise, all technical and scientific terms used herein have the same meanings as commonly understood by one of ordinary skill in the art to which the present disclosure belongs. Any materials and methods similar or equivalent to those described herein can be used to practice the present invention. The practice of the present invention may employ conventional techniques of molecular biology (including recombinant techniques), microbiology, cell biology, biochemistry and immunology, which are within the skill of the art. Such techniques are explained fully in the literature, such as Molecular Cloning: A Laboratory Manual, second edition (Sambrook et al, 1989) Cold Spring Harbor Press; Oligonucleotide Synthesis (MJ. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and CC. Blackwell, eds.); Gene Transfer Vectors for Mammalian Cells (J. M. Miller and M. P. Calos, eds., 1987); Current Protocols in Molecular Biology (F. M. Ausubel et al, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (CA. Janeway and P. Travers, 1997); Antibodies (P. Finch, 1997); Antibodies: a practical approach (D. Catty., ed., TRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al, eds., J. B. Lippincott Company, 1993).

The terms “in vitro”, “ex vivo”, and “in vivo” are intended herein to have their normal scientific meanings. Accordingly, e.g., “in vitro” is meant to refer to experiments or reactions that occur with isolated cellular components, such as, e.g., an enzymatic reaction performed in a test tube using an appropriate substrate, enzyme, donor, and optionally buffers/cofactors. “Ex vivo” is meant to refer to experiments or reactions carried out using functional organs or cells that have been removed from or propagated independently of an organism. “in vivo” is meant to refer to experiments or reactions that occur within a living organism in its normal intact state.

As used herein, “mammal” includes humans and both domestic animals such as laboratory animals and household pets, (e.g., cats, dogs, swine, cattle, sheep, goats, horses, primates, rodents, and rabbits), and non-domestic animals such as wildlife and the like.

As used herein, “subject,” includes any animal that exhibits a disease or symptom, or is at risk for exhibiting a disease or symptom, which can be treated with an agent of the invention. Suitable subjects include laboratory animals (such as mouse, rat, rabbit, or guinea pig), farm animals, and domestic animals or pets (such as a cat or dog). Non-human primates and, preferably, human patients, are included.

As used herein, “proliferation” refers to the ability of a cell or population of cells to divide and grow. As used herein, “expansion” refers to the ability of a cell or population of cells to increase in numbers. As used herein, “differentiate” or “differentiated” or “differentiation” are used to refer to the process and conditions by which immature (unspecialized) cells acquire characteristics becoming mature (specialized) cells thereby acquiring particular form and function. Stem cells (unspecialized) are often exposed to varying conditions (e.g. growth factors and morphogenic factors) to induce specified lineage commitment, or differentiation, of said stem cells. For example, a naive T cell that transitions to a effector memory cell is differentiated.

Cellular differentiation is accompanied by loss of ‘stemness’—the capability of cells to be multipotent and self-renewing. Along the pathway of T cell differentiation (naive T cell to stem cell memory T cell (TSCM), then to central memory (TCM), then to effector memory T cells (TEM), and then to effector T cells (TEFF), there is a graduate decrease of the expression of the transcription factor TCF-1. Likewise, in chronical viral infection and in tumor, TCF-1 expression is decreased when CD8+ precursor exhausted T cell (Tex precursor) cells differentiate to generate progenitor exhausted T cells (Tpex), which then differentiated to form terminally exhausted T cells (Ttex) (https://pubmed.ncbi.nlm.nih.gov/31606264/).

Less-differentiated T cell subsets such as stem cell memory (TSCM) and central memory (TCM) have reduced levels of ROS whereas terminally differentiated effector memory and effector T cells (TEM and TEFF) display increased ROS levels that are required for their effector function such as cytotoxicity. Increased oxidative stress and DNA damage as a result of ROS accumulation may directly drive CD8+ T cells towards terminal differentiation with characteristics of loss of T cell proliferation, T cell effector function and impaired self-renewal.

As used herein, “uncouples T cell expansion from differentiation” is used to refer to the phenomenon of cells proliferating without undergoing differentiation, as cells that lack differentiation often progress toward states of differentiation as proliferation occurs, wherein expansion and differentiation are coupled. Thus, T cells that experience expansion uncoupled (e.g. become disconnected) from differentiation will continue to grow in number while maintaining stemness. As used herein, “stemness” refers to a cells ability to self-renew and generate daughter cells that are capable of differentiation. A number of illustrative techniques used to expand various undifferentiated cells in vitro while maintaining stemness are described, for example, in Shuai et al. (2016) Theranostics. 6(11):1899-1917; Zhang et al. (2015) Biomaterials. 41:15-25; and Zhang & Wang. (2013) PLoS ONE. 8(4): e61424.

As used herein, the term “CD8+” and “CD4+” are used to refer to cells that express the either the CD8 or the CD4 surface markers, wherein “+” denotes presence and “−” denotes absence. Thus, alternatively, “Tim3−TCF-1+” is used to refer to cells that lack, or have little to no, expression of Tim3 and have expression of TCF-1. Further, “PD-1+” is used to refer to cells that express PD-1.

As used herein, activated T cells refer to any T cells that have encountered with antigens presented by antigen-presenting cells or stimulated by anti-CD3 and CD-28 antibodies. Nonlimiting examples of activated T cells include, e.g., stem memory T cells (Tscm), central memory T cells (Tcm), effector memory T cells, effector T cells, progenitor exhausted T cells (Tpex), and terminally exhausted T cells (Ttex).

As used herein, the phrase “uncoupling T cell expansion from differentiation” refers to the phenomenon of cells proliferating without undergoing differentiation or with less degree of differentiation. Normally, nondifferentiated or less differentiated cells progress toward different states of differentiation while going through the proliferation process, i.e., cell expansion and differentiation are coupled. Under certain conditions as described herein, T cells can experience expansion while uncoupled (e.g. become disconnected) from differentiation. Such cells will continue to grow in number while maintaining stemness. As used herein, “stemness” refers to a cells ability to self-renew and generate daughter cells that are capable of differentiation. A number of illustrative techniques used to expand various undifferentiated cells in vitro while maintaining stemness are described in the art. See, e.g., Gurusamy et al., (2020), Cancer Cell, 37, 818-833; Shuai et al. (2016) Theranostics. 6(11):1899-1917; Zhang et al. (2015) Biomaterials. 41:15-25; and Zhang & Wang. (2013) PLoS ONE. 8(4): e61424.

As used herein, the phrase “in vivo persistence” of adoptively transferred T cells refers to the prolonged survival and proliferation ability of donor T cells in the recipient. The “in vivo persistence” can be quantitatively compared between different donor T cells by analyzing the donor T cell number at given time points (i.e. 7, 14, 30 or more days after adoptive transfer) in the recipient's peripheral blood, tumor site, lymph nodes, spleen, liver, or any other organs where they reside. Donor T cells with superior “in vivo persistence” are expected to have higher numbers in above mentioned sites. Relative to the untreated control T cells, the compound treated T cells are more than 150%, 200%, 250%, 300%, 400%, 500% or more persistent in vivo after adoptive transfer.

Enhancer of zeste homolog 2 (EZH2), the functional enzymatic component of the Polycomb Repressive Complex 2 (PRC2), is a histone-lysine N-methyltransferase enzyme encoded by Ezh2 gene. It mediates gene repression by catalyzing trimethylation of histone H3 at Lys27 (H3K27me3). Tazemetostat (Taz) is a S-adenosyl methionine (SAM) competitive inhibitor of EZH2 approved by FDA in 2020 for the treatment of adults and adolescents aged 16 years and older with metastatic or locally advanced epithelioid sarcoma not eligible for complete resection. See, e.g., Qi et al., Proc Natl Acad Sci USA 2012; 109:21360-5; Knutson et al., Proc Natl Acad Sci USA 2013; 110:7922-7; Knutson et al., Mol. Cancer Ther. 2014; 13(4):842-54; and Mondello and Ansell, Expert Opin. Pharmacother. 2021; 1-7. doi: 10.1080/14656566.2021.2014815.

Kirsten rat sarcoma (KRAS) oncogene, a member of the RAS family, encodes a signaling GTPase that switches between the active GTP-bound and inactive GDP-bound conformations. It is commonly mutated in a broad spectrum of cancers. The KRAS-G12C mutation is the most common genetic abnormality associated with non-small-cell lung cancer (NSCLC), and is also found in several other cancer types (albeit at lower frequency), such as pancreatic ductal adenocarcinoma (PDAC) and colorectal adenocarcinoma. The detection of this biomarker can provide insight into the prognosis of the disease, as well as its response to treatment. Several small molecule inhibitor compounds of KRAS(G12C), including KRAS (G12C) inhibitor 12, are known. See, e.g., Ostrem et al., Nature 503:548-51, 2013; Wang et al., Oncotarget 2016; 7(9):10064-72; and Liu et al., Cancer Gene Ther (2021) https://doi.org/10.1038/s41417-021-00383-9. These compounds are able to lock KRAS in the inactive state to arrest cell proliferation by selectively forming a covalent bond with cysteine 12.

Splenocytes are white blood cells that originate from splenic tissues. Splenocytes comprise a variety of cell populations (e.g. T and B lymphocytes, dendritic cells and macrophages). Naïve cells are cells that are considered immature, wherein naïve T cells have not encountered a cognate antigen within the periphery, unlike activated or memory T cells. Naïve T cells are able to interact with antigen presenting cells (APCs), which use an MHC molecule to present an antigen. Upon recognition of a specific antigen the T cell will proliferate and differentiate into effector T cells of a particular type. To carry out immune functionality effector T cells will interact with host cells.

As used herein, “activation” or “activated” refers to a change from a naïve, or unprimed, T cell, wherein a naïve T cell contacts particular molecules resulting in reorganization of signaling molecules of the T cell culminating in selective proliferation of antigen specific T cells. The process of activation commonly includes antigen processing and presentation by antigen presenting cells that display antigens as peptides bound to MHC; specific binding of the T cell receptor to the antigen simultaneously with binding of CD4 and CD8 coreceptors; costimulation of the T cell by antigen presenting cells through interaction between B7 (CD80/CD86) on dendritic cells and CD28 on T cells; and differentiation through cytokine signaling pathways at the time of activation. As used herein, “an activating agent” is used describe any compound or molecule capable of stimulating activation of a T cell. Activating agents include, but are not limited to, a peptide antigen, antigen-presenting cells, anti-CD3, anti-CD28, Phorbol 12-myristate 13-acetate (PMA), and ionomycin.

The adaptive immune system comprises specific immune cells which include T cells, or T lymphocytes. These cells function in antigen recognition, immune response regulation, production of cytokines, activation of other immune cells, and neutralizing target cells. T cells include regulatory, helper, cytotoxic, or memory T cells. T cells are derived from hematopoietic stem cells that are generated in bone marrow, then travel to the thymus for maturation. After maturation T cells travel to peripheral tissues and circulate in lymphatic or blood systems. Naïve T cells from peripheral blood, upon antigen encounter, undergo activation and differentiate into stem cell memory (Tscm), central memory (Tcm), effector memory (Tem), and effector T (Teff) cells. T cells exhibiting greater differentiation possess greater senescence, exhaustion, and effecter function yet limited therapeutic efficacy, self-renewal, and survival. Conversely, T cells exhibiting lesser differentiation possess greater therapeutic efficacy, self-renewal, and survival yet limited senescence, exhaustion, and effector function. Recent investigation of naïve T cell differentiation from the expansion of tumor infiltrating lymphocytes (TILs) has revealed exhausted-like memory (Tpex), and exhausted CD8 (Ttex) T cells, wherein Tpex cells exhibit limited differentiation allowing for a balance between therapeutic efficacy, self-renewal, survival, senescence, exhaustion, and effector function. T cells are primarily found within lymphoid tissues (e.g., bone marrow, spleen, tonsils, and lymph nodes) with large numbers also present in mucosal sites (e.g., lungs and intestines), skin, and peripheral blood. Naïve T cells are found within the blood, lymph, and secondary lymphoid organs. As used herein, “T cell-containing tissue” refers to any tissue from a subject comprising T cells. In some embodiments, T cell-containing tissue comprises splenocytes, lymph node tissue, or peripheral blood mononuclear cells (PBMCs). In some embodiments, the T cell-containing tissue comprises naïve T cells. In some embodiments, T cell-containing tissue comprises tumor tissue or tumor draining lymph nodes of tumor tissue. In some embodiments, the T cell-containing tissue comprises tumor tissues or T cells from a subject having, or believed to have, a chronic infection or a malignancy.

As used herein, “less differentiated state” refers to the state of T cells possessing relatively higher levels of TCF-1 expression or the state of T cells in the earlier stage of differentiation trajectory. For example, if T cell A expresses 10% higher TCF-1 than T cell B, T cell A is considered less differentiated than T cell B. Likewise, progenitor exhausted T cells (Tpex) are less differentiated than terminally exhausted T cells (Ttex). Stem cell memory T cells (Tscm) and central memory T cells (Tcm) are less differentiated than effector T cells (Teff). Memory T cells exhibit CD3+ and CD4+ or CD8+. Tcm cells are antigen-specific T cells that remain in biological systems for prolonged periods of time after primary exposure to an antigen and may be converted into effector T cells upon re-exposure to an antigen. In humans, Tcm cells may present with characteristic markers comprising CCR7+, CD45RA−, CD45RO+, CD62L+, and CD27+. In mice, Tcm cells may present with characteristic markers comprising CC44+ and CD62L+. Tscm cells are progenitor cells that are multipotent and can both self-renew and function to provide more differentiated subsets of memory T cells. In humans, Tscm cells may present with characteristic markers comprising CD45RA+, CD45RO−, CCR7+, CD62L+, CD27+, CD28+, CD95+, and IL-7Ra+. In mice, Tscm cells may present with characteristic markers comprising CD44⁻ and CD62L+. In some embodiments, a “less differentiated state” of T cells can refer to a state of T cells possessing relatively higher levels of TCF-1 expression or a state of T cells in the earlier stage of differentiation trajectory. For example, if T cell A expresses 10% higher TCF-1 than T cell B, T cell A is considered less differentiated than T cell B. Likewise, Tscm and Tcm cells are less differentiated than Teff cells. Progenitor exhausted T cells (Tpex) are less differentiated than terminally exhausted T cells (Ttex). As described herein, several compounds are able to maintain activated T cells in a less differentiated state. In comparison to the untreated control T cells, the compound treated T cells can be less than 75%, 60%, 50%, 40%, 30%, 20%, or 10% less differentiated.

As used herein, the term “exhausted” or “exhaustion”, unless otherwise stated, generally refers to effector T cells with a reduced capacity to secrete cytokines (e.g. IL-2), a reduced capacity to proliferation, and increased expression of inhibitory receptors (e.g. PD-1, Tim-3, Lag-3). Exhaustion is characterized by progressive loss of T cell effector functions, wherein under certain conditions (i.e., persistent exposure to antigens), T cells become incapable of elaborating effector-related activities including the production of effector and memory T cell populations. Exhausted T-cell responses have been observed under various circumstances including, but not limited to, Lymphocytic choriomeningitis virus (LCMV) infection, polyoma virus infection, adenovirus infection, Friend leukemia virus infection, mouse hepatitis virus infection, human immunodeficiency virus (HIV) infection, hepatitis B virus (HBV) infection, hepatitis C virus (HCV) infection, and have also been reported in subjects with malignancies.

Progenitor exhausted T cells or exhausted-like memory (Tpex) cells comprise cells that are Tim3−TCF-1+. They represent a sub-population of dysfunctional CD8⁺ T cells identified in viral-infected animal models and patients and tumors that express transcription factor TCF-1 without the expression of the immune regulatory protein Tim-3 or with basal levels of Tim-3 expression. These T cells display stem cell-like properties: they can produce terminally differentiated cells and reproduce themselves during cell division (self-renewal) within the tumor microenvironment. Progenitor exhausted T cells provide the proliferative burst and effector function following anti-PD-1/PD-L1 therapy, whereas terminal exhausted T cells that express high levels of Tim-3 without the expression of TCF-1 (TCF-1⁻Tim-3⁺) are non-responsive to PD-1 blockade and short-lived. CD8+ T cells that are Tim-3-TCF-1⁺ differentiate into a transient state that is Tim3⁻TCF-1⁻ first, then into Tim-3⁺ TCF-1⁻ T cells.

As used herein, the term “isolated” is used to refer to molecules or cells that are removed from native environments. As used herein, the term “non-naturally occurring” is used to refer to isolated molecules or cells that possess markedly different structures than counterparts found in nature.

As used herein, a composition containing a “cell population” or “purified cell population” or “purified cell composition” comprising a particular cell means that at least 30%, 50%, 60%, typically at least 70%, and more preferably 80%, 90%, 95%, 98%, 99%, or more of the cells in the composition are of the identified type.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, may mean that the number or numeral ranges plus or minus 1%, plus or minus 5%, plus or minus 10%.

The term “polynucleotide” or “nucleic acid” are used interchangeably herein to refer to a polymer of nucleotides, which can be mRNA, RNA, cRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

The terms “polypeptide”, “peptide”, or “protein” are used interchangeably herein to designate a linear series of amino acid residues connected one to the other by peptide bonds between the alpha-amino and carboxyl groups of adjacent residues. The amino acid residues are usually in the natural “L” isomeric form. However, residues in the “D” isomeric form can be substituted for any L-amino acid residue, as long as the desired functional property is retained by the polypeptide.

As used herein, “antibody” is understood to mean any antigen-binding molecule or molecular complex comprising at least one complementarity determining region (CDR) that binds specifically to, or interacts specifically with, the target antigen. The term “antibody” includes full-length immunoglobulin molecules comprising two heavy (H) chains and two light (L) chains inter-connected by disulfide bonds, as well as multimers thereof (e.g., IgM). Each heavy chain comprises a heavy chain variable region (which may be abbreviated as HCVR, VH or VH) and a heavy chain constant region. The heavy chain constant region typically comprises three domains—CH1, CH2 and CH3. Each light chain comprises a light chain variable region (which may be abbreviated as LCVR, VL, VK, VK or VL) and a light chain constant region. The light chain constant region will typically comprise one domain (CL1). The VH and VL regions can be further subdivided into regions of hypervariability, termed complementarity determining regions (CDRs), interspersed with regions that are more conserved, also referred to as framework regions (FR).

The terms “host”, “host cell”, “host cell line” and “host cell culture” are used interchangeably and refer to cells into which exogenous nucleic acid has been introduced, including the progeny of such cells. Host cells include “transformants” or “transformed cells” or “engineered cells”, which include the primary transformed cell and progeny derived therefrom without regard to the number of passages. Progeny may not be completely identical in nucleic acid content to a parent cell and may contain mutations. Mutant progeny that have the same function or biological activity as screened or selected for in the originally transformed cell are included herein. A host cell is any type of cellular system that can be used to generate the antigen binding molecules of the present invention. Host cells include cultured cells, e.g., mammalian cultured cells, such as, but not limited to, T cells.

As used herein, the terms “vector” and “construct”, which are used interchangeably, may be nucleic acid molecules, preferably DNA molecules derived, for example, from a plasmid, bacteriophage, or virus, into which a nucleic acid sequence may be inserted or cloned. A vector may contain one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art. In some embodiments, vectors are used to generate the engineered NK cell or the engineered macrophage cell of the current invention.

As used herein, unless as otherwise described with regard to viral vectors, “vector” means a nucleic acid molecule capable of transporting another nucleic acid to which it has been linked. Exemplary vectors include plasmids, minicircles, transposons, yeast artificial chromosomes, self-replicating RNAs, and viral genomes. Certain vectors can autonomously replicate in a host cell, while other vectors can be integrated into the genome of a host cell and thereby are replicated with the host genome. In addition, certain vectors are referred to herein as “recombinant expression vectors” (or simply, “expression vectors”), which contain nucleic acid sequences that are operatively linked to an expression control sequence and, therefore, are capable of directing the expression of those sequences. In certain embodiments, expression constructs are derived from plasmid vectors. Illustrative constructs include modified pNASS vector (Clontech, Palo Alto, CA), which has nucleic acid sequences encoding an ampicillin resistance gene, a polyadenylation signal and a T7 promoter site; pDEF38 and pNEF38 (CMC ICOS Biologies, Inc.), which have a CHEF1 promoter; and pD18 (Lonza), which has a CMV promoter. Other suitable mammalian expression vectors are well known (see, e.g., Ausubel et al, 1995; Sambrook et al, supra; see also, e.g., catalogs from Invitrogen, San Diego, CA; Novagen, Madison, Wl; Pharmacia, Piscataway, NJ).

As used herein, an “expression construct” refers to a nucleic acid molecule which comprises coding sequences for the therapeutic protein, promoter, and may include other regulatory sequences therefor, which cassette may be engineered into a genetic element and/or packaged into the capsid of a viral vector (e.g., a viral particle). Typically, such an expression cassette for generating a viral vector contains the construct sequences described herein flanked by packaging signals of the viral genome and other expression control sequences such as those described herein. Any of the expression control sequences can be optimized for a specific species using techniques known in the art including, e.g., codon optimization.

By “control element”, “control sequence”, “regulatory sequence” and the like, as used herein, mean a nucleic acid sequence (e.g., DNA) necessary for expression of an operably linked coding sequence in a particular host cell. The control sequences that are suitable for prokaryotic cells for example, include a promoter, and optionally a cis-acting sequence such as an operator sequence and a ribosome binding site. Control sequences that are suitable for eukaryotic cells include transcriptional control sequences such as promoters, polyadenylation signals, transcriptional enhancers, translational control sequences such as translational enhancers and internal ribosome binding sites (IRES), nucleic acid sequences that modulate mRNA stability, as well as targeting sequences that target a product encoded by a transcribed polynucleotide to an intracellular compartment within a cell or to the extracellular environment.

As used herein “gene of interest” or “gene” or “nucleic acid of interest” refers to a transgene to be expressed in the target transfected cell. While the term “gene” may be used, this is not to imply that this is a gene as found in genomic DNA and is used interchangeably with the term “nucleic acid”. Generally, the nucleic acid of interest provides suitable nucleic acid for encoding a therapeutic agent and may comprise cDNA or DNA and may or may not include introns, but generally does not include introns. As noted elsewhere, the nucleic acid of interest is operably linked to expression control sequences to effectively express the protein of interest in the target cell. In some embodiments, the vectors described herein may comprise one or more genes of interest, and may include 2, 3, 4, or 5 or more genes of interest.

Thus, this disclosure provides polynucleotides (isolated or purified or pure polynucleotides) encoding therapeutic agents (e.g., proteins of interest) of this disclosure for genetically modifying progenitor exhausted T cells expanded using the methods describe herein, vectors (including cloning vectors and expression vectors) comprising such polynucleotides, and cells (e.g., host cells) transformed or transfected with a polynucleotide or vector according to this disclosure. In certain embodiments, a polynucleotide (DNA or RNA) encoding a protein of interest of this disclosure is contemplated. Expression cassettes encoding proteins of interest are also contemplated herein.

The present disclosure also relates to vectors that include a polynucleotide of this disclosure and, in particular, to recombinant expression constructs. In one embodiment, this disclosure contemplates a vector comprising a polynucleotide encoding a protein of this disclosure, along with other polynucleotide sequences that cause or facilitate transcription, translation, and processing of such a protein-encoding sequences. Appropriate cloning and expression vectors for use with prokaryotic and eukaryotic hosts are described, for example, in Sambrook et al, Molecular Cloning: A Laboratory Manual, Second Edition, Cold Spring Harbor, NY, (1989). Exemplary cloning/expression vectors include cloning vectors, shuttle vectors, and expression constructs, that may be based on plasmids, phagemids, phasmids, cosmids, viruses, artificial chromosomes, or any nucleic acid vehicle known in the art suitable for amplification, transfer, and/or expression of a polynucleotide contained therein.

Generally, recombinant expression vectors will include origins of replication and selectable markers permitting transformation of the host cell, and a promoter derived from a highly-expressed gene to direct transcription of a downstream structural sequence, as described above. A vector in operable linkage with a polynucleotide according to this disclosure yields a cloning or expression construct. Exemplary cloning/expression constructs contain at least one expression control element, e.g., a promoter, operably linked to a polynucleotide of this disclosure. Additional expression control elements, such as enhancers, factor-specific binding sites, terminators, and ribosome binding sites are also contemplated in the vectors and cloning/expression constructs according to this disclosure. The heterologous structural sequence of the polynucleotide according to this disclosure is assembled in appropriate phase with translation initiation and termination sequences. Thus, for example, encoding nucleic acids as provided herein may be included in any one of a variety of expression vector constructs (e.g., minicircles) as a recombinant expression construct for expressing such a protein in a host cell.

The appropriate DNA sequence(s) may be inserted into a vector, for example, by a variety of procedures. In general, a DNA sequence is inserted into an appropriate restriction endonuclease cleavage site(s) by procedures known in the art. Standard techniques for cloning, DNA isolation, amplification and purification, for enzymatic reactions involving DNA ligase, DNA polymerase, restriction endonucleases and the like, and various separation techniques are contemplated. A number of standard techniques are described, for example, in Ausubel et al. (Current Protocols in Molecular Biology, Greene Publ. Assoc. Inc. & John Wiley & Sons, Inc., Boston, MA, 1993); Sambrook et al. (Molecular Cloning, Second Ed., Cold Spring Harbor Laboratory, Plainview, NY, 1989); Maniatis et al. (Molecular Cloning, Cold Spring Harbor Laboratory, Plainview, NY, 1982); Glover (Ed.) (DNA Cloning Vol. I and II, IRL Press, Oxford, U K, 1985); Hames and Higgins (Eds.) (Nucleic Acid Hybridization, IRL Press, Oxford, U K, 1985); and elsewhere.

The DNA sequence in the expression vector is operatively linked to at least one appropriate expression control sequence (e.g., a constitutive promoter or a regulated promoter) to direct mRNA synthesis. Representative examples of such expression control sequences include promoters of eukaryotic cells or their viruses, as described above. Promoter regions can be selected from any desired gene using CAT (chloramphenicol transferase) vectors, kanamycin vectors, or other vectors with selectable markers. Eukaryotic promoters include CMV immediate early, HSV thymidine kinase, early and late SV40, LTRs from retrovirus, EEK, EF1alpha, and mouse metallothionein-1. Selection of the appropriate vector and promoter is well within the level of ordinary skill in the art, and preparation of certain particularly preferred recombinant expression constructs comprising at least one promoter or regulated promoter operably linked to a nucleic acid encoding a protein or polypeptide according to this disclosure is described herein.

Variants of the polynucleotides of this disclosure are also contemplated. Variant polynucleotides are at least 60%, 65%, 70%, 75%, 80%, 85%, 90%, and preferably 95%, 96%, 97%, 98%, 99%, or 99.9% identical to one of the polynucleotides of defined sequence as described herein, or that hybridizes to one of those polynucleotides of defined sequence under stringent hybridization conditions of 0.015M sodium chloride, 0.0015M sodium citrate at about 65-68° C. or 0.015M sodium chloride, 0.0015M sodium citrate, and 50% formamide at about 42° C. The polynucleotide variants retain the capacity to encode a binding domain or fusion protein thereof having the functionality described herein.

In some embodiments, cells are transfected or otherwise engineered (e.g., via a targeted integration of a transgene) prior to activation. In some embodiments, cells are transfected or otherwise engineered (e.g., via a targeted integration of a transgene) during activation. In some embodiments, cells are transfected or otherwise engineered (e.g., via a targeted integration of a transgene) after activation. In some embodiments, cells are transfected or otherwise engineered (e.g., via a targeted integration of a transgene) prior to differentiation. In some embodiments, cells are transfected or otherwise engineered (e.g., via a targeted integration of a transgene) during differentiation. In some embodiments, cells are transfected or otherwise engineered (e.g., via a targeted integration of a transgene) after differentiation.

In some embodiments, a non-viral vector is used to deliver DNA or RNA to T cells. For example, systems that may facilitate transfection of T cells without the need of a viral integration system include, without limitation, transposons, zinc-finger nucleases (ZFNs), transcription activator-like effector nucleases (TALENs), clustered regularly interspaced short palindromic repeats (CRISPRs) (including but not limited to Cas9 or CasX), meganucleases, minicircles, replicons, artificial chromosomes (e.g., bacterial artificial chromosomes, mammalian artificial chromosomes, and yeast artificial chromosomes), plasmids, cosmids, and bacteriophage.

In some embodiments, non-viral-dependent vector systems may also be delivered via a viral vector known in the art or described below. For example, in some embodiments, a viral vector (e.g., a retrovirus, lentivirus, adenovirus, adeno-associated virus), is utilized to deliver one or more non-viral vector (such as, e.g., one or more of the above-mentioned zinc-finger nucleases (ZFNs), transcription activator-like effector nucleases (TALENs), clustered regularly interspaced short palindromic repeats (CRISPRs) meganucleases, or any other enzyme/complementary vectors, polynucleotides, and/or polypeptides capable of facilitating the targeted integration. Accordingly, in some embodiments, a cell (e.g., T cell) may be engineered to express an exogenous sequence via a targeted integration method. Such methods are known in the art and may comprise cleaving an endogenous locus in the cell using one or more nucleases (e.g., ZFNs, TALENs, CRISPR/Cas, meganucleases) and administering the transgene to the cell such that it is integrated into the endogenous locus and expressed in the cell. The transgene may be comprised in a donor sequence that is integrated into the host cell's DNA at or near the point of a cleavage by the nuclease. In some embodiments, the T cells were prepared by gene editing of the T cell genome or by targeted integration into the genome of the T cell of a polynucleotide sequence.

In some embodiments, the targeted integration comprises a zinc finger nuclease-mediated gene integration, CRISPR-mediated gene integration or gene editing, TALE-nuclease-mediated gene integration, or meganuclease-mediated gene integration. In some embodiments, the targeted integration of polynucleotide occurred via homologous recombination. In some embodiments, the targeted integration comprises a viral vector-mediated delivery of a nuclease capable of inducing a DNA cleavage at a target site. In some embodiments, the nuclease is a zinc finger nuclease, a Cas nuclease, a TALE-nuclease, or a meganuclease.

The integration of the exogenous sequence may occur via recombination. As would be clear to one of skill in the art, “Recombination” refers to a process of exchange of genetic information between two polynucleotides, including but not limited to, donor capture by non-homologous end joining (NHEJ) and homologous recombination. The recombination may be homologous recombination. For the purposes of this disclosure, “homologous recombination (HR)” refers to the specialized form of such exchange that takes place, for example, during repair of double-strand breaks in cells via homology-directed repair mechanisms. This process utilizes nucleotide sequence homology, whereby a “donor” molecule (e.g., donor polynucleotide sequence or donor vector comprising such a sequence) is utilized by a cell's DNA-repair machinery as a template to repair of a “target” molecule (i.e., the one that experienced the double-strand break), and by these means causes the transfer of genetic information from the donor to the target. In some embodiments of HR-directed integration, the donor molecule may contain at least 2 regions of homology to the genome (“homology arms”). In some embodiments, the homology arms may be, e.g., of least 50-100 base pairs in length. The homology arms may have substantial DNA homology to a region of genomic DNA flanking the cleavage site wherein the targeted integration is to occur. The homology arms of the donor molecule may flank the DNA that is to be integrated into the target genome or target DNA locus. Breakage of the chromosome followed by repair using the homologous region of the plasmid DNA as a template may results in the transfer of the intervening transgene flanked by the homology arms into the genome. See, e.g., Roller et al. (1989) Proc. Natl. Acad Sci. USA. 86(22): 8927-8931; Thomas et al. (1986) Cell 44(3):419-428. The frequency of this type of homology-directed targeted integration can be increased by up to a factor of 105 by deliberate creation of a double-strand break in the vicinity of the target region (Hockemeyer et al. (2009) Nature Biotech. 27(9):851-857; Lombardo et al. (2007) Nature Biotech. 25(11): 1298-1306; Moehle et al. (2007) Proc. Natl. Acad. Sci. USA 104(9):3055-3060: Rouet et al. (1994) Proc. Natl. Acad. Sci. USA 91 (13):6064-6068.

Any nuclease capable of mediating the targeted cleavage of a genomic locus such that a trans gene may be integrated into the genome of a target cell (e.g., by recombination such as HR) may be utilized in engineering a cell (e.g., a T cell) according to the present disclosure.

A double-strand break (DSB) or nick can be created by a site-specific nuclease such as a zinc-finger nuclease (ZFN), a TAL effector domain nuclease (TALEN), a meganuclease, or using the CRISPR-mediated system with an engineered crRNA/tract RNA (single guide RNA) to guide specific cleavage. See. for example, Burgess (2013) Nature Reviews Genetics 14:80-81, pMov et al. (2010) Nature 435(7042):646-51; U.S. Pat. Pub. Nos. 2003/0232410; 2005/0208489; 2005/0026157, 2005/0064474; 2006/0188987, 2009/0263900; 2009/0117617; 2010/0047805; 2011/0207221; 2011/0301073 and Int'l Pat. Pub. No. WO 2007/014275, the disclosures of which are incorporated by reference in their entireties for all purposes.

In some embodiments, the cell (e.g., a T cell) is engineered via Zinc Finger Nuclease-mediated targeted integration of a donor construct. A zinc finger nuclease (ZFN) is an enzyme that is able to recognize and cleave a target nucleotide sequence with specificity due to the coupling of a “zinc finger DNA binding protein” (ZFP) (or binding domain), which binds DNA in a sequence-specific manner through one or more zinc fingers, and a nuclease enzyme. ZFNs may comprise any suitable cleavage domains (e.g., a nuclease enzyme) operatively linked to a ZFP DNA-binding domain to form a engineered ZFN that can facilitate site-specific cleavage of a target DNA sequence (see, e.g., Kim et al. (1996) Proc Natl Acad Sci USA 93(3): 1156-1160). For example, ZFNs may comprise a target-specific ZFP linked to a FORI enzyme or a portion of a FOK1 enzyme. In some embodiments, ZFN used in a ZFN-mediated targeted integration approach utilize two separate molecules, each comprising a subunit of a FOK1 enzyme each bound to a ZFP, each ZFP with specificity for a DNA sequence flanking a target cleavage site, and when the two ZFPs bind to their respective target DNA sites the FOK1 enzyme subunits are brought into proximity with one another and they bind together activating the nuclease activity which cleaves the target cleavage site. ZFNs have been used for genome modification in a variety of organisms (e.g., United States Patent Publications 20030232410; 20050208489; 20050026157; 20050064474; 20060188987; 20060063231; and International Publication WO 07/014,275, incorporated herein by reference in their entirety) Custom ZFPs and ZFNs are commercially available from, e.g., Sigma Aldrich (St. Louis, MO), and any location of DNA may be routinely targeted and cleaved using such custom ZFNs.

In some embodiments, the cell (e.g., T cell) is engineered via CRISPR-mediated (e.g., CRISPR/Cas) Nuclease-mediated integration of a donor construct. A CRISPR (Clustered Regularly Interspaced Short Palindromic Repeats)/Cas (CRISPR Associated) nuclease system is an engineered nuclease system based on a bacterial system that may be used for genome engineering. It is based on part of the adaptive immune response of many bacteria and archea. When a virus or plasmid invades a bacterium, segments of the invader's DNA are converted into CRISPR RNAs (crRNA) by the ‘immune’ response. This crRNA then associates, through a region of partial complementarity, with another type of RNA called tracrRNA to guide the Cas (e.g. Cas9) nuclease to a region homologous to the crRNA in the target DNA called a “protospacer”. Cas cleaves the DNA to generate blunt ends at the DSB at sites specified by a 20-nucleotide guide sequence contained within the crRNA transcript. Cas requires both the crRNA and the tracrRNA for site specific DNA recognition and cleavage. This system has now been engineered such that the crRNA and tracrRNA can be combined into one molecule (the “single guide RNA”), and the crRNA equivalent portion of the single guide RNA can be engineered to guide the Cas nuclease to target any desired sequence (see Jinek et al (2012) Science 337, p. 816-821, Jinek et al, (2013), eLife 2:e00471, and David Segal, (2013) eLife 2:e00563). Thus, the CRISPR/Cas system can be engineered to create a DSB at a desired target in a genome, and repair of the DSB can be influenced by the use of repair inhibitors to cause an increase in error prone repair. In some embodiments, the CRISPR/Cas nuclease-mediated integration utilizes a Type II CRISPR. The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas to the target DNA via Watson-Crick base-pairing between the spacer on the crRNA and the protospacer on the target DNA next to a protospacer adjacent motif (PAM), an additional requirement for target recognition. Forth, Cas mediates cleavage of target DNA to create a double-stranded break within the protospacer.

The Cas related CRISPR/Cas system comprises two RNA non-coding components: tracrRNA and a pre-crRNA array containing nuclease guide sequences (spacers) interspaced by identical direct repeats (DRs). To use a CRISPR/Cas system to accomplish genome engineering, both functions of these RNAs must be present (see Cong et al, (2013) Sciencexpress 1/10.1126/science 1231143). In some embodiments, the tracrRNA and pre-crRNAs are supplied via separate expression constructs or as separate RNAs. In other embodiments, a chimeric RNA is constructed where an engineered mature crRNA (conferring target specificity) is fused to a tracrRNA (supplying interaction with the Cas) to create a chimeric cr-RNA-tracrRNA hybrid (also termed a single guide RNA). (see Jinek ibid and Cong, ibid).

In some embodiments, a single guide RNA containing both the crRNA and tracrRNA may be engineered to guide the Cas nuclease to target any desired sequence (e.g., Jinek et al (2012) Science 337, p. 816-821, Jinek et al, (2013), eLife 2:e00471, David Segal, (2013) eLife 2:e00563). Thus, the CRISPR/Cas system may be engineered to create a DSB at a desired target in a genome.

Custom CRISPR/Cas systems are commercially available from, e.g., Dharmacon (Lafayette, CO), and any location of DNA may be routinely targeted and cleaved using such custom single guide RNA sequences. Single stranded DNA templates for recombination may be synthesized (e.g., via oligonucleotide synthesis methods known in the art and commercially available) or provided in a vector, e.g., a viral vector such as an AAV. In some embodiments, the cell (e.g., a T cell) is engineered via TALE-Nuclease (TALEN) mediated targeted integration of a donor construct. A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. TAL-effectors may contain a nuclear localization sequence, an acidic transcriptional activation domain and a centralized domain of tandem repeats where each repeat contains approximately 34 amino acids that are key to the DNA binding specificity of these proteins, (e.g., Schornack S, et al (2006) J Plant Physiol 163(3): 256-272). TAL effectors depend on the sequences found in the tandem repeats which comprises approximately 102 bp and the repeats are typically 91-100% homologous with each other (e.g., Bonas et al (1989) Mol Gen Genet 218: 127-136). These DNA binding repeats may be engineered into proteins with new combinations and numbers of repeats, to make artificial transcription factors that are able to interact with new sequences and activate the expression of a non-endogenous reporter gene (e.g., Bonas et al (1989) Mol Gen Genet 218: 127-136). Engineered TAL proteins may be linked to a Fokl cleavage half domain to yield a TAL effector domain nuclease fusion (TALEN) to cleave target specific DNA sequence (e.g., Christian et al (2010) Genetics epub 10.1534/genetics. 110.120717).

Custom TALEN are commercially available from, e.g., Thermo Fisher Scientific (Waltham, MA), and any location of DNA may be routinely targeted and cleaved.

In some embodiments, the cell (e.g., T cell) is engineered via meganuclease-mediated targeted integration of a donor construct. A meganuclease (or “homing endonuclease”) is an endonuclease that binds and cleaves double-stranded DNA at a recognition sequence that is greater than 12 base pairs. Naturally occurring meganucleases may be monomelic (e.g., I-Scel) or dimeric (e.g., I-Crel). Naturally occurring meganucleases recognize 15-40 base-pair cleavage sites and are commonly grouped into four families: the LAGLIDADG family, the GIY-YIG family, the His-Cyst box family and the HNH family. Exemplary homing endonucleases include I-Scel, I-Ceul, PI-PspI, PI-Sce, I-SceIV, I-Csml, I-Panl, I-Scell, I-Ppol, I-SceIII, I-Crel, I-Tevl, I-TevII and I-TevIII. Their recognition sequences are known. See also U.S. Pat. Nos. 5,420,032; 6,833,252; Belfort et al. (1997) Nucleic Acids Res. 25:3379-3388; Dujon et al. (1989) Gene 82: 115-118; Perler et al. (1994) Nucleic Acids Res. 22, 1125-1127; Jasin (1996) Trends Genet. 12:224-228; Gimble et al. (1996) J. Mol. Biol. 263: 163-180; Argast et al. (1998) J. Mol. Biol. 280:345-353 and the New England Biolabs catalogue. The term “Meganuclease” includes monomeric meganucleases, dimeric meganucleases and monomers that associate to form dimeric meganucleases.

In some embodiments, the methods and compositions described herein make use of a nuclease that comprises an engineered (non-naturally occurring) homing endonuclease (meganuclease). The recognition sequences of homing endonucleases and meganucleases such as I-Scel, I-Ceul, PI-PspI, PI-Sce, I-SceIV, I-Csml, I-Panl, I-SceII, I-Ppol, I-SceIII, I-Crel, I-Tevl, I-TevII and I-TevIII are known. See also U.S. Pat. Nos. 5,420,032; 6,833,252; Belfort et al. (1997) Nucleic Acids Res. 25:3379-3388; Dujon et al. (1989) Gene 82: 115-118; Perler et al. (1994) Nucleic Acids Res. 22, 1125-1127; Jasin (1996) Trends Genet. 12:224-228; Gimble et al. (1996) J. Mol. Biol. 263: 163-180; Argast et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication No. 20070117128. The DNA-binding domains of the homing endonucleases and meganucleases may be altered in the context of the nuclease as a whole (i.e., such that the nuclease includes the cognate cleavage domain) or may be fused to a heterologous cleavage domain. Custom meganuclease are commercially available from, e.g., New England Biolabs (Ipswich, MA), and any location of DNA may be routinely targeted and cleaved.

The engineering of the T cell may comprise administering one or more nucleases (e.g., ZFNs, TALENs, CRISPR/Cas, meganuclease) to a T cell, e.g., via one or more vectors encoding the nucleases, such that the vectors comprising the encoded nucleases are taken up by the T cell. The vectors may be viral vectors.

In some embodiments, the nucleases cleave a specific endogenous locus (e.g. safe harbor gene or locus of interest) in the cell (e.g., T cell) and one or more exogenous (donor) sequences (e.g., transgenes) are administered (e.g. one or more vectors comprising these exogenous sequences). The nuclease may induce a double-stranded (DSB) or single-stranded break (nick) in the target DNA. In some embodiments, targeted insertion of a donor transgene may be performed via homology directed repair (HDR), non-homology repair mechanisms (e.g., NHEJ-mediated end capture), or insertions and/or deletion of nucleotides (e.g. endogenous sequence) at the site of integration of a transgene into the cell's genome.

In some embodiments, the T cells are contacted with a vector comprising a nucleic acid of interest operably linked to a promoter, under conditions sufficient to transfect at least a portion of the T cells. In some embodiments the T cells are contacted with a vector comprising a nucleic acid of interest operably linked to a promoter, under conditions sufficient to transfect at least 5% of the T cells. In some embodiments, the T cells are contacted with a vector under conditions sufficient to transfect at least 5%, 10%, 15%, 20%, 25%, 30%, 35%, 40%, 45%, 50%, 55%, 60%, 70%, 75%, 80%, 85%, 90%, 95%, 96%, 97%, 98%, 99% or even 100% of the T cells. In some embodiments, the T cells, cultured in vitro as described herein, are transfected, in which case the cultured T cells are contacted with a vector as described herein under conditions sufficient to transfect at least 5%, 10% 15%, 20%, 25%, 30%, 35%, 40%, 45%, 50%, 55%, 60%, 70%, 75%, 80%, 85%, 90%, 95%, 96%, 97%, 98%, 99% or even 100% of the T cells.

Viral vectors may be employed to transduce T cells. Examples of viral vectors include, without limitation, adenovirus-based vectors, adeno-associated virus (AAV)-based vectors, retroviral vectors, retroviral-adenoviral vectors, and vectors derived from herpes simplex viruses (HSVs), including amplicon vectors, replication-defective HSV and attenuated HSV (see, e.g., Krisky, Gene Ther. 5: 1517-30, 1998; Pfeifer, Annu. Rev. Genomics Hum. Genet. 2: 177-211, 2001, each of which is incorporated by reference in its entirety). In some embodiments, cells are transduced with a viral vector (e.g., a lentiviral vector) on day 1, 2, 3, 4, 5, 6, 7, 8, or 9 of in vitro culture. In some embodiments, cells are transduced with a viral vector on day 5 of in vitro culture. In some embodiments, the viral vector is a lentivirus. In some embodiments, cells are transduced with a measles virus pseudotyped lentivirus on day 1 of in vitro culture.

In some embodiments, T cells are transduced with retroviral vectors using any of a variety of known techniques in the art (see, e.g., Science 12 Apr. 1996 272: 263-267; Blood 2007, 99:2342-2350; Blood 2009, 1 13: 1422-1431; Blood 2009 Oct. 8; 1 14(15):3173-80; Blood. 2003; 101 (6):2167-2174; Current Protocols in Molecular Biology or Current Protocols in Immunology, John Wiley & Sons, New York, N.Y. (2009)).

Some embodiments relate to the use of retroviral vectors, or vectors derived from retroviruses. “Retroviruses” are enveloped RNA viruses that are capable of infecting animal cells, and that utilize the enzyme reverse transcriptase in the early stages of infection to generate a DNA copy from their RNA genome, which is then typically integrated into the host genome. Examples of retroviral vectors Moloney murine leukemia virus (MLV)-derived vectors, retroviral vectors based on a Murine Stem Cell Virus, which provides long-term stable expression in target cells such as hematopoietic precursor cells and their differentiated progeny (see, e.g., Hawley et al., PNAS USA 93: 10297-10302, 1996; Keller et al., Blood 92:877-887, 1998), hybrid vectors (see, e.g., Choi, et al, Stem Cells 19:236-246, 2001), and complex retrovirus-derived vectors, such as lentiviral vectors.

In some embodiments, the T cells are contacted with a retroviral vector comprising a nucleic acid of interest operably linked to a promoter, under conditions sufficient to transduce at least a portion of the T cells. In one embodiment the T cells are contacted with a retroviral vector comprising a nucleic acid of interest operably linked to a promoter, under conditions sufficient to transduce at least 2% of the T cells. In some embodiments, the T cells are contacted with a vector under conditions sufficient to transduce at least 2%, 3%, 4%, 5%, 10%, 15%, 20%, 25%, 30%, 35%, 40%, 45%, 50%, 55%, 60%, 70%, 75%, 80%, 85%, 90%, 95%, 96%, 97%, 98%, 99% or even 100% of the resting T cells. In some embodiments, the differentiated and activated T cells, cultured in vitro as described herein, are transduced, in which case the cultured differentiated/activated T cells are contacted with a vector as described herein under conditions sufficient to transduce at least 2%, 3%, 4%, 5%, 10% 15%, 20%, 25%, 30%, 35%, 40%, 45%, 50%, 55%, 60%, 70%, 75%, 80%, 85%, 90%, 95%, 96%, 97%, 98%, 99% or even 100% of the differentiated and activated T cells.

As noted above, some embodiments employ lentiviral vectors. The term “lentivirus” refers to a genus of complex retroviruses that are capable of infecting both dividing and non-dividing cells. Examples of lentiviruses include HIV (human immunodeficiency virus; including HIV type 1, and HIV type 2), visna-maedi, the caprine arthritis-encephalitis virus, equine infectious anemia virus, feline immunodeficiency virus (FIV), bovine immune deficiency virus (BIV), and simian immunodeficiency virus (SIV). Lentiviral vectors can be derived from any one or more of these lentiviruses (see, e.g., Evans et al, Hum Gene Ther. 10: 1479-1489, 1999; Case et al, PNAS USA 96:2988-2993, 1999; Uchida et al, PNAS USA 95: 1 1939-1 1944, 1998; Miyoshi et al, Science 283:682-686, 1999; Sutton et al, J Virol 72:5781-5788, 1998; and Frecha et al, Blood. 1 12:4843-52, 2008, each of which is incorporated by reference in its entirety).

It has been documented that resting T and B cells can be transduced by a VSVG-coated LV carrying most of the HIV accessory proteins (vif, vpr, vpu, and nef) (see e.g., Frecha et al, 2010 Mol. Therapy 18: 1748). In certain embodiments the retroviral vector comprises certain minimal sequences from a lentivirus genome, such as the HIV genome or the SIV genome. The genome of a lentivirus is typically organized into a 5′ long terminal repeat (LTR) region, the gag gene, the pol gene, the env gene, the accessory genes (e.g., nef, vif, vpr, vpu, tat, rev) and a 3′ LTR region. The viral LTR is divided into three regions referred to as U3, R (repeat) and U5. The U3 region contains the enhancer and promoter elements, the U5 region contains the polyadenylation signals, and the R region separates the U3 and U5 regions. The transcribed sequences of the R region appear at both the 5′ and 3′ ends of the viral RNA (see, e.g., “RNA Viruses: A Practical Approach” (Alan J. Cann, Ed., Oxford University Press, 2000); O Narayan, J. Gen. Virology. 70: 1617-1639, 1989; Fields et al, Fundamental Virology Raven Press., 1990; Miyoshi et al, J Virol. 72:8150-7, 1998; and U.S. Pat. No. 6,013,516, each of which is incorporated by reference in its entirety). Lentiviral vectors may comprise any one or more of these elements of the lentiviral genome, to regulate the activity of the vector as desired, or, they may contain deletions, insertions, substitutions, or mutations in one or more of these elements, such as to reduce the pathological effects of lentiviral replication, or to limit the lentiviral vector to a single round of infection.

Typically, a minimal retroviral vector comprises certain 5′LTR and 3′LTR sequences, one or more genes of interest (to be expressed in the target cell), one or more promoters, and a cis-acting sequence for packaging of the RNA. Other regulatory sequences can be included, as described herein and known in the art. The viral vector is typically cloned into a plasmid that may be transfected into a packaging cell line, such as a eukaryotic cell (e.g., 293-HEK), and also typically comprises sequences useful for replication of the plasmid in bacteria.

In certain embodiments, the viral vector comprises sequences from the 5′ and/or the 3′ LTRs of a retrovirus such as a lentivirus. The LTR sequences may be LTR sequences from any lentivirus from any species. For example, they may be LTR sequences from HIV, SIV, FIV or BIV. Preferably the LTR sequences are HIV LTR sequences. In certain embodiments, the viral vector comprises the R and U5 sequences from the 5′ LTR of a lentivirus and an inactivated or “self-inactivating” 3′ LTR from a lentivirus. A “self-inactivating 3′ LTR” is a 3′ long terminal repeat (LTR) that contains a mutation, substitution or deletion that prevents the LTR sequences from driving expression of a downstream gene. A copy of the U3 region from the 3′ LTR acts as a template for the generation of both LTR's in the integrated provirus. Thus, when the 3′ LTR with an inactivating deletion or mutation integrates as the 5′ LTR of the provirus, no transcription from the 5′ LTR is possible. This eliminates competition between the viral enhancer/promoter and any internal enhancer/promoter. Self-inactivating 3′ LTRs are described, for example, in Zufferey et al, J Virol. 72:9873-9880, 1998; Miyoshi et al, J Virol. 72:8150-8157, 1998; and Iwakuma et al., J Virol. 261: 120-132, 1999, each of which is incorporated by reference in its entirety. Self-inactivating 3′ LTRs may be generated by any method known in the art. In certain embodiments, the U3 element of the 3′ LTR contains a deletion of its enhancer sequence, preferably the TATA box, Spl and/or NF-kappa B sites. As a result of the self-inactivating 3′ LTR, the provirus that is integrated into the host cell genome will comprise an inactivated 5′ LTR.

The vectors provided herein typically comprise a gene that encodes a protein (or other molecule, such as siRNA) that is desirably expressed in one or more target cells. In a viral vector, the gene of interest is preferably located between the 5′ LTR and 3′ LTR sequences. Further, the gene of interest is preferably in a functional relationship with other genetic elements, for example, transcription regulatory sequences such as promoters and/or enhancers, to regulate expression of the gene of interest in a particular manner once the gene is incorporated into the target cell. In certain embodiments, the useful transcriptional regulatory sequences are those that are highly regulated with respect to activity, both temporally and spatially.

In some embodiments, one or more additional genes may be incorporated as a safety measure, mainly to allow for the selective killing of transfected target cells within a heterogeneous population, such as within a human subject. In some embodiments, the selected gene is a thymidine kinase gene (TK), the expression of which renders a target cell susceptible to the action of the drug gancyclovir. In some embodiments, the suicide gene is a caspase 9 suicide gene activated by a dimerizing drug (see, e.g., Tey et al, Biology of Blood and Marrow Transplantation 13:913-924, 2007). In certain embodiments, a gene encoding a marker protein may be placed before or after the primary gene in a viral or non-viral vector to allow for identification and/or selection of cells that are expressing the desired protein. Certain embodiments incorporate a fluorescent marker protein, such as green fluorescent protein (GFP) or red fluorescent protein (RFP), along with the primary gene of interest. If one or more additional reporter genes are included, IRES sequences or 2A elements may also be included, separating the primary gene of interest from a reporter gene and/or any other gene of interest.

Certain embodiments may employ genes that encode one or more selectable markers. Examples include selectable markers that are effective in a eukaryotic cell or a prokaryotic cell, such as a gene for a drug resistance that encodes a factor necessary for the survival or growth of transformed host cells grown in a selective culture medium. Exemplary selection genes encode proteins that confer resistance to antibiotics or other toxins, e.g., G418, hygromycin B, puromycin, zeocin, ouabain, blasticidin, ampicillin, neomycin, methotrexate, or tetracycline, complement auxotrophic deficiencies, or supply may be present on a separate plasmid and introduced by co-transfection with the viral vector. In one embodiment, the gene encodes for a mutant dihydrofolate reductase (DHFR) that confers methotrexate resistance. Certain other embodiments may employ genes that encode one or cell surface receptors that can be used for tagging and detection or purification of transfected cells (e.g., low-affinity nerve growth factor receptor (LNGFR) or other such receptors useful as transduction tag systems. See e.g., Lauer et al., Cancer Gene Ther. 2000 March; 7(3):430-7.

Certain viral vectors such as retroviral vectors employ one or more heterologous promoters, enhancers, or both. In some embodiments, the U3 sequence from a retroviral or lentiviral 5′ LTR may be replaced with a promoter or enhancer sequence in the viral construct. Certain embodiments employ an “internal” promoter/enhancer that is located between the 5′ LTR and 3′ LTR sequences of the viral vector, and is operably linked to the gene of interest.

A “functional relationship” and “operably linked” mean, without limitation, that the gene is in the correct location and orientation with respect to the promoter and/or enhancer, such that expression of the gene will be affected when the promoter and/or enhancer is contacted with the appropriate regulatory molecules. Any enhancer/promoter combination may be used that either regulates (e.g., increases, decreases) expression of the viral RNA genome in the packaging cell line, regulates expression of the selected gene of interest in an infected target cell, or both.

A promoter is an expression control element formed by a DNA sequence that permits polymerase binding and transcription to occur. Promoters are untranslated sequences that are located upstream (5′) of the start codon of a selected gene of interest (typically within about 100 to 1000 bp) and control the transcription and translation of the coding polynucleotide sequence to which they are operably linked. Promoters may be inducible or constitutive. Inducible promoters initiate increased levels of transcription from DNA under their control in response to some change in culture conditions, such as a change in temperature. Promoters may be unidirectional or bidirectional. Bidirectional promoters can be used to co-express two genes, e.g., a gene of interest and a selection marker. Alternatively, a bidirectional promoter configuration comprising two promoters, each controlling expression of a different gene, in opposite orientation in the same vector may be utilized.

A variety of promoters are known in the art, as are methods for operably linking the promoter to the polynucleotide coding sequence. Both native promoter sequences and many heterologous promoters may be used to direct expression of the selected gene of interest. Certain embodiments employ heterologous promoters, because they generally permit greater transcription and higher yields of the desired protein as compared to the native promoter.

Certain embodiments may employ heterologous viral promoters. Examples of such promoters include those obtained from the genomes of viruses such as polyoma virus, fowlpox virus, adenovirus, bovine papilloma virus, avian sarcoma virus, cytomegalovirus, a retrovirus, hepatitis-B virus and Simian Virus 40 (SV40). Certain embodiments may employ heterologous mammalian promoter, such as the actin promoter, an immunoglobulin promoter, a heat-shock promoter, or a promoter that is associated with the native sequence of the gene of interest. Typically, the promoter is compatible with the target cell, such as a T cell.

Certain embodiments may employ one or more of the RNA polymerase II and III promoters. A suitable selection of RNA polymerase III promoters can be found, for example, in Paule and White. Nucleic Acids Research., Vol. 28, pp 1283-1298, 2000, which is incorporated by reference in its entirety. RNA polymerase II and III promoters also include any synthetic or engineered DNA fragments that can direct RNA polymerase II or III, respectively, to transcribe its downstream RNA coding sequences. Further, the RNA polymerase II or III (Pol II or III) promoter or promoters used as part of the viral vector can be inducible. Any suitable inducible Pol II or III promoter can be used with the methods described herein. Exemplary Pol II or III promoters include the tetracycline responsive promoters provided in Ohkawa and Taira, Human Gene Therapy, Vol. 11, pp 577-585, 2000; and Meissner et al, Nucleic Acids Research, Vol. 29, pp 1672-1682, 2001, each of which is incorporated by reference in its entirety.

Non-limiting examples of constitutive promoters that may be used include the promoter for ubiquitin, the CMV promoter (see, e.g., Karasuyama et al, J. Exp. Med. 169: 13, 1989), the β-actin (see, e.g., Gunning et al., PNAS USA 84:4831-4835, 1987), the elongation factor-1 alpha (EF-1 alpha) promoter, the CAG promoter, and the pgk promoter (see, e.g., Adra et al, Gene 60:65-74, 1987); Singer-Sam et al, Gene 32:409-417, 1984; and Dobson et al, Nucleic Acids Res. 10:2635-2637, 1982, each of which is incorporated by reference). Non-limiting examples of tissue specific promoters include the lck promoter (see, e.g., Garvin et al, Mol. Cell Biol. 8:3058-3064, 1988; and Takadera et al, Mol. Cell Biol. 9:2173-2180, 1989), the myogenin promoter (Yee et al, Genes and Development 7: 1277-1289. 1993), and the thyl promoter (see, e.g., Gundersen et al., Gene 1 13:207-214, 1992).

Additional examples of promoters include the ubiquitin-C promoter, the human heavy chain promoter or the Ig heavy chain promoter (e.g., MH), and the human K light chain promoter or the Ig light chain promoter (e.g., EEK), which are functional in B-lymphocytes. The MH promoter contains the human heavy chain promoter preceded by the iEμ enhancer flanked by matrix association regions, and the EEK promoter contains the x light chain promoter preceded an intronic enhancer (iEκ), a matrix associated region, and a 3′ enhancer (3Ex) (see, e.g., Luo et al, Blood. 1 13: 1422-1431, 2009, and U.S. Patent Application Publication No. 2010/0203630). Accordingly, certain embodiments may employ one or more of these promoter or enhancer elements.

In some embodiments, one promoter drives expression of a selectable marker and a second promoter drives expression of the gene of interest. As noted above, certain embodiments employ enhancer elements, such as an internal enhancer, to increase expression of the gene of interest. Enhancers are cis-acting elements of DNA, usually about 10 to 300 bp in length, that act on a promoter to increase its transcription. Enhancer sequences may be derived from mammalian genes (e.g., globin, elastase, albumin, a-fetoprotein, insulin), such as the enhancer, the intronic enhancer, and the 3′ enhancer. Also included are enhancers from a eukaryotic virus, including the SV40 enhancer on the late side of the replication origin (bp 100-270), the cytomegalovirus early promoter enhancer, the polyoma enhancer on the late side of the replication origin, and adenovirus enhancers. Enhancers may be spliced into the vector at a position 5′ or 3′ to the antigen-specific polynucleotide sequence, but are preferably located at a site 5′ from the promoter. Persons of skill in the art will select the appropriate enhancer based on the desired expression partem.

In some embodiments, promoters are selected to allow for inducible expression of the gene. A number of systems for inducible expression are known in the art, including the tetracycline responsive system and the lac operator-repressor system. It is also contemplated that a combination of promoters may be used to obtain the desired expression of the gene of interest. The skilled artisan will be able to select a promoter based on the desired expression pattern of the gene in the organism and/or the target cell of interest.

Certain viral vectors contain cis-acting packaging sequences to promote incorporation of the genomic viral RNA into the viral particle. Examples include psi-sequences. Such cis-acting sequences are known in the art. In certain embodiments, the viral vectors described herein may express two or more genes, which may be accomplished, for example, by incorporating an internal promoter that is operably linked to each separate gene beyond the first gene, by incorporating an element that facilitates co-expression such as an internal ribosomal entry sequence (IRES) element (U.S. Pat. No. 4,937,190, incorporated by reference) or a 2A element, or both. Merely by way of illustration, IRES or 2A elements may be used when a single vector comprises sequences encoding each chain of an immunoglobulin molecule with a desired specificity. For instance, the first coding region (encoding either the heavy or light chain) may be located immediately downstream from the promoter, and the second coding region (encoding the other chain) may be located downstream from the first coding region, with an IRES or 2A element located between the first and second coding regions, preferably immediately preceding the second coding region. In some embodiments, an IRES or 2A element is used to co-express an unrelated gene, such as a reporter gene, a selectable marker, or a gene that enhances immune function. Examples of IRES sequences that can be used include, without limitation, the IRES elements of encephalomyelitis virus (EMCV), foot- and-mouth disease virus (FMDV), Theiler's murine encephalomyelitis virus (TMEV), human rhinovirus (HRV), coxsackievirus (CSV), poliovirus (POLIO), Hepatitis A virus (HAV), Hepatitis C virus (HCV), and Pestiviruses (e.g., hog cholera virus (HOCV) and bovine viral diarrhea virus (BVDV)) (see, e.g., Le et al, Virus Genes 12: 135-147, 1996; and Le et al, Nuc. Acids Res. 25:362-369, 1997, each of which is incorporated by reference in their entirety). One example of a 2A element includes the F2A sequence from foot-and-mouth disease virus.

In some embodiments, the vectors provided herein also contain additional genetic elements to achieve a desired result. For example, certain viral vectors may include a signal that facilitates nuclear entry of the viral genome in the target cell, such as an HIV-1 flap signal. As a further example, certain viral vectors may include elements that facilitate the characterization of the provirus integration site in the target cell, such as a tRNA amber suppressor sequence. Certain viral vectors may contain one or more genetic elements designed to enhance expression of the gene of interest. For example, a woodchuck hepatitis virus responsive element (WRE) may be placed into the construct (see, e.g., Zufferey et al, J. Virol. 74:3668-3681, 1999; and Deglon et al, Hum. Gene Ther. 11: 179-190, 2000, each of which is incorporated by reference in its entirety). As another example, a chicken β-globin insulator may also be included in the construct. This element has been shown to reduce the chance of silencing the integrated DNA in the target cell due to methylation and heterochromatinization effects. In addition, the insulator may shield the internal enhancer, promoter and exogenous gene from positive or negative positional effects from surrounding DNA at the integration site on the chromosome. Certain embodiments employ each of these genetic elements. In another embodiment, the viral vectors provided herein may also contain a Ubiquitous Chromatin Opening Element (UCOE) to increase expression (see e.g., Zhang F, et al, Molecular Therapy: The journal of the American Society of Gene Therapy 2010 Sep.; 18(9): 1640-9.).

In some embodiments, the viral vectors (e.g., retroviral, lentiviral) provided herein are “pseudo-typed” with one or more selected viral glycoproteins or envelope proteins, mainly to target selected cell types. Pseudo-typing refers to generally to the incorporation of one or more heterologous viral glycoproteins onto the cell-surface virus particle, often allowing the virus particle to infect a selected cell that differs from its normal target cells. A “heterologous” element is derived from a virus other than the virus from which the RNA genome of the viral vector is derived. Typically, the glycoprotein-coding regions of the viral vector have been genetically altered such as by deletion to prevent expression of its own glycoprotein. Merely by way of illustration, the envelope glycoproteins gp41 and/or gp120 from an HIV-derived lentiviral vector are typically deleted prior to pseudo-typing with a heterologous viral glycoprotein.

In some embodiments, the viral vector is pseudo-typed with a heterologous viral glycoprotein that targets T lymphocytes. In some embodiments, the viral glycoprotein allows selective infection or transduction of resting or quiescent T lymphocytes. In some embodiments, the viral glycoprotein allows selective infection of T cells. In one embodiment, the viral vector is pseudo-typed with VSV-G. In some embodiments, the heterologous viral glycoprotein is derived from the glycoprotein of the measles virus, such as the Edmonton measles virus. In some embodiments, pseudo-type the measles virus glycoproteins hemagglutinin (H), fusion protein (F), or both (see, e.g., Frecha et al, Blood. 1 12:4843-52, 2008; and Frecha et al, Blood. 1 14:3173-80, 2009, each of which is incorporated by reference in its entirety). In some embodiments, the viral vector is pseudo-typed with gibbon ape leukemia virus (GALV). In some embodiments, the viral vector is pseudo-typed with cat endogenous retrovirus (RD 114). In some embodiments, the viral vector is pseudo-typed with baboon endogenous retrovirus (BaEV). In some embodiments, the viral vector is pseudo-typed with murine leukemia virus (MLV). In some embodiments, the viral vector comprises an embedded antibody binding domain, such as one or more variable regions (e.g., heavy and light chain variable regions) which serves to target the vector to a particular cell type.

Generation of viral vectors can be accomplished using any suitable genetic engineering techniques known in the art, including, without limitation, the standard techniques of restriction endonuclease digestion, ligation, transformation, plasmid purification, PCR amplification, and DNA sequencing, for example as described in Sambrook et al. (Molecular Cloning: A Laboratory Manual. Cold Spring Harbor Laboratory Press, N.Y. (1989)), Coffin et al. (Retroviruses. Cold Spring Harbor Laboratory Press, N.Y. (1997)) and “RNA Viruses: A Practical Approach” (Alan J. Cann, Ed., Oxford University Press, (2000)).

Any variety of methods known in the art may be used to produce suitable retroviral particles whose genome comprises an RNA copy of the viral vector. As one method, the viral vector may be introduced into a packaging cell line that packages the viral genomic RNA based on the viral vector into viral particles with a desired target cell specificity. The packaging cell line typically provides in trans the viral proteins that are required for packaging the viral genomic RNA into viral particles and infecting the target cell, including the structural gag proteins, the enzymatic pol proteins, and the envelope glycoproteins.

In some embodiments, the packaging cell line stably expresses certain necessary or desired viral proteins (e.g., gag, pol) (see, e.g., U.S. Pat. No. 6,218,181, herein incorporated by reference). In some embodiments, the packaging cell line is transiently transfected with plasmids that encode certain of the necessary or desired viral proteins (e.g., gag, pol, glycoprotein), including the measles virus glycoprotein sequences described herein. In some embodiments, the packaging cell line stably expresses the gag and pol sequences, and the cell line is then transfected with a plasmid encoding the viral vector and a plasmid encoding the glycoprotein. Following introduction of the desired plasmids, viral particles are collected and processed accordingly, such as by ultracentrifugation to achieve a concentrated stock of viral particles. Exemplary packaging cell lines include 293 (ATCC CCL X), HeLa (ATCC CCL 2), D17 (ATCC CCL 183), MDCK (ATCC CCL 34), BHK (ATCC CCL-10) and Cf2Th (ATCC CRL 1430) cell lines.

As used herein, “biological activity” or “bioactivity” refers to any response induced in an in vitro assay or in a cell, tissue, organ, or organism, (e.g., an animal, or a mammal, or a human) as the result of administering any compound, agent, polypeptide, conjugate, pharmaceutical composition contemplated herein. Biological activity may refer to agonistic actions or antagonistic actions. The biological activity may be a beneficial effect; or the biological activity may not be beneficial, i.e. a toxicity. In some embodiments, biological activity will refer to the positive or negative effects that a drug or pharmaceutical composition has on a living subject, e.g., a mammal such as a human. Accordingly, the term “biologically active” is meant to describe any compound possessing biological activity, as herein described. Biological activity may be assessed by any appropriate means currently known to the skilled artisan. Such assays may be qualitative or quantitative. The skilled artisan will readily appreciate the need to employ different assays to assess the activity of different polypeptides; a task that is routine for the average researcher. Such assays are often easily implemented in a laboratory setting with little optimization requirements, and more often than not, commercial kits are available that provide simple, reliable, and reproducible readouts of biological activity for a wide range of polypeptides using various technologies common to most labs. When no such kits are available, ordinarily skilled researchers can easily design and optimize in-house bioactivity assays for target polypeptides without undue experimentation; as this is a routine aspect of the scientific process.

“Therapeutic agent” refers to any compound that, when administered to a subject, (e.g., preferably a mammal, more preferably a human), in a therapeutically effective amount is capable of effecting treatment of a disease or condition as defined below.

As used herein, the term “treat” or “treating” or “treatment” embraces at least an amelioration of the symptoms associated with a disease or condition in the patient, where amelioration is used in a broad sense to refer to at least a reduction in the magnitude of a parameter, e.g. a symptom associated with the condition being treated. As such, “treatment” as used herein covers the treatment of the disease or condition of interest in a subject, preferably a human, having the disease or condition of interest, and includes: (i) preventing or inhibiting the disease or condition from occurring in a subject, in particular, when such subject is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition. As used herein, the terms “disease,” “disorder,” and “condition” may be used interchangeably or may be different in that the particular malady, injury or condition may not have a known causative agent (so that etiology has not yet been worked out), and it is, therefore, not yet recognized as an injury or disease but only as an undesirable condition or syndrome, wherein a more or less specific set of symptoms have been identified by clinicians.

As used herein, “therapeutically effective” refers to an amount of progenitor exhausted T cells expanded using the methods described herein, that is sufficient to treat or ameliorate, or in some manner reduce the symptoms associated with a disease or disorder, such as enzyme deficiency, protein deficiency, hormone deficiency, inflammation, cancer, autoimmunity, or infection. When used with reference to a method, the method is sufficiently effective to treat or ameliorate, or in some manner reduce the symptoms associated with a disease or condition. For example, an effective amount in reference to a disease is that amount which is sufficient to block or prevent its onset; or if disease pathology has begun, to palliate, ameliorate, stabilize, reverse or slow progression of the disease, or otherwise reduce pathological consequences of the disease. In any case, an effective amount may be given in single or divided doses.

The term “combination” refers to either a fixed combination in one dosage unit form, or a kit of parts for the combined administration where progenitor exhausted T cells expanded using the methods described herein and a combination partner (e.g., another drug as explained below, also referred to as “therapeutic agent” or “co-agent”) may be administered independently at the same time or separately within time intervals. In some circumstances the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein “adoptive cell therapy” refers to a treatment wherein any composition comprising cells suitable for adoptive cell transfer are administered to a subject. In one embodiment of the invention the adoptive cell therapy comprises a cell type selected from a group consisting of a tumor infiltrating lymphocyte (TIL), TCR (i.e. heterologous T-cell receptor) modified lymphocytes and CAR (i.e. chimeric antigen receptor) modified lymphocytes. In another embodiment of the invention, the adoptive cell therapeutic composition comprises a cell type selected from a group consisting of T-cells, CD8+ cells, progenitor exhausted T cells, CD4+ cells, NK-cells, delta-gamma T-cells, regulatory T-cells and peripheral blood mononuclear cells. In some embodiments, the adoptive cell therapy comprises TILs, T-cells, CD8+ cells, CD4+ cells, NK-cells, delta-gamma T-cells, regulatory T-cells or peripheral blood mononuclear cells. In some embodiments, the adoptive cell therapy comprises T cells. In some embodiments, the adoptive cell therapy comprises progenitor exhausted T cells expanded using the methods described herein. In some embodiments, the adoptive cell therapy comprises CD8+ progenitor exhausted T cells expanded using the methods described herein. As used herein “tumor-infiltrating lymphocytes” or TILs refer to white blood cells that have left the bloodstream and migrated into a tumor. Lymphocytes can be divided into three groups including B cells, T cells and natural killer cells. In some embodiments, the adoptive cell therapy comprises T-cells which have been modified with target-specific chimeric antigen receptors or specifically selected T-cell receptors. As used herein “T-cells” refers to CD3+ cells, including CD4+ helper cells, CD8+ cytotoxic T-cells and TS T cells.

### High-Throughput Screening Platform

In an aspect, the disclosure provides a method of screening for compounds that promote proliferation of progenitor exhausted T cells, comprising: (a) providing splenocytes from a transgenic subject comprising a polynucleotide encoding a labeled TCF-1 protein and a polynucleotide encoding a T cell receptor specific to a peptide antigen in complex with a major histocompatibility complex (MHC) molecule; (b) activated CD8+ T cells from the splenocytes in the presence of a compound; maintaining said culturing for a time period sufficient to permit proliferation of T cells, wherein T cells from the splenocytes have undergone stimulating by an activating agent, wherein stimulating occurs prior to or concurrently with culturing; (c) measuring: (i) an amount of labeled Tcf7+ T cells; and/or (ii) an amount of TCF-1 expression, wherein an increase in (i) and (ii) relative to splenocytes not treated with the compound identifies the compound as promoting proliferation of progenitor exhausted T cells.

Evaluating a compound being screened for its ability to decouple proliferation from differentiation may be done on CD8+ T cells, therefore T cells may undergo contact with the compound during activation or prior to activation of the T cells. In some embodiments, stimulating occurs prior to culturing. In some embodiments, stimulating occurs concurrently with culturing. In some embodiments, stimulating occurs prior to culturing or concurrently with culturing.

In some embodiments, the activating agent comprises one or more of the peptide antigen, antigen-presenting cells, anti-CD3, anti-CD28, Phorbol 12-myristate 13-acetate (PMA), and ionomycin.

In some embodiments, the proliferation of the T cells promoted by the compound uncouples T cell expansion from differentiation.

In some embodiments, the transgenic subject is a mammal. In some embodiments, the transgenic subject is a mouse.

The label of said labeled TCF-1 may comprise a fluorescent tag, wherein the tag is detectable upon irradiation, typically within defined wavelengths of light in the ultraviolet, visible, or infrared range (e.g., 200-800 nm), and can be detected by appropriate instrumentation (e.g., flow cytometer, plate reader, or microfluidics chip). In some embodiments, the label of said labeled TCF-1 comprises a fluorescent molecule, wherein when irradiated provides a distinguishable fluorescence emission spectrum. In one embodiment, the fluorescent molecule may be green fluorescent protein (GFP). In another embodiment, the fluorescent molecule may be mCherry. In another embodiment, the fluorescent molecule may be tdTomato. In another embodiment, the fluorescent molecule may be KeimaRed. In another embodiment, the fluorescent molecule may be yellow fluorescent protein (YFP). In another embodiment, the fluorescent molecule may be cyan fluorescent protein (CFP). Labeling can include, but is not limited to, the above intracellularly-expressed fluorescent proteins. Such proteins examples are discussed for GFP in Chalfie et al., (1994) Science 263:802-805. In some embodiments, the label of said labeled TCF-1 comprises enhanced green fluorescent protein (EGFP).

In some embodiments, the splenocytes comprise naïve splenocytes.

In some embodiments, the T cell receptor specific to a peptide antigen in complex with a major histocompatibility complex (MHC) molecule comprises a transgenic T cell receptor (TCR). In some embodiments, the transgenic TCR specifically recognizes an antigen peptide. In some embodiments, the transgenic TCR specifically recognizes an antigen peptide specific to a disease or disorder. In some embodiments, the transgenic TCR specifically recognizes an antigen peptide specific to cancer. In some embodiments, the transgenic TCR specifically recognizes an antigen peptide specific to infection. In some embodiments, the transgenic TCR specifically recognizes a ovalbumin (OVA) peptide. In some embodiments, the transgenic TCR specifically recognizes ovalbumin OVA257-264 presented by a MHC I molecule.

In some embodiments, culturing comprises suspending splenocytes in a culture medium. In some embodiments, media for use in the methods described herein includes, but is not limited to Iscove modified Dulbecco medium (with or without fetal bovine or other appropriate serum). Illustrative media also includes, but is not limited to, IMDM, RPMI 1640, AIM-V, DMEM, MEM, a-MEM, F-12, X-Vivo 15, and X-Vivo 20. In some embodiments, the medium may comprise a surfactant, an antibody, plasmanate or a reducing agent, one or more antibiotics, and/or additives such as insulin, transferrin, sodium selenite and cyclosporin.

In some embodiments, the culture medium comprises an antigen peptide. In some embodiments, the culture medium comprises an antigen peptide specific to a disease or disorder. In some embodiments, the culture medium comprises an antigen specific to cancer.

In some embodiments, the culture medium comprises an antigen specific to infection. In some embodiments, the culture medium comprises an antigen peptide, wherein the peptide is a ovalbumin peptide. In some embodiments, the ovalbumin peptide is OVA257-264. In some embodiments, the OVA257-264 peptide comprises a polypeptide having a sequence that is identical to SEQ ID NO: 1 (e.g., SIINFEKL). In some embodiments, the OVA257-264 peptide may comprise alternate derivatives, wherein the peptide comprises a sequence that is at least 70%, 75%, 80%, 85%, or 90% identical to SEQ ID NO: 1. In some embodiments, the OVA257-264 peptide may comprise alternate derivatives, wherein the peptide comprises a polypeptide having a sequence that is identical to SEQ ID NO: 2 (e.g., SAINFEKL) or SEQ ID NO: 3 (e.g., SIITFEKL).

In some embodiments, the antigen peptide is at a concentration of at least 10, 50, 100, 250, 500 nM, 1 μM, 2 μM, 5 μM, or more. In some embodiments, the antigen peptide is at a concentration between about 10 nM and about 100 nM. In some embodiments, the antigen peptide is at a concentration between about 100 nM and about 250 nM. In some embodiments, the antigen peptide is at a concentration between about 250 nM and about 500 nM. In some embodiments, the antigen peptide is at a concentration between about 500 nM and about 1 μM. In some embodiments, the antigen peptide is at a concentration between 1 μM and 2 μM. In some embodiments, the antigen peptide is at a concentration between 2 μM and 5 μM. In some embodiments, the antigen peptide is at a concentration between 5 μM and 10 μM. In some embodiments, the antigen peptide concentration is 250 nM or at least 250 nM. In some embodiments, the antigen peptide concentration is 500 nM or at least 500 nM. In some embodiments, the antigen peptide concentration is 750 nM or at least 750 nM. In some embodiments, the antigen peptide concentration is 1 μM or at least 1 μM. In some embodiments, the antigen peptide concentration is 1.5 μM or at least 1.5 μM. In some embodiments, the antigen peptide concentration is 3 μM or at least 3 μM.

In some embodiments, the ovalbumin peptide is at a concentration of at least 10 nM, 50 nM, 100 nM, 200 nM, 300 nM, 400 nM, 500 nM, 600 nM, 700 nM, 800 nM, 900 nM, 1 μM, or more. In some embodiments, the ovalbumin peptide is at a concentration between about 10 nM and about 100 nM. In some embodiments, the ovalbumin peptide is at a concentration between about 100 nM and about 250 nM. In some embodiments, the ovalbumin peptide is at a concentration between about 250 nM and about 500 nM. In some embodiments, the ovalbumin peptide is at a concentration between about 500 nM and about 1 μM. In some embodiments, the ovalbumin peptide is at a concentration between 1 μM and 2 μM. In some embodiments, the ovalbumin peptide is at a concentration between 2 μM and 5 μM. In some embodiments, the ovalbumin peptide is at a concentration between 5 μM and 10 μM. In some embodiments, the ovalbumin peptide is at a concentration of about 400 nM or at least 400 nM. In some embodiments, the ovalbumin peptide is at a concentration of about 500 nM or at least 500 nM. In some embodiments, the ovalbumin peptide is at a concentration of about 600 nM or at least 600 nM. In some embodiments, the ovalbumin peptide is at a concentration of about 700 nM or at least 700 nM. In some embodiments, the ovalbumin peptide is at a concentration of about 800 nM or at least 800 nM. In some embodiments, the ovalbumin peptide is at a concentration of about 900 nM or at least 900 nM. In some embodiments, the ovalbumin peptide is at a concentration of about 1 μM or at least 1 μM. In some embodiments, the ovalbumin peptide is at a concentration of about 1.1 μM or at least 1.1 μM.

In some embodiments, the culture media further comprises activating factors that may be added to the in vitro cell culture at various concentrations to achieve the desired outcome (e.g., expansion decoupled from differentiation). It is contemplated that a T cell activating factor may be utilized in the culture media for expanding the T cells and not in differentiating the T cells. For example, T cells may be cultured with one or more T cell activating factors including but not limited to IL-2. In some embodiments, the culture medium further comprises IL-2. In some embodiments, the amount of IL-2 is at least about 10, 20, 30, 40, 50, 60, 70, 80, 90, 100, 110, 120, 130, 140, 150, 160, 170, 180, 190, 200, 210, 220, 230, 240, 250, 300, 350, 400, 500, or 1000 IU/mL. In some embodiments, the amount of IL-2 is between about 10 and 100 IU/mL. In some embodiments, the amount of IL-2 is between about 100 and 200 IU/mL. In some embodiments, the amount of IL-2 is between about 200 and 500 IU/mL. In some embodiments, the amount of IL-2 is between about 500 and 1000 IU/mL. In some embodiments, the amount of IL-2 is about 10 IU/mL or at least 10 IU/mL. In some embodiments, the amount of IL-2 is about 20 IU/mL or at least 20 IU/mL. In some embodiments, the amount of IL-2 is about 30 IU/mL or at least 30 IU/mL. In some embodiments, the amount of IL-2 is about 40 IU/mL or at least 40 IU/mL. In some embodiments, the amount of IL-2 is about 50 IU/mL or at least 50 IU/mL. In some embodiments, the amount of IL-2 is about 60 IU/mL or at least 60 IU/mL. In some embodiments, the amount of IL-2 is about 70 IU/mL or at least 70 IU/mL. In some embodiments, the amount of IL-2 is about 90 IU/mL or at least 90 IU/mL. In some embodiments, the amount of IL-2 is about 10 IU/mL or at least 10 IU/mL. In some embodiments, the amount of IL-2 is about 100 IU/mL or at least 100 IU/mL. In some embodiments, the amount of IL-2 is about 110 IU/mL or at least 110 IU/mL. In some embodiments, the amount of IL-2 is about 120 IU/mL or at least 120 IU/mL. In some embodiments, the amount of IL-2 is about 130 IU/mL or at least 130 IU/mL. In some embodiments, the amount of IL-2 is about 140 IU/mL or at least 140 IU/mL. In some embodiments, the amount of IL-2 is about 150 IU/mL or at least 150 IU/mL. In some embodiments, the amount of IL-2 is about 160 IU/mL or at least 160 IU/mL. In some embodiments, the amount of IL-2 is about 170 IU/mL or at least 170 IU/mL. In some embodiments, the amount of IL-2 is about 180 IU/mL or at least 180 IU/mL. In some embodiments, the amount of IL-2 is about 190 IU/mL or at least 190 IU/mL. In some embodiments, the amount of IL-2 is about 200 IU/mL or at least 200 IU/mL. In some embodiments, the amount of IL-2 is about 210 IU/mL or at least 210 IU/mL. In some embodiments, the amount of IL-2 is about 220 IU/mL or at least 220 IU/mL. In some embodiments, the amount of IL-2 is about 230 IU/mL or at least 230 IU/mL. In some embodiments, the amount of IL-2 is about 240 IU/mL or at least 240 IU/mL. In some embodiments, the amount of IL-2 is about 250 IU/mL or at least 250 IU/mL. In some embodiments, the amount of IL-2 is about 60 IU/mL.

In some embodiments, culturing comprises adding the compound being screened at about on day 0 of culturing. In some embodiments, culturing comprises adding the compound being screened at about day 1, 2, 3, 4, 5, 6, 7, 8, 9, 10, or more after the start of culturing. In some embodiments, culturing comprises adding the compound being screened at about day 0 of culturing or at about day 3 after the start of culturing. In some embodiments, culturing comprises adding the compound being screened at about day 0 of culturing or at about day 5 after the start of culturing. In some embodiments, culturing comprises adding the compound being screened at about day 0 of culturing. In some embodiments, culturing comprises adding the compound being screened at about day 1 after the start of culturing. In some embodiments, culturing comprises adding the compound being screened at about day 2 after the start of culturing. In some embodiments, culturing comprises adding the compound being screened at about day 3 after the start of culturing. In some embodiments, culturing comprises adding the compound being screened at about day 4 after the start of culturing. In some embodiments, culturing comprises adding the compound being screened at about day 5 after the start of culturing. In some embodiments, culturing comprises adding the compound being screened at about day 6 after the start of culturing.

In some embodiments, the time period sufficient to permit stimulation and/or proliferation of T cells during culturing is about 1, 2, 3, 4, 5, 6, 7, 8, 9, 10, or more days. In some embodiments, the time period sufficient to permit stimulation and/or proliferation of T cells during culturing is about 1 day. In some embodiments, the time period sufficient to permit stimulation and/or proliferation of T cells during culturing is about 2 days. In some embodiments, the time period sufficient to permit stimulation and/or proliferation of T cells during culturing is about 3 days. In some embodiments, the time period sufficient to permit stimulation and/or proliferation of T cells during culturing is about 4 days. In some embodiments, the time period sufficient to permit stimulation and/or proliferation of T cells during culturing is about 5 days. In some embodiments, the time period sufficient to permit stimulation and/or proliferation of T cells during culturing is about 6 days. In some embodiments, the time period sufficient to permit stimulation and/or proliferation of T cells during culturing is about 7 days. In some embodiments, the time period sufficient to permit stimulation and/or proliferation of T cells during culturing is about 8 days. In some embodiments, the time period sufficient to permit stimulation and/or proliferation of T cells during culturing is about 9 days. In some embodiments, the time period sufficient to permit stimulation and/or proliferation of T cells during culturing is about 10 days. In some embodiments, the time period sufficient to permit stimulation and/or proliferation of T cells during culturing is between about 1 and about 3 days, wherein at which time T cells undergo the measuring of (c). In some embodiments, the time period sufficient to permit stimulation and/or proliferation of T cells during culturing is between about 3 and about 5 days, wherein at which time T cells undergo the measuring of (c). In some embodiments, the time period sufficient to permit stimulation and/or proliferation of T cells during culturing is between about 5 and about 10 days, wherein at which time T cells undergo the measuring of (c).

In an aspect, the disclosure provides a method of screening for compounds that promote proliferation of antigen-specific progenitor exhausted T cells, comprising: (a) providing splenocytes from a transgenic subject comprising a polynucleotide encoding a T cell receptor specific to a peptide antigen; (b) culturing activated CD8+ T cells from the splenocytes in the presence of a compound; maintaining said culturing for a time period sufficient to permit proliferation of T cells, wherein T cells from the splenocytes have undergone stimulating by an activating agent, wherein stimulating occurs prior to or concurrently with culturing; (c) contacting a detection antibody with a subsample of T cells, wherein the detection antibody comprises a detectable label, wherein the antibody is specific to PD-1 or Tim3, thereby generating a mixture comprising the detection antibody and the T cells, and maintaining said mixture for a time period sufficient to permit the detection antibody to bind to the specific antigens present on the T cells; (d) washing said mixture to remove unbound detection antibody from the T cells; (e) measuring: (i) an amount of T cells bound by the detection antibody; and/or (ii) an amount of signal from the detection antibody; (f) re-stimulating the T cells in the culture, and maintaining said culturing for a time period sufficient to permit stimulation and/or proliferation of T cells; (g) expanding the T cells in a culture medium, and maintaining said expanding for a time period sufficient to permit proliferation of T cells; (h) contacting a detection antibody with a subsample of expanded T cells, wherein the detection antibody comprises a detectable label, wherein the antibody is specific to PD-1 or Tim3, thereby generating a mixture comprising the detection antibody and the T cells, and maintaining said mixture for a time period sufficient to permit the detection antibody to bind to the specific antigens present on the T cells; (i) washing said mixture to remove unbound detection antibody from the T cells; (j) measuring: (i) an amount of T cells bound by the detection antibody; and/or (ii) an amount of signal from the detection antibody, wherein an increase in either the amount of T cells bound by the detection antibody and the amount of signal from the detection antibody of (e) or the amount of T cells bound by the detection antibody and the amount of signal from the detection antibody of ( ) for PD-1 and TCF-1 and a decrease, or absence, in either the amount of T cells bound by the detection antibody and the amount of signal from the detection antibody of (e) or the amount of T cells bound by the detection antibody and the amount of signal from the detection antibody of ( ) for Tim3 relative to splenocytes not treated with the compound identifies the compound as promoting proliferation of antigen-specific progenitor exhausted T cells.

In some embodiments, the activating agent comprises one or more of the peptide antigen, antigen-presenting cells, anti-CD3, anti-CD28, Phorbol 12-myristate 13-acetate (PMA), and ionomycin.

In some embodiments, the proliferation of the T cells promoted by the compound uncouples T cell expansion from differentiation.

In some embodiments, the transgenic subject is a mammal. In some embodiments, the transgenic subject is a mouse.

In some embodiments, the label of said detection antibody may comprise a fluorescent tag, wherein the tag is detectable upon irradiation, typically within defined wavelengths of light in the ultraviolet, visible, or infrared range (e.g., 200-800 nm), and can be detected by appropriate instrumentation (e.g., flow cytometer, plate reader, or microfluidics chip). In some embodiments, the label of said detection antibody comprises a fluorescent molecule, wherein when irradiated provides a distinguishable fluorescence emission spectrum. In one embodiment, the fluorescent molecule may be green fluorescent protein (GFP). In another embodiment, the fluorescent molecule may be mCherry. In another embodiment, the fluorescent molecule may be tdTomato. In another embodiment, the fluorescent molecule may be KeimaRed. In another embodiment, the fluorescent molecule may be yellow fluorescent protein (YFP). In another embodiment, the fluorescent molecule may be cyan fluorescent protein (CFP). Labeling can include, but is not limited to, the above fluorescent proteins. Such proteins examples are discussed for GFP in Chalfie et al., (1994) Science 263:802-805. In some embodiments, the label of said detection antibody comprises enhanced green fluorescent protein (EGFP).

In some embodiments, the T cell receptor specific to a peptide antigen comprises a transgenic T cell receptor (TCR). In some embodiments, the transgenic TCR specifically recognizes an antigen peptide. In some embodiments, the transgenic TCR specifically recognizes an antigen peptide specific to a disease or disorder. In some embodiments, the transgenic TCR specifically recognizes an antigen peptide specific to cancer. In some embodiments, the transgenic TCR specifically recognizes an antigen peptide specific to infection. In some embodiments, the transgenic TCR specifically recognizes a glycoprotein of Lymphocytic choriomeningitis virus peptide (GP). In some embodiments, the transgenic TCR specifically recognizes GP33-41.

In some embodiments, culturing comprises suspending splenocytes in a culture medium. In some embodiments, culturing comprises suspending splenocytes in a culture medium. In some embodiments, media for use in the methods described herein includes, but is not limited to Iscove modified Dulbecco medium (with or without fetal bovine or other appropriate serum). Illustrative media also includes, but is not limited to, IMDM, RPMI 1640, AIM-V, DMEM, MEM, a-MEM, F-12, X-Vivo 15, and X-Vivo 20. In some embodiments, the medium may comprise a surfactant, an antibody, plasmanate or a reducing agent, one or more antibiotics, and/or additives such as insulin, transferrin, sodium selenite and cyclosporin.

In some embodiments, the culture medium comprises an antigen peptide. In some embodiments, the culture medium comprises an antigen peptide specific to a disease or disorder. In some embodiments, the culture medium comprises an antigen specific to cancer.

In some embodiments, the culture medium comprises an antigen specific to infection. In some embodiments, the culture medium comprises an antigen peptide, wherein the peptide is a glycoprotein of Lymphocytic choriomeningitis virus peptide. In some embodiments, the glycoprotein of Lymphocytic choriomeningitis virus peptide is GP33-41. In some embodiments, the GP33-41 peptide comprises a polypeptide having a sequence that is identical to SEQ ID NO: 4 (e.g., KAVYNFATM). In some embodiments, the GP33-41 peptide may comprise alternate derivatives, wherein the peptide comprises a sequence that is at least 70%, 75%, 80%, 85%, or 90% identical to SEQ ID NO: 4.

In some embodiments, the glycoprotein of Lymphocytic choriomeningitis virus peptide is at a concentration of about at least 10 nM, 50 nM, 100 nM, 200 nM, 300 nM, 400 nM, 500 nM, 600 nM, 700 nM, 800 nM, 900 nM, 1 μM, 2 μM, 3 μM, 4 μM, 5 μM or more. In some embodiments, the glycoprotein of Lymphocytic choriomeningitis virus peptide is at a concentration between about 10 nM and about 100 nM. In some embodiments, the glycoprotein of Lymphocytic choriomeningitis virus peptide is at a concentration between about 100 nM and about 250 nM. In some embodiments, the glycoprotein of Lymphocytic choriomeningitis virus peptide is at a concentration between about 250 nM and about 500 nM. In some embodiments, the glycoprotein of Lymphocytic choriomeningitis virus peptide is at a concentration between about 500 nM and about 1 μM. In some embodiments, the glycoprotein of Lymphocytic choriomeningitis virus peptide is at a concentration between about 1 μM and about 2 μM. In some embodiments, the glycoprotein of Lymphocytic choriomeningitis virus peptide is at a concentration between about 2 μM and about 3 μM. In some embodiments, the glycoprotein of Lymphocytic choriomeningitis virus peptide is at a concentration between about 3 μM and about 4 μM. In some embodiments, the glycoprotein of Lymphocytic choriomeningitis virus peptide is at a concentration between about 4 μM and about 5 μM. In some embodiments, the glycoprotein of Lymphocytic choriomeningitis virus peptide is at a concentration of about 400 nM or at least 400 nM. In some embodiments, the glycoprotein of Lymphocytic choriomeningitis virus peptide is at a concentration of about 500 nM or at least 500 nM. In some embodiments, the glycoprotein of Lymphocytic choriomeningitis virus peptide is at a concentration of about 600 nM or at least 600 nM. In some embodiments, the glycoprotein of Lymphocytic choriomeningitis virus peptide is at a concentration of about 700 nM or at least 700 nM. In some embodiments, the glycoprotein of Lymphocytic choriomeningitis virus peptide is at a concentration of about 800 nM or at least 800 nM. In some embodiments, the glycoprotein of Lymphocytic choriomeningitis virus peptide is at a concentration of about 900 nM or at least 900 nM. In some embodiments, the glycoprotein of Lymphocytic choriomeningitis virus peptide is at a concentration of about 1 μM or at least 1 μM. In some embodiments, the glycoprotein of Lymphocytic choriomeningitis virus peptide is at a concentration of about 1.1 μM or at least 1.1 μM. In some embodiments, the glycoprotein of Lymphocytic choriomeningitis virus peptide is at a concentration of about 1.2 μM or at least 1.2 μM. In some embodiments, the glycoprotein of Lymphocytic choriomeningitis virus peptide is at a concentration of about 1.3 μM or at least 1.3 μM. In some embodiments, the glycoprotein of Lymphocytic choriomeningitis virus peptide is at a concentration of about 1.4 μM or at least 1.4 μM. In some embodiments, the glycoprotein of Lymphocytic choriomeningitis virus peptide is at a concentration of about 1.5 μM or at least 1.5 μM. In some embodiments, the glycoprotein of Lymphocytic choriomeningitis virus peptide is at a concentration of about 1.0 μM.

In some embodiments, the culture media further comprises activating factors that may be added to the in vitro cell culture at various concentrations to achieve the desired outcome (e.g., expansion decoupled from differentiation). It is contemplated that a T cell activating factor may be utilized in the culture media for expanding the T cells and not in differentiating the T cells. For example, T cells may be cultured with one or more T cell activating factors including but not limited to IL-2. In some embodiments, the culture media comprises IL-2. In some embodiments, the amount of IL-2 is at least about 10, 20, 30, 40, 50, 60, 70, 80, 90, 100, 110, 120, 130, 140, 150, 160, 170, 180, 190, 200, 210, 220, 230, 240, 250, 300, 350, 400, 500, 1000 IU/mL, or more. In some embodiments, the amount of IL-2 is between about 10 and 100 IU/mL. In some embodiments, the amount of IL-2 is between about 100 and 200 IU/mL. In some embodiments, the amount of IL-2 is between about 200 and 500 IU/mL. In some embodiments, the amount of IL-2 is between about 500 and 1000 IU/mL. In some embodiments, the amount of IL-2 is about 10 IU/mL or at least 10 IU/mL. In some embodiments, the amount of IL-2 is about 20 IU/mL or at least 20 IU/mL. In some embodiments, the amount of IL-2 is about 30 IU/mL or at least 30 IU/mL. In some embodiments, the amount of IL-2 is about 40 IU/mL or at least 40 IU/mL. In some embodiments, the amount of IL-2 is about 50 IU/mL or at least 50 IU/mL. In some embodiments, the amount of IL-2 is about 60 IU/mL or at least 60 IU/mL. In some embodiments, the amount of IL-2 is about 70 IU/mL or at least 70 IU/mL. In some embodiments, the amount of IL-2 is about 90 IU/mL or at least 90 IU/mL. In some embodiments, the amount of IL-2 is about 10 IU/mL or at least 10 IU/mL. In some embodiments, the amount of IL-2 is about 100 IU/mL or at least 100 IU/mL. In some embodiments, the amount of IL-2 is about 110 IU/mL or at least 110 IU/mL. In some embodiments, the amount of IL-2 is about 120 IU/mL or at least 120 IU/mL. In some embodiments, the amount of IL-2 is about 130 IU/mL or at least 130 IU/mL. In some embodiments, the amount of IL-2 is about 140 IU/mL or at least 140 IU/mL. In some embodiments, the amount of IL-2 is about 150 IU/mL or at least 150 IU/mL. In some embodiments, the amount of IL-2 is about 160 IU/mL or at least 160 IU/mL. In some embodiments, the amount of IL-2 is about 170 IU/mL or at least 170 IU/mL. In some embodiments, the amount of IL-2 is about 180 IU/mL or at least 180 IU/mL. In some embodiments, the amount of IL-2 is about 190 IU/mL or at least 190 IU/mL. In some embodiments, the amount of IL-2 is about 200 IU/mL or at least 200 IU/mL. In some embodiments, the amount of IL-2 is about 210 IU/mL or at least 210 IU/mL. In some embodiments, the amount of IL-2 is about 220 IU/mL or at least 220 IU/mL. In some embodiments, the amount of IL-2 is about 230 IU/mL or at least 230 IU/mL. In some embodiments, the amount of IL-2 is about 240 IU/mL or at least 240 IU/mL. In some embodiments, the amount of IL-2 is about 250 IU/mL or at least 250 IU/mL. In some embodiments, the amount of IL-2 is about 60 IU/mL. In some embodiments, the amount of IL-2 is about 200 IU/mL.

In some embodiments, culturing comprises adding the compound being screened at about day 0 of culturing, wherein the concentration of the compound is maintained throughout culturing. In some embodiments, culturing comprises adding the compound being screened at about day 0 of culturing, wherein the concentration of the compound is increased throughout culturing. In some embodiments, culturing comprises adding the compound being screened at about day 0 of culturing, wherein the concentration of the compound is decreased throughout culturing. In some embodiments, culturing comprises adding the compound being screened at about day 0 of culturing. In some embodiments, culturing comprises adding the compound being screened at about day 1, 2, 3, 4, 5, 6, 7, 8, 9, 10, or more after the start of culturing. In some embodiments, culturing comprises adding the compound being screened at about day 0 of culturing or at about day 3 after the start of culturing. In some embodiments, culturing comprises adding the compound being screened at about day 0 of culturing or at about day 5 after the start of culturing. In some embodiments, culturing comprises adding the compound being screened at about day 0 of culturing. In some embodiments, culturing comprises adding the compound being screened at about day 1 after the start of culturing. In some embodiments, culturing comprises adding the compound being screened at about day 2 after the start of culturing. In some embodiments, culturing comprises adding the compound being screened at about day 3 after the start of culturing. In some embodiments, culturing comprises adding the compound being screened at about day 4 after the start of culturing. In some embodiments, culturing comprises adding the compound being screened at about day 5 after the start of culturing. In some embodiments, culturing comprises adding the compound being screened at about day 6 after the start of culturing.

In some embodiments, the time period sufficient to permit stimulation and/or proliferation of T cells during culturing is about 1, 2, 3, 4, 5, 6, 7, 8, 9, 10, or more days. In some embodiments, the time period sufficient to permit stimulation and/or proliferation of T cells during culturing is about 1 day. In some embodiments, the time period sufficient to permit stimulation and/or proliferation of T cells during culturing is about 2 days. In some embodiments, the time period sufficient to permit stimulation and/or proliferation of T cells during culturing is about 3 days. In some embodiments, the time period sufficient to permit stimulation and/or proliferation of T cells during culturing is about 4 days. In some embodiments, the time period sufficient to permit stimulation and/or proliferation of T cells during culturing is about 5 days In some embodiments, the time period sufficient to permit stimulation and/or proliferation of T cells during culturing is about 6 days. In some embodiments, the time period sufficient to permit stimulation and/or proliferation of T cells during culturing is about 7 days. In some embodiments, the time period sufficient to permit stimulation and/or proliferation of T cells during culturing is about 8 days. In some embodiments, the time period sufficient to permit stimulation and/or proliferation of T cells during culturing is about 9 days. In some embodiments, the time period sufficient to permit stimulation and/or proliferation of T cells during culturing is about 10 days. In some embodiments, the measuring of (e) occurs at about day 1, 2, 3, 4, 5, 6, 7, 8, 9, 10, or more days after the start of culturing. In some embodiments, the measuring of (e) occurs at about day 1 after the start of culturing. In some embodiments, the measuring of (e) occurs at about day 2 after the start of culturing. In some embodiments, the measuring of (e) occurs at about day 3 after the start of culturing. In some embodiments, the measuring of (e) occurs at about day 4 after the start of culturing. In some embodiments, the measuring of (e) occurs at about day 5 after the start of culturing. In some embodiments, the measuring of (e) occurs at about day 6 after the start of culturing. In some embodiments, the measuring of (e) occurs at about day 7 after the start of culturing. In some embodiments, the measuring of (e) occurs at about day 8 after the start of culturing. In some embodiments, the measuring of (e) occurs at about day 9 after the start of culturing. In some embodiments, the measuring of (e) occurs at about day 10 after the start of culturing.

In some embodiments, re-stimulating the T cells in the culture comprises spiking the culture medium with anti-CD3, anti-CD28, and/or IL-2. In some embodiments, the amount of IL-2 is at least about 10, 20, 30, 40, 50, 60, 70, 80, 90, 100, 110, 120, 130, 140, 150, 160, 170, 180, 190, 200, 210, 220, 230, 240, 250, 300, 350, 400, 500, 1000 IU/mL, or more. In some embodiments, the amount of IL-2 is between about 10 and 100 IU/mL. In some embodiments, the amount of IL-2 is between about 100 and 200 IU/mL. In some embodiments, the amount of IL-2 is between about 200 and 500 IU/mL. In some embodiments, the amount of IL-2 is between about 500 and 1000 IU/mL. In some embodiments, the amount of IL-2 is about 10 IU/mL or at least 10 IU/mL. In some embodiments, the amount of IL-2 is about 20 IU/mL or at least 20 IU/mL. In some embodiments, the amount of IL-2 is about 30 IU/mL or at least 30 IU/mL. In some embodiments, the amount of IL-2 is about 40 IU/mL or at least 40 IU/mL. In some embodiments, the amount of IL-2 is about 50 IU/mL or at least 50 IU/mL. In some embodiments, the amount of IL-2 is about 60 IU/mL or at least 60 IU/mL. In some embodiments, the amount of IL-2 is about 70 IU/mL or at least 70 IU/mL. In some embodiments, the amount of IL-2 is about 90 IU/mL or at least 90 IU/mL. In some embodiments, the amount of IL-2 is about 10 IU/mL or at least 10 IU/mL. In some embodiments, the amount of IL-2 is about 100 IU/mL or at least 100 IU/mL. In some embodiments, the amount of IL-2 is about 110 IU/mL or at least 110 IU/mL. In some embodiments, the amount of IL-2 is about 120 IU/mL or at least 120 IU/mL. In some embodiments, the amount of IL-2 is about 130 IU/mL or at least 130 IU/mL. In some embodiments, the amount of IL-2 is about 140 IU/mL or at least 140 IU/mL. In some embodiments, the amount of IL-2 is about 150 IU/mL or at least 150 IU/mL. In some embodiments, the amount of IL-2 is about 160 IU/mL or at least 160 IU/mL. In some embodiments, the amount of IL-2 is about 170 IU/mL or at least 170 IU/mL. In some embodiments, the amount of IL-2 is about 180 IU/mL or at least 180 IU/mL. In some embodiments, the amount of IL-2 is about 190 IU/mL or at least 190 IU/mL. In some embodiments, the amount of IL-2 is about 200 IU/mL or at least 200 IU/mL. In some embodiments, the amount of IL-2 is about 210 IU/mL or at least 210 IU/mL. In some embodiments, the amount of IL-2 is about 220 IU/mL or at least 220 IU/mL. In some embodiments, the amount of IL-2 is about 230 IU/mL or at least 230 IU/mL. In some embodiments, the amount of IL-2 is about 240 IU/mL or at least 240 IU/mL. In some embodiments, the amount of IL-2 is about 250 IU/mL or at least 250 IU/mL. In some embodiments, the amount of IL-2 is about 200 IU/mL.

In some embodiments, the time period sufficient to permit stimulation and/or proliferation of T cells during the re-simulating of (d) is between about 12 and 24 hours. In some embodiments, the time period sufficient to permit stimulation and/or proliferation of T cells during the re-simulating of (d) is between about 24 and 36 hours. In some embodiments, the time period sufficient to permit stimulation and/or proliferation of T cells during the re-simulating of (d) is between about 36 and 48 hours. In some embodiments, the time period sufficient to permit stimulation and/or proliferation of T cells during the re-simulating of (d) is between about 48 and 60 hours. In some embodiments, the time period sufficient to permit stimulation and/or proliferation of T cells during the re-simulating of (d) is between about 60 and 72 hours. In some embodiments, the time period sufficient to permit stimulation and/or proliferation of T cells during the re-simulating of (d) is between about 24 and 72 hours. In some embodiments, the time period sufficient to permit stimulation and/or proliferation of T cells during the re-simulating of (d) is at least about 10, 20, 40, 50, 60, or 70 hours. In some embodiments, the time period sufficient to permit stimulation and/or proliferation of T cells during the re-simulating of (d) is about 38 hours. In some embodiments, the time period sufficient to permit stimulation and/or proliferation of T cells during the re-simulating of (d) is about 48 hours. In some embodiments, the time period sufficient to permit stimulation and/or proliferation of T cells during the re-simulating of (d) is about 58 hours.

In some embodiments, the time period sufficient to permit proliferation of T cells during the expanding of (e) is about 24 hours.

In some embodiments, the time period sufficient to permit proliferation of T cells during the expanding of (e) is between about 6 and 12 hours. In some embodiments, the time period sufficient to permit proliferation of T cells during the expanding of (e) is between about 12 and 18 hours. In some embodiments, the time period sufficient to permit proliferation of T cells during the expanding of (e) is between about 18 and 24 hours. In some embodiments, the time period sufficient to permit proliferation of T cells during the expanding of (e) is between about 24 and 30 hours. In some embodiments, the time period sufficient to permit proliferation of T cells during the expanding of (e) is between about 12 and 36 hours. In some embodiments, the time period sufficient to permit proliferation of T cells during the expanding of (e) is at least about 5, 10, 15, 20, or 25 hours. In some embodiments, the time period sufficient to permit proliferation of T cells during the expanding of (e) is about 18 hours. In some embodiments, the time period sufficient to permit proliferation of T cells during the expanding of (e) is about 24 hours. In some embodiments, the time period sufficient to permit proliferation of T cells during the expanding of (e) is about 30 hours.

In some embodiments, the measuring of 0) occurs at about day 5, 6, 7, 8, 9, 10, 11, 12, 13, 14, 15, 16, 17, 18, 19, 20 after the start of culturing. In some embodiments, the measuring of 0) occurs between about day 5 or about day 9 after the start of culturing. In some embodiments, the measuring of 0) occurs between about day 9 or about day 12 after the start of culturing. In some embodiments, the measuring of 0) occurs between about day 12 or about day 15 after the start of culturing. In some embodiments, the measuring of 0) occurs between about day 15 or about day 18 after the start of culturing. In some embodiments, the measuring of (j) occurs at about day 7 after the start of culturing. In some embodiments, the measuring of (j) occurs at about day 8 after the start of culturing. In some embodiments, the measuring of (j) occurs at about day 9 after the start of culturing. In some embodiments, the measuring of (j) occurs at about day 10 after the start of culturing. In some embodiments, the measuring of (j) occurs at about day 11 after the start of culturing. In some embodiments, the measuring of (j) occurs at about day 12 after the start of culturing.

Use of Sugars and Other Compounds that Uncouple T Cell Expansion from Differentiation

In an aspect, the disclosure provides methods for expanding activated T cells to produce T cells with enhanced in vivo persistence after adoptive transfer, utilizing one of the compounds described herein that are able to uncouple T cell expansion from differentiation. In an related aspect, the invention provides methods for maintaining the less-differentiated status (e.g., ‘stemness”) of a population of activated T cells (e.g., progenitor exhausted T cells). In some embodiments, the employed compound for practicing methods of the invention can be selected from, e.g., sugar compounds such as GlcNAc and Neu5Ac, EZH2 inhibitors, or KRAS (G12C) inhibitors or analogues. In some embodiments, the employed KRAS (G12C) inhibitor is Inhibitor 9 or Inhibitor 12 as exemplified herein. In some embodiments, the employed KRAS (G12C) inhibitor is an analogue of KRAS (G12C) Inhibitor 9 or 12. Typically, like Inhibitor 9 or 12, the analogue contains a cysteine-reactive group (sulphonamide or acrylamide) to enable covalent binding to a cysteine residue in a target protein. Examples of such analogues are compounds Z7 and Z6 as described herein.

In some other embodiments, the employed compound can be selected from purine and pyrimidine biosynthesis intermediates and end products, as well as inhibitor compounds of glucose-6-phosphate dehydrogenase, as described herein. Various activated T cells are amenable to expansion with the methods. These include, e.g., stem memory T cells (Tscm), central memory T cells (Tcm), effector memory T cells, effector T cells, progenitor exhausted T cells (Tpex), or terminally exhausted T cells (Ttex). In some embodiments, the produced T cells can be assessed for a less differentiated state relative to the activated T cells not treated with the compound. In some embodiments, the methods are directed to promoting expansion and/or proliferation of progenitor exhausted T cell with enhanced expansion and/or persistence, comprising contacting a cell population comprising progenitor exhausted T cells with an effective amount of a compound. In some other embodiments, the invention provides methods of using the same compounds (e.g., sugar derivative compounds such as Neu5Ac and GlcNAc) to maintain TCF positive T cells, or to convert TCF-1 negative T cells into TCF-1 positive or partially positive. Some of these methods are directed to reversing the TCF negative phenotype of tumor infiltrating T cells such as terminal exhausted Tim-3⁺TCF⁻ cells.

In some embodiments, the proliferation of the T cells promoted by the compound uncouples T cell expansion from differentiation. In some embodiments, the effective amount is an amount sufficient to cause the proliferation of a cell or cell population.

In some embodiments, the compound is a sugar. In some embodiments, the sugar is trehalose, sucrose, lactose, glucose, galactose, fructose, neuraminic acid, or a derivative thereof. In some embodiments, the sugar is at a concentration above physiological levels. In some embodiments, the concentration is at least about 1 mM, 2 mM, 3 mM, 4 mM, 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 150 mM, 200 mM, 250 mM, 300 mM, 350 mM, 400 mM, 450 mM 500 mM, or more. In some embodiments, the concentration is at least about 10 mM, 20 mM, 30 mM, 40 mM 50 mM, 60 mM, 70 mM, 80 mM, 90 mM, 100 mM, 110 mM, 120 mM, 130 mM, 140 mM, or 150 mM. In some embodiments, the concentration is between about 10 mM and 500 mM. In some embodiments, the concentration is between about 10 mM and 250 mM. In some embodiments, the concentration is between about 10 mM and 150 mM. In some embodiments, the concentration is between about 10 mM and 100 mM. In some embodiments, the concentration is 40 mM or at least 50 mM. In some embodiments, the concentration is 50 mM or at least 50 mM. In some embodiments, the concentration is 60 mM or at least 60 mM. In some embodiments, the concentration is 70 mM or at least 70 mM. In some embodiments, the concentration is 80 mM or at least 80 mM. In some embodiments, the concentration is 90 mM or at least 90 mM. In some embodiments, the concentration is 100 mM or at least 100 mM.

In some embodiments, contacting the cell population comprising progenitor exhausted T cells with and effective amount of the compound is maintained for a period of time sufficient to promote proliferation of progenitor exhausted T cells. In some embodiments, the T cells are contacted under conditions and for sufficient time periods such that 10%, 15%, 20%, 25%, 30%, 35%0, 40%, 45%, 50%, 55%, 60%, 65%, 70%, 75%, 80%, 85%, 90%, 95%, or even 100% of the T cells are expanded as desired. T cells are contacted under conditions and for sufficient time periods to achieve expansion as desired. In some embodiments, the period of time is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, or more days. In some embodiments, the period of time is between about 3 days and 5 days. In some embodiments, the period of time is between about 3 days and 7 days. In some embodiments, the period of time is between 3 days and 10 days. In some embodiments, the period of time is about 1 day or at least 1 day. In some embodiments, the period of time is about 2 days or at least 2 days. In some embodiments, the period of time is about 3 days or at least 3 days. In some embodiments, the period of time is about 4 days or at least 4 days. In some embodiments, the period of time is about 5 days or at least 5 days. In some embodiments, the period of time is about 6 days or at least 6 days. In some embodiments, the period of time is about 7 days or at least 7 days. In some embodiments, the period of time is about 8 days or at least 8 days. In some embodiments, the period of time is about 9 days or at least 9 days. In some embodiments the period of time is about 10 days or at least 10 days. In some embodiments, contacting the cell population comprising progenitor exhausted T cells with an effective amount of the compound is maintained for a period of about 3 days. In some embodiments, contacting the cell population comprising progenitor exhausted T cells with an effective amount of the compound is maintained for a period of about 4 days. In some embodiments, contacting the cell population comprising progenitor exhausted T cells with an effective amount of the compound is maintained for a period of about 5 days. In some embodiments, contacting the cell population comprising progenitor exhausted T cells with an effective amount of the compound is maintained for a period of about 6 days. In some embodiments, contacting the cell population comprising progenitor exhausted T cells with an effective amount of the compound is maintained for a period of about 7 days. In some embodiments, contacting the cell population comprising progenitor exhausted T cells with an effective amount of the compound is maintained for a period of about 8 days. In some embodiments, contacting the cell population comprising progenitor exhausted T cells with an effective amount of the compound is maintained for a period of about 9 days. In some embodiments, contacting the cell population comprising progenitor exhausted T cells with an effective amount of the compound is maintained for a period of about 10 days. In some embodiments, contacting the cell population comprising progenitor exhausted T cells with an effective amount of the compound is maintained for a period of about 11 days. In some embodiments, contacting the cell population comprising progenitor exhausted T cells with an effective amount of the compound is maintained for a period of about 12 days.

In some embodiments, contacting T cells with the compound comprises contacting T cells with a culture media comprising the compound. In some embodiments, contacting T cells with a culture media comprises culturing in vitro. The compound of the present methods may be used with any variety of culture media as would be known to the skilled person (see e.g., Current Protocols in Cell Culture, 2000-2009 by John Wiley & Sons, Inc.). In some embodiments the medium comprises RPMI 1640 Medium, GlutaMAX™ Supplement (Gibco Cat No: 61870-036) supplemented with 10% FBS (HyClone, Cat No: SH30396.03), 1% Pen Strep (Gibco, Cat No: 15140-122), 1% MEM NEAA (Gibco, Cat No: 1140-050), 1 mM Sodium Pyruvate (Gibco, Cat No: 11360-070), 10 mM HEPES (Gibco, Cat No: 15630-080), and 55 μM 2-Mercaptoethanol (Gibco, Cat No: 21985-023).

In certain embodiments, after a period of time, such as from 2, 3, 4, 5, 6, 7, 8, 9, or more days, an additional volume of culture medium may be added to T cells being cultured. In some embodiments, after 1 day an additional volume of culture medium may be added to T cells being cultured. In some embodiments, after 2 days an additional volume of culture medium may be added to T cells being cultured. In some embodiments, after 3 days an additional volume of culture medium may be added to T cells being cultured. In some embodiments, after 4 days an additional volume of culture medium may be added to T cells being cultured. In some embodiments, after 5 days an additional volume of culture medium may be added to T cells being cultured. In some embodiments, after 6 days an additional volume of culture medium may be added to T cells being cultured. In some embodiments, after 7 days an additional volume of culture medium may be added to T cells being cultured. In some embodiments, after 8 days an additional volume of culture medium may be added to T cells being cultured. In some embodiments, after 9 days an additional volume of culture medium may be added to T cells being cultured. In some embodiments, after 10 days an additional volume of culture medium may be added to T cells being cultured.

In certain embodiments, T cells cultured with the compound exhibit greater expansion compared to T cells not cultured with the compound. In certain embodiments, T cells cultured with the compound exhibit at least 1, 2, 3, 4, 5, 6, 7, 8, 9, 10, 11, 12, 13, 14, 15, 16, 17, 18, 19, or 20-fold greater expansion compared to T cells not cultured with the compound. In certain embodiments, T cells cultured with the compound exhibit 1- to 2-fold greater expansion compared to T cells not cultured with the compound. In certain embodiments, T cells cultured with the compound exhibit 7- to 14-fold greater expansion compared to T cells not cultured with the compound.

In certain embodiments, the T cells are cultured under conditions and for sufficient time periods such that the number of cells is 1-, 2-, 3-, 4-, 5-, 6-, 7-, 8-, 9-, 10-, 20-, 30-, 40-, 50-, 60-, 70-, 80-, 90-, 100-fold or more greater than the number of T cells at the start of culture. In some embodiments, the number of cells is 10 to 1000-fold greater, including consecutive integers therein, than the number of T cells at the start of culture. In some embodiments, the expanded T cell population is at least 10-fold greater than the initial isolated T cell population. In another embodiment, the expanded T cell population is at least 100-fold greater than the initial isolated T cell population. In some embodiments, the expanded T cell population is at least 500-fold greater than the initial isolated T cell population. In some embodiments, the expanded T cell population is at least 1000-fold greater than the initial isolated T cell population.

It is contemplated that a T cell may have been transfected or engineered to express one or more proteins of interest and are harvested from culture after expansion in vitro. In some embodiments, the T cell is transfected or engineered prior to culturing the T cell with the compound. In some embodiments the T cell is transfected or engineered 1 day prior to culturing the T cell with the compound. In some embodiments the T cell is transfected or engineered 2 days prior to culturing the T cell with the compound. In some embodiments the T cell is transfected or engineered 3 days prior to culturing the T cell with the compound. In some embodiments the T cell is transfected or engineered 4 days prior to culturing the T cell with the compound. In some embodiments the T cell is transfected or engineered 5 days prior to culturing the T cell with the compound. In some embodiments the T cell is transfected or engineered 6 days prior to culturing the T cell with the compound. In some embodiments the T cell is transfected or engineered 7 days prior to culturing the T cell with the compound. In some embodiments the T cell is transfected or engineered 8 days prior to culturing the T cell with the compound. In some embodiments the T cell is transfected or engineered 9 days prior to culturing the T cell with the compound. In some embodiments the T cell is transfected or engineered 10 days prior to culturing the T cell with the compound. In some embodiments the T cell is transfected or engineered about 1 to about 10 days prior to culturing the T cell with the compound. In some embodiments the T cell is transfected or engineered about 1 to about 5 days prior to culturing the T cell with the compound. In some embodiments, the T cell is transfected or engineered after culturing the T cell with the compound. In some embodiments, the T cell is transfected or engineered 1 day after culturing the T cell with the compound. In some embodiments, the T cell is transfected or engineered 2 days after culturing the T cell with the compound. In some embodiments, the T cell is transfected or engineered 3 days after culturing the T cell with the compound. In some embodiments, the T cell is transfected or engineered 4 days after culturing the T cell with the compound. In some embodiments, the T cell is transfected or engineered 5 days after culturing the T cell with the compound. In some embodiments, the T cell is transfected or engineered 6 days after culturing the T cell with the compound. In some embodiments, the T cell is transfected or engineered 7 days after culturing the T cell with the compound. In some embodiments, the T cell is transfected or engineered 8 days after culturing the T cell with the compound. In some embodiments, the T cell is transfected or engineered 9 days after culturing the T cell with the compound. In some embodiments, the T cell is transfected or engineered 10 days after culturing the T cell with the compound. In some embodiments, the T cell is transfected or engineered about 1 to about 10 days after culturing the T cell with the compound. In some embodiments, the T cell is transfected or engineered about 1 to about 5 days after culturing the T cell with the compound.

In some embodiments, contacting T cells with the compound comprises a treatment in vivo comprising contacting the compound with T cells in vivo. In some embodiments, the treatment in vivo comprises administering the compound to a subject, wherein the compound contacts a T cell. In some embodiments, the treatment in vivo comprises administering the compound and one or more pharmaceutically acceptable excipients or diluents. In some embodiments, the compound, alone or with a target cell population (e.g., a antigen-specific or a transfected or otherwise engineered and expanded T cell population) of the present disclosure, may be administered collectively to a subject to support a T cell adoptive therapy. In some embodiments, the compound is administered to a subject after administering T cells, wherein T cells are being used for adoptive cell therapy. In some embodiments, the compound is administered to a subject simultaneously with T cells, wherein T cells are being used for adoptive cell therapy. In some embodiments, the treatment in vivo enhances in vivo expansion of transferred T cells used for adoptive cell therapy compared to T cells transferred without administration of the compound. In some embodiments, the treatment in vivo maintains greater stemness of transferred T cells used for adoptive cell therapy compared to T cells transferred without administration of the compound. In some embodiments, the treatment in vivo enhances in vivo persistence of transferred T cells used for adoptive cell therapy compared to T cells transferred without administration of the compound. In some embodiments, the compound is administered to a subject in simultaneously with T cells, wherein T cells are being used for adoptive cell therapy. In some embodiments, the compound contacts T cells in vitro prior to transfer as well as in vivo after transferring T cells to a subject. In some embodiments, the compound does not contact T cells in vitro prior to transfer and does contact T cells in vivo after transferring T cells to a subject.

The administration of the treatment in vivo may be performed a number of ways depending upon whether local or systemic administration is desired. The compound is typically suitable for parenteral administration, wherein administration includes any route of administration characterized by physical breaching of a tissue of a subject and administration of the compound through the breach in the tissue, thus generally resulting in the direct administration into the blood stream, into muscle, or into an internal organ. Parenteral administration thus includes, but is not limited to, administration of the compound by injection, by application of the compound through a surgical incision, by application of the compound through a tissue-penetrating non-surgical wound, and the like. In particular, parenteral administration is contemplated to include, but is not limited to, subcutaneous, intraperitoneal, intramuscular, intrastemal, intravenous, intranasal, intratracheal, intraarterial, intrathecal, intraventricular, intraurethral, intracranial, intratumoral, intraocular, intradermal, intrasynovial injection or infusions, and intra-tumoral techniques. In some embodiments, the compound comprises intravenous administration.

### Culture Formulation

In an aspect, the disclosure provides a culture medium for promoting expansion and/or proliferation of progenitor exhausted T cells with enhanced expansion and/or persistence, comprising an effective amount of a compound, wherein the compound is identified in the methods of screening for compounds that promote proliferation of progenitor exhausted T cells.

In some embodiments, media for use in the methods described herein includes, but is not limited to Iscove modified Dulbecco medium (with or without fetal bovine or other appropriate serum). Illustrative media also includes, but is not limited to, IMDM, RPMI 1640, AIM-V, DMEM, MEM, a-MEM, F-12, X-Vivo 15, and X-Vivo 20. In some embodiments, the medium may comprise a surfactant, an antibody, plasmanate or a reducing agent, one or more antibiotics, and/or additives such as insulin, transferrin, sodium selenite and cyclosporin. In some embodiments, the media comprises RPMI 1640 Medium, GlutaMAX™ Supplement (Gibco Cat No: 61870-036) supplemented with 10% FBS (HyClone, Cat No: SH30396.03), 1% Pen Strep (Gibco, Cat No: 15140-122), 1% MEM NEAA (Gibco, Cat No: 1140-050), 1 mM Sodium Pyruvate (Gibco, Cat No: 11360-070), 10 mM HEPES (Gibco, Cat No: 15630-080), and 55 μM 2-Mercaptoethanol (Gibco, Cat No: 21985-023).

In some embodiments, the proliferation of the T cells promoted by the compound uncouples T cell expansion from differentiation. In some embodiments, the effective amount is an amount sufficient to cause proliferation of the T cell population.

Example 5 describes a transcriptomic analysis between T cells cultured in vitro with or without an identified compound that decouples proliferation from differentiation. Numerous genes were identified to be significantly differentially expressed in T cells treated with a compound (e.g., GlcNac or Neu5Ac) compared to those without treatment a compound identified to decouple proliferation from differentiation. Furthermore, there was notable similarity between secondary PCA analyses from T cells exposed to different compounds identified to decouple proliferation from differentiation. Therefore, the significantly differentially regulated genes found between samples treated with a compound identified to decouple proliferation from differentiation likely play a significant role in the molecular mechanisms responsible for decoupling. Thus, a compound that modulates of one or more of Cd24a, lgfbp7, Tcf7, lgfbp4, Adcy5, Nt5e, Hck, Lif, Apol9b, Gm4951, ligp1, Gzmk, Serpina3f, Nt5dc2, Maged1, Bmf, Cacnb3, Fut4, Egr1, Slc6a12, Fbxo2, Egr2, Fos, Plxnb2, Marcksl1, Ikzf2, Filip1I, Trerf1, Stard10, Pls1, Gm13546, Ccr7, Igha, Itgae, Hic, Klrh1, Als2cl, Tanc2, Slco4a1, Piwil4, Slc25a23, Itga4, Ckb, Actn1, Sema7a, Gm24187, Mir6236, H4c12, Gzmc, Prf1, Csf1, Gzmd, Tspan32, Atp6v0a1, Map6, Lmna, Rxra, Gpr141, Gm20559, Adam8, Anxa1, Stc2, Fosl2, Akrlc13, Cdkn2a, Crmp1, Tnfrsf21, Kctdl2, Ccr2, Samd9I, Sytl2, Ccr5, Tnfrsf9, Mme, Asns, Eomes, Oas3, Ly6a2, Ifi204, Ifit1, Cdh1, Ifit3, Isg15, Rtp4, Mx1, Oasl2, Rpl12, Rrp1, Rrp1b, Rrp12, Rrp15, mt.Rnr2, CT010467.2, Gm23935, Ct010467.1, and Lars2 genes is expected to impact decoupling of differentiation and proliferation of T cells. In some embodiments, the compound is a compound that modulates of one or more of Cd24a, lgfbp7, Tcf7, lgfbp4, Adcy5, Nt5e, Hck, Lif, Apol9b, Gm4951, ligp1, Gzmk, Serpina3f, Nt5dc2, Maged1, Bmf, Cacnb3, Fut4, Egr1, Slc6a12, Fbxo2, Egr2, Fos, Plxnb2, Marcksl1, Ikzf2, Filip1I, Trerf1, Stard10, Pls1, Gm13546, Ccr7, Igha, Itgae, Hic1, Klrh1, Als2cl, Tanc2, Slco4al, Piwil4, Slc25a23, Itga4, Ckb, Actn1, Sema7a, Gm24187, Mir6236, H4c12, Gzmc, Prf1, Csfa, Gzmd, Tspan32, Atp6v0al, Map6, Lmna, Rxra, Gpr141, Gm20559, Adam8, Anxa1, Stc2, Fosl2, Akr1c13, Cdkn2a, Crmp1, Tnfrsf21, Kctdl2, Ccr2, Samd9I, Sytl2, Ccr5, Tnfrsf9, Mme, Asns, Eomes, Oas3, Ly6a2, Ifi204, Ifit1, Cdh1, Ifit3, Isg15, Rtp4, Mx1, Oasl2, Rpl12, Rrp1, Rrp1b, Rrp12, Rrp15, mt.Rnr2, CT010467.2, Gm23935, Ct010467.1, and Lars2 genes.

In some embodiments, the compound is a sugar. In some embodiments, the sugar is trehalose, sucrose, lactose, glucose, galactose, fructose, neuraminic acid, or a derivative thereof. In some embodiments, the sugar is at a concentration above physiological levels. In some embodiments, the concentration is at least about 1 mM, 2 mM, 3 mM, 4 mM, 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 150 mM, 200 mM, 250 mM, 300 mM, 350 mM, 400 mM, 450 mM 500 mM, or more. In some embodiments, the concentration is at least about 10 mM, 20 mM, 30 mM, 40 mM 50 mM, 60 mM, 70 mM, 80 mM, 90 mM, 100 mM, 110 mM, 120 mM, 130 mM, 140 mM, or 150 mM. In some embodiments, the concentration is between about 10 mM and 500 mM. In some embodiments, the concentration is between about 10 mM and 250 mM. In some embodiments, the concentration is between about 10 mM and 150 mM. In some embodiments, the concentration is between about 10 mM and 100 mM. In some embodiments, the concentration is 40 mM or at least 50 mM. In some embodiments, the concentration is 50 mM or at least 50 mM. In some embodiments, the concentration is 60 mM or at least 60 mM. In some embodiments, the concentration is 70 mM or at least 70 mM. In some embodiments, the concentration is 80 mM or at least 80 mM. In some embodiments, the concentration is 90 mM or at least 90 mM. In some embodiments, the concentration is 100 mM or at least 100 mM. I

In some embodiments, the culture media further comprises activating factors that may be added to the in vitro cell culture at various concentrations to achieve the desired outcome (e.g., expansion decoupled from differentiation). It is contemplated that a T cell activating factor may be utilized in the culture media for expanding the T cells and not in differentiating the T cells. For example, T cells may be cultured with one or more T cell activating factors including but not limited to IL-2. In some embodiments, the culture media comprises IL-2. In some embodiments, the amount of IL-2 is at least about 10, 20, 30, 40, 50, 60, 70, 80, 90, 100, 110, 120, 130, 140, 150, 160, 170, 180, 190, 200, 210, 220, 230, 240, 250, 300, 350, 400, 500, 1000 IU/mL. In some embodiments, the amount of IL-2 is between about 10 to about 100 IU/mL. In some embodiments, the amount of IL-2 is between about 100 to about 200 IU/mL. In some embodiments, the amount of IL-2 is between about 200 to about 500 IU/mL. In some embodiments, the amount of IL-2 is between about 500 to about 1000 IU/mL. In some embodiments, the amount of IL-2 is about 10 IU/mL or at least 10 IU/mL. In some embodiments, the amount of IL-2 is about 20 IU/mL or at least 20 IU/mL. In some embodiments, the amount of IL-2 is about 30 IU/mL or at least 30 IU/mL. In some embodiments, the amount of IL-2 is about 40 IU/mL or at least 40 IU/mL. In some embodiments, the amount of IL-2 is about 50 IU/mL or at least 50 IU/mL. In some embodiments, the amount of IL-2 is about 60 IU/mL or at least 60 IU/mL. In some embodiments, the amount of IL-2 is about 70 IU/mL or at least 70 IU/mL. In some embodiments, the amount of IL-2 is about 90 IU/mL or at least 90 IU/mL. In some embodiments, the amount of IL-2 is about 10 IU/mL or at least 10 IU/mL. In some embodiments, the amount of IL-2 is about 100 IU/mL or at least 100 IU/mL. In some embodiments, the amount of IL-2 is about 110 IU/mL or at least 110 IU/mL. In some embodiments, the amount of IL-2 is about 120 IU/mL or at least 120 IU/mL. In some embodiments, the amount of IL-2 is about 130 IU/mL or at least 130 IU/mL. In some embodiments, the amount of IL-2 is about 140 IU/mL or at least 140 IU/mL. In some embodiments, the amount of IL-2 is about 150 IU/mL or at least 150 IU/mL. In some embodiments, the amount of IL-2 is about 160 IU/mL or at least 160 IU/mL. In some embodiments, the amount of IL-2 is about 170 IU/mL or at least 170 IU/mL. In some embodiments, the amount of IL-2 is about 180 IU/mL or at least 180 IU/mL. In some embodiments, the amount of IL-2 is about 190 IU/mL or at least 190 IU/mL. In some embodiments, the amount of IL-2 is about 200 IU/mL or at least 200 IU/mL. In some embodiments, the amount of IL-2 is about 210 IU/mL or at least 210 IU/mL. In some embodiments, the amount of IL-2 is about 220 IU/mL or at least 220 IU/mL. In some embodiments, the amount of IL-2 is about 230 IU/mL or at least 230 IU/mL. In some embodiments, the amount of IL-2 is about 240 IU/mL or at least 240 IU/mL. In some embodiments, the amount of IL-2 is about 250 IU/mL or at least 250 IU/mL. In some embodiments, the amount of IL-2 is about 60 IU/mL. In some embodiments, the amount of IL-2 is about 200 IU/mL.

### FucoID and Use of Sugars

In an aspect, the disclosure provides a method of identifying progenitor exhausted T cell specific to an antigen, comprising: (a) culturing a cell population comprising progenitor exhausted T cell in a culture medium; (b) contracting the cell population with a modified dendritic cell in the presence of a donor sugar nucleotide that is conjugated to a label, wherein (i) the modified dendritic cell has been engineered to comprise on its cell surface an active fucosyltransferase that is capable of catalyzing the glycosylation of a cell surface glycan on the T cell using the donor sugar nucleotide; and (ii) the modified dendritic cell has been primed with one or more antigens; and (c) analyzing the cell surface of the population of progenitor exhausted T cells, after the contacting, to determine whether any label is present; wherein, the presence of the label on the cell surface of the progenitor exhausted T cell indicates that the progenitor exhausted T cell is specific to an antigen with specificity for at least one of the one or more antigens.

In some embodiments, the culture medium comprises an effective amount of a sugar. In some embodiments, the sugar is trehalose, sucrose, lactose, glucose, galactose, fructose, neuraminic acid, or a derivative thereof. In some embodiments, the sugar is at a concentration above physiological levels. In some embodiments, the concentration is at least about 1 mM, 2 mM, 3 mM, 4 mM, 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 150 mM, 200 mM, 250 mM, 300 mM, 350 mM, 400 mM, 450 mM 500 mM, or more. In some embodiments, the concentration is at least about 10 mM, 20 mM, 30 mM, 40 mM 50 mM, 60 mM, 70 mM, 80 mM, 90 mM, 100 mM, 110 mM, 120 mM, 130 mM, 140 mM, or 150 mM. In some embodiments, the concentration is between about 10 mM and 500 mM. In some embodiments, the concentration is between about 10 mM and 250 mM. In some embodiments, the concentration is between about 10 mM and 150 mM. In some embodiments, the concentration is between about 10 mM and 100 mM. In some embodiments, the concentration is 40 mM or at least 50 mM. In some embodiments, the concentration is 50 mM or at least 50 mM. In some embodiments, the concentration is 60 mM or at least 60 mM. In some embodiments, the concentration is 70 mM or at least 70 mM. In some embodiments, the concentration is 80 mM or at least 80 mM. In some embodiments, the concentration is 90 mM or at least 90 mM. In some embodiments, the concentration is 100 mM or at least 100 mM.

In some embodiments, the culture media further comprises activating factors that may be added to the in vitro cell culture at various concentrations to achieve the desired outcome (e.g., expansion decoupled from differentiation). It is contemplated that a T cell activating factor may be utilized in the culture media for expanding the T cells and not in differentiating the T cells. For example, T cells may be cultured with one or more T cell activating factors including but not limited to IL-2. In some embodiments, the culture media comprises IL-2. In some embodiments, the amount of IL-2 is at least about 10, 20, 30, 40, 50, 60, 70, 80, 90, 100, 110, 120, 130, 140, 150, 160, 170, 180, 190, 200, 210, 220, 230, 240, 250, 300, 350, 400, 500, 1000 IU/mL. In some embodiments, the amount of IL-2 is between about 10 to about 100 IU/mL. In some embodiments, the amount of IL-2 is between about 100 to about 200 IU/mL. In some embodiments, the amount of IL-2 is between about 200 to about 500 IU/mL. In some embodiments, the amount of IL-2 is between about 500 to about 1000 IU/mL. In some embodiments, the amount of IL-2 is about 10 IU/mL or at least 10 IU/mL. In some embodiments, the amount of IL-2 is about 20 IU/mL or at least 20 IU/mL. In some embodiments, the amount of IL-2 is about 30 IU/mL or at least 30 IU/mL. In some embodiments, the amount of IL-2 is about 40 IU/mL or at least 40 IU/mL. In some embodiments, the amount of IL-2 is about 50 IU/mL or at least 50 IU/mL. In some embodiments, the amount of IL-2 is about 60 IU/mL or at least 60 IU/mL. In some embodiments, the amount of IL-2 is about 70 IU/mL or at least 70 IU/mL. In some embodiments, the amount of IL-2 is about 90 IU/mL or at least 90 IU/mL. In some embodiments, the amount of IL-2 is about 10 IU/mL or at least 10 IU/mL. In some embodiments, the amount of IL-2 is about 100 IU/mL or at least 100 IU/mL. In some embodiments, the amount of IL-2 is about 110 IU/mL or at least 110 IU/mL. In some embodiments, the amount of IL-2 is about 120 IU/mL or at least 120 IU/mL. In some embodiments, the amount of IL-2 is about 130 IU/mL or at least 130 IU/mL. In some embodiments, the amount of IL-2 is about 140 IU/mL or at least 140 IU/mL. In some embodiments, the amount of IL-2 is about 150 IU/mL or at least 150 IU/mL. In some embodiments, the amount of IL-2 is about 160 IU/mL or at least 160 IU/mL. In some embodiments, the amount of IL-2 is about 170 IU/mL or at least 170 IU/mL. In some embodiments, the amount of IL-2 is about 180 IU/mL or at least 180 IU/mL. In some embodiments, the amount of IL-2 is about 190 IU/mL or at least 190 IU/mL. In some embodiments, the amount of IL-2 is about 200 IU/mL or at least 200 IU/mL. In some embodiments, the amount of IL-2 is about 210 IU/mL or at least 210 IU/mL. In some embodiments, the amount of IL-2 is about 220 IU/mL or at least 220 IU/mL. In some embodiments, the amount of IL-2 is about 230 IU/mL or at least 230 IU/mL. In some embodiments, the amount of IL-2 is about 240 IU/mL or at least 240 IU/mL. In some embodiments, the amount of IL-2 is about 250 IU/mL or at least 250 IU/mL. In some embodiments, the amount of IL-2 is about 60 IU/mL. In some embodiments, the amount of IL-2 is about 200 IU/mL.

In some embodiments, the present disclosure provides for the use of compositions and methods to identify and enrich for tumor-specific antigen (TSA) reactive T cells from tumor infiltrating lymphocytes (TILs) or circulating T cells and/or to identify and enrich for T cells that recognize antigens of a particular disease.

As used herein, the term “FucoID” or “FucoID labeling” refers to a method to identify and enrich for antigen-specific T cells using a fucosyltransferase. FucoID comprises contacting a population of T cells with a modified dendritic cell in the presence of a donor sugar nucleotide that is conjugated to a label, (i) wherein the modified dendritic cell has been engineered to comprise on its cell surface an active glycosyltransferase that is capable of catalyzing the glycosylation of a cell surface glycan on the T cell using the donor sugar nucleotide; and (i) wherein the modified dendritic cell has been primed with one or more antigen; and (b) analyzing the cell surface of the population of T cells, after the contacting, to determine whether any label is present; wherein, the presence of the label on the cell surface of the T cell indicates that the T cell is a reactive T cell with specificity for at least one of the one or more of the antigens.

The terms “fucosyltransferase,” “FucT,” and “FT are used interchangeably herein and refer to an enzyme (e.g., a glycosyltransferase) that catalyzes the transfer of fucose from GDP-β-L-fucose (“GDP-fucose”) to an acceptor substrate. The acceptor substrate can be on an oligosaccharide and/or a protein. For example, fucosyltransferases can transfer fucose to the innermost GlcNAc (N-acetylglucosamine) residue present in an N-glycan (e.g., Type 1: Galβ1,3GlcNAc) resulting in an α-1,6-fucosylation known as “core fucosylation.” Fucosyltransferases can also catalyze “terminal fucosylation” by which fucose is attached to terminal galactose residues on oligosaccharides such as Galβ1,4GlcNAc (Type II, also called LacNAc) or Galβ1,3GlcNAc (Type III). Thirteen types of fucosyltransferases have been described to-date (including FUT1-FUT11, POFUT1, and POFUT2), which catalyze the different types of fucosylation. For example, FUT1 (NM_000148.1) and FUT2 (NM_000511.1) catalyze the synthesis of α-1,2-fucosylation; FUT3 (NM_000149.1) catalyzes the synthesis of α-1,3- and α-1,4-fucosylation (α-1,3/4 fucosylation); FUT4 (NM_002033.1), FUT5 (NM_002034.1), FUT6 (NM_000150.1) FUT7 (NM_004479.1), FUT9 (NM_006581.1), FUT10 (NM_032664.2) and FUT11 (NM_173540.1) catalyze α-1,3-fucosylation; FUT8 (NM_004480.1) catalyzes α-1,6-fucosylation; and POFUT1 (NM_015352.1) and POFUT2 (NM_015227.1) catalyze the addition of fucose directly to polypeptides via 0-glycosidic linkage to serine and threonine residues. Each of the sequences corresponding to the above NCBI Reference Sequence numbers for the thirteen fucosyltransferase enzymes are incorporated herein by reference in their entirety.

The term LacNAc refers to N-acetyllactosamine and referred to interchangeably as Gal 1,4 GlcNAc.

The terms sLacNAc and sialyl LacNAc, refer to a2,3-sialylated LacNAc, also referred to herein as sialyllactosamine.

In various embodiments, the compositions and methods of the present disclosure provide cells that have been engineered to have an enzyme on their cell surface such that, upon contacting another cell, the enzyme on the engineered cell catalyzes a labeling reaction that attaches a label (or “tag”) on the other cell. This process is referred to herein as “interaction-dependent labeling”; and the engineered cell that catalyzes the labeling reaction is referred to as a “bait cell” and the cell that is labeled by the bait cell is referred to as a “prey cell.” In some embodiments, by using the techniques described in herein, fucosyltransferase (FT)- (or other enzyme-) modified autologous iDCs primed with antigen, e.g., tumor lysate, can be used as the bait cells that induce proximity-based transfer of biotin (or other) tags to the surface of cells that interact with the DCs, e.g., TILs or circulating T cells. Using fluorescence activated cell sorting (FACS), (for example, or other enrichment methods) the biotinylated T cells (or otherwise tagged cells) can be isolated as bona fide antigen reactive, e.g., TSA-reactive TILs. These isolated cells may also express currently used surface markers designating prospective TSA reactivity (e.g. PD-1, CD134, or CD137) or other markers such as CXCR5 and/or TIM3. In such embodiments, because the proximity-based biotinylation only takes place on cells that interact with antigen-presenting DCs, using this method, bystander CD8+ T cells found in human tumor infiltrates that also express PD-1, CD134, CD137, CXCR5, and/or TIM330 are effectively excluded. Thus, in various embodiments, the present disclosure includes compositions (including pharmaceutical compositions) of TSA-reactive TILs or T cells that have been isolated using the proximity labeling methods disclosed herein, as well as expanded populations of such TSA-reactive TILs or T cells. Such compositions may be used in treating or preventing diseases such as, (e.g., cancer).

In various embodiments, the bait cells of the present disclosure comprise an enzyme on their cell surface for catalyzing the transfer of a label (or “tag”) to a target prey cell when the bait and prey cells come into contact. Suitable enzymes for use on the surface of a bait cell are disclosed herein, and include without limitation glycosyltransferases (e.g., fucosyltransferases and sialyltransferases), sortases, and promiscuous biotin ligases.

Any cell that is capable of contacting another cell may be used as the bait cell, provided that it may be engineered to have a suitable enzyme on its surface for catalyzing contact-induced interaction-dependent labeling of a contacted prey cell. In some embodiments, the bait cell is an antigen presenting cell (APC). In some instances the bait cell is a professional APC. In some instances the bait cell is a nonprofessional APC. In some instances the bait cell expresses an MHC class I molecule. In some instances the bait cells expresses an MHC class II molecule. In some instances the bait cell is a leukocyte. In some instances, the bait cell is an atypical APC. In some instances the bait cell is a cell selected from a DC, a macrophage, a B cell, a monocyte, a granulocyte, a mast cell, a neutrophil, an endothelial cell, an epithelial cell. In some embodiments, the bait cell is an engineered APC, e.g., an artificial APC (“aAPC”). Such aAPCs are known in the art. The aAPC may be a cell-based aAPC or a non-cell-based aAPC. In some instances, the non-cell-based aAPC comprises signal 1 and signal 2 and optionally signal 3 on a nanoparticle or microparticle aAPCs include two signals: a major histocompatibility complex (MHC) signal and a co-stimulatory molecule. In some instances, the MHC signal in the aAPC is MHC class I. In some instances, the MHC signal in the aAPC is MHC class II. In some instances, the co-stimulatory signal in the aAPC is generated by CD80 (B7.1) or CD86 (B7.2). The aAPC may also comprise a third signal that stimulates cytokine secretion to promote T cell stimulation and expansion. In some instances, the aAPC comprises a third signal that stimulates IL-2 secretion after the aAPC binds to a T cell. In some instances the aAPC is a fibroblast engineered to express the first signal and the second signal and optionally the third signal. The fibroblast may also express ICAM-1 and/or LFA-3. For example, in certain embodiments, the bait cell is a dendritic cell and the prey cell is an effector cell. The dendritic cell may be an immature dendritic cell. The dendritic cell may be primed with an antigen. In some embodiments, the antigen is from a cancer, a pathogenic infection, an autoimmune disease, an inflammatory disease, or a genetic disorder. In some embodiments, effector cells used as prey cells in the present invention in combination with bait cells that are dendritic cells include effector cells selected from a naive T cell, a memory stem cell T cell, a central memory T cell, an effector memory T cell, a helper T cell, a CD4+ T cell, a CD8+ T cell, a CD8/CD4+ T cell, an αβ T cell, a γδ T cell, a cytotoxic T cell, a natural killer T cell, a natural killer cell, and a macrophage. In such methods, autologous immature DCs primed with tumor lysates may in some embodiments be modified with an enzyme that induces proximity-based transfer of tags (e.g., biotin) to the surface of prey cells (i.e. TILs or circulating T cells) that interact with the bait cell DCs. Using fluorescence activated cell sorting (FACS) (or other suitable isolation methods), the tagged (e.g., biotinylated) T cells that may also express currently used surface markers designating prospective TSA reactivity (e.g. PD-1, CD134, or CD137)28-30 or other markers such as CXCR5 and/or TIM3 may be isolated. The isolated T cells are highly enriched for antigen reactive T cells, e.g., tumor-reactive T cells with reactivity directed toward TSAs. Similarly, DCs primed with tissue lysates from autoimmune patient biopsies may be modified with an enzyme that induces proximity-based transfer of tags (e.g., biotin) to the surface of prey cells (circulating autoreactive T cells) that interact with the bait cell DCs. From this assay, tagged (e.g., biotin+) CD8+ T cells that are prospective auto-reactive T cells, and tagged (e.g., biotin+) CD4+, CD25+, FOXP3+ T cells that are prospective antigen specific regulatory T cells may be isolated.

In some embodiments, the bait cell is a B cell. In some embodiments, the bait cell is a B cell and the prey cell is a T cell. In some embodiments, the bait cell is a naïve B cell or a germinal center B cell.

Similarly, in some embodiments, the bait cell is such an effector cell (e.g., a naive T cell, a memory stem cell T cell, a central memory T cell, an effector memory T cell, a helper T cell, a CD4+ T cell, a CD8+ T cell, a CD8/CD4+ T cell, an αβ T cell, a γδ T cell, a cytotoxic T cell, a natural killer T cell, a natural killer cell, or a macrophage) and the effector cell is, thus, engineered to have a suitable enzyme on its surface for catalyzing contact-induced interaction-dependent labeling of a contacted prey cell. In some embodiments, the bait cell is a T cell expressing a suitable enzyme on its surface for catalyzing contact-induced interaction-dependent labeling of a contacted prey cell and the prey cell is a B cell. In some embodiments, the bait cell is a T cell expressing a suitable enzyme on its surface for catalyzing contact-induced interaction-dependent labeling of a contacted prey cell and the prey cell is a naïve B cell a germinal center B cell. In some embodiments, the bait cell is a T cell expressing a suitable enzyme on its surface for catalyzing contact-induced interaction-dependent labeling of a contacted prey cell and the prey cell is a dendritic cell. In certain embodiments of the present disclosure, the bait cell is a B cell expressing a suitable enzyme on its surface for catalyzing contact-induced interaction-dependent labeling of a contacted prey cell and the prey cell is a CD4+ T cell.

Any enzyme capable of catalyzing contact-induced interaction-dependent labeling of a contacted prey cell utilizing a tagged substrate may be disposed on the surface of a bait cell and utilized in the present invention. Such an enzyme is referred to herein as a “suitable enzyme.” In some embodiments, suitable enzymes for use on a bait cell to facilitate interaction-dependent labeling in accordance with the present invention (i) possess high Km toward the acceptor substrate found on the surface of the prey cells such that background labeling is minimal; and (ii) possess high kcat to trigger interaction-dependent labeling when an interaction between bait and prey cells takes place. The enzyme may be a human enzyme. The enzyme may be a non-human enzyme. The enzyme may be a recombinant enzyme. The enzyme may be an isolated enzyme. The enzyme may be a native enzyme. The enzyme may be a non-native enzyme. The enzyme may comprise a tag (e.g., an epitope tag). Such tags are well known in the art and may be used in the present invention including, without limitation, any tag described herein.

In some instances, the enzyme on the surface of the bait cell is a transferase. In general, transferases catalyze the transfer of one molecular group from one molecule to another. For instance, such molecular groups include phosphate, amino, methyl, acetyl, acyl, phosphatidyl, phosphoribosyl, among other groups, and suitable transferases for use in the present invention include any transferase that is capable of catalyzing the transfer of one molecule to another, even if the molecule has been labeled with a tag (e.g., a tag known in the art or described herein).

For example, in some embodiments, the present disclosure provides bait cells having an enzyme on their surface that is a glycosyltransferase. Glycosyltransferases catalyze the transfer of sugar nucleotide donors to acceptor molecules, which may include, e.g., proteins and other sugar moieties (glycans), e.g., on glycoproteins, glycolipids, oligosaccharides, etc.

In certain embodiments, the present disclosure provides bait cells having a fucosyltransferase or a sialyltransferase on their surface.

Fucosyltransferases catalyze the transfer of fucose from GDP-Fuc to Gal in a α1,2-linkage and to GlcNAc in a α1,3-, α1,4-, or α1,6-linkage. Since known fucosyltransferases utilize the same nucleotide sugar, it is believed that their specificity resides in the recognition of the acceptor and in the type of linkage formed. On the basis of protein sequence similarities, these enzymes have been classified into four distinct families: (1) the alpha-2-fucosyltransferases, (2) the alpha-3-fucosyltransferases, (3) the mammalian alpha-6-fucosyltransferases, and (4) the bacterial alpha-6-fucosyltransferases. Conserved structural features, as well as a consensus peptide motif have been identified in the catalytic domains of all alpha-2 and alpha-6-fucosyltranferases, from prokaryotic and eukaryotic origin. Based on these sequence similarities, alpha-2 and alpha-6-fucosyltranferases have been grouped into one superfamily. In addition, a few amino acids were found strictly conserved in this superfamily, and two of these residues have been reported to be essential for enzyme activity for a human alpha-2-fucosyltransferase. The alpha-3-fucosyltransferases constitute a distinct family as they lack the consensus peptide, but some regions display similarities with the alpha-2 and alpha-6-fucosyltranferases. All these observations strongly suggest that the fucosyltransferases share some common structural and/or catalytic features. In humans, at least 11 fucosyltransferases have been described, and these are encoded by human genes FUT1; FUT2; FUT3; FUT4; FUT5; FUT6; FUT7; FUT8; FUT9; FUT10; and FUT11.

Sialyltransferases are glycosyltransferases responsible for the terminal sialylation of carbohydrate groups of glycoproteins, glycolipids and oligosaccharides which contain a conserved region of homology in the catalytic domain. Members of the sialyltransferase gene family comprise Gal/31,3GalNAc α2,3 sialyltransferase and Gall,3(4)GlcNAc α2,3 sialyltransferase. Sialylation refers to the transfer of sialic acid to a terminal position on sugar chains of glycoproteins, glycolipids, oligosaccharides and the like. Examples of enzymatically functional sialyltransferases are those capable of transferring sialic acid from CMP-sialic acid to an acceptor oligosaccharide, where the oligosaccharide acceptor varies depending upon the particular sialyltransferase. Numerous sialyltransferases are known in the art and include, without limitation human sialyltransferases SIAT4C; SIAT9; ST3GAL1; ST3GAL2; ST3GAL3; ST3GAL4; ST3GAL5; ST3GAL6; ST3GalIII; ST6GAL1; ST6GAL2; ST6Gal; ST8SIA1; ST8SIA2; ST8SIA3; ST8SIA4; ST8SIA5; ST8SIA6; and ST8Sia.

Typically, glycosyltransferases have stringent donor substrate specificities; however the present inventors have discovered and recently reported that H. pylori α1,3fucosyltransferase has remarkable substrate tolerance (PCT/US2018/016503, published as WO2018/144769, the content of which is incorporated herein by reference in its entirety) and will essentially permit anything desirable to be conjugated, e.g., via a linker, to its GDP-Fucose substrate and still be capable of catalyzing fucosylation reaction. The present inventors have similarly shown in their prior work that ST6Gal1; Pasteurella multocida α(2,3) sialyltransferase M144D mutant (Pm2,3ST-M144D); and Photobacterium damsel α(2,6) sialyltransferase (Pd2,6ST)) are permissive to functionalized CMP-sialic acid donor substrates. Id. and Hong, S, et al., Bacterial glycosyltransferase-mediated cell-surface chemoenzymatic glycan modification. Nature Communications 10, Article number: 1799 (2019), incorporated herein by reference in its entirety. Additionally, we show herein that H. pylori α1,3/1,4 fucosyltransferase; human α1,3 fucosyltransferase (FUT6); and human a2,6asialyltransferase (ST6Gal1), are also similarly permissive to conjugated donor substrates; as is the human α1,3 fucosyltransferase FUT9 (data not shown).

Thus, some embodiments of the present invention provide bait cells comprising a fucosyltransferase or sialyltransferase disposed on its surface and methods of using such bait cells to achieve interaction-dependent labeling, thereby detecting whether the bait cells have contacted another cell. In certain aspects, if a bait cell bearing a fucosyltransferase or a sialyltransferase disposed on its surface comes into contact with a prey cell comprising a suitable glycan acceptor moiety in the presence of a tagged conjugate of the appropriate donor sugar nucleotide for the respective glycosyltransferases (i.e., GDP-fucose for fucosyltransferases and CMP-Neu5Ac for the sialyltransferases), then the enzyme on the bait cell will catalyze a glycosyltransferase reaction that results in the attachment of the respective tagged donor sugar nucleotide to the prey cell; thus resulting in a lasting indicator that a cell-cell interaction has occurred between the bait and prey cells.

In one embodiment, the present disclosure provides bait cells comprising a human fucosyltransferase enzyme on their surface, and methods of using the same in interaction-dependent labeling a prey cell. In one embodiment, the human fucosyltransferase enzyme is human a 1,3-fucosyltransferase. In one embodiment, the human a 1,3-fucosyltransferase is recombinantly prepared. In one embodiment, the present disclosure provides bait cells comprising an H. pylori fucosyltransferase enzyme on their surface. In one embodiment, the H. pylori fucosyltransferase is H. pylori a 1,3-fucosyltransferase. In one embodiment, the H. pylori fucosyltransferase is H. pylori a 1,3/1,4-fucosyltransferase.

In one embodiment, the present disclosure provides bait cells comprising a human sialyltransferase enzyme on their surface, and methods of using the same in interaction-dependent labeling a prey cell. In one embodiment, the human sialyltransferase is ST6GAL1. In one embodiment, the human sialyltransferase is ST6GalNAc1. In one embodiment of the present disclosure, the enzyme on the surface of the bait cell is a non-human sialyltransferase. In one embodiment the non-human sialyltransferase is Pasteurella multocida α (2,3) sialyltransferase M144D mutant (Pm2,3ST-M144D) or Photobacterium damsela α (2,6) sialyltransferase (Pd2,6ST).

In one embodiment, the present disclosure provides bait cells comprising a sortase enzyme on their surface, and methods of using the same in interaction-dependent labeling a prey cell. Sortases are a family of enzymes capable of carrying out a transpeptidation reaction conjugating the C-terminus of a first protein to the N-terminus of second protein via transamidation. If a sortase enzyme comprised on the surface of a bait cell comes into contact with a prey cell having on its surface a polypeptide comprising a sortase acceptor peptide (e.g., a GGG residue) at its N-terminus in the presence of a tagged peptide comprising a sortase recognition sequence (e.g., LPXTG (SEQ ID NO: 5)), wherein X is any amino acid for sortase A), then the sortase will catalyze the attachment of the tagged peptide to the N-terminus of the polypeptide comprising the sortase recognition sequence; thereby attaching the tag to the prey cell. Any sortase known in the art or disclosed herein may be utilized in connection with the present invention, as a suitable enzyme for conjugation to the surface of a bait cell for the purpose of facilitating interaction-dependent labeling of a prey cell as described herein.

Sortases are also referred to as transamidases, and typically exhibit both a protease and a transpeptidation activity. Various sortases from prokaryotic organisms have been identified. For example, some sortases from Gram-positive bacteria cleave and translocate proteins to proteoglycan moieties in intact cell walls. Among the sortases that have been isolated from Staphylococcus aureus, are sortase A (Srt A) and sortase B (Srt B). Thus, in certain embodiments, a transamidase used in accordance with the interaction-dependent labeling methods described herein is a sortase A, e.g., from S. aureus, also referred to herein as SrtAaureus. In other embodiments, a transamidase is a sortase B, e.g., from S. aureus, also referred to herein as SrtBaureus.

Sortases have been classified into four classes, designated A, B, C, and D (i.e., sortase A, sortase B, sortase C, and sortase D, respectively) based on sequence alignment and phylogenetic analysis of 61 sortases from Gram-positive bacterial genomes (Dramsi et al., Res Microbiol. 156(3):289-97, 2005; the entire contents of which are incorporated herein by reference). These classes correspond to the following subfamilies, into which sortases have also been classified by Comfort and Clubb (Comfort et al., Infect Immun., 72(5):2710-22, 2004; the entire contents of which are incorporated herein by reference): Class A (Subfamily 1), Class B (Subfamily 2), Class C (Subfamily 3), and Class D (Subfamilies 4 and 5). The aforementioned references disclose numerous sortases and their recognition motifs. See also Pallen et al., TRENDS in Microbiology, 2001, 9(3), 97-101; the entire contents of which are incorporated herein by reference). Those skilled in the art will readily be able to assign a sortase to the correct class based on its sequence and/or other characteristics such as those described in Drami, et al., supra.

The term “sortase A” is used herein to refer to a class A sortase, usually named SrtA in any particular bacterial species, e.g., SrtA from S. aureus. Likewise “sortase B” is used herein to refer to a class B sortase, usually named SrtB in any particular bacterial species, e.g., SrtB from S. aureus. The present disclosure encompasses embodiments relating to any of the sortase classes known in the art (e.g., a sortase A from any bacterial species or strain, a sortase B from any bacterial species or strain, a class C sortase from any bacterial species or strain, and a class D sortase from any bacterial species or strain).

In some embodiments, the sortase used in the interaction-dependent labeling methods described herein is a wild-type enzyme. In other embodiments, the sortase is a modified version which may possess a superior feature as compared to the wild-type counterpart (e.g., higher catalytic activity). In some examples, the sortase can be a mutant of SrtA, which may comprise one or more of the following positions: P94, S102, A104, E105, K138, K152, D160, K162, T164, D165, K173, I182, K190, and K196. For example, a SrtA mutant may comprise one or more of the following mutations: P94R or P94S, S102C, A104H, E105D, K138P, K152I, D160K or D160N, K162H, T164N, D165A, K173E, 1182V, K190E, and K196S or K196T. In one example, the sortase is a triple mutant P94S/D160N/K196T of SrtA from S. aureus.

In other embodiments, modified sortase having altered substrate specificity can be used in the intercellular labeling methods described herein. For example, sortase A mutants having one or more mutations at positions S102 (e.g., S102C), A104 (e.g., A104H or A104V), E105 (e.g., E105D), K138 (e.g., K138P), K152 (e.g., K152I), N162 (e.g., N162N), T164 (e.g., T164N), K173 (e.g., K173E), 1182 (e.g., I182V), T196 (e.g., T196S), N98 (e.g., N98D), A118 (e.g., A118T), F122 (e.g., F122A), K134 (e.g., K134R), F144 (e.g., F144L), and E189 (e.g., E189F). Such a modified sortase may recognize sequences such as LAXTG (SEQ ID NO: 6) and/or LPXSG (SEQ ID NO: 7), in which X can be any amino acid residue. Examples include mutant S102C/A104H/E105D/K138P/K152I/N162N/T164N/K173E/I182V/T196S, and mutant N98D/A104V/A118T/F122A/K134R/F144L/E189F. Additional sortase mutants having altered substrate specificity are disclosed in US20140057317 and Dorr et al., PNAS 111 (37):13343-13348 (2014), the relevant disclosures therein are incorporated by reference herein.

A modified version of a wild-type sortase may share at least 85% (e.g., 90%, 95%, 98%, or above) sequence identity to the wild-type counterpart. It may contain mutations at one or more positions corresponding to those described above, which can be identified by analyzing the amino acid sequence of a wild-type sortase with the amino acid sequence of a SrtA. The “percent identity” of two amino acid sequences can be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the interaction-dependent labeling methods can use an active fragment of a sortase. Such a fragment of a specific sortase can be identified based on knowledge in the art or by comparing the amino acid sequence of that sortase with a sortase having known structure/function correlation (e.g., active domain being identified). In some examples, the sortase used herein can be an active fragment of a sortase A such as SrtA from S. aureus, e.g., a sortase A fragment lacking the N-terminal 59 or 60 amino acid residues, or a functional variants thereof, which may contain one or more of the mutations described herein.

Amino acid sequences of Srt A and Srt B and the nucleotide sequences that encode them are known to those of skill in the art and are disclosed in a number of references cited herein, the entire contents of all of which are incorporated herein by reference. See, e.g., GenBank accession numbers NP_375640 and YP_043193. The amino acid sequences of S. aureus SrtA and SrtB are homologous, sharing, for example, 22% sequence identity and 37% sequence similarity. The amino acid sequence of a sortase-transamidase from Staphylococcus aureus also has substantial homology with sequences of enzymes from other Gram-positive bacteria, and such transamidases can be utilized in the ligation processes described herein. For example, for SrtA there is about a 31% sequence identity (and about 44% sequence similarity) with best alignment over the entire sequenced region of the S. pyogenes open reading frame. There is about a 28% sequence identity with best alignment over the entire sequenced region of the A. naeslundii open reading frame. It will be appreciated that different bacterial strains may exhibit differences in sequence of a particular polypeptide, and the sequences herein are exemplary.

In certain embodiments a transamidase bearing 18% or more sequence identity, 20% or more sequence identity, or 30% or more sequence identity with an S. pyogenes, A. naeslundii, S. mutans, E. faecalis or B. subtilis open reading frame encoding a sortase can be screened, and enzymes having transamidase activity comparable to Srt A or Srt B from S. aureus can be utilized (e.g., comparable activity sometimes is 10% of Srt A or Srt B activity or more).

In some embodiments, the interaction-dependent labeling methods described herein use a sortase A (SrtA) or an active fragment thereof. SrtA recognizes the motif LPXTX (SEQ ID NO: 8); wherein each occurrence of X represents independently any amino acid residue), with common recognition motifs being, e.g., LPKTG (SEQ ID NO: 9), LPATG (SEQ ID NO: 10), or LPNTG (SEQ ID NO: 11). In some embodiments LPETG (SEQ ID NO: 12) is used as the sortase recognition motif. However, motifs falling outside this consensus may also be recognized. For example, in some embodiments the motif comprises an ‘A’ rather than a ‘T’ at position 4, e.g., LPXAG (SEQ ID NO: 13), or LPNAG (SEQ ID NO: 14). In some embodiments the motif comprises an ‘A’ rather than a ‘G’ at position 5, e.g., LPXTA (SEQ ID NO: 15), or LPNTA (SEQ ID NO: 16). In some embodiments the motif comprises a ‘G’ rather than ‘P’ at position 2, e.g., LGXTG (SEQ ID NO: 17) or LGATG (SEQ ID NO: 18). In some embodiments the motif comprises an ‘I’ rather than ‘L’ at position 1, e.g., IPXTG (SEQ ID NO: 19), IPNTG (SEQ ID NO: 20) or IPETG (SEQ ID NO: 21). Additional suitable sortase recognition motifs will be apparent to those of skill in the art, and the invention is not limited in this respect. It will be appreciated that the terms “recognition motif” and “recognition sequence”, with respect to sequences recognized by a transamidase or sortase, are used interchangeably. In some embodiments, the SrtA is a mutant as described herein, which may possess improved enzymatic activity relative to the wild-type counterpart. Such a mutant may recognize LAETG (SEQ ID NO: 22) and use a peptide comprising the recognition sequence as a substrate. Such sortase recognition motifs can be used in any of the methods described herein.

In some embodiments of the invention the sortase is a sortase B (SrtB) or an active fragment thereof, e.g., a sortase B of S. aureus, B. anthracis, or L. monocytogenes. Motifs recognized by sortases of the B class (SrtB) often fall within the consensus sequences NPXTX, e.g., NP[Q/K]-[T/s]-[N/G/s] (SEQ ID NO: 23), such as NPQTN (SEQ ID NO: 24) or NPKTG (SEQ ID NO: 25). For example, sortase B of S. aureus or B. anthracis cleaves the NPQTN (SEQ ID NO: 24) or NPKTG (SEQ ID NO: 25) motif of IsdC in the respective bacteria (see, e.g., Marraffini et al., Journal of Bacteriology, 189(17): 6425-6436, 2007). Other recognition motifs found in putative substrates of class B sortases are NSKTA (SEQ ID NO: 26), NPQTG (SEQ ID NO: 27), NAKTN (SEQ ID NO: 28), and NPQSS (SEQ ID NO: 29). For example, SrtB from L. monocytogenes recognizes certain motifs lacking P at position 2 and/or lacking Q or K at position 3, such as NAKTN (SEQ ID NO: 28) and NPQSS (SEQ ID NO: 29) (Mariscotti et al., J Biol Chem. 2009 Jan. 7). Such sortase recognition motifs can also be used in any of the methods described herein.

In one embodiment, the sortase enzyme is selected from a sortase A, a sortase B, a sortase C, or a sortase D, or an active fragment thereof.

The sortase acceptor peptide may comprise sortase recognition sequence (e.g., LPTXG) (SEQ ID NO: 5) for sortase A in which X is any amino acid residue), wherein the peptide is associated with a detectable label or tag, e.g., biotin or a fluorescent dye.

In some embodiments, the sortase disposed on the surface of the bait cell is a mutant sortase (e.g., a mutant sortase A) that exhibits improved catalytic activity as compared to its wild-type counterpart. In some examples, the mutant sortase A (SrtA) comprises one or more mutations of P94R or P94S, S102C, A104H, E105D, K138P, K152I, D160K or D160N, K162H, T164N, D165A, K173E, I182V, K190E, and K196S or K196T. In one example, the mutant SrtA includes mutations P94S, D160N, and K196T.

In one embodiment, the sortase enzyme is selected from sortase A: (5M) and mgSrtA.

In some particular embodiments, a bate cell is engineered to comprise on its cell surface a sortase enzyme described above. In one particular embodiment, the bate cell is engineered to comprise the sortase enzyme described above on its surface by means of a glycoconjugation method as described herein, whereby the GDP-Fuc-Enzyme conjugate is a GDP-Fuc-Sortase enzyme conjugate, which is used as the donor nucleotide substrate to conjugate the sortase enzyme onto the surface of the cell. In one particular embodiment, the bate cell is engineered to comprise the sortase enzyme described above on its surface by means of a glycoconjugation method as described herein, except that instead of utilizing a fucosyltransferase to attach a GDP-Fuc-Sortase enzyme conjugate, the method utilizes a sialyltransferase enzyme according and a CMP-NeuAc-Sortase enzyme conjugate to attach the sortase onto the surface of the cell via a sialyation reaction.

In some particular embodiments, a sortase described above is chemically conjugated to the surface of a bait cell.

In some particular embodiments, the sortase is disposed on the surface of the bait cell via a method that does not comprise expressing the sortase enzyme via genetic modification of the bait cell.

In one embodiment, the present disclosure provides bait cells comprising an enzyme on their surface, wherein the enzyme is a promiscuous biotin ligase selected from TurboID, miniTurbo, BioID, and BioID2; and methods of using the same in interaction-dependent labeling a prey cell. In the case of the promiscuous biotin ligases, interaction-dependent labeling occurs when bait cells contacting prey cells with vicinal proteins on their surface in the presence of biotin; in which case the promiscuous biotin ligase will transfer biotin to the vicinal proteins.

As will be clear to a person of ordinary skill in the art, the nature of the acceptor molecule on the prey cell and the donor sugar nucleotide-tag-conjugate or other tagged substrate that is attached to the prey cell by the bait cell is in various embodiments driven by the enzyme that is disposed on the bait cell. For example, in various embodiments, when the bait cell comprises a fucosyltransferase on its cell surface, the acceptor molecule on the prey cell is a fucose acceptor capable of being fucosylated by the fucosyltransferase, and the donor sugar nucleotide-tag conjugate is a GDP-fucose conjugate comprising a tag. Such fucose acceptors are known in the art and include LacNAc and α2,3-sialylated LacNAc (sLacNAc), which are commonly found in complex and hybrid N-glycans decorating most cell surfaces.

Similarly, when the bait cell comprises a sialyltransferase on its cell surface, the acceptor molecule on the prey cell is a sialic acid (NeuAc)-acceptor capable of being sialylated by the sialyltransferase, and the donor sugar nucleotide-tag conjugate is a CMP-Neu5Ac conjugate comprising a tag. Such NeuAc acceptors are known in the art and include Galactose and N-acetylgalactosamine GalNAc.

In various embodiments, the compositions and methods disclosed herein utilize donor nucleotide sugar substrates that are tagged; tagged sortase acceptor peptides comprising sortase recognition sequences; or other tagged substrates. Any suitable tag may be conjugated to such substrates to enable detection of a proximity label transfer. Such tags may include, without limitation, mono- or poly-histidine sequences (e.g. 6×His), FLAG-tag, myc-tag, HA-tag, V5, VSVG, GFP (and variants thereof), horseradish peroxidase (HRP); alkaline phosphatase (AP), glucose oxidase, maltose binding protein; SUMO tag, thioredoxin, poly(NANP), poly-Arg, calmodulin binding protein, PurF fragment, ketosteroid isomerase, PaP3.30, TAF12 histone fold domain, FKBP-tag, SNAP tag, Halo-tag, immunoglobulin Fc portions (and variants thereof), biotin, streptavidin, avidin, calmodulin, S-tag, SBP, CBP, softag 1, softag 3, Xpress, isopeptag, spytag, BCCP, glutathione-S-transferase (GST), maltose binding protein (MBP), Nus, thioredocin, NANP, TC, Ty, GCN4, fluorescent molecules or probes (e.g., Alexa Fluors; fluoresceins such as, e.g., FAM), fluorescent probe Cy2, Cy3, Cy3B, Cy3.5, Cy5, Cy5.5, Cy7 (or other Cyanine dyes), a peridinin chlorophyll protein complex, a fluorescent protein (e.g., a green fluorescent protein (GFP) or red fluorescent protein (RFP)), phycoerythrin (PE); and the like. If desired, the tag may be cleavable and, thus, able to be removed, e.g., by a protease. In some embodiments, this is achieved by including a protease cleavage site in the tag, e.g., adjacent or linked to a functional portion of the tag. Non-limiting examples of protease tags that may be used in this manner include thrombin, TEV protease, Factor Xa, and PreScission protease. In some embodiments, the tag is biotin. Additionally, in some embodiments, the substrate and the tag are one and the same. For example, if the enzyme that is disposed on the surface of the bait cell is a promiscuous biotin ligase, then the substrate is biotin. As noted above, biotin is a suitable tag. Thus, no conjugation of a tag to the biotin substrate is needed to facilitate detection of the presence of the substrate on the prey cell after a contact-induced conjugation of the tagged substrate onto the prey cell.

The bait cells of the present invention may be engineered to have the enzyme on their surface via any suitable method. In certain embodiments, the bait cells comprise an enzyme bound to their cell surface via conjugation. The conjugation may be via any suitable method. For example, in some embodiments, the conjugation is via chemical or enzymatic conjugation. Such conjugations methods are known in the art and disclosed herein. In some embodiments, the enzyme is expressed on the surface of the bait cell via genetic modification.

For example, in some embodiments, the bait cells comprise an enzyme bound to their cell surface via a glycosylation-based conjugation method such as, e.g., we have previously described in international application PCT/US2018/016503 (published as WO2018/144769), the content of which is incorporated herein by reference in its entirety. Suitable glycosylation-based conjugations include the methods disclosed herein. H. pylori α1,3fucosyltransferase has remarkable substrate tolerance, and essentially anything desirable may be conjugated, e.g., via a linker, to a GDP-Fucose and still be utilized by the enzyme in a glycosylation reaction. Thus, in some embodiments, the enzyme may be conjugated to the surface of a bait cell by a method comprising the following steps: First, an enzyme is linked with a “clickable” group such as tetrazine, azide or alkyne by amine-coupling or site-specific modification (such as aldehyde tag or unnatural amino acid modification). Then the enzyme is further linked with easily accessible GDP-Fucose derivatives bearing complementary “clickable” groups to form GDP-Fuc-Enzyme via click chemistry. Finally, the GDP-Fuc-Enzyme is transferred onto a cell surface catalyzed by H. pylori α1,3fucosyltransferase, which glycosylates glyco-acceptors on the surface of the cell such a LacNAc/sialylLacNAc glycans. Similarly, in some embodiments, other fucosyltransferases may be used to catalyze the transfer of GDP-Fuc-Enzyme onto the surface of the bait cell. For example, in certain embodiments, Helicobacter mustelae al-2-fucosyltransferase (Hm1,2FT), H. pylori α1,3/1,4 fucosyltransferase; or human α1,3 fucosyltransferase (FUT6) is used to catalyze the transfer of GDP-FUC-Enzyme onto the surface of a bait cell. Similarly, in certain embodiments, a sialyltransferase is used to catalyze the transfer CMP-NeuAc-Enzyme onto the surface of a bait cell. For example, in certain embodiments, ST6GAL1; ST6GalNAc1; ST3Gal1; Pasteurella multocida a (2,3) sialyltransferase M144D mutant (Pm2,3ST-M144D); or Photobacterium damsela a (2,6) sialyltransferase (Pd2,6ST) is used to catalyze the transfer of CMP-NeuAc-Enzyme onto the surface of a bait cell.

In some embodiments, the enzyme is conjugated to the surface of a bait cell by a chemical conjugation method. In certain embodiments, the enzyme is chemically conjugated to the surface of the bait cell comprising the methods disclosed herein: first enzyme is linked with tetrazine by amine-coupling to form enzyme-tetrazine conjugates (Enzyme-Tz). Next, the cells that are to be conjugated with the enzyme are treated with TCO-NHS ester to introduce TCO moieties onto their cell surfaces. Finally, the enzyme-Tz conjugates are reacted with the TCO-NHS moieties on the cell surfaces by biorthogonal reaction to form cell-enzyme surface conjugates.

In some embodiments, the enzyme is expressed on the surface of the bait cell via genetic modification. Methods for genetically altering a cell are well known in the art, and any suitable method may be used to engineer a bait cell of the present invention. In some embodiments, the enzyme is expressed on the surface of the bait cell using standard recombinant techniques in molecular biology that are well known, (e.g., see, Joseph Sambrook, et al., Molecular Cloning: A Laboratory Manual, 2nd ed., 1.53 [Cold Spring Harbor Laboratory Press 1989], incorporated herein). The methodology is not limited to any particular cloning strategy. A person of ordinary skill in the art will fully appreciate that one may use any variety of cloning strategies to produce an expression vector containing an enzyme for expression on a bait cell in accordance with the present disclosure. For example, one or more polynucleotides encoding the enzyme may be cloned into an expression vector along with suitable vector elements to drive expression of the enzyme onto the surface of the bait cell. In some instances, once the nucleotide sequence for the enzyme has been determined (optionally the ‘codon optimized’ sequence) the gene (or “polynucleotide,” interchangeably) is synthesized de novo to form a cDNA that contains the gene plus additional nucleotide sequences containing unique restriction enzyme cleavage sites on the 5′ and 3′ ends of the gene. Several direct gene synthesis methods can be employed for this purpose, these methods are well known to those in the art (Montague M G, Lartigue C, Vashee S. (2012) Synthetic genomics: potential and limitations. Curr. Opin. Biotechnol. 23(5):659-665, incorporated herein). Polynucleotides representing the entire enzyme gene, plus the aforementioned additional nucleotide sequences, can be directly synthesized, or alternatively subfragments of the gene may be directly synthesized followed by ligation of these fragments using PCR primers to create the full-length gene. Following gene synthesis the nucleotide sequence of the novel gene with the flanking restriction sites, and optional regulatory elements, may be confirmed by direct gene sequencing of the cDNA that are well known to those skilled in the art (Pettersson E, Lundeberg J, Ahmadian A. (2009) Generations of sequencing technologies. Genomics 93:(2)105-111, incorporated herein). Once the cDNA sequence has been confirmed the cDNA is cloned into a recombinant protein expression vector using standard recombinant techniques in molecular biology. Expression vectors are typically selected to match the particular host cell used for expressing the recombinant protein in order to optimize the quantity and quality of recombinant protein expressed. Expression vectors are typically engineered with nucleotide sequences that represent additional elements needed to optimize the expression of the novel gene in a particular host cell, including but not limited to, cloning sites to facilitate insertion of the cDNA containing the novel gene, a promoter/enhancer element to allow efficient, high-level gene expression, primer sites to allow sequencing of the cDNA insert, a polyadenylation signal to allow efficient transcription termination and polyadenylation of the novel gene's mRNA, and selection genes to allow for selection of transformants in bacterial and mammalian cells. The expression vector may be a eukaryotic expression vector. The expression vector may be a mammalian expression vector. The expression vector may be a viral expression vector. The expression vector may be a lentiviral expression vector. The methods may further comprise validating the cloning of the one or more polynucleotides encoding the enzyme into the expression vector comprising sequencing the expression vector, running gel electrophoresis of the vector and/or viewing the enzyme on an SDS page gel. The methods may further comprise amplifying a polynucleotide encoding the enzyme and cloning the enzyme into the expression vector. Amplifying the polynucleotide encoding the enzyme may comprise synthesizing oligonucleotides at least partially complementary to the gene. The oligonucleotides may be sufficiently complementary to the gene to anneal to the polynucleotide. The oligonucleotides may comprise linker sequences. Many suitable linkers are known in the art and are suitable for use in the present invention.

The methods may comprise transfecting or infecting a cell with the expression vector. The methods may further comprise expressing the enzyme in the cell. The methods may further comprise expressing the enzyme in a cell free system. The methods may further comprise producing a virus comprising the expression vector. The methods may further comprise propagating the virus. The methods may further comprise infecting a cell with the virus comprising the expression vector. The methods may further comprise propagating the cell.

In some embodiments, the enzyme is expressed using the lentiviral vector, wherein human FUT6 is expressed on the surface of a bait cell. This construct includes from N- to C-terminus a membrane alanyl aminopeptidase transmembrane domain (TMD) linked to an HA tag via two glycine amino acids; and a Linker (containing a TEV cleavage site) linking the HA tagged TMD to the FUT6 ectodomain. The protein sequence of the FUT6 lentivirus construct insert is 416aa and is predicted to encode a 47.21 kDa protein with the following amino acid sequence:

(SEQ ID NO: 30)
MAKGFYISKSLGILGILLGVAAVCTIIALSVVYSQEKNKNANSSPVAST
TPSASATTNPASATTLGGYPYDVPDYAEFASTSLYKKAGSENLYFQGD
PTVYPNGSRFPDSTGTPAHSIPLILLWTWPFNKPIALPRCSEMVPGTAD
CNITADRKVYPQADAVIVHHREVMYNPSAQLPRSPRRQGQRWIWFSM
ESPSHCWQLKAMDGYFNLTMSYRSDSDIFTPYGWLEPWSGQPAHPPL
NLSAKTELVAWAVSNWGPNSARVRYYQSLQAHLKVDVYGRSHKPLP
QGTMMETLSRYKFYLAFENSLHPDYITEKLWRNALEAWAVPVVLGPS
RSNYERFLPPDAFIHVDDFQSPKDLARYLQELDKDHARYLSYFRWRET
LRPRSFSWALAFCKACWKLQEESRYQTRGIAAWFT

The FUT6 lentivirus construct insert may be encoded by the following DNA sequence (1251 bp).

(SEQ ID NO: 31)
ATGGCCAAGGGCTTCTATATTTCCAAGTCCCTGGGCATCCTGGGGA
TCCTCCTGGGCGTGGCAGCCGTGTGCACAATCATCGCACTGTCAGT
GGTGTACTCCCAGGAGAAGAACAAGAACGCCAACAGCTCCCCCGT
GGCCTCCACCACCCCGTCCGCCTCAGCCACCACCAACCCCGCCTCG
GCCACCACCTTGGGCGGCTACCCATACGATGTTCCAGATTACGCTG
AGTTCGCCAGCACCAGCCTGTACAAGAAGGCCGGCAGCGAGAACC
TGTACTTCCAGGGCGATCCCACTGTGTACCCTAATGGGTCCCGCTT
CCCAGACAGCACAGGGACCCCCGCCCACTCCATCCCCCTGATCCTG
CTGTGGACGTGGCCTTTTAACAAACCCATAGCTCTGCCCCGCTGCT
CAGAGATGGTGCCTGGCACGGCTGACTGCAACATCACTGCCGACC
GCAAGGTGTATCCACAGGCAGACGCGGTCATCGTGCACCACCGAG
AGGTCATGTACAACCCCAGTGCCCAGCTCCCACGCTCCCCGAGGCG
GCAGGGGCAGCGATGGATCTGGTTCAGCATGGAGTCCCCAAGCCA
CTGCTGGCAGCTGAAAGCCATGGACGGATACTTCAATCTCACCATG
TCCTACCGCAGCGACTCCGACATCTTCACGCCCTACGGCTGGCTGG
AGCCGTGGTCCGGCCAGCCTGCCCACCCACCGCTCAACCTCTCGGC
CAAGACCGAGCTGGTGGCCTGGGCAGTGTCCAACTGGGGGCCAAA
CTCCGCCAGGGTGCGCTACTACCAGAGCCTGCAGGCCCATCTCAAG
GTGGACGTGTACGGACGCTCCCACAAGCCCCTGCCCCAGGGAACC
ATGATGGAGACGCTGTCCCGGTACAAGTTCTATCTGGCCTTCGAGA
ACTCCTTGCACCCCGACTACATCACCGAGAAGCTGTGGAGGAACGC
CCTGGAGGCCTGGGCCGTGCCCGTGGTGCTGGGCCCCAGCAGAAG
CAACTACGAGAGGTTCCTGCCACCCGACGCCTTCATCCACGTGGAC
GACTTCCAGAGCCCCAAGGACCTGGCCCGGTACCTGCAGGAGCTG
GACAAGGACCACGCCCGCTACCTGAGCTACTTTCGCTGGCGGGAG
ACGCTGCGGCCTCGCTCCTTCAGCTGGGCACTCGCTTTCTGCAAGG
CCTGCTGGAAACTGCAGGAGGAATCCAGGTACCAGACACGCGGCA
TAGCGGCTTGGTTCACCTGA

The FUT6 lentivirus construct with the insert in the lentiviral expression vector may be encoded by the following DNA sequence (8622 bp)

(SEQ ID NO: 32)
caggtggcacttttcggggaaatgtgcgcggaacccctatttgtttatttttctaaatacattcaaatatgtatccgct
catgagacaataaccctgataaatgcttcaataatattgaaaaaggaagagtatgagtattcaacatttccgtgtcg
cccttattcccttttttgcggcattttgccttcctgtttttgctcacccagaaacgctggtgaaagtaaaagatgctga
agatcagttgggtgcacgagtgggttacatcgaactggatctcaacagcggtaagatccttgagagttttcgccc
cgaagaacgttttccaatgatgagcacttttaaagttctgctatgtggcgcggtattatcccgtattgacgccgggc
aagagcaactcggtcgccgcatacactattctcagaatgacttggttgagtactcaccagtcacagaaaagcatc
ttacggatggcatgacagtaagagaattatgcagtgctgccataaccatgagtgataacactgcggccaacttac
ttctgacaacgatcggaggaccgaaggagctaaccgcttttttgcacaacatgggggatcatgtaactcgccttg
atcgttgggaaccggagctgaatgaagccataccaaacgacgagcgtgacaccacgatgcctgtagcaatgg
caacaacgttgcgcaaactattaactggcgaactacttactctagcttcccggcaacaattaatagactggatgga
ggcggataaagttgcaggaccacttctgcgctcggcccttccggctggctggtttattgctgataaatctggagc
cggtgagcgtgggtctcgcggtatcattgcagcactggggccagatggtaagccccccgtatcgtagttatcta
cacgacggggagtcaggcaactatggatgaacgaaatagacagatcgctgagataggtgcctcactgattaag
cattggtaactgtcagaccaagtttactcatatatactttagattgatttaaaacttcatttttaatttaaaaggatctag
gtgaagatcctttttgataatctcatgaccaaaatcccttaacgtgagttttcgttccactgagcgtcagaccccgta
gaaaagatcaaaggatcttcttgagatcctttttttctgcgcgtaatctgctgcttgcaaacaaaaaaaccaccgct
accagcggtggtttgtttgccggatcaagagctaccaactctttttccgaaggtaactggcttcagcagagcgca
gataccaaatactgtccttctagtgtagccgtagttaggccaccacttcaagaactctgtagcaccgcctacatac
ctcgctctgctaatcctgttaccagtggctgctgccagtggcgataagtcgtgtcttaccgggttggactcaagac
gatagttaccggataaggcgcagcggtcgggctgaacggggggttcgtgcacacagcccagcttggagcga
acgacctacaccgaactgagatacctacagcgtgagctatgagaaagcgccacgcttcccgaagggagaaag
gcggacaggtatccggtaagcggcagggtcggaacaggagagcgcacgagggagcttccagggggaaac
gcctggtatctttatagtcctgtcgggtttcgccacctctgacttgagcgtcgatttttgtgatgctcgtcagggggg
cggagcctatggaaaaacgccagcaacgcggcctttttacggttcctggccttttgctggccttttgctcacatgtt
ctttcctgcgttatcccctgattctgtggataaccgtattaccgcctttgagtgagctgataccgctcgccgcagcc
gaacgaccgagcgcagcgagtcagtgagcgaggaagcggaagagcgcccaatacgcaaaccgcctctccc
cgcgcgttggccgattcattaatgcagctggcacgacaggtttcccgactggaaagcgggcagtgagcgcaac
gcaattaatgtgagttagctcactcattaggcaccccaggctttacactttatgcttccggctcgtatgttgtgtgga
attgtgagcggataacaatttcacacaggaaacagctatgaccatgattacgccaagegcgcaattaaccctcac
taaagggaacaaaagctggagctgcaagcttaatgtagtcttatgcaatactcttgtagtcttgcaacatggtaac
gatgagttagcaacatgccttacaaggagagaaaaagcaccgtgcatgccgattggtggaagtaaggtggtac
gatcgtgccttattaggaaggcaacagacgggtctgacatggattggacgaaccactgaattgccgcattgcag
agatattgtatttaagtgcctagctcgatacaataaacgggtctctctggttagaccagatctgagcctgggagctc
tctggctaactagggaacccactgcttaagcctcaataaagcttgccttgagtgcttcaagtagtgtgtgcccgtct
gttgtgtgactctggtaactagagatccctcagacccttttagtcagtgtggaaaatctctagcagtggcgcccga
acagggacctgaaagcgaaagggaaaccagagctctctcgacgcaggactcggcttgctgaagcgcgcacg
gcaagaggcgaggggcggcgactggtgagtacgccaaaaattttgactagcggaggctagaaggagagag
atgggtgcgagagcgtcagtattaagcgggggagaattagatcgcgatgggaaaaaattcggttaaggccag
ggggaaagaaaaaatataaattaaaacatatagtatgggcaagcagggagctagaacgattcgcagttaatcct
ggcctgttagaaacatcagaaggctgtagacaaatactgggacagctacaaccatcccttcagacaggatcag
aagaacttagatcattatataatacagtagcaaccctctattgtgtgcatcaaaggatagagataaaagacaccaa
ggaagctttagacaagatagaggaagagcaaaacaaaagtaagaccaccgcacagcaagcggccgctgatc
ttcagacctggaggaggagatatgagggacaattggagaagtgaattatataaatataaagtagtaaaaattgaa
ccattaggagtagcacccaccaaggcaaagagaagagtggtgcagagagaaaaaagagcagtgggaatag
gagctttgttccttgggttcttgggagcagcaggaagcactatgggcgcagcctcaatgacgctgacggtacag
gccagacaattattgtctggtatagtgcagcagcagaacaatttgctgagggctattgaggcgcaacagcatctg
ttgcaactcacagtctggggcatcaagcagctccaggcaagaatcctggctgtggaaagatacctaaaggatca
acagctcctggggatttggggttgctctggaaaactcatttgcaccactgctgtgccttggaatgctagttggagt
aataaatctctggaacagattggaatcacacgacctggatggagtgggacagagaaattaacaattacacaagc
ttaatacactccttaattgaagaatcgcaaaaccagcaagaaaagaatgaacaagaattattggaattagataaat
gggcaagtttgtggaattggtttaacataacaaattggctgtggtatataaaattattcataatgatagtaggaggct
tggtaggtttaagaatagtttttgctgtactttctatagtgaatagagttaggcagggatattcaccattatcgtttcag
acccacctcccaaccccgaggggacccgacaggcccgaaggaatagaagaagaaggtggagagagagac
agagacagatccattcgattagtgaacggatctcgacggttaacttttaaaagaaaaggggggattggggggta
cagtgcaggggaaagaatagtagacataatagcaacagacatacaaactaaagaattacaaaaacaaattaca
aaaattcaaaattttatcgagctttgcaaagatggataaagttttaaacagagaggaatctttgcagctaatggacc
ttctaggtcttgaaaggagtgcctcgtgaggctccggtgcccgtcagtgggcagagcgcacatcgcccacagt
ccccgagaagttggggggaggggtcggcaattgaaccggtgcctagagaaggtggcgcggggtaaactgg
gaaagtgatgtcgtgtactggctccgcctttttcccgaggggggggagaaccgtatataagtgcagtagtcgcc
gtgaacgttctttttcgcaacgggtttgccgccagaacacaggtaagtgccgtgtgtggttcccgcgggcctggc
ctctttacgggttatggcccttgcgtgccttgaattacttccacctggctgcagtacgtgattcttgatcccgagcttc
gggttggaagtgggtgggagagttcgaggccttgcgcttaaggagccccttcgcctcgtgcttgagttgaggcc
tggcctgggcgctggggccgccgcgtgcgaatctggtggcaccttcgcgcctgtctcgctgctttcgataagtct
ctagccatttaaaatttttgatgacctgctgcgacgctttttttctggcaagatagtcttgtaaatgcgggccaagatc
tgcacactggtatttcggtttttggggccgcgggcggcgacggggcccgtgcgtcccagcgcacatgttcggc
gaggggggcctgcgagcgcggccaccgagaatcggacgggggtagtctcaagctggccggcctgctctg
gtgcctggcctcgcgccgccgtgtatcgccccgccctgggcggcaaggctggcccggtcggcaccagttgc
gtgagcggaaagatggccgcttcccggccctgctgcagggagctcaaaatggaggacgcggcgctcggga
gagcgggcgggtgagtcacccacacaaaggaaaagggcctttccgtcctcagccgtcgcttcatgtgactcca
cggagtaccgggcgccgtccaggcacctcgattagttctcgagcttttggagtacgtcgtctttaggttgggggg
aggggttttatgcgatggagtttccccacactgagtgggtggagactgaagttaggccagcttggcacttgatgt
aattctccttggaatttgccctttttgagtttggatcttggttcattctcaagcctcagacagtggttcaaagtttttttctt
ccatttcaggtgtcgtgaggaattcggtaccgcggccgcccggggatccatggccaagggcttctatatttccaa
gtccctgggcatcctggggatcctcctgggcgtggcagccgtgtgcacaatcatcgcactgtcagtggtgtact
cccaggagaagaacaagaacgccaacagctcccccgtggcctccaccaccccgtcegcctcagccaccacc
aaccccgcctcggccaccaccttgggcggctacccatacgatgttccagattacgctgagttcgccagcacca
gcctgtacaagaaggccggcagcgagaacctgtacttccagggcgatcccactgtgtaccctaatgggtcccg
cttcccagacagcacagggacccccgcccactccatccccctgatcctgctgtggacgtggccttttaacaaac
ccatagctctgccccgctgctcagagatggtgcctggcacggctgactgcaacatcactgccgaccgcaaggt
gtatccacaggcagacgcggtcatcgtgcaccaccgagaggtcatgtacaaccccagtgcccagctcccacg
ctccccgaggcggcaggggcagcgatggatctggttcagcatggagtccccaagccactgctggcagctgaa
agccatggacggatacttcaatctcaccatgtcctaccgcagcgactccgacatcttcacgccctacggctggct
ggagccgtggtccggccagcctgcccacccaccgctcaacctctcggccaagaccgagctggtggcctggg
cagtgtccaactgggggccaaactccgccagggtgcgctactaccagagcctgcaggcccatctcaaggtgg
acgtgtacggacgctcccacaagcccctgccccagggaaccatgatggagacgctgtcccggtacaagttcta
tctggccttcgagaactccttgcaccccgactacatcaccgagaagctgtggaggaacgccctggaggcctgg
gccgtgcccgtggtgctgggccccagcagaagcaactacgagaggttcctgccacccgacgccttcatccac
gtggacgacttccagagccccaaggacctggcccggtacctgcaggagctggacaaggaccacgcccgcta
cctgagctactttcgctggcgggagacgctgcggcctcgctccttcagctgggcactcgctttctgcaaggcctg
ctggaaactgcaggaggaatccaggtaccagacacgcggcatagcggcttggttcacctgagtcgacaatcaa
cctctggattacaaaatttgtgaaagattgactggtattcttaactatgttgctccttttacgctatgtggatacgctgc
tttaatgcctttgtatcatgctattgcttcccgtatggctttcattttctcctccttgtataaatcctggttgctgtctctttat
gaggagttgtggcccgttgtcaggcaacgtggcgtggtgtgcactgtgtttgctgacgcaacccccactggttg
gggcattgccaccacctgtcagctcctttccgggactttcgctttccccctccctattgccacggcggaactcatc
gccgcctgccttgcccgctgctggacaggggctcggctgttgggcactgacaattccgtggtgttgtcggggaa
gctgacgtcctttccatggctgctcgcctgtgttgccacctggattctgcgcgggacgtccttctgctacgtccctt
cggccctcaatccagcggaccttccttcccgcggcctgctgccggctctgcggcctcttccgcgtcttcgccttc
gccctcagacgagtcggatctccctttgggccgcctccccgcctggaattcgagctcggtacctttaagaccaat
gacttacaaggcagctgtagatcttagccactttttaaaagaaaaggggggactggaagggctaattcactccca
acgaagacaagatctgctttttgcttgtactgggtctctctggttagaccagatctgagcctgggagctctctggct
aactagggaacccactgcttaagcctcaataaagcttgccttgagtgcttcaagtagtgtgtgcccgtctgttgtgt
gactctggtaactagagatccctcagacccttttagtcagtgtggaaaatctctagcagtagtagttcatgtcatctt
attattcagtatttataacttgcaaagaaatgaatatcagagagtgagaggaacttgtttattgcagcttataatggtt
acaaataaagcaatagcatcacaaatttcacaaataaagcatttttttcactgcattctagttgtggtttgtccaaact
catcaatgtatcttatcatgtctggctctagctatcccgcccctaactccgcccagttccgcccattctccgccccat
ggctgactaattttttttatttatgcagaggccgaggccgcctcggcctctgagctattccagaagtagtgaggag
gcttttttggaggcctaggcttttgcgtcgagacgtacccaattcgccctatagtgagtcgtattacgcgcgctcac
tggccgtcgttttacaacgtcgtgactgggaaaaccctggcgttacccaacttaatcgccttgcagcacatcccc
ctttcgccagctggegtaatagcgaagaggcccgcaccgatcgcccttcccaacagttgcgcagcctgaatgg
cgaatggcgcgacgcgccctgtagcggcgcattaagcgcggcgggtgtggtggttacgcgcagegtgaccg
ctacacttgccagcgccctagcgcccgctcctttcgctttcttcccttcctttctcgccacgttegccggetttcccc
gtcaagctctaaatcgggggctccctttagggttccgatttagtgctttacggcacctegaccccaaaaaacttga
ttagggtgatggttcacgtagtgggccatcgccctgatagacggtttttcgccctttgacgttggagtccacgttctt
taatagtggactcttgttccaaactggaacaacactcaaccctatctcggtctattcttttgatttataagggattttgc
cgatttcggcctattggttaaaaaatgagctgatttaacaaaaatttaacgcgaattttaacaaaatattaacgtttac
aatttcc

In some embodiments, the present disclosure provides a method for interaction-dependent labeling a prey cell with a bait cell, the method comprising contacting a prey cell with a bait cell in the presence of a suitable tagged substrate. Reference herein to a “suitable tagged substrate” means a substrate that may be utilized in an interaction-dependent labeling reaction by an enzyme that is bound on the surface of a bait cell, wherein the substrate comprises a tag, as disclosed herein. So, e.g., but not to be limited in any way, if the enzyme disposed on the bait cell is a fucosyltransferase, then reference to a “suitable tagged substrate” means a tagged-GDP-Fucose (e.g., GDP-Fuc-Biotin). Similarly, if the enzyme disposed on the bait cell is a sialyltransferase, then reference to a “suitable tagged substrate” means a tagged CMP-sialic acid (e.g., CMP-NeuAc-Biotin). Due to the presence of the enzyme on the surface of the bait cell, such a contacting event results in interaction-dependent labeling; i.e., the contact-induced conjugation of the tagged substrate onto the prey cell. As discussed above, the choice of a suitable substrate will be clear to a person of skill in the art and will depend on the enzyme that is engineered on the bait cell.

The presence of a tag on a prey cell (i.e., “the labeled cell”) may be determined by any suitable means including, e.g., without limitation via immunofluorescence; immunohistochemistry; immunoblot; flow cytometry; FACS; microarray analysis, SDS page; mass spectrometry; HPLC: The labeled cell may be enriched for, e.g., utilizing FACS sorting for the presence of the label. The labeled calls may be further sorted by the existence or lack of existence of other markers, e.g., cell surface markers such as e.g. PD-1, CD134, CD137, CXCR5, and/or TIM3 and/or additional markers for indicating cell type, e.g., CD45, CD8, CD4, and the like.

In one embodiment, the present disclosure provides compositions and methods by which an enzyme, for example, fucosyltransferase (FT) is conjugated to its substrate GDP-Fuc via a short PEG linker to form GDP-Fuc-FT. GDP-Fuc-FT serves as the self-catalyst to transfer Fuc-FT to LacNAc in the cell-surface glycocalyx in approximately 15 mins. The cell-FT conjugate is capable of transferring probe molecules (e.g., GDP-Fuc-biotin or GDP-Fuc-tag) to the surface glycans of contact prey cells, for the detection of a cell-cell interaction. This technique has several advantages: (1) It is suitable for different cell types since GDP-fucose acceptor-glycans LacNAc or a2,3-sialylated LacNAc (sLacNAc) are commonly found in complex and hybrid N-glycans decorating most cell surfaces; (2) no time-consuming and complicated genetic modification is necessary; and (3) common laboratory techniques such as fluorescent microscopic imaging and flow cytometry/FACS are used to detect/monitor cell-cell interactions/sort for cells that have been proximity labeled in this manner.

In some embodiments, the present disclosure provides a method for tagging a specific antigen. In some embodiments, the present disclosure provides a method for tagging virus-specific antigen. In some embodiments, the present disclosure provides a method for tagging a tumor-specific antigen (TSA) reactive T cell present in a population of tumor infiltrating lymphocytes (TTLs).

In some embodiments, the method comprises cells wherein, (a) any cells comprising the label and exhibiting both CD8+ expression and PD-1+ expression are antigen reactive cytotoxic T cells; and (b) any cells comprising the label and exhibiting both CD4+ expression and PD-1+ expression are antigen reactive helper T cells. In some embodiments, the magnitude of label present on the T cell is indicative of the binding affinity of a T cell receptor expressed on the surface of the T cell for the antigen. In some embodiments, the magnitude of label present on the T cell is indicative of the enriched T cell's ability to kill other cells expressing the antigen and/or of the enriched T cell's ability to become activated in the presence of the antigen.

In some embodiments, the TSA reactive T cells are substantially or entirely CD4+. In some embodiments, the TSA reactive T cells are substantially or entirely CD8+.

In some embodiments, the method further comprises sequencing a cell having label on its cell surface by single cell T cell receptor (TCR) sequencing to identify an antigen-specific TCR expressed by the cell. In some embodiments, the method further comprises expanding the enriched cells for subject specific immune cell therapy.

In some embodiments, the analyzing comprises enriching for cells comprising the label via Fluorescence-activated cell sorting (FACS). In some embodiments, the analyzing further comprises determining whether the cells comprising the label further comprise other markers indicative of antigen reactivity. In some embodiments, the method further comprises enriching for cells comprising the other markers indicative of antigen reactivity via FACS. For example, in some embodiments, the cells are sorted to enrich for tagged cells expressing T cell markers (i.e., enrich for CD8+ cells for cytotoxic T-cells; CD4+ for helper T cells); to exclude cells expressing DC markers (i.e., enrich for CD45.1−/− cells); and/or to enrich for markers indicative of prospective TSA reactivity (i.e., enrich for cells that are PD-1+, CD134+, CD137+) and or other markers such as CXCR5+, and/or TIM3+. In some embodiments, the other markers include one or more of PD-1 expression, TCF-1 expression, and TIM3 expression. In some embodiments, the method comprises enriching for cells comprising both the label and PD-1 expression. In some embodiments, the method further comprises enriching for cells comprising CD8+ and/or CD4+ expression.

In some embodiments of the method, the enzyme is a fucosyltransferase and the tagged substrate is GDP-fucose conjugated to a tag. In some embodiments, the fucosyltransferase is H. pylori α1,3fucosyltransferase. In some embodiments, the donor sugar nucleotide is GDP-fucose. In some embodiments, the tag is any one of the tags disclosed herein. In some embodiments, the tag is biotin. In some embodiment, the fucosyltransferase enzyme is a human fucosyltransferase. In some embodiment, the fucosyltransferase enzyme is human α 1,3-fucosyltransferase. In one embodiment, the human α 1,3-fucosyltransferase is recombinantly prepared. In some embodiment, the fucosyltransferase enzyme is not a human fucosyltransferase. In some embodiment, the fucosyltransferase enzyme is an H. pylori fucosyltransferase. In one embodiment, the H. pylori fucosyltransferase is H. pylori α 1,3-fucosyltransferase. In one embodiment, the H. pylori fucosyltransferase is H. pylori α 1,3/1,4-fucosyltransferase. In some embodiments of the method, the enzyme is a sialyltransferase and the tagged substrate is CMP-Neu5Ac conjugated to a tag. In some embodiments, the tag is biotin. In some embodiments, the sialyltransferase enzyme is a human sialyltransferase. In some embodiments, the sialyltransferase enzyme is human ST6GAL1. In one embodiment, the human ST6GAL1 is recombinantly prepared. In some embodiment, the sialyltransferase enzyme is human ST6GalNAc1. In one embodiment, the human ST6GalNAc1 is recombinantly prepared. In some embodiments, the sialyltransferase enzyme is not a human sialyltransferase. In some embodiments, the sialyltransferase enzyme is Pasteurella multocida α (2,3) sialyltransferase M144D mutant (Pm2,3ST-M144D) or Photobacterium damsela α (2,6) sialyltransferase (Pd2,6ST). In some embodiments, the bait cell is engineered to comprise the enzyme on its surface via conjugation of the enzyme to the cell's surface or via recombinant expression of the enzyme in the cell. In some embodiments, the conjugation is a chemical conjugation. In some embodiments, the conjugation is via enzymatic conjugation of the enzyme to the cell surface. In some embodiments, the enzymatic conjugation is via fucosylation of the cell with a GDP-Fuc-Enzyme conjugate. In particular embodiments, the fucosylation enzyme catalyzing the conjugation of the enzyme to the surface of the cell is H. pylori α1,3fucosyltransferase enzyme. In some embodiments, the fucosylation enzyme catalyzing the conjugation of the enzyme to the surface of the cell is human α1,3fucosyltransferase (FUT6) or H. pylori α1,3/4fucosyltransferase). In some embodiments, the enzymatic conjugation is performed as described herein, except that instead of using a fucosylation reaction with a fucosyltransferase enzyme and a GDP-Fuc-Enzyme conjugate as the donor nucleotide substrate to conjugate the enzyme onto the surface of the cell, the method utilizes a sialyltransferase enzyme according to and a CMP-NeuAc-Enzyme to conjugate the enzyme onto the surface of the cell. In some embodiments, the sialyltransferase enzyme is a human sialyltransferase. In some embodiments, the sialyltransferase enzyme is human ST6GAL1 human ST6GalNAc1. In some embodiments, the sialyltransferase enzyme is not a human sialyltransferase. In some embodiments, the sialyltransferase enzyme is Pasteurella multocida α (2,3) sialyltransferase M144D mutant (Pm2,3ST-M144D) or Photobacterium damsela α (2,6) sialyltransferase (Pd2,6ST).

In some embodiments, the label is selected from a small molecule, a polynucleotide, a polypeptide, an antibody, a chemical or biological marker and/or probe. In some embodiments, the chemical or biological moiety is biotin, a biotin probe, a fluorescent molecule, a probe comprising a fluorescent molecule, a dye, a probe comprising a dye, a dye-labeled single strand DNA, a FLAG tag, or a Strep tag.

In some embodiments, the cell surface glycan is selected from Gal, LacNAc, and sialyl LacNAc. In some embodiments, the fucosyltransferase is not native to the second bait cell. In some embodiments, the fucosyltransferase is conjugated to the cell surface of the second cell. In some embodiments, the fucosyltransferase is covalently bound to the cell surface of the second cell. In some embodiments, the fucosyltransferase is covalently bound to a second cell surface glycan present on the surface of the second cell. In some embodiments, the fucosyltransferase and the second cell surface glycan are covalently bound via a glycosylation reaction. In some embodiments, the fucosyltransferase is attached to the second donor sugar nucleotide via a linker moiety. In some embodiments, the fucosyltransferase is recombinantly expressed on the cell surface of the second bait cell.

In some embodiments, the expression of the fucosyltransferase is driven by a conditionally activated promoter. In some embodiments, the conditionally activated promoter is activated in the presence of an exogenous compound. In some embodiments, the exogenous compound is a small molecule or polypeptide.

In some embodiments, the method takes less than two weeks to complete. In some embodiments, the method does not comprise identifying antigen candidates prior to enriching for the antigen-reactive T cells. In some embodiments, the method comprises excluding non-enriched T cells from the enriched T cells.

Additionally, in some embodiments, the methods disclosed above for tagging and isolating TSA reactive and autoreactive T cells are easily adapted to apply to any antigen without undue experimentation. For example, in order to tag and isolate T cells with specificity for another antigens, e.g., pathogenic antigens such as bacterial or viral antigens, one need only follow the same protocols set forth above, but use relevant antigen sources to prime the bait cell dendritic cells and then use appropriate sources of tissue infiltrating or circulating T cells from appropriate sources such that these sources contain one or more T cells with TCR-specificity for the primed antigens. So, briefly, to identify T cells with virus specific reactivity, one need only incubate a bait cell dendritic cell with either virus specific antigens or a source of the same (e.g., diseased tissue cell lysate) to prime the DCs, then the primed DC-enzyme conjugates are mixed with the tissue infiltrating or circulating T cells in the presence of the suitable tagged substrate and tagged T cells are detected and enriched for as described above. In various embodiments, the same applies for any antigen of interest with only minor changes to the methods described with respect to the methods of isolating suitable antigens/antigen sources for priming dendritic cells and minor changes to the methods of isolating relevant populations of T cells that comprise antigen-specific T cells to be identified and isolated using the interaction-dependent labeling methods described herein.

For example, in order to identify prospective TSA-specific T cells for hematologic malignancies, such as AML; ALL; CLL; the following protocol may in some embodiments be followed. Cancer cells are isolated from a patient (bone marrow or blood) and lysed for priming iDCs derived from the same patient. The primed iDCs or un-primed (control) iDCs are stained with CellTracker™ Green CMFDA, and conjugated with fucosyltransferase (FT) on the cell surface, and cultured with autologous PBMC of the same patient at different ratios for 1-2 hours. Then GDP-Fuc-biotin (50 μM) is added and incubated for another 30 min. After quenching the reaction with N-Acetyl-D-lactosamine (LacNAc), the cell mixture is stained with Alexa Fluor 647-streptavidin and cell identity markers, and subjected to flow cytometry analysis. CD4+ (Foxp3−) and or CD8+ T cells that are also Alexa Fluor 647+ will be isolated as prospective TSA-specific T cells.

Similarly, to identify prospective TSA-specific T cells for solid tumors in human, the following protocol may in some embodiments be followed. Tumors isolated from a patient are cross-cut into small pieces, minced to prepare tumor lysates or prepare single cell suspensions. Preparation of single cell suspensions are performed using pre-established Ficoll-paque density gradient centrifugation protocols that are well-known in the art. See, e.g., Tan Y. S., Lei Y. L. (2019) Isolation of Tumor-Infiltrating Lymphocytes by Ficoll-Paque Density Gradient Centrifugation. In: Allen I. (eds) Mouse Models of Innate Immunity. Methods in Molecular Biology, vol 1960. Humana Press, New York, NY; incorporated herein by reference in its entirety. Tumor lysates are used to prime iDCs derived from the same patient. The primed iDCs or unprimed (negative control) iDCs or iDC primed with normal tissue lysates (negative control) are stained with CellTracker™ Green CMFDA, conjugated with fucosyltransferase (FT) on the cell surface, and cultured with autologous single cell suspension isolated from the tumor at different ratios (autologous single cells:DC=5:1 to 10:1) for 1-2 hours. Then GDP-Fuc-biotin (50 μM) is added and incubated for another 30 min. After quenching the reaction with LacNAc, the cell mixture is stained with Alexa Fluor 647-streptavidin and cell identity and phenotype markers, and subjected to FACs. CD3+/CD4+/CD25−/Alexa Fluor 647+ or CD3+/CD8+/PD-1+/Alexa Fluor 647+ cells are isolated as prospective TSA-specific CD4 or CD8 T cells, respectively.

In particular embodiments, the T cells express a TSA specific T cell receptor (TCR), or an antigen-binding fragment thereof, comprising a Vα and a Vβ derived from a wild type T cell receptor, wherein the Vα and Vβ each comprise a complementarity determining region 1 (CDR-1), a complementarity determining region 2 (CDR-2), and a complementarity determining region 3 (CDR-3), wherein the Vα CDR-3 comprises an amino acid sequence selected from the group consisting of:

SEQ ID NO: 33
ASGTDYAEQF
SEQ ID NO: 34
ASSPQLGGRREQY
SEQ ID NO: 35
ASSIGTANTEVF
SEQ ID NO: 36
AWSGNTEVF
SEQ ID NO: 37
ASRSGGSAETLY
SEQ ID NO: 38
ASSFVSSAETLY
SEQ ID NO: 39
ASSSDRGSAETLY
SEQ ID NO: 40
ASSDRGGQDTQY
SEQ ID NO: 41
ASSSGTDTEVF
SEQ ID NO: 42
AWRDWGGAEQF
SEQ ID NO: 43
ASSGLGETLY
SEQ ID NO: 44
ASSLDNSGNTLY
SEQ ID NO: 45
ASSLDRVQDTQY
SEQ ID NO: 46
AWTEVF
SEQ ID NO: 47
ASSFGQNYAEQF
SEQ ID NO: 48
ASSDGTSAETLY
SEQ ID NO: 49
ASRPGSAETLY
SEQ ID NO: 50
ASSPQLYEQY
SEQ ID NO: 51
ASSDGLGVNQDTQY
SEQ ID NO: 52
ASSDGGGGTEVF
SEQ ID NO: 53
AWSLRLGGTYEQY
SEQ ID NO: 54
ASSLTISNERLF
SEQ ID NO: 55
ASSFWGRQDTQY
SEQ ID NO: 56
ASSFWGRGNTLY
SEQ ID NO: 57
ASGGPGQGFAEQF
SEQ ID NO: 58
ASSPTGAIMNS
SEQ ID NO: 59
ASSLYRDRGYAEQF
SEQ ID NO: 60
AWSLPLGQSYEQY
SEQ ID NO: 61
ASSFRGYEQY
SEQ ID NO: 62
ASSDDTYEQY
SEQ ID NO: 63
ASSDGDRYEQY
SEQ ID NO: 64
ASSDNYNSPLY
SEQ ID NO: 65
ASRDWGGRAETLY
SEQ ID NO: 66
ASSLELGGREQY
SEQ ID NO: 67
ASSDPGAANTEVF
SEQ ID NO: 68
ASSLDGADSDYT
SEQ ID NO: 69
ASSMNNERLF
SEQ ID NO: 70
ASSQVGGASETLY
SEQ ID NO: 71
ASGDATDYSGNTLY
SEQ ID NO: 72
ASGEGPANTEVF

In some embodiments, such T cells are engineered to express a TCR. In some, embodiments, such T cells are engineered to express a chimeric TCR. In some embodiments, such T cells are engineered to express a single chain TCR comprising the structure Vα-linker-Vβ or Vβ-linker-Vα. In some embodiments, the TCR is reactive to a specific antigen. In some embodiments, such T cells are engineered to express the Vα CDR-3 sequence selected from the above group (SEQ ID NO: 33-SEQ ID NO:72). In some embodiments, the engineered T cell may express a chimeric TCR comprising the Vα CDR-3 sequence selected from the above group (SEQ ID NO: 33-SEQ ID NO:72).

The antigen-specific TCR may be a bispecific T cell receptor comprising the TCR or an antigen binding fragment thereof and an antibody. The TCR may comprise a Vα CDR-3 comprising an amino acid sequence selected from (SEQ ID NO: 33-SEQ ID NO:72).

### Sugar Expanded Cells and Compositions

In an aspect, the disclosure provides a cell population comprising generating the cell population with the method of promoting proliferation of progenitor exhausted T cell with enhanced expansion and persistence, comprising contacting a cell population comprising progenitor exhausted T cells with an effective amount of a compound, wherein the cell population comprises progenitor exhausted T cells.

In some embodiments, the present invention provides an in vitro or ex vivo expanded population of antigen-reactive T cells, wherein the T cells were isolated using a method described herein. The antigen reactive T cells may in some embodiments recognize an antigen from a cancer, a pathogenic infection, autoimmune disease, inflammatory disease, or a genetic disorder.

In some embodiments, the present invention provides an in vitro or ex vivo expanded population of TSA reactive T cells, wherein the T cells were isolated using a method described herein.

In some embodiments, the cell population is derived from a naïve splenocyte.

In some embodiments, the naïve splenocyte optionally comprises a engineered antibody-based binding agent, wherein the antibody-based binding agent specifically binds an antigen identified in the method of identifying progenitor exhausted T cell specific to an antigen, comprising contracting the cell population with a modified dendritic cell in the presence of a donor sugar nucleotide that is conjugated to a label.

In some embodiments, the binding agent specifically binds a virus-specific antigen. In some embodiments, the virus-specific antigen is an antigen of Lymphocytic choriomeningitis virus (LCMV), hepatitis C virus (HCV), or human immunodeficiency virus (HIV). In some embodiments, the binding agent specifically binds a tumor-specific antigen (TSA). In some embodiments, the tumor-specific antigen (TSA) is an antigen of ovalbumin.

In some embodiments, the binding agent comprises a ligand binding domain comprising the complementarity determine regions CDR-H1, CDR-H2, CDR-H3, CDR-L1, CDR-L2, and CDR-L3. In some embodiments, the binding domain comprises VH and VL. In some embodiments, the ligand binding domain comprises a single chain variable fragment. In some embodiments, the binding agent comprises a single polypeptide chain. In some embodiments, the binding agent is a chimeric antigen receptor.

In some embodiments, the cell population is CD8+. In some embodiments, the cell population is TCF-1+, PD-1+, and Tim3−.

In some embodiments, the cell population exhibits enhanced expansion and persistence.

In an aspect, the disclosure provides a pharmaceutical composition comprising the cell population and one or more pharmaceutically acceptable excipients or diluents.

The T cells, and expanded populations of the same, may be formulated into a pharmaceutical composition. The pharmaceutical composition may be any composition disclosed herein.

As used herein the term “pharmaceutical composition” refers to a pharmaceutical acceptable composition, wherein the composition comprises progenitor exhausted T cells, and in some embodiments further comprises a pharmaceutically acceptable carrier.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carriers” or “pharmaceutically effective excipients” refers to an excipient, diluent, preservative, solubilizer, emulsifier, adjuvant, and/or vehicle with which a progenitor exhausted T cell, is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

Formulations of a pharmaceutical composition suitable for administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. Formulations may also include aqueous solutions which may contain excipients such as salts, carbohydrates and buffering agents or sterile, pyrogen-free, water. Exemplary administration forms may include solution s or suspensions in sterile aqueous solutions, for example, aqueous propylene glycol or dextrose solutions. Such dosage forms can be suitably buffered, if desired.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, and/or aromatic substances and the like which do not deleteriously interact with the formulation.

### Use of Cells and Compositions

In an aspect, the disclosure provides a method of treating a disease or disorder in a subject in need thereof, comprising administering an effective amount of the cell population or the pharmaceutical composition to the subject.

In some embodiments, any disease or disorder that results in the expression of a disease-specific antigen on the surface of a cell may be treated with an antigen-reactive progenitor exhausted T cell isolated via the methods described herein or a pharmaceutical composition comprising the same. The methods may comprise administering to a subject in need thereof one or more different populations of progenitor exhausted T cells isolated and expanded via the methods described herein and methods known in the art. For example, a population of progenitor exhausted T cells directed to a particular antigen may be identified via the methods disclosed herein and expanded and another population of progenitor exhausted T cells directed to another different antigen may be identified via the methods disclosed herein and expanded and both populations may be administered to a subject in need thereof.

In general, administration may be topical, parenteral, or enteral. The compositions of the disclosure are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrastemal, intravenous, intranasal, intratracheal, intraarterial, intrathecal, intraventricular, intraurethral, intracranial, intratumoral, intraocular, intradermal, intrasynovial injection or infusions; and kidney dialytic infusion techniques. In some embodiments, the cells and compositions of the present disclosure comprise intravenous administration. In some embodiments, the cells and compositions of the present disclosure comprise intramuscular administration. In some embodiments, the cells and compositions of the present disclosure comprise subcutaneous administration.

In some embodiments, administering comprises parenteral administration. In some embodiments, administering comprises intravenous administration.

In some embodiments, the methods may comprise administering about 1, 2, 3, 4, 5, 6, 7, 8, 9, 10, 12, 15, 20, 24, 30, 35, 48, 50, 55, 60, 65, 70, 75, 80, 85, 90, 96, 100, 120, 150, 200, 300, 384, 400, 500, 600, 700, 800, 900, 1000 or more populations of antigen-reactive progenitor exhausted T cells isolated and expanded via the methods described herein.

In some embodiments, the progenitor exhausted T cells expanded using the methods describe herein can be administered as a single dosage. In some embodiments, the progenitor exhausted T cells expanded using the methods described herein can be administered as multiple dosages.

The optimal dosage and treatment regime for a particular subject can be determined by one skilled in the art of medicine by monitoring the patient for signs of disease and adjusting the treatment accordingly. The treatment may also be adjusted after measuring the levels of a therapeutic agent (e.g., number of progenitor exhausted T cells) in a biological sample (e.g., body fluid or tissue sample) can also be used to assess the treatment efficacy, and the treatment may be adjusted accordingly to increase or decrease.

In some embodiments, a single dose of progenitor exhausted T cells, expanded using the methods described herein, is administered to a subject. In some embodiments, two or more doses of progenitor exhausted T cells, expanded using the methods described herein, are administered sequentially to a subject. In some embodiments, three doses of progenitor exhausted T cells, expanded using the methods described herein, are administered sequentially to a subject. In some embodiments, a dose of progenitor exhausted T cells, expanded using the methods described herein, is administered weekly, biweekly, monthly, bimonthly, quarterly, semiannually, annually, or biannually to a subject. In some embodiments, a second or subsequent dose of progenitor exhausted T cells, expanded using the methods described herein, is administered to a subject when an amount of progenitor exhausted T cells, expanded using the methods described herein, decreases.

In one embodiment, an antigen reactive T cell or a population of antigen reactive T cells are identified and enriched via a method described herein and are expanded and administered to a patient to treat a disease or condition. The disease or condition may be a cell proliferative disorder. The cell proliferative disorder may be selected from a solid tumor, a lymphoma, a leukemia and a liposarcoma. The cell proliferative disorder may be acute, chronic, recurrent, refractory, accelerated, in remission, stage I, stage II, stage III, stage IV, juvenile or adult. The cell proliferative disorder may be selected from myelogenous leukemia, lymphoblastic leukemia, myeloid leukemia, an acute myeloid leukemia, myelomonocytic leukemia, neutrophilic leukemia, myelodysplastic syndrome, B-cell lymphoma, burkitt lymphoma, large cell lymphoma, mixed cell lymphoma, follicular lymphoma, mantle cell lymphoma, hodgkin lymphoma, recurrent small lymphocytic lymphoma, hairy cell leukemia, multiple myeloma, basophilic leukemia, eosinophilic leukemia, megakaryoblastic leukemia, monoblastic leukemia, monocytic leukemia, erythroleukemia, erythroid leukemia and hepatocellular carcinoma. The cell proliferative disorder may comprise a hematological malignancy. The hematological malignancy may comprise a B cell malignancy. The cell proliferative disorder may comprise a chronic lymphocytic leukemia. The cell proliferative disorder may comprise an acute lymphoblastic leukemia. The cell proliferative disorder may comprise a CD19-positive Burkitt's lymphoma.

The disease or condition may be a cancer, a pathogenic infection, autoimmune disease, inflammatory disease, or genetic disorder.

The cancer may comprise a recurrent and/or refractory cancer. Examples of cancers include, but are not limited to, sarcomas, carcinomas, lymphomas or leukemias. The cancer may comprise a neuroendocrine cancer. The cancer may comprise a pancreatic cancer. The cancer may comprise an exocrine pancreatic cancer. The cancer may comprise a thyroid cancer. The thyroid cancer may comprise a medullary thyroid cancer. The cancer may comprise a prostate cancer. The cancer may comprise an epithelial cancer. The cancer may comprise a breast cancer. The cancer may comprise an endometrial cancer. The cancer may comprise an ovarian cancer. The ovarian cancer may comprise a stromal ovarian cancer. The cancer may comprise a cervical cancer. The cancer may comprise a skin cancer. The skin cancer may comprise a neo-angiogenic skin cancer. The skin cancer may comprise a melanoma. The cancer may comprise a kidney cancer. The cancer may comprise a lung cancer. The lung cancer may comprise a small cell lung cancer. The lung cancer may comprise a non-small cell lung cancer. The cancer may comprise a colorectal cancer. The cancer may comprise a gastric cancer. The cancer may comprise a colon cancer. The cancer may comprise a brain cancer. The brain cancer may comprise a brain tumor. The cancer may comprise a glioblastoma. The cancer may comprise an astrocytoma. The cancer may comprise a blood cancer. The blood cancer may comprise a leukemia. The leukemia may comprise a myeloid leukemia. The cancer may comprise a lymphoma. The lymphoma may comprise a non-Hodgkin's lymphoma. The cancer may comprise a sarcoma. The sarcoma may comprise an Ewing's sarcoma.

Sarcomas are cancers of the bone, cartilage, fat, muscle, blood vessels, or other connective or supportive tissue. Sarcomas include, but are not limited to, bone cancer, fibrosarcoma, chondrosarcoma, Ewing's sarcoma, malignant hemangioendothelioma, malignant schwannoma, bilateral vestibular schwannoma, osteosarcoma, soft tissue sarcomas (e.g., alveolar soft part sarcoma, angiosarcoma, cystosarcoma phylloides, dermatofibrosarcoma, desmoid tumor, epithelioid sarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma).

Carcinomas are cancers that begin in the epithelial cells, which are cells that cover the surface of the body, produce hormones, and make up glands. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penile cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. In some instances, the cancer is a skin cancer, such as a basal cell carcinoma, squamous, melanoma, nonmelanoma, or actinic (solar) keratosis.

In some instances, the cancer is a lung cancer. Lung cancer may start in the airways that branch off the trachea to supply the lungs (bronchi) or the small air sacs of the lung (the alveoli). Lung cancers include non-small cell lung carcinoma (NSCLC), small cell lung carcinoma, and mesotheliomia. Examples of NSCLC include squamous cell carcinoma, adenocarcinoma, and large cell carcinoma. The mesothelioma may be a cancerous tumor of the lining of the lung and chest cavity (pleura) or lining of the abdomen (peritoneum). The mesothelioma may be due to asbestos exposure. The cancer may be a brain cancer, such as a glioblastoma.

Alternatively, the cancer may be a central nervous system (CNS) tumor. CNS tumors may be classified as gliomas or nongliomas. The glioma may be malignant glioma, high grade glioma, diffuse intrinsic pontine glioma. Examples of gliomas include astrocytomas, oligodendrogliomas (or mixtures of oligodendroglioma and astocytoma elements), and ependymomas. Astrocytomas include, but are not limited to, low-grade astrocytomas, anaplastic astrocytomas, glioblastoma multiforme, pilocytic astrocytoma, pleomorphic xanthoastrocytoma, and subependymal giant cell astrocytoma. Oligodendrogliomas include low-grade oligodendrogliomas (or oligoastrocytomas) and anaplastic oligodendriogliomas. Nongliomas include meningiomas, pituitary adenomas, primary CNS lymphomas, and medulloblastomas. In some instances, the cancer is a meningioma.

The leukemia may be an acute lymphocytic leukemia, acute myelocytic leukemia, chronic lymphocytic leukemia, or chronic myelocytic leukemia. Additional types of leukemias include hairy cell leukemia, chronic myelomonocytic leukemia, and juvenile myelomonocytic leukemia.

Lymphomas are cancers of the lymphocytes and may develop from either B or T lymphocytes. The two major types of lymphoma are Hodgkin's lymphoma, previously known as Hodgkin's disease, and non-Hodgkin's lymphoma. Hodgkin's lymphoma is marked by the presence of the Reed-Sternberg cell. Non-Hodgkin's lymphomas are all lymphomas which are not Hodgkin's lymphoma. Non-Hodgkin lymphomas may be indolent lymphomas and aggressive lymphomas. Non-Hodgkin's lymphomas include, but are not limited to, diffuse large B cell lymphoma, follicular lymphoma, mucosa-associated lymphatic tissue lymphoma (MALT), small cell lymphocytic lymphoma, mantle cell lymphoma, Burkitt's lymphoma, mediastinal large B cell lymphoma, Waldenstrom macroglobulinemia, nodal marginal zone B cell lymphoma (NMZL), splenic marginal zone lymphoma (SMZL), extranodal marginal zone B cell lymphoma, intravascular large B cell lymphoma, primary effusion lymphoma, and lymphomatoid granulomatosis.

The cancer may comprise a solid tumor. The cancer may comprise a sarcoma. The cancer may be selected from a group consisting of a bladder cancer, a breast cancer, a colon cancer, a rectal cancer, an endometrial cancer, a kidney cancer, a lung cancer, melanoma, a myeloma, a thyroid cancer, a pancreatic cancer, a glioma, a malignant glioma of the brain, a glioblastoma, an ovarian cancer, and a prostate cancer. The cancer may have non-uniform antigen expression. The cancer may have modulated antigen expression. The antigen may be a surface antigen. The cancer may not comprise a myeloma. The cancer may not comprise a melanoma. The cancer may not comprise a colon cancer. The cancer may be acute lymphoblastic leukemia (ALL). The cancer may be relapsed ALL. The cancer may be refractory ALL. The cancer may be relapsed, refractory ALL. The cancer may be chronic lymphocytic leukemia (CLL). The cancer may be relapsed CLL. The cancer may be refractory CLL. The cancer may be relapsed, refractory CLL.

The cancer may comprise a breast cancer. The breast cancer may be triple positive breast cancer (estrogen receptor, progesterone receptor and Her2 positive). The breast cancer may be triple negative breast cancer (estrogen receptor, progesterone receptor and Her2 negative). The breast cancer may be estrogen receptor positive. The breast cancer may be estrogen receptor negative. The breast cancer may be progesterone receptor positive. The breast cancer may be progesterone receptor negative. The breast cancer may comprise a Her2 negative breast cancer. The breast cancer may comprise a low-expressing Her2 breast cancer. The breast cancer may comprise a Her2 positive breast cancer. Cell lines expressing Her2 have been well-characterized for antigen density, reflecting clinical immunohistochemistry characterization which classifies malignancies as 0 (<20,000 Her2 antigens per cell), 1+ (100,000 Her2 antigens per cell), 2+ (500,000 Her2 antigens per cell), and 3+ (>2,000,000 Her2 antigens per cell). The present invention provides for methods of treating breast cancers of these classifications. The breast cancer may comprise a breast cancer classified as Her2 0. The breast cancer may comprise a breast cancer classified as Her2 1+. The breast cancer may comprise a breast cancer classified as Her2 2+. The breast cancer may comprise a breast cancer classified as a Her2 3+.

The disease or condition may be a pathogenic infection. Pathogenic infections may be caused by one or more pathogens. In some instances, the pathogen is a bacterium, fungi, virus, or protozoan.

Exemplary pathogens include but are not limited to: Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, or Yersinia. In some cases, the disease or condition caused by the pathogen is tuberculosis and the heterogeneous sample comprises foreign molecules derived from the bacterium Mycobacterium tuberculosis and molecules derived from the subject. In some instances, the disease or condition is caused by a bacterium is tuberculosis, pneumonia, which may be caused by bacteria such as Streptococcus and Pseudomonas, a foodborne illness, which may be caused by bacteria such as Shigella, Campylobacter and Salmonella, and an infection such as tetanus, typhoid fever, diphtheria, syphilis and leprosy. The disease or condition may be bacterial vaginosis, a disease of the vagina caused by an imbalance of naturally occurring bacterial flora. Alternatively, the disease or condition is a bacterial meningitis, a bacterial inflammation of the meninges (e.g., the protective membranes covering the brain and spinal cord). Other diseases or conditions caused by bacteria include, but are not limited to, bacterial pneumonia, a urinary tract infection, bacterial gastroenteritis, and bacterial skin infection. Examples of bacterial skin infections include, but are not limited to, impetigo which may be caused by Staphylococcus aureus or Streptococcus pyogenes; erysipelas which may be caused by a streptococcus bacterial infection of the deep epidermis with lymphatic spread; and cellulitis which may be caused by normal skin flora or by exogenous bacteria.

The pathogen may be a fungus, such as, Candida, Aspergillus, Cryptococcus, Histoplasma, Pneumocystis, and Stachybotrys. Examples of diseases or conditions caused by a fungus include, but are not limited to, jock itch, yeast infection, ringworm, and athlete's foot.

The pathogen may be a virus. Examples of viruses include, but are not limited to, adenovirus, coxsackievirus, Epstein-Barr virus, Hepatitis virus (e.g., Hepatitis A, B, and C), herpes simplex virus (type 1 and 2), cytomegalovirus, herpes virus, HIV, influenza virus, measles virus, mumps virus, papillomavirus, parainfluenza virus, poliovirus, respiratory syncytial virus, rubella virus, and varicella-zoster virus. Examples of diseases or conditions caused by viruses include, but are not limited to, cold, flu, hepatitis, AIDS, chicken pox, rubella, mumps, measles, warts, and poliomyelitis.

The pathogen may be a protozoan, such as Acanthamoeba (e.g., A. astronyxis, A. castellanii, A. culbertsoni, A. hatchetti, A. polyphaga, A. rhysodes, A. healyi, A. divionensis), Brachiola (e.g., B connori, B. vesicularum), Cryptosporidium (e.g., C. parvum), Cyclospora (e.g., C. cayetanensis), Encephalitozoon (e.g., E. cuniculi, E. hellem, E. intestinalis), Entamoeba (e.g., E. histolytica), Enterocytozoon (e.g., E. bieneusi), Giardia (e.g., G. lamblia), Isospora (e.g, I. belli), Microsporidium (e.g., M africanum, M ceylonensis), Naegleria (e.g., N. fowleri), Nosema (e.g., N. algerae, N. ocularum), Pleistophora, Trachipleistophora (e.g., T anthropophthera, T. hominis), and Vittaforma (e.g., V corneae).

The disease or condition may be an autoimmune disease or autoimmune related disease. An autoimmune disorder may be a malfunction of the body's immune system that causes the body to attack its own tissues. Examples of autoimmune diseases and autoimmune related diseases include, but are not limited to, Addison's disease, alopecia areata, ankylosing spondylitis, antiphospholipid syndrome (APS), autoimmune aplastic anemia, autoimmune hemolytic anemia, autoimmune hepatitis, autoimmune myocarditis, Behcet's disease, celiac sprue, Crohn's disease, dermatomyositis, eosinophilic fasciitis, erythema nodosum, giant cell arteritis (temporal arteritis), Goodpasture's syndrome, Graves' disease, Hashimoto's disease, idiopathic thrombocytopenic purpura (ITP), IgA nephropathy, juvenile arthritis, diabetes, juvenile diabetes, Kawasaki syndrome, Lambert-Eaton syndrome, lupus (SLE), mixed connective tissue disease (MCTD), multiple sclerosis, myasthenia gravis, pemphigus, polyarteritis nodosa, type I, II, & III autoimmune polyglandular syndromes, polymyalgia rheumatica, polymyositis, psoriasis, psoriatic arthritis, Reiter's syndrome, relapsing polychondritis, rheumatoid arthritis, sarcoidosis, scleroderma, Sjogren's syndrome, sperm & testicular autoimmunity, stiff person syndrome, Takayasu's arteritis, temporal arteritis/giant cell arteritis, ulcerative colitis, uveitis, vasculitis, vitiligo, and Wegener's granulomatosis.

The disease or condition may be an inflammatory disease. Examples of inflammatory diseases include, but are not limited to, alveolitis, amyloidosis, angiitis, ankylosing spondylitis, avascular necrosis, Basedow's disease, Bell's palsy, bursitis, carpal tunnel syndrome, celiac disease, cholangitis, chondromalacia patella, chronic active hepatitis, chronic fatigue syndrome, Cogan's syndrome, congenital hip dysplasia, costochondritis, Crohn's Disease, cystic fibrosis, De Quervain's tendinitis, diabetes associated arthritis, diffuse idiopathic skeletal hyperostosis, discoid lupus, Ehlers-Danlos syndrome, familial mediterranean fever, fascitis, fibrositis/fibromyalgia, frozen shoulder, ganglion cysts, giant cell arteritis, gout, Graves' Disease, HIV-associated rheumatic disease syndromes, hyperparathyroid associated arthritis, infectious arthritis, inflammatory bowel syndrome/irritable bowel syndrome, juvenile rheumatoid arthritis, lyme disease, Marfan's Syndrome, Mikulicz's Disease, mixed connective tissue disease, multiple sclerosis, myofascial pain syndrome, osteoarthritis, osteomalacia, osteoporosis and corticosteroid-induced osteoporosis, Paget's Disease, palindromic rheumatism, Parkinson's Disease, Plummer's Disease, polymyalgia rheumatica, polymyositis, pseudogout, psoriatic arthritis, Raynaud's Phenomenon/Syndrome, Reiter's Syndrome, rheumatic fever, rheumatoid arthritis, sarcoidosis, sciatica (lumbar radiculopathy), scleroderma, scurvy, sickle cell arthritis, Sjogren's Syndrome, spinal stenosis, spondyloisthesis, Still's Disease, systemic lupus erythematosis, Takayasu's (Pulseless) Disease, Tendinitis, tennis elbow/golf elbow, thyroid associated arthritis, trigger finger, ulcerative colitis, Wegener's Granulomatosis, and Whipple's Disease.

In some embodiments, the methods may comprise titrating the progenitor exhausted T cell or population of progenitor exhausted T cells for a desired effect. Titrating the progenitor exhausted T cell or population of progenitor exhausted T cells may enable antigen density discrimination. For example, the fatal on-target, off-tumor reactivity for Her2 targeted CAR-T cells to low levels of Her2 expression in the lung has tempered the application of CAR-T cells to solid tumors in the clinic. In the clinic this may be used to titrate therapy to an appropriate therapeutic index.

In some embodiments, a pathogenic-antigen reactive progenitor exhausted T cell identified and enriched via a method described herein is expanded and administered to a patient to treat a pathogenic infection. In some embodiments, the pathogen is a virus, parasite, or bacteria. In some embodiments, the T cell is administered as a pharmaceutical composition.

In one embodiment, an auto-antigen reactive regulatory T cell identified and enriched via a method described herein is expanded and administered to a patient to treat an autoimmune disease or disorder. In one embodiment, the disease is Polymyositis. In some embodiments, the T cell is administered as a pharmaceutical composition.

In some embodiments, treating comprises reducing a severity of at least one sign or symptom of the disease or disorder. In some embodiments, the disease or disorder is a cancer.

In some embodiments, the cancer is selected from a melanoma tumor; a breast cancer tumor; and a tumor selected from the group consisting of Pilocytic astrocytoma; AML; ALL; Thyroid; Kidney chromophobe; CLL; Medulloblastoma; Neuroblastoma; Glioma low grade; Glioblastoma; Prostate; Ovary; Myeloma; Pancreas; Kidney papillary; Lymphoma B-cell; Kidney clear cell; Head and neck; Liver; Cervix; Uterus; Bladder; Colorectum; Lung small cell; Esophagus; Stomach; Lung adeno; and Lung squamous. In some embodiments, the administration is as a pharmaceutical composition. In some embodiments, the disease or disorder is an infection.

In some embodiments, the cell composition or pharmaceutical composition is assessed for purity prior to administration. In some embodiments, the cell composition or pharmaceutical composition is tested for sterility. In some embodiments, the cell composition or pharmaceutical composition is screened to confirm it matches the recipient subject.

In some embodiments, the subject is a mammal. In some embodiments, the subject is a human.

In some embodiments, the cell population is allogeneic to the subject. In some embodiments, the cell population is autologous to the subject.

In some embodiments, administering comprises combined administration with IL-2.

Immune checkpoint inhibitors may be used in combination with adoptive cell transfer (ACT)-based therapies. Successful anti-tumor immune responses following PD-1/PD-L1 blockade are believed to require re-activation and clonal-proliferation of neoantigen-specific T cells present in the tumor microenvironment. Inadequate generation of neoantigen-specific T cells, suppression of the effector function of neoantigen-specific T cells, and impaired formation of memory T cell are major factors responsible for the failure of checkpoint inhibitor therapies.

In some embodiments, administering comprises combined administration with a compound identified using the method of screening for compounds that promote proliferation of progenitor exhausted T cells as disclosed herein. In some embodiments, administering comprises combined administration with a cell surface receptor programmed cell death 1 (PD-1) inhibitor or a programmed death-ligand 1 (PD-L1) inhibitor. In some embodiments, the PD1 inhibitor or PD-L1 inhibitor comprises a monoclonal antibody that specifically binds (PD-1) or (PD-L1), respectively. In some embodiments, the monoclonal antibody that specifically binds PD-1 is pembrolizumab, nivolumab, or cemiplimab. In some embodiments, the monoclonal antibody that specifically binds PD-L1 is atezolizumab, avelumab, or durvalumab. In some embodiments, the cell population or the pharmaceutical composition to the subject for use in a method of treatment.

In an aspect, the disclosure provides a kit comprising the cell population or the pharmaceutical composition and instructions for use.

In some embodiments, progenitor exhausted T cells expanded using the methods described herein, or other methods known in the art, are administered to a patient in conjunction with (e.g. before, simultaneously or following) any number of relevant treatment modalities, including but not limited to treatment with agents such as antiviral agents, chemotherapy, radiation, immunosuppressive agents, such as cyclosporin, bisulfin, bortezomib, azathioprine, methotrexate, mycophenolate, and FK506, antibodies, or other immunoablative agents such as CAMPATH, anti-CD3 antibodies or other antibody therapies, cytoxin, fiudaribine, cyclosporin, FK506, rapamycin, mycophenolic acid, steroids, FR901228, cytokines, and irradiation. These drugs inhibit either the calcium dependent phosphatase calcineurin (cyclosporine and FK506), the proteasome (bortezomib), or inhibit the p70S6 kinase that is important for growth factor induced signaling (rapamycin). (Liu et al, Cell 66:807-815, 1991; Henderson et al, Immun. 73:316-321, 1991; Bierer et al, Curr. Opin. Immun. 5:763-773, 1993; Isoniemi (supra)).

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

The section headings used herein are for organizational purposes only and are not to be construed as limiting the subject matter described.

While illustrative embodiments have been illustrated and described, it will be appreciated that various changes can be made therein without departing from the spirit and scope of the invention.

### EXAMPLES

The disclosure is now described with reference to the following Examples. These Examples are provided for the purpose of illustration only and the disclosure should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the methods of the present disclosure and practice the claimed methods. The following working examples therefore, specifically point out embodiments of the present disclosure, and are not to be construed as limiting in any way the remainder of the disclosure.

The materials and methods employed in these experiments are now described.

### Example 1: Development of a High Throughput Assay to Screen for Compounds that Uncouple T Cell Expansion from Differentiation

### Background

Adoptive cell transfer therapies have limited efficacy in reducing an established malady (e.g., tumor burden or infection). Short persistence of T cells in vivo after transfer correlates with the absence of sustained clinical responses. T cells (TCR-engineered and isolated antigen-specific T cells) need to be expanded in vitro to produce large numbers of cells for patient infusion (e.g., adoptive cell transfer). Presumably, the absence of persistent T cells observed in subjects undergoing T cell transfer treatments is due to the extensive expansion regimens used to produce sufficient numbers of T cells, wherein T cells are driven into terminal differentiation and exhibit limited or absent replication potential.

Tpex cells (Tim3−TCF-1+) provide a proliferative burst and effector function by forming terminally exhausted T cells (Ttex, Tim3+TCF-1−) following anti-PD-1/PD-L1 therapy. Evaluation of patient survival rates via meta-analysis of The Cancer Genome Atlas (TCGA) database supports evidence that Tcf7/Pdcd1 signatures in tumor infiltrating lymphocytes (TILs) correlate with differential survival rates in patients.

Therefore, this study aims to employ a novel assay to identify small molecule modulators that can be used during in vitro expansion to improve the quality of isolated T cells by boosting the numbers of anti-PD-1 responsive CD8+ T cells with less differentiated phenotypes.

### Methods

OT-I-Tcf7GFP mice were bred by crossing the B6(Cg)-Tcf7tmiHhx/J (Tcf7GFP) mice expressing reporter EGFP from the endogenous Tcf7 locus with the OT-I TCR-Tg mice whose CD8+ T cells have a transgenic TCR that recognizes OVA257-264 presented by the MHC I molecule. Naïve OT-1-Tcf7GFP splenocytes were stimulated in vitro with OVA257-264 peptide (500 nM) and IL-2 (60 IU/mL) in the presence or absence of an added compound for 3 days at a density of 1 million/mL (100 μL/well in 96/well plate), then expanded in culture medium containing 60 IU/mL IL-2 for 4 days while maintaining the presence or absence of an added compound. Cell number was recorded on day 3 and day 7. On day 7, cells were stained with anti-CD8a (PerCP-Cy5.5) and anti-Tim3 (BV421) antibodies for flow cytometry or plate reader analysis.

### Results

Because TCF1 expression positively correlates with T cell stemness, the greater signal of EGFP a T cell expresses, the less differentiated the T cell is. Furthermore, if the compound also facilitates T cells proliferation, there would be more T cells per well in comparison to the untreated control. Therefore, a compound that not only maintains TCF-1 expression but also facilitates T cell growth would produce the strongest green fluorescence intensity. Various sugars were found to increase the number of T cells capable of maintaining TCF-1 expression. Specific sugars were found to improve both proliferation and Tim3−TCF-1+ ratio of OT-1 cells (Table 1), while other sugars were found to increase Tim3−TCF-1+ ratio but inhibit or do not significantly change the proliferation of OT-1 cells (Table 1). GlcA stands for glucuronic acid.

TABLE 1
Sugars improve Tim3−TCF-1+ ratio of OT-1 cells

| | | GlcNAc | Neu5Ac | Fructose | Glucose | (60 mM) | Mannose | Sucrose | Lactose | Trehalose |
|---|---|---|---|---|---|---|---|---|---|---|
| | | (60 mM) | (50 mM) | (80 mM) | (60 mM) | GlcA | (60 mM) | (80 mM) | (60 mM) | (80 mM) |
| Day7 | w/o | Increase proliferation | | | No changes | | Inhibit proliferation | | | |
| Proliferation fold, | 1.00 | 1.45 | 1.43 | 1.09 | 0.97 | 0.97 | 0.30 | 0.86 | 0.63 | 0.84 |
| Normalized to W/O | | | | | | | | | | |
| Tim3⁻TCF-1⁺ ratio of | 2.6 | 28.8 | 59.6 | 43.9 | 12.0 | 65.3 | 8.4 | 41.2 | 25.6 | 35.4 |
| CD8⁺ T cells (%) | | | | | | | | | | |

### Discussion

This study demonstrates that this method of screening can be used to evaluate compounds of interest that promote the proliferation of T cells without loss of stemness.

### Example 2: Screening for Small Molecule Modulators that can Increase TCF-1+ Cells During In Vitro Activation and Expansion of Antigen-Specific P14 CD8+ T Cells

### Background

Adoptive cell transfer therapies have limited efficacy in reducing an established malady (e.g., tumor burden or infection). Short persistence of T cells in vivo after transfer correlates with the absence of sustained clinical responses. T cells (TCR-engineered and isolated antigen-specific T cells) need to be expanded in vitro to produce large numbers of cells for patient infusion (e.g., adoptive cell transfer). Presumably, the absence of persistent T cells observed in subjects undergoing T cell transfer treatments is due to the extensive expansion regimens used to produce sufficient numbers of T cells, wherein T cells are driven into terminal differentiation and exhibit limited or absent replication potential.

Tpex cells (Tim3−TCF-1+) provide a proliferative burst and effector function by forming terminally exhausted T cells (Ttex, Tim3+TCF-1−) following anti-PD-1/PD-L1 therapy. Evaluation of patient survival rates via meta-analysis of The Cancer Genome Atlas (TCGA) database supports evidence that Tcf7/Pdcd1 signatures in tumor infiltrating lymphocytes (TILs) correlate with differential survival rates in patients.

Therefore, this study aims to employ a novel assay to identify small molecule modulators that can be used during in vitro expansion to improve the quality of isolated T cells by boosting the numbers of anti-PD-1 responsive CD8+ T cells with less differentiated phenotypes.

### Methods

CD8+ T cells from P14 TCR-Tg mice with a transgenic T cell antigen receptor specific for the glycoprotein 33-41 antigen (GP33-41) of Lymphocytic choriomeningitis virus (LCMV) were used. P14 splenocytes were stimulated in vitro with GP33 peptide (1 μM) and IL-2 (60 IU/mL) in the presence or absence of an added compound for 3 days at a density of 1 million/mL (100 uL/well in 96/well plate), then expanded in culture medium containing 60 IU/mL IL-2 for 4 days. The secondary re-stimulation was done for 48 h (from day 6 to day 8) with plate-bound anti-CD3 (1 μg/mL), soluble anti-CD28 (1 μg/mL) and 200 IU/mL IL-2 at a cell density of 1 million/mL (100 μL/well in 96/well plate), and further expanded in culture medium containing 200 IU/mL IL-2 for 24 h. Compounds were kept at the same concentration during the entire course of the treatment. For treated cells, compounds were added at all the activation and expansion stage. Cell number was recorded on day 3, day 6 and day 9. On day 6, cells were stained with anti-CD8a (PE-Cy7), anti-Tim3 (BV421) and anti-TCF-1 (AF488) antibodies for flow cytometry analysis.

### Results

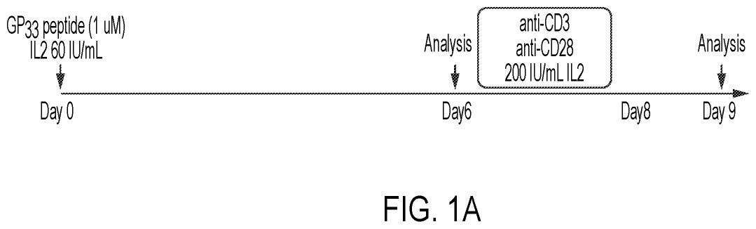

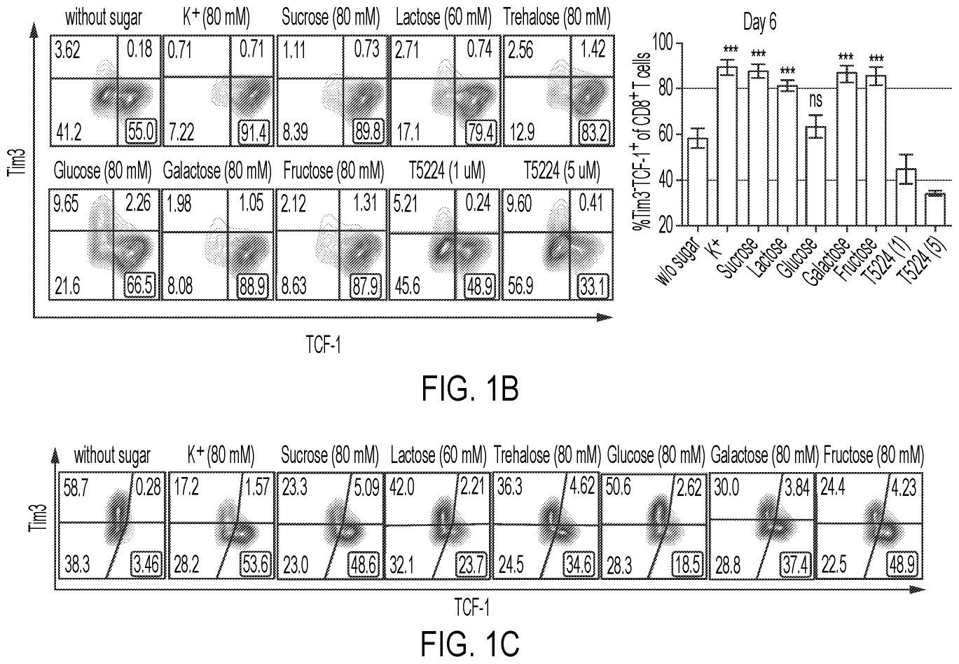

On day 6, for T cells treated with fructose, sucrose, lactose and trehalose exhibited approximately a 1.4-1.6-fold increase in the percentage and total number of virus-specific Tim3−TCF-1+CD8+ T cells in comparison to the T cells cultured without additional sugar supplement and T cells cultured with glucose, respectively (FIG. 1i). The percentage of Tim3−TCF-1+CD8+ T increased along with increasing the sugar concentrations.

On day 9, T cells treated with fructose, sucrose, trehalose, and lactose exhibited 7-14-fold increase in the percentage and total number of virus-specific Tim3−TCF-1+CD8+ T cells in comparison to the untreated T cells (FIG. 1C).

The addition of a number of sugars, including trehalose, sucrose, lactose, glucose, galactose and fructose did not inhibit but significantly enhanced cell proliferation (1.1-2 fold). This was in sharp contrast to K+, a known metabolic modulators of T cell differentiation, that slightly slowed down T cell proliferation by 10-20% compared to that of untreated T cells.

Specific sugars were found to improve both proliferation and Tim3−TCF-1+ ratio of OT-1 cells (Table 2).

TABLE 2
Sugars improving both proliferation and Tim3−TCF-1+ ratio of P14 cells

| | | | GlcNAc | Neu5Ac | Sucrose | Fructose | Lactose | Trehalose | Glucose | Galactose |
|---|---|---|---|---|---|---|---|---|---|---|
| | | w/o | (60 mM) | (50 mM) | (80 mM) | (80 mM) | (60 mM) | (80 mM) | (80 mM) | (80 mM) |
| Day6 | Fold of proliferation, | 1.00 | 2.47 | 2.29 | 1.80 | 2.10 | 1.87 | 1.42 | 1.41 | 1.59 |
| | Normalized to W/O | | | | | | | | | |
| | Tim3⁻TCF-1⁺ ratio of | 55.0 | 89.2 | 97.4 | 89.8 | 87.9 | 79.4 | 83.2 | 66.5 | 88.9 |
| | CD8⁺ T cells (%) | | | | | | | | | |
| Day9 | Fold of proliferation, | 1.00 | 1.44 | 1.40 | 2.85 | 2.94 | 1.76 | 1.47 | 2.33 | 1.87 |
| | Normalized to W/O | | | | | | | | | |
| | Tim3⁻TCF-1⁺ ratio of | 3.5 | 21.2 | 71.3 | 48.6 | 48.9 | 23.7 | 34.6 | 18.5 | 37.4 |
| | CD8⁺ T cells (%) | | | | | | | | | |

### Discussion

This study demonstrates that this method of screening can be used to evaluate compounds of interest that promote the proliferation of T cells without loss of stemness.

### Example 3: Sugar-Treated Virus-Specific CD8+ T Cells Expanded Better In Vivo than the Untreated T Cells

### Background

Tim3−TCF-1+ CD8+ T cells have been shown to experience differential expansion in vitro under varying conditions (shown in Example 1 and Example 2). In particular, cells treated with sugars (compounds discovered in Example 1 and Example 2) displayed enhanced cell expansion (e.g., greater number of T cells with less differentiated phenotypes) compared to cells that were not treated with sugars.

This study aims to evaluate the in vivo expansion of transferred Tim3−TCF-1+ CD8+ T cells initially expanded in vitro using sugars.

### Methods

Naïve P14 splenocytes (Thy1.1+/+ or Thy1+/−) were stimulated in vitro with GP33 peptide (1 μM) and TL-2 (60 IU/mL) in the presence or absence of an added compound for 3 days at a density of 1 million/mL (100 μL/well in 96/well plate), then expanded in culture medium containing 60 IU/mL TL-2 for 4 days. The secondary re-stimulation was done for 48 h (from day 6 to day 8) with plate-bound anti-CD3 (1 μg/mL), soluble anti-CD28 (1 μg/mL) and 200 IU/mL TL-2 at a cell density of 1 million/mL (100 μL/well in 96/well plate), and further expanded in culture medium containing 200 IU/mL TL-2 for 24 h.

For in vivo evaluations, a cell mixture (1:1 number ratio) of untreated (Thy1.1+/+) and sucrose (Thy1.1+/−) treated cells or a cell mixture (1:1 number ratio) of untreated (Thy1.1+/−) and sucrose (Thy1.1+/+) treated cells were transferred to C57BL/6J mice (Thy1.2+/+) by tail vein intravenous injection and the mice were infected with LCMV-Cl13 (2.0×10⁶ CFU/mouse). Blood was drawn and analyzed on day 7 after infection.

On day 9, cells were stained with anti-CD8a (PE-Cy7), anti-Tim3 (BV421) and anti-TCF-1 (AF488) antibodies for flow cytometry analysis. For in vivo expansion, a cell mixture (1:1 number ratio) of untreated (Thy1.1+/−) and sucrose (Thy1.1+/+) treated cells (or lactose/fructose treated cells Thy1.1+/+) were transferred to C57BL/6J mice (Thy1.2+/+) by tail vein intravenous injection and the mice were infected with LCMV-C113 (2.0×10⁶ CFU/mouse). The cell mixture before transfer, blood (day 7), blood (day 14) and spleen (day 14) after infection were analyzed by flow cytometry.

### Results

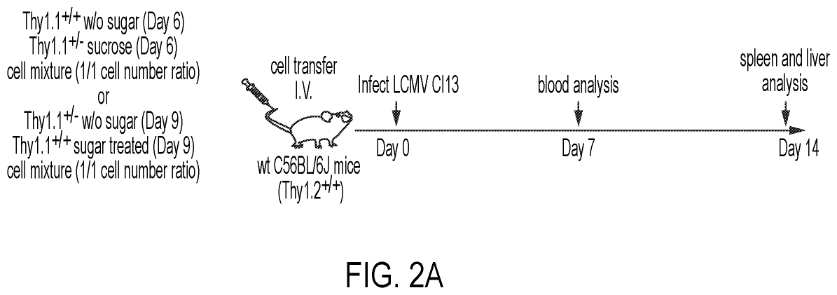

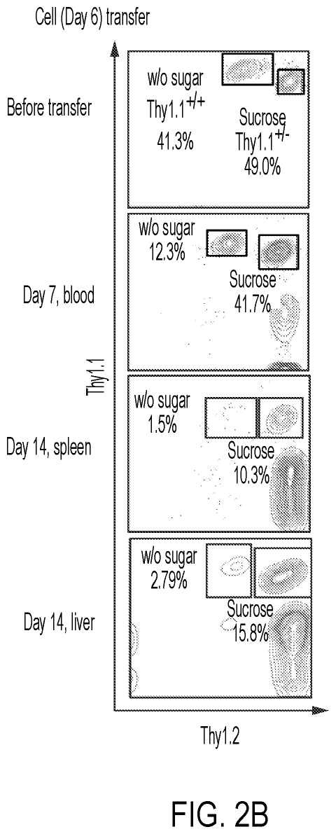

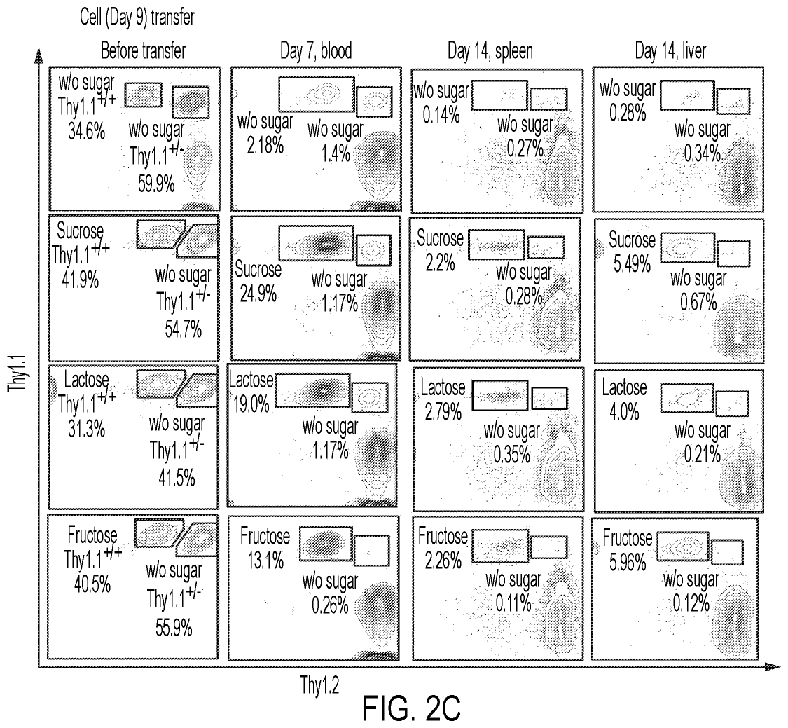

At 7 days post infection (dpi), significantly higher percentages and total numbers of the expanded virus-specific (Thy1.1+/+) P14 CD8+ T cells that were pre-treated with sucrose than the untreated Thy1.1+/−P14 cells in the blood were observed (FIG. 2A). Likewise, significantly higher percentages and total numbers of the expanded virus-specific P14 CD8+ T cells (sucrose, lactose and fructose treated) were found in the spleen and liver on 14 dpi (FIG. 2B).

### Discussion

This study demonstrates that the use of sugars during in vitro expansion of progenitor exhausted T cells allows for better expansion of T cells in vivo.

### Example 4: Sugar-Treated Antigen-Specific OT-1 CD8+ T Cells Exhibit Significantly Better Capabilities to Suppress Tumor Growth In Vivo than the Untreated T Cells

### Background

Contacting Tim3−TCF-1+CD8+ T cells with sugars (compounds discovered in Example 1 and Example 2) has been shown to increase proliferation while maintaining expression of TCF-1 and continued stemness. T cells expanded in vitro using sugars exhibit better expansion in vivo after being transferred to a subject (shown in Example 3).

This study aims to evaluate whether the use of sugars during in vitro expansion enhances the efficacy of adoptive transfer T cell therapies.

### Methods

Naïve splenocytes from OT-1 mice were stimulated in vitro with OVA257-264 (500 nM) and IL-2 (60 IU/mL) in the presence or absence of an added compound for 3 days at a density of 1 million/mL (100 uL/well in 96/well plate), then expanded for 4 days. On day 7, PD-1, Tim3 and TCF-1 expressions were assessed by flow cytometry by staining cells with anti-CD8a (PerCP-Cy5.5), anti-Tim3 (BV421) and anti-TCF-1 (AF488) antibodies. For adoptive transfer, 0.6M B16-OVA cells (6×10⁵) were inoculated subcutaneously 3 to 5 days before transfer in C57BL/6. On the day of adoptive transfer, mice were irradiated with 5 Gy X-ray, randomized according to tumor size, and intravenously injected with 0.2-0.3M of CD8+OT-1 T cells (2×10⁵) expanded under different conditions, followed by administration of 50000 IU IL-2 every 12 h for four days. In the control group, mice were injected with cell-free vehicle only. For assessment of adoptive transfer of OT-1 CD8+ T cells activated and expanded by contacting the cells with fructose, on day 9 after transfer, anti-PD-1 (100 ug/mouse) was administered to the mice that had received T cell transfer. For assessment of adoptive transfer of OT-1 CD8+ T cells activated and expanded by contacting cells with Neu5Ac, on day 14 and day 21 after transfer, anti-PD-1 (100 ug/mouse) was administered to the mice that had received T cell transfer. The tumor growth was monitored every 3 to 4 days by measuring tumor size. Percent survival was also monitored and recorded within a period of 40 days post treatment. To evaluate tumor infiltrating OT-1 T cells, 0.6M B16-OVA cells (6×10⁵) were inoculated subcutaneously 3 to 5 days before transfer in C57BL/6. On the day of adoptive transfer, mice were irradiated with 5 Gy X-ray, randomized according to tumor size, and intravenously injected with 0.2-0.3M of CD8+OT-1 T cells (2×10⁵) expanded under different conditions, followed by administration of 50000 IU IL-2 every 12 h for four days. Tumors were then isolated and assessed for percentage of OT-I T cells from the total CD8+ T cells and OT-1 T cell number per gram of tumor tissue. To evaluate apoptosis, Naïve OT-1 splenocytes were stimulated by OVA257-264 (500 nM) without IL-2 in the presence of absence of sugars for 5 days followed by using Annexin V Apoptosis detection kit (Biolegend). To evaluate IL-2 production, naïve OT-1 splenocytes were stimulated by OVA257-264 (500 nM) without IL-2 in the presence or absence of sugars for 5 days and measured. To evaluate other cellular characteristics, naïve OT-1 splenocytes were stimulated by OVA257-264 (500 nM) with IL-2 (60 IU/mL) in the presence or absence of sugars for 7 or 8 days. On day 7 or 8 cells were taken and analyzed by assessment of ROS, NADPH/NADP+ ratio, and GSH/GSSG ratio.

### Results

T cells treated with both fructose or sucrose exhibited a 6-7-fold increase in the percentage and total number of virus-specific Tim3−TCF-1+ CD8+ T cells in comparison to the untreated T cells. Adding lactose, glucose and galactose also increased the percentage of Tim3−TCF-1+ CD8+ T cells but at a lower level. In the presence of 80 mM of galactose, T cell proliferation is significantly suppressed (FIG. 3B).

Upon adoptive transfer, the lactose-treated and fructose-treated T cells exhibited significantly better capabilities to control tumor growth in comparison to the untreated T cells. T cells treated with sugars and transferred to mice showed a significant decrease in tumor volume compared to T cells that were not treated with sugar (FIG. 3D). T cells treated with sugar have enhanced viability and differential apoptotic characteristics compared to T cells not treated with sugar (FIG. 3E).

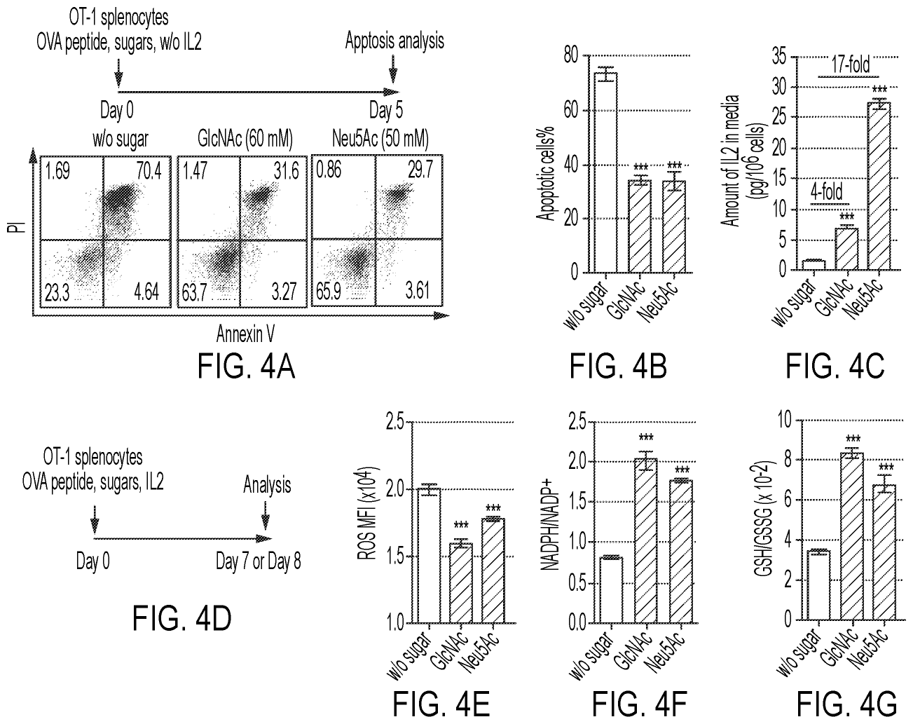

Similarly, T cells treated with both GlcNAc or Neu5Ac exhibited enhanced viability and differential apoptotic characteristics compared to T cells not treated with sugar (FIG. 4B). GlcNAc-treated and Neu5Ac-treated T cells also exhibited significantly greater production of IL-2 (FIG. 4C). Additionally, analysis of ROS, NADPH/NADP+ ratio, and GSH/GSSG ratio revealed that both GlcNAc-treated and Neu5Ac-treated cells have significantly less ROS (FIG. 4E), greater NADPH/NADP+ ratio (FIG. 4F), and greater GSH/GSSG ratio (FIG. 4G) compared to T cells not treated with sugar.

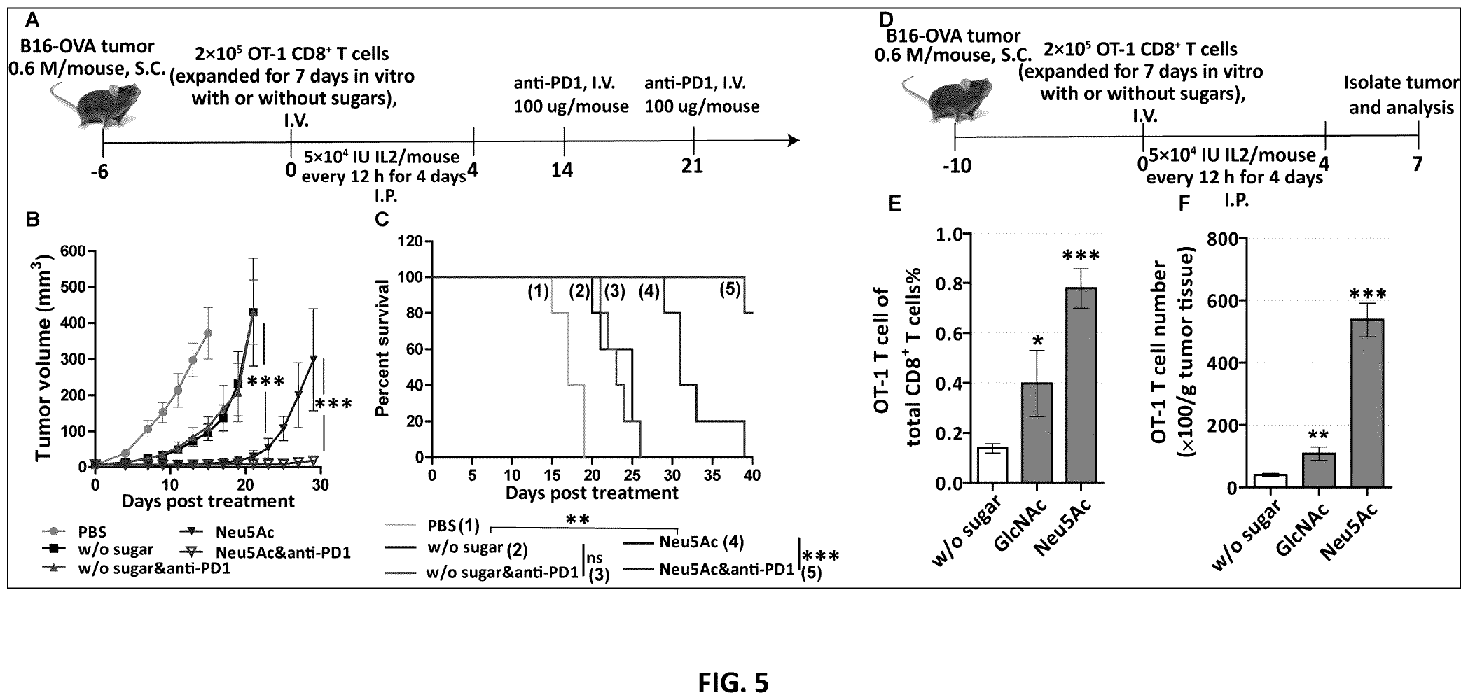

Upon adoptive transfer, T cells treated with Neu5Ac exhibit significantly better capabilities to control tumor growth in comparison to the untreated T cells by way of tumor volume (FIG. 5B) and survival rate (FIG. 5C) post treatment when used with anti-PD-1 antibody treatment. Additionally, T cells treated with GlcNAc and Neu5Ac and transferred to mice both showed increased tumor infiltrating OT-1 T cell numbers (FIGS. 5E and 5F).

### Discussion

This study demonstrates that the use of sugars during in vitro expansion enhances the efficacy of adoptive transfer T cell therapies.

### Example 5: Sugar-Treated OT-1 CD8+ T Cells Exhibit Significantly Differentiated Regulated Genes Compared to Untreated T Cells

### Background

Contacting Tim3−TCF-1+ CD8+ T cells with sugars (compounds discovered in Example 1 and Example 2) has been shown to increase proliferation while maintaining expression of TCF-1 and continued stemness. Elucidating how exposing T cells to particular compounds influences global gene expression could provide further insight into how individual compounds function to decouple proliferation from differentiation during in vitro expansion.

This study aims to investigate and identify transcriptomic differences in T cell populations treated with small molecule modulators (identified in Example 1 and Example 2) that decouple proliferation from differentiation during in vitro expansion.

### Methods

Naïve OT-1 splenocytes were stimulated in vitro with OVA257-264 peptide (500 nM) and TL-2 (60 IU/mL) in the presence or absence of an added compound (untreated (U), GlcNAc (GN), and Neu5Ac (SA)) for 3 days at a density of 1 million/mL (100 uL/well in 96/well plate), then expanded in culture medium containing 60 IU/mL TL-2 for 4 days while maintaining the presence or absence of an added compound. On day 7, dead cells were removed, 80,000 live cells were collected for RNA extraction and subjected to bulk RNA seq analysis. RNA seq design included three samples of each treatment (untreated, GlcNAc-treated, and Neu5Ac-treated). Pairwise DEG analysis was performed using DESeq2. Global gene expression analysis was conducted by sample-only PCA plot, PCA biplot, and heatmap of hierarchical clustering of the top genes by variance between samples.

### Results

PCA revealed that samples within treatment groups (untreated, GlcNAc-treated, and Neu5Ac-treated T cells) exhibited similar clustering of differential gene expression. The individual treatments had limited variability between samples and each treatment resulted in unique and distinguishable differential gene expression within the top differentially regulated genes. Secondary PCA analysis revealed that both GlcNaC-treated and Neu5Ac-treated cells had a degree of overlap, wherein differentially regulated genes were similarly affected compared to untreated cells suggesting that exposure to these compounds modulates gene expression of T cells in a similar fashion. Different compounds identified to decouple T cell proliferation from differentiation exhibit similar expression of differentially regulated genes compared to untreated T cells. Expression patterning of the top 100 differentially regulated genes show similar expression patterning when exposed to GlcNAc and Neu5Ac compared to cells not exposed to either compound.

Further, these genes likely play a role in decoupling proliferation from differentiation. Thus, modulation of one or more of Cd24a, lgfbp7, Tcf7, lgfbp4, Adcy5, Nt5e, Hck, Lif, Apol9b, Gm4951, ligp1, Gzmk, Serpina3f, Nt5dc2, Maged1, Bmf, Cacnb3, Fut4, Egr1, Slc6a12, Fbxo2, Egr2, Fos, Plxnb2, Marcksl1, Ikzf2, Filip1I, Trerf1, Stard10, Pls1, Gm13546, Ccr7, Igha, Itgae, Hic1, Klrh1, Als2cl, Tanc2, Slco4a1, Piwil4, Slc25a23, Itga4, Ckb, Actn1, Sema7a, Gm24187, Mir6236, H4c12, Gzmc, Prf1, Csf1, Gzmd, Tspan32, Atp6v0a1, Map6, Lmna, Rxra, Gpr141, Gm20559, Adam8, Anxa1, Stc2, Fosl2, Akr1c13, Cdkn2a, Crmp1, Tnfrsf21, Kctdl2, Ccr2, Samd9I, Sytl2, Ccr5, Tnfrsf9, Mme, Asns, Eomes, Oas3, Ly6a2, Ifi204, Ifit1, Cdh1, Ifit3, Isg15, Rtp4, Mx1, Oasl2, Rpl12, Rrp1, Rrp1b, Rrp12, Rrp15, mt.Rnr2, CT010467.2, Gm23935, Ct010467.1, and Lars2 genes is expected to impact decoupling of differentiation and proliferation of T cells.

### Discussion

This study demonstrates that the use of sugars, and likely, or other compounds (discovered using the screening methods used in Example 1 and Example 2), during in vitro expansion results in differential regulation of specific genes and pathways compared to untreated cells and are important for the decoupling of differentiation and proliferation.

### Example 6: Sugars Increase TCF-1+ Cells During In Vitro Expansion of CD8+ TSA-Reactive CD8+ TILs

### Background

Cell to cell interactions are essential in numerous biological processes, including immune responses. A technique for monitoring the dynamics of cell to cell interactions is required for researchers to better understand these biological processes. To date, there are limited methods for monitoring cell to cell interaction in vitro and in vivo. As the most practiced cell-engineering approach, genetic engineering is limited by technical complications and safety concerns, such as the viral transduction resistance of primary cells, heterogeneous expression levels, and the potential for endogenous gene disruption. To address these issues, engineering cell surfaces from “outside” using chemical biology tools has emerged as a complementary and generally-applicable approach.

This study aims to evaluate a method to functionalize cell surface with glycan editing enzymes. This enzyme functionalized cell will serve as a “detector” to transfer probe (e.g., biotin, tags, fluorescent molecules) to adjacent cells in an interaction-dependent manner. This technique is intended to be used to identify T cells specific to an antigen. Further, after identifying T cells specific to an antigen, cells are subjected to expansion using sugars (compounds discovered in Example 1 and Example 2) and evaluated for efficacy in cell transfer adoptive therapy.

### Methods

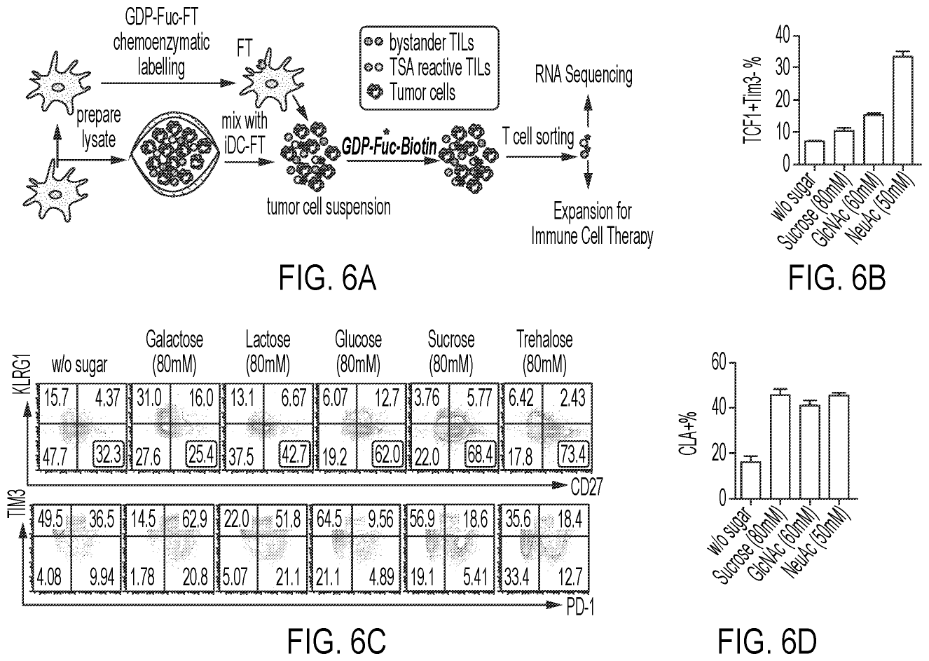

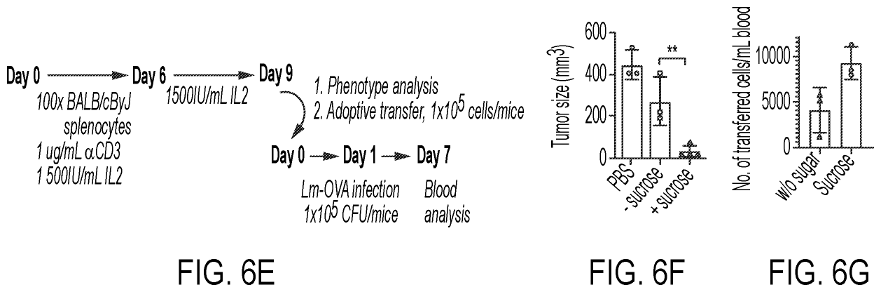

To test the effect of sugars during in vitro expansion of tumor-infiltrating T cells, tumors were mechanically dissociated into single cell suspension and TSA-reactive CD8+ tumor-infiltrating T cells were isolated from MC38 or B16-OVA tumors by FucoID (FIG. 6A) or H-2Kb-SIINFEKL tetramer staining, respectively, and then expanded using a rapid expansion protocol in the presence of allogenic (BALB/cByJ) splenocytes as feeder cells (feeder cell: T cell=100:1), together with 0.5 ng/mL anti-CD3 and 1500 IU/mL IL-2. Sugars were added at the beginning or during the expansion of the TILs, and kept till the end of expansion. On expansion day 9, phenotypes of the expanded T cells were assessed, and the cells were used for adoptive transfer (FIG. 6E). For adoptive transfer, 0.5 million cells (B16-OVA or MC38) were inoculated subcutaneously on each mouse on day 0, Listeria monocytogenes-OVA (LM-OVA) infection 0.5 million CFU/mice on day 1, and blood analysis was recorded on day 7.

### Results

Similar to P14 and OT-I CD8+ T cells, an increase in Tim3−TCF-1+ population in CD8+ TILs expanded with several added sugars was observed (FIG. 6B) in MC38 TILs after expansion.

In accordance with the increase in TCF-1 expression, a significant decrease of KLRG1, Tim3 and PD-1, as well as increased CD27 was observed, indicating that sugar-treated TILs kept a less differentiated, progenitor-exhausted phenotype during expansion (FIG. 6C). Additionally, upon treatment with sucrose, GlcNAc and Neu5Ac, a significant increase in cell surface anti-CLA (Cutaneous Lymphocyte-associated Antigen) staining was observed (FIG. 6D). Since CLA is a skin lymphocyte homing receptor for the vascular lectin endothelial cell-leukocyte adhesion molecule 1, increased CLA may facilitate tumor infiltration of adoptively transferred T cells to improve tumor treatment efficacy. When used for adoptive transfer, OVA-specific TILs isolated from B16-OVA tumors expanded in the presence of sucrose exhibited higher activities in tumor control (FIG. 6F). When these expanded cells were transferred into naïve mice followed by LM-OVA challenge (FIG. 6G), they showed better proliferation compared to the T cells expanded without the additionally added sugars.

### Discussion

This study demonstrates that selected compounds may be used to improve adhesion, homing, and engraftment of adoptively transferred cells, which were identified and enriched by monitoring cell to cell interactions using FucoID method. FucoID can also be used to identify and enrich antigen-specific cytotoxic or regulatory T cells from mouse spleen, human tumor, tumor draining lymph node, and peripheral blood. Further sugars may be used during in vitro expansion of said specific regulatory T cells to enhance the efficacy of adoptive transfer T cell therapies.

### Example 7: Screening for Small Molecule Modulators that can Increase TCF-1+ Cells During In Vitro Expansion of Human T Cells

### Background

Expansion of progenitor exhausted T cells (Tim3−TCF-1+CD8+) using sugars (compounds discovered in Example 1 and Example 2) results in enhanced expansion in vivo using mice (shown in Example 3), consequentially resulting in significantly better capabilities to suppress tumor growth in vivo compared to T cells not treated with sugars (shown in Example 4).

To evaluate whether these sugars had similar effect of preserving the less-differentiated status of T cells in human, this study aims to evaluate selected sugars during the expansion of human peripheral blood T cells.

### Methods

Human PBMC was isolated from whole blood using Ficoll gradient centrifuge. Monocytes were removed by adherence. Non-adherent cells were seeded at 1 million/mL, with anti-human CD3/CD28 dynabeads added at 1:1 ratio, and TL-2 at 300 IU/mL. Cells were stimulated for 3 days, and dynabeads were removed after stimulation. Cells were then expanded by maintaining a concentration of 0.5 to 2 million/mL. Secondary stimulation could be performed between day 7-20. Compounds (sugars) were added either at the beginning of initial stimulation or at the beginning of the secondary activation. Cells during different time points were stained and analyzed by flow cytometry.

### Results

Evaluation of TCF-1+ population of expanded human peripheral CD8+ T cells revealed that several sugars were able to increase the expression of TCF-1 in human T cells during primary activation and expansion. Sucrose and trehalose increased the TCF-1+ population in expanded human peripheral blood CD8 from 10% to 20˜50%.

Detailed analysis of different populations within the CD4+ and CD8+ T cells revealed that sugar treatment altered the components of total T cells. The naïve (T_{N}, CCR7+CD45RA+) and central memory (T_{CM}, CCR7+CD45RA−) population were increased in sugar-treated groups, while the terminal effector population (T_{EMRA}, CCR7−CD45RA+) was decreased. The extent of this effect is dependent on the initial T cell phenotype of the donor PBMCs, and was observed in some, but not all donors.

Sugars added during the secondary activation of human T cells or expansion of isolated human tumor infiltrating T cells were observed to reduce exhaustion and maintain TCF-1 expression. Flow cytometry analysis of TCF-1 and CCR7 or Tim3 showed that sucrose, fructose, NeuAc, or GlcNAc-treatment of T cells could increase the percentage of TCF-1⁺CCR7⁺ and TCF-1⁺Tim3⁻ CD8+ and CD4+ population by a factor of 1.5-2, compared to the untreated group.

### Discussion

This study demonstrates that the use of sugars during in vitro expansion of human T cells results in preservation of less-differentiated status of T cells. Together, Example 1 through Example 7, provides supporting evidence that the screening methods herein may be used to identify compounds that decouple T cell expansion and proliferation, and that those compounds can be used to expand T cells with maintained stemness for the treatment using adoptive cell therapies. Further, the identified compounds can also be used in expansion of human CAR- or TCR-expressing T cells, tumor-infiltrating T cells, or draining lymph node T cells to preserve their progenitor-exhausted or less-differentiated phenotype.

### Example 8: Screening for Small Molecule Modulators that can Increase TCF-1+ Cells During In Vitro Expansion of Human T Cells

We also tested two small molecules (KCl and p38i) reported in papers. (Science 2019, 363, (6434); Cancer Cell 2020, 37, (6), 818-833 e9). K⁺ increased the Tim3−TCF-1+ ratio of OT-1 cells and slightly inhibit the proliferation of OT-1 cells. p38i caused no change in either proliferation or Tim3−TCF-1+ ratio of OT-1 cells.

TABLE 3
Proliferation and Tim3⁻TCF-1⁺ ratio of
OT-1 cells treated with K⁺ or p38iC

| Day 7 | W/O | K⁺(40 mM) | p38i (500 nM) |
|---|---|---|---|
| Proliferation fold, | 1.00 | 0.87 | 1.19 |
| Normalized to W/O | | | |
| Tim3⁻TCF-1⁺ ratio of CD8⁺ | 9.5 | 80.7 | 10.0 |
| T cells (%) | | | |

### Example 9: Sugars Enhance CAR-T Cell Proliferation

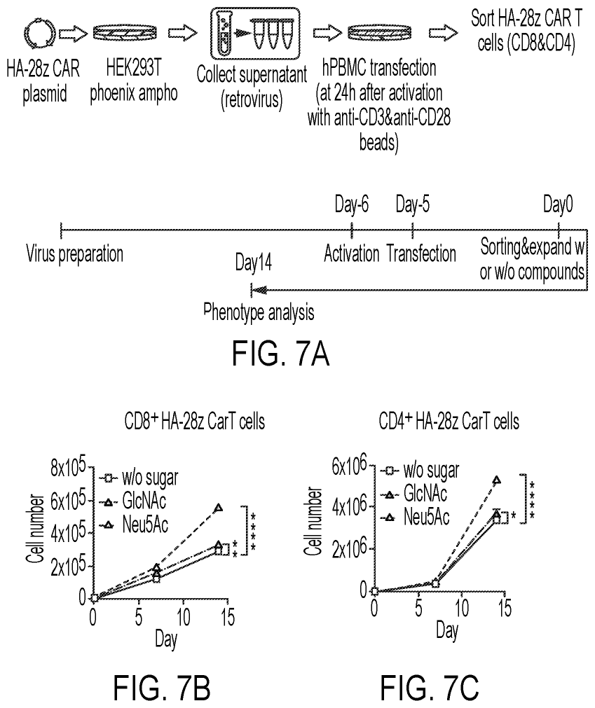

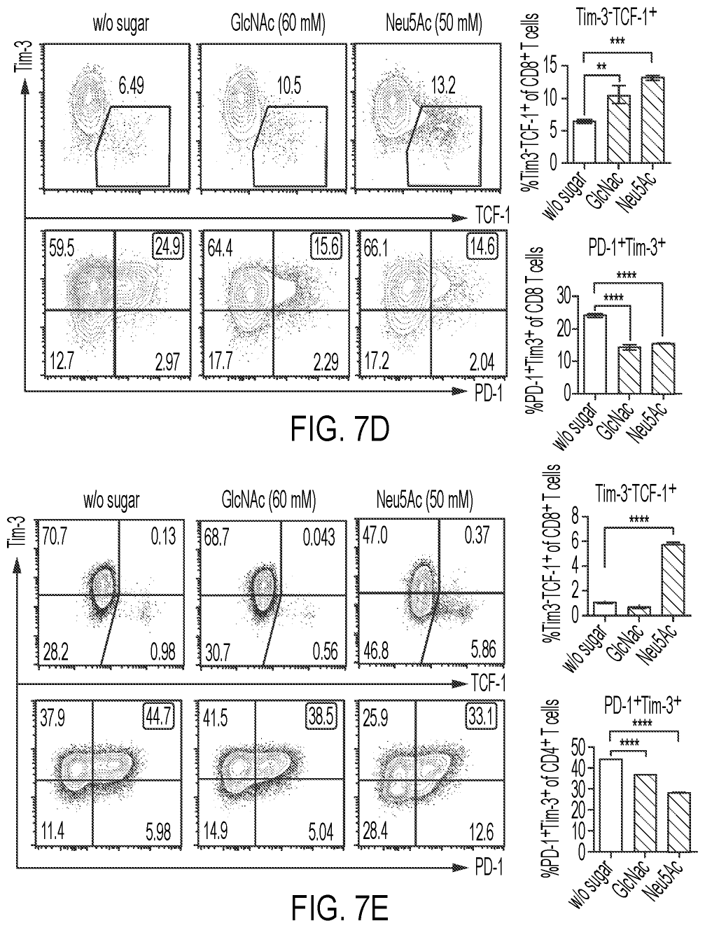

T cell exhaustion has been increasingly incriminated to cause Chimeric antigen receptor (CAR)-T cell dysfunction, which has raised the prospect that maintaining CAR T cells stemness could improve in vivo response. For example, CAR T cells incorporating the disialoganglioside (GD2)-specific 14g2a scFv, CD3ζ and CD28 signaling domains (GD2-28z) develop profound features of exhaustion including reduced expansion in culture, increased expression of inhibitory receptors during in vitro culture before adoptive transfer, as a result of receptor clustering and tonic signaling caused by scFv self-interactions via an antigen-independent way (see, e.g., Weber et al., Science 372(6537):eaba1786, 2021; and Long et al., Nat. Med. 21(6):581-90, 2015). Here, we constructed HA-28z CAR T cells (FIG. 7A) and cultured the sorted CD8⁺ or CD4⁺ CAR T cells with sugars (N-acetylglucosamine, GlcNAc 60 mM or N-Acetylneuraminic acid, Neu5Ac 50 mM) for 14 days. Both CD8⁺ (FIG. 7B) and CD4⁺ (FIG. 7C) CAR T cells treated with Neu5Ac proliferated 1.5-2 times faster than the cells expanded without sugars in regular T cell medium, and showed an increase of progenitor exhausted T cells (Tim-3-TCF-1⁺) and the concomitant decrease of inhibitory receptors (e.g. PD-1, Tim-3) expression (FIGS. 7D-7E).

### Example 10: Effect of a Small Molecule on Generation and Expansion of Effector T Cells

As detailed below, we observed that a small molecule compound facilitates the generation of progenitor-like (Tpex, Tim-3−, TCF-1+) CD8+ T cells without compromising cell growth during in vitro expansion. Structure and some biological activities of this compound, K-Ras(G12C) inhibitor 12, are known in the art. See, e.g., Ostrem et al., Nature 503:548-51, 2013. We decided to assess whether K-Ras(G12C) inhibitor 12 is capable of modulating T cell properties in vitro. We first analyzed phenotypes of OT-I CD8+ T cells which express a transgenic TCR specific for the SIINFEKL peptide (OVA₂₅₇₋₂₆₄) presented on MHC-I. OT-I splenocytes were isolated from OT-I TCR transgenic mice, primed with a single administration of 500 μM SIINFEKL peptide on day 0 to induce activation, cultured in medium containing 60 IU/mL interleukin-2 (IL2) to induce proliferation, and treated with or without 5 μM concentration K-Ras(G12C) inhibitor 12 (Selleckchem, Houston, TX, USA). At 5 μM concentration, K-Ras(G12C) inhibitor 12 does not compromise cell expansion.

CD8+ T cell exhaustion was marked by upregulation of the inhibitory receptors PD-1 and Tim-3, whereas progenitor-like phenotypes were marked by upregulation of the transcription factor TCF-1 and downregulation of Tim-3. On day 5, 23.8% of untreated CD8⁺ OT-I T cells were terminally exhausted (PD-1⁺, Tim-3⁺) and 38.6% exhibited the progenitor-like phenotype (Tim-3⁻, TCF-1⁺). Conversely, on day 5 analysis, about 0.46% of K-Ras(G12C) inhibitor 12 treated CD8⁺ OT-I T cells were terminally exhausted (PD-1⁺, Tim-3⁺), and 91.6% demonstrated the progenitor-like phenotype (Tim-3⁻, TCF-1⁺).

We further observed that K-Ras(G12C) inhibitor 12 prevents the generation of terminally exhausted (PD-1⁺, Tim-3⁺) antigen-specific CD8⁺ tumor infiltrating lymphocytes (TILs) and facilitates production of progenitor-like (Tₚₑₓ, Tim-3⁻, TCF-1⁺) antigen-specific CD8⁺ TILs during in vitro expansion. Specifically, to elucidate the ability of K-Ras(G12C) inhibitor 12 to modulate properties of tumor infiltrating lymphocytes (TILs) in vitro, we utilized an MC38 murine colonic cancer model. C57BL/6J (B6) mice were inoculated with MC38 cells. Tumors were grown for 14 days and then harvested. Antigen specific (H-2K^{b}-restricted MuLV p15E₆₀₄₋₆₁₁-specific, henceforth M8 tetramer⁺) CD8⁺ TILs were isolated. M8 tetramer⁺ CD8⁺ TILs were subjected to rapid expansion. On day 1 of rapid expansion, TILs were added 1:100 with irradiated BALB/c splenocytes and incubated with 1500 IU/mL IL2 for proliferation, 0.5 mg/mL anti-CD3 (mouse) antibody for activation, and with or without K-Ras(G12C) inhibitor 12 (5 μM).

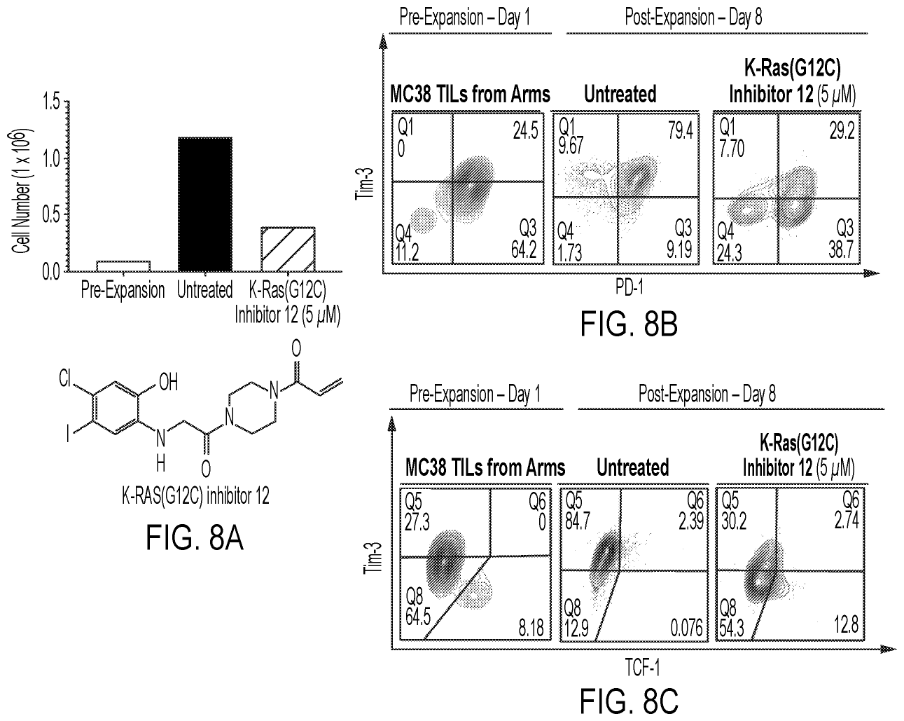

Additionally, M8 tetramer⁺ CD8⁺ TILs treated with K-Ras(G12C) inhibitor 12 (5 μM) experience smaller cell expansion (0.397×10⁶ TILs) compared to the untreated control (1.18×10⁶ TILs) (FIG. 8A). However, K-Ras(G12C) inhibitor 12 (5 μM) effectively prevented the generation of terminally exhausted (PD-1⁺ Tim-3⁺) M8 tetramer⁺ CD8⁺ TILs through rapid expansion. On day 8, post rapid expansion, 79.4% of untreated CD8+ cells displayed the terminally exhausted phenotype, while only 29.2% of K-Ras(G12C) inhibitor 12 (5 μM)-treated CD8+ cells displayed the terminally exhausted phenotype (FIG. 8B). Additionally, K-Ras(G12C) inhibitor 12 (5 μM) maintains and significantly upregulates progenitor-like (Tim-3⁻, TCF-1⁺) M8 tetramer⁺ CD8⁺ TILs through rapid expansion. Post-expansion, 0.076% of untreated CD8⁺ cells displayed the progenitor-like phenotype, while 12.8% of K-Ras(G12C) inhibitor 12 (5 μM)-treated CD8⁺ cells displayed the progenitor-like phenotype (FIG. 8C).

Further studies indicate that K-Ras(G12C) inhibitor 12 facilitates the generation of progenitor-like (TCF-1⁺ Tim-3⁻) CD8⁺ and CD4⁺ T cells from human peripheral blood mononuclear cells (PBMCs) without compromising cell growth during in vitro expansion. PBMCs encompass a heterogeneous cell population with cell frequencies that vary between individuals. Additionally, the phenotypic ratios of PBMC subtypes (i.e., naïve vs memory T cells, TCF-1 expression, etc.) differ greatly between individuals and are related to a multitude of environmental factors. This highlights the importance of comparing K-Ras(G12C) inhibitor 12's activity in several different donors. Specifically, we compared cell expansion and phenotypic modulation in two different donors; henceforth, “donor 3” and “donor 4.”

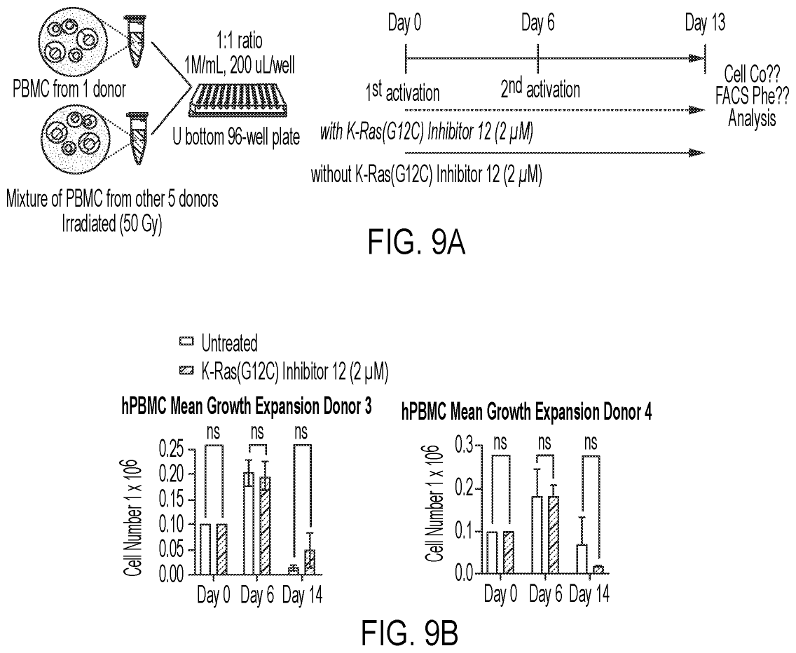

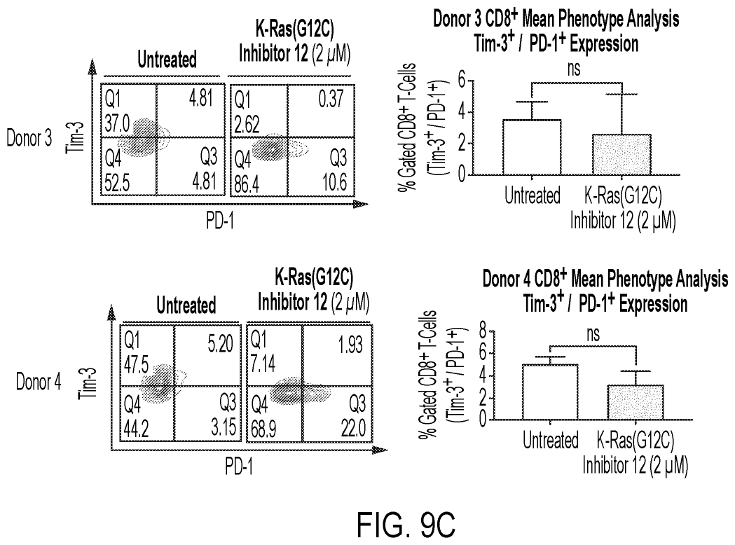

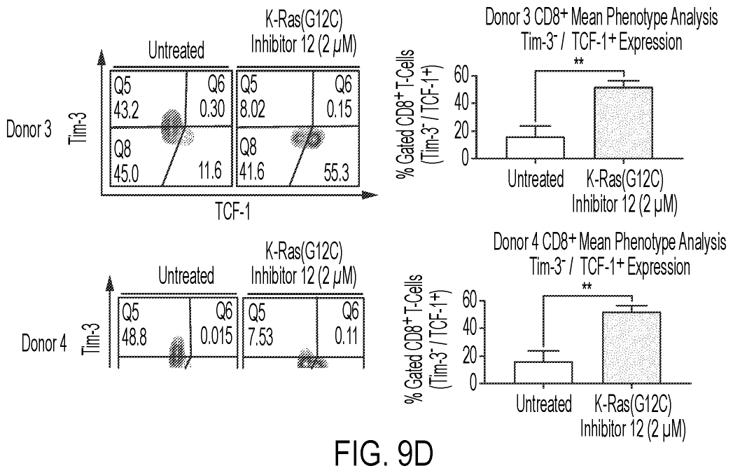

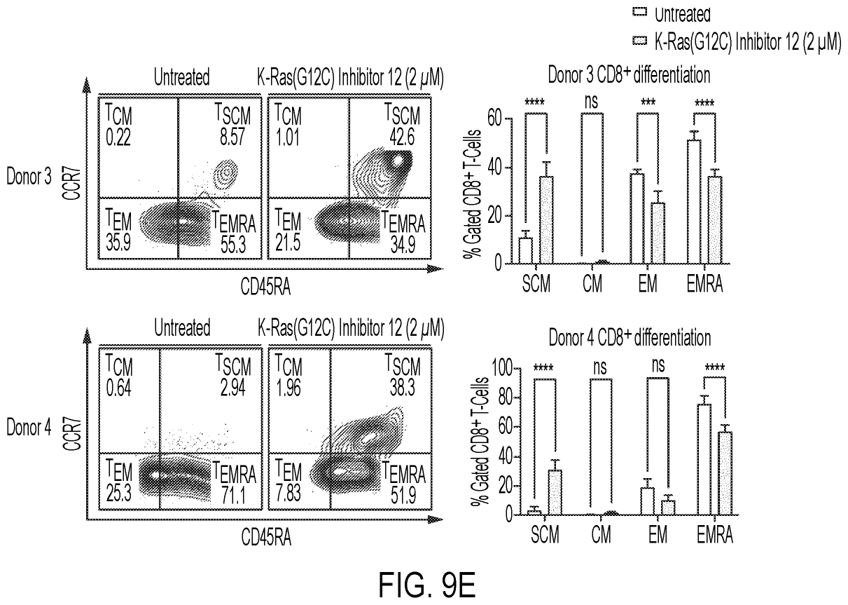

Donor hPBMC cells were co-cultured 1:1 with an irradiated (50 Gy) mixture of hPBMCs from five different donors (henceforth, feeder cells). Irradiation arrests cell division, yet these feeder cells retain the ability to provide extracellular secretions to help donor cells proliferate. Cytokine signaling and HLA mismatch provides for robust donor hPBMC activation. Donor hPBMCs were activated twice with the irradiated feeder cell mixture, initially on day 0 and then similarly on day 6 (FIG. 9A). Beginning on day 0, donor hPBMCs were continuously cultured with 300 IU/mL IL2 and with or without K-Ras(G12C) inhibitor 12 (2 μM) (FIG. 9A). K-Ras(G12C) inhibitor 12 (2 μM)-treated hPBMCs from both donors experience no statistically significant compromise in mean cell expansion (FIG. 9B).

On day 13 immunophenotype analysis indicated that 3.52% of untreated donor 3 CD8⁺ hPBMCs and 5.01% of untreated donor 4 CD8⁺ hPBMCs were terminally exhausted (PD-1⁺, Tim-3⁺), respectively. By contrast, 2.6% of K-Ras(G12C) inhibitor 12 (2 μM)-treated donor 3 CD8⁺ hPBMCs and 3.27% of K-Ras(G12C) inhibitor 12 (2 μM)-treated donor 4 CD8⁺ hPBMCs were terminally exhausted (PD-1⁺, Tim-3⁺), respectively (FIG. 9C). K-Ras(G12C) inhibitor 12 (2 μM) also upregulated progenitor-like (Tim-3⁻, TCF-1⁺) phenotypes in both donor 3 and donor 4 CD8⁺ hPBMCs. On day 13, 15.9% of untreated donor 3 CD8⁺ hPBMCs displayed characteristics of the progenitor-like phenotype, while 51.5% of K-Ras(G12C) inhibitor 12 (2 μM)-treated donor 3 CD8⁺ hPBMCs demonstrated the progenitor-like phenotype. Additionally, 6.78% of untreated donor 4 CD8⁺ hPBMCs showed the progenitor-like phenotype, while 39.7% of K-Ras(G12C) inhibitor 12 (2 μM)-treated donor 4 CD8⁺ hPBMCs exhibited the progenitor-like phenotype (FIG. 9D).

Furthermore, the K-Ras(G12C) inhibitor 12 (2 μM) treatment produced less-differentiated stem-like memory CD8⁺ T cells (T_{SCM}, CCR7⁺, CD45RA⁺) and downregulated CD8⁺ terminally differentiated effector memory cells re-expressing CD45RA (T_{EMRA}, CCR7⁻, CD45RA⁺) in both donor 3 and donor 4 CD8⁺ hPBMCs (FIG. 9E).

In addition to CD8⁺ T cells, K-Ras(G12C) inhibitor 12 (2 μM) was also found to have similar in vitro modulatory properties on CD4⁺ hPBMCs from both donors. On day 13, 15.7% of untreated donor 3 CD4⁺ hPBMCs and 14.7% of untreated donor 4 CD4⁺ hPBMCs were terminally exhausted (PD-1⁺, Tim-3⁺), respectively. In K-Ras(G12C) inhibitor 12 (2 μM)-treated hPBMCs, 0.83% of donor 3 CD4⁺ hPBMCs and 7.9% of donor 4 CD4⁺ hPBMCs were terminally exhausted on day 13, respectively. Analogous to the results presented earlier with CD8⁺ hPBMCs from both donors, K-Ras(G12C) inhibitor 12 (2 μM) facilitated the generation of progenitor-like (Tim-3⁻, TCF-1⁺) phenotypes in both donor 3 and donor 4 CD4⁺ hPBMCs. On day 13, 33.3% of untreated donor 3 CD4⁺ hPBMCs and 53.3% of untreated donor 4 CD4⁺ hPBMCs demonstrated the progenitor-like phenotype, respectively. Dissimilarly, 78.4% of K-Ras(G12C) inhibitor 12 (2 μM)-treated donor 3 CD4⁺ hPBMCs and 78.7% of K-Ras(G12C) inhibitor 12 (2 μM)-treated donor 4 CD4⁺ hPBMCs exhibited the progenitor-like phenotype. Finally, K-Ras(G12C) inhibitor 12 (2 μM) upregulated less-differentiated stem-like memory CD4⁺ T cells (T_{SCM}, CCR7⁺, CD45RA⁺) and downregulated terminally differentiated effector memory cells re-expressing CD45RA (T_{EMRA}, CCR7⁻, CD45RA⁺) in donor 3 CD4⁺ hPBMCs or highly differentiated effector memory CD4+ T cells (T_{EM}, CCR7⁻, CD45RA⁻) in donor 4 CD4⁺ hPBMCs.

Because healthy T cells do not express K-Ras(G12C), it is likely that K-Ras(G12C) inhibitor 12 induces phenotypic modulation in T cell populations via off-target binding. To confirm this, we utilized the previously described OT-I culture model to test two highly specific K-Ras (G12C) inhibitors—Sotorasib and MRTX849—at various concentrations against 5 μM K-Ras(G12C) inhibitor 12. The results indicate that, while K-Ras(G12C) inhibitor 12 effectively promoted the generation of progenitor-like T cells, the highly specific mutant K-Ras (G12C) inhibitors had no activity in modulating CD8+OT-I TCR transgenic T cells during in vitro expansion. Specifically, it was observed that on day 5 mean cell expansion analysis, highly specific mutant K-Ras inhibitors showed no positive effect in increasing cell proliferation, with about the same outcome as that observed with the untreated control at all concentrations tested. Additionally, the highly specific mutant K-Ras inhibitors were not effective at downregulating terminally exhausted (PD-1⁺, Tim-3⁺) CD8⁺ OT-I T cells or upregulating progenitor-like (Tim-3⁻, TCF-1⁺) CD8⁺ OT-I T cells. The results from this study provided further support that K-Ras(G12C) inhibitor 12 modulates T cell properties via off target binding.

### Example 11: EZH2 Inhibitor Tazemetostat Promotes Expansion and Anti-Tumor Activity of Progenitor-Like T Cells

### Introduction

Adoptive cell therapy (ACT)-based cancer immunotherapies have induced remarkable clinical responses in patients with metastatic cancer. However, long-lasting responses are limited to a subset of individuals. In addition, reproducible efficacy against solid tumors is rarely reported (pmid: 25319501, pmid: 31501612). The occurring of T cell exhaustion and the functional impairment of cells used for ACT represent two major roadblocks that limit the successful application of ACT to treat solid tumors. During chronic infections and cancer, persistent antigen exposure drives T cell exhaustion, whose manifestation includes progressive and hierarchical loss of effector functions, sustained upregulation and co-expression of multiple inhibitory receptors, altered expression and use of key transcription factors, metabolic derangements, and a failure to transition to quiescence and acquire antigen-independent memory T cell homeostatic responsiveness. Likewise, during in vitro expansion regimens used to grow a small fraction of reactive T cells into large numbers for patient infusion, T cells are often driven into exhaustion. Consequently, replication potential after transfer is very limited or absent.

Recent studies revealed that two distinct populations of exhausted T cells exist: progenitor exhausted T cells that have stem cell-like properties and give rise to terminally exhausted CD8+ T cells (Tex-term) that possess cytotoxic functions. Progenitor exhausted TILs can respond to anti-PD-1 therapy, but terminally exhausted TILs cannot. T cell activation and differentiation are accompanied by epigenetic changes that switch genes on or off and determine which proteins are transcribed. Such changes in turn play pivotal roles in the determination of T cell fate and function. DNA methylation and histone post-translational modifications represent two major epigenetic mechanisms (https://pubmed.ncbi.nlm.nih.gov/31813765/).

Previous studies using acute viral infection models uncovered that Ezh2 deficiency led to a compromised CD8 effector program without periodizing memory cell formation. Analyzing Ezh2 targets whose expression decreased during the differentiation of effector cells but not during the differentiation of memory cells revealed that many genes encode products that have been linked to the differentiation of memory cells but not to the differentiation of effector cells (https://pubmed.ncbi.nlm.nih.gov/28218746/). These Ezh2 targets included genes encoding memory-associated transcription factors, such as Tcf7 and Eomes; molecules that mediate TGF-signaling, such as Smad2, whose product has been linked to CD8+ that control T cell survival and homing, such as Klf2; as well as Opa1, which encodes a regulator of mitochondrial fusion with a critical role in differentiating memory CD8⁺ T lymphocytes. Although the role of EZH2 in the establishment of the epigenetic state of exhausted T cells remains to be explored, many genes highly expressed in progenitor exhausted T cells overlap with the aforementioned EZH2 targets that are linked to the differentiation of memory cells but not to the differentiation of effector cells. Moreover, many genes associated with the reversible exhaustion state are also associated with proliferation (notably TCF-1, MYC, NF-κB, and genes enabling glycolysis). These observations suggest that transient inhibition of EZH2 in the early stage of T cell differentiation toward exhaustion may facilitate the generation of progenitor exhausted T cells with “stem-like” properties that are highly desirable for cancer immunotherapy in the settings of immune checkpoint blockade and ACT.

As detailed below, we discovered that culturing CD8 T cells in vitro with Taz inhibited the formation of H3K27Me3 and produced T cells with progenitor-like/memory properties without compromising cell proliferation. Treating T cells with Taz confines inhibition of EZH2 to in vitro expansion, thus avoiding any long-term functional EZH2 loss after adoptive transfer. Upon adoptive transfer, in the absence of Taz, EZH2 activity would be recovered, enabling the transferred T cells to differentiate into cells with effector-like functions for better tumor control.

### Results:

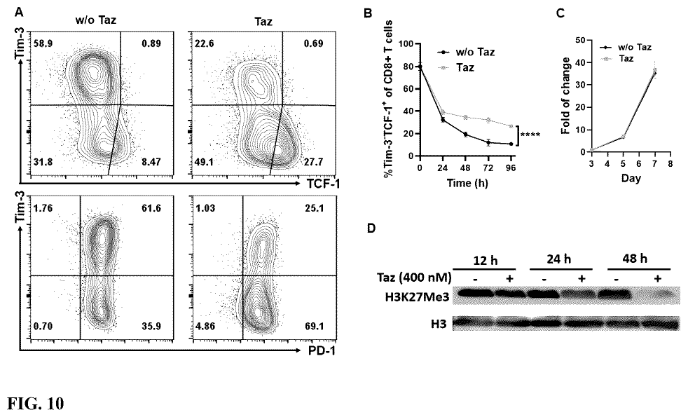

Transient inhibition of EZH2 with Taz promotes progenitor-like features of T cells without compromising cell expansion: To determine if transient inhibition of EZH2 with Taz could promote pro-memory or progenitor-like features of T cells, we developed an assay platform to analyze phenotypes of in vitro expanded OT-I CD8 T cells that express a transgenic TCR specific for the SIINFEKL peptide (OVA₂₅₇₋₂₆₄) of chicken ovalbumin presented on MHC-I. In this assay, OT-I splenocytes were primed with high concentrations of OVA₂₅₇₋₂₆₄ peptide (500 nM), which resulted in pronounced T cell exhaustion by day 7 as evidenced by up-regulation of PD-1 and Tim-3. At this time point, 58.9% OT-I T cells were terminally exhausted (Tim3⁺TCF-1⁻) and 8.47% exhibited progenitor exhausted phenotype (Tim3⁻TCF-1⁺) (FIGS. 10A-10B). We first evaluate the impact of Taz on T cell proliferation by adding Taz at different time points following T cell activation. When Taz was added to the culture on day 0 together with OVA₂₅₇₋₂₆₄ peptide, all cells were found dead. By contrast, adding Taz on day 3 and maintaining it at the same concentration during in vitro culture had no inhibition on cell expansion (FIG. 10C). At approximately 24 hr upon the addition of Taz, notable downregulation of H3K27Me3 was detected. And by 48 hr, the formation of H3K27Me3 was almost fully suppressed (FIG. 10D). On day 7 of the T cell expansion, T cell treated with Taz (3-7) were found to have 2.5-3.5 fold increase in progenitor exhausted cells 27.7% (Tim3⁻TCF-1⁻) (FIGS. 10A-10B).

Transient EZH2 inhibition improves in vivo proliferation and recall response of transferred T cells: To determine the long-term functional effects of Taz on T cell function after in vivo transfer, we assessed an in vivo homeostasis and the recall response of OT-I T cells cultured with Taz. OT-II cells were cultured with Taz for 4 days after activation. On Day 7, after removal of Taz from T cells, we transferred equivalent numbers of Taz-treated and untreated OT-I cells (CD45.2+/+) to congenically marked C57BL/6 mice (CD45.1+/+ or CD45.1+/−) (FIG. 11A). To assess the homeostasis of OT-I cells, these mice were infected with LM-OVA (10⁴ CFU/mouse) on day 30 after the adoptive transfer of cells. After LM-OVA challenge, OT-I CD8 T cells treated Taz during in vitro expansion showed a stronger response evidenced by more OT-I cells were found in blood (FIGS. 111B-11C) and spleen (FIG. 11D). Therefore, transient EZH2 inhibition in vitro of OT-I cells can provide better homeostasis and recall response upon primary challenge with LM-OVA in vivo.

Transient EZH2 inhibition enhances anti-tumor efficacy of transferred T cells in a mouse model of ACT: To determine the capabilities of Taz-treated T cells for treating solid tumors in vivo, we intravenously infused untreated and OT-I T cells that had been expanded with Taz into C57BL/6 mice bearing established B16-OVA tumors (FIG. 12A), followed by anti-PD-1 administration on day14 and day 21. In the control groups, mice were treated with PBS only. Although OT-I (expanded without Taz) or anti-PD-1&OT-I (expanded without Taz) treatments only showed modest tumor control, OT-I (expanded with Taz) significantly slowed down tumor growth with median tumor size being only 1/8 of that treated with OT-I (expanded without Taz) (treatment day 20) (FIG. 12B). Even better tumor control was realized when a combination of anti-PD-1&OT-I (expanded with Taz) was used (FIG. 12,B). Moreover, whereas no mice in any control group survived up to day 24, >80% mice treated with OT-I (expanded with Taz) were still alive by day 30 (FIG. 12C). These results indicated that compared to the untreated OT-I cells, cells expanded with Taz possess markedly higher activities to control tumor growth in vivo. These Taz pre-treated cells consist of a higher fraction of stem cell-like progenitor exhausted CD8+ T cells whose effector functions are successfully unleashed upon PD-1 blockade.

Transient EZH2 inhibition increases progenitor-like cells in human PBMC and CAR-T cells: To assess the transient EZH2 inhibition on human T cells, we activated human peripheral blood mononuclear cells (hPBMCs) with a mixture of irradiated (50 Gy) hPBMCs from another five HLA-mismatched donors (FIG. 13A). After activated for 3 days, hPBMC cells were expanded for 3 days with Taz (1 μM). Taz was also kept with the same concentration for the second activation and expansion till day13 after 1^{st} activation (FIG. 13A). On Day 13, compared to hPBMCs expanded in regular T cell medium, hPBMCs cultured with Taz showed more progenitor-like cells phenotype (FIGS. 13B and 13E), stem cell memory- and effector memory-like features (FIGS. 13,C and F) without scarification of proliferation (FIGS. 13D and 13G) for both donor A and donor B. These Taz treated hPBMCs also exhibited a higher cytokine production after activation (FIGS. 13H and 13K).

A recent study by Mackall and coworkers demonstrated that transient rest could restore functionality in exhausted CAR-T cells (reference: Science, 2021, 372 (6537), 49, DOI: 10.1126/science.aba1786). To explore if transient EZH2 inhibition during CAR-T cell expansion by treating cells with Taz may generate similar outcomes, we transduced donor PMBCs with a tonically signaling GD2-targeting CAR (H28) that is known to undergo antigen-independent, tonic CAR signaling as a result of spontaneous receptor clustering and cultured the sorted CAR-T cell in the presence of Taz for 14 days (FIG. 14A).

HA28 CAR-T cells manifest extremely robust tonic signaling and acquired functional, transcriptomic and epigenetic hallmarks of exhaustion by day 5 after sorting (day 11 after activation). On day 14 after expansion, compared to CAR-T cells cultured in normal medium, cells cultured in the presence of Taz exhibited decreased inhibitory receptor expression (FIGS. 14C-14F) and concomitant increased TCF-1 expression (FIG. 1,B).

### Example 12: Sugars Maintain Progenitor Exhausted Features and Reverse Terminal Exhausted TILs from B16-OVA Tumor Back to Progenitor Exhausted T Cells

To investigate which population that sugars mainly changed on T cells, B16-OVA cancer cells (7×10⁵ cells) were subcutaneously inoculated into the left or right flank of TCF7^{GFP} mice expressing an EGFP fluorescent reporter from the endogenous Tcf7 locus, which enable us to track the expression of TCF-1 in live cells.

Two populations of cells, Tim-3⁻TCF-GFP⁺ (progenitor exhausted) and Tim-3⁺TCF-GFP⁻ (terminal exhausted), from the TILs isolated from B16-OVA tumors inoculated in Tcf7^{GFP} mice were sorted and in vitro expanded following the rapid expansion protocol for 7 days (FIG. 15A). For sorted Tim-3-TCF-GFP⁺ TILs, compared to the normal culture condition without sugars, media supplemented with both GlcNac and Neu5Ac can maintain more Tim-3⁻TCF/GFP⁺ cells with progenitor exhausted features, including a higher ratio of Tim-3⁻TCF-GFP⁺ cell population and a lower expression of inhibitory receptors (FIG. 15B). Significantly, Neu5Ac can partially reverse the sorted Tim-3⁺TCF-GFP⁻ back to Tim-3⁻TCF-GFP⁺ phenotype (FIG. 15C).

### Example 13: Some Purine and Pyrimidine Biosynthesis End Products Help to Maintain the T Cell Progenitor Exhausted Phenotype

We discovered that treating CD8+ T cells with N-Acetylneuraminic acid upregulated purine and pyrimidine biosynthesis end products and certain intermediates in these two pathways. We therefore tested if purine and pyrimidine nucleotides have similar effects as N-Acetylneuraminic acid to help maintain less differentiated phenotypes of T cells during expansion. OT-I splenocytes were cultured with 500 nM SIINFEKL peptide (OVA₂₅₇₋₂₆₄) for three days at initial cell density of 1×10⁶/mL to activate and expand the CD8+ T cell population. Different concentrations of the products or intermediates in purine or pyrimidine de novo synthesis pathways were added on day 3. The tested compounds include, e.g., guanosine, deoxyguanosine, adenosine, deoxyadenosine, inosine, xanthosine, orotidine, uridine, cytidine, deoxycytidine, thymidine, deoxythymidine, 5-Aminoimidazole-4-carboxamide ribonucleotide (AICAR), orotate, and dihydroorotate. The phenotype of expanded T cells were analyzed on day 5. We found that guanosine and AICAR were able to increase the Tim3−TCF1+ progenitor exhausted population from 20% to over 50% at concentrations as low as 50-250 μM. Adding these two compounds together with N-Acetylneuraminic acid on day 3 further enhanced the proportion of Tim3−TCF1+ T cells by a factor of 1.5 to 2 when analyzed on day 5 without significant impact on cell proliferation. Finally, G6PDi-1, an inhibitor of glucose-6-phosphate dehydrogenase (Ghergurovich et al., Nat Chem Biol. 2020 July; 16(7): 731-739) showed similar effects through modulation of the pentose phosphate pathway. Adding G6PDi-1 on day 3 of OT-I culture increase the proportion of Tim3−TCF1+ T cells by a factor of 1.5 when analyzed on day 5 without significant impact on cell proliferation.

### Example 14: Further Studies of KRAS (G12C) Inhibitors and Derivatives

We performed additional studies to examine the observed activities of KRAS (G12C) inhibitors in modulating T cell properties. First, OT-I splenocytes were activated with OVA₂₅₇₋₂₆₄ peptide (500 nM) for 3 days in the presence of 60 IU/mL IL-2 and cultured for another 7 days. Inhibitor 9 (i9) or inhibitor 12 (i12) was added on Day0 (D0) upon activation at 5 uM and the concentration was kept till day 7 or added on Day3 (D3) after activation at 5 uM or 10 uM and the concentration was kept till day 7 for analysis. As shown in FIGS. 16A and 16B, i9 or i12 treatment significantly increased the ratio of progenitor exhausted T cells (Tim-3-TCF-1l) and decreased the expression of inhibitory receptors (Tim-3+PD-1).

In further studies, 6-7-week-old female C57BL/6J mice were subcutaneously implanted with 6×10⁵ B16-OVA cells (day −6). Five days (day −1) after the inoculation of B16-OVA cells, the mice were irradiated with a dose of 5 Gy for lymphodepletion. OT-I cells (2.5×10⁵ cells/mouse) from a female OT-I mouse in vitro expanded with or without inhibitor 9 were transferred into the mice bearing B16-OVA tumors (day 0) followed by IL-2 (5 x105 IU suspended in 200 uL DPBS for each mouse) infusions every 12h for 4 days via intraperitoneal (IP) injection. Anti-PD-1 antibody infusions were performed on day 14 and day 21 after the adoptive transfer of OT-I cells with a dose of 100 ug/mouse by IV injection. As shown in FIG. 17, the results from these studies indicate that OT-I cells treated with K-Ras(G12C) Inhibitor 9 provided improved tumor control and enhanced response to PD-1 blockade.

Other than inhibitors 9 and 12, we also tested activities of two analogues of these KRAS (G12C) inhibitors. The analogues, Z7 and Z6, are derived from Inhibitor 9 and Inhibitor 12, respectively. They have an alkynyl group for click reaction which is used for ABPP analysis to find the targets of the inhibitors (FIG. 18A). In the studies, OT-I splenocytes were activated with OVA257-264 peptide (500 nM) for 3 days in the presence of 60 IU/mL IL-2 and cultured for another 7 days. 5 uM i2, i9, z6 or z7 were added on Day0 (DO) upon activation. Cells treated with Z6 and Z7 showed similar progenitor features and expression level of inhibitory receptors to cells treated with inhibitor 12 and inhibitor 9, demonstrating that Z6 and Z7 have similar activity to i9 and i12 in T cells (FIG. 18B).

As noted above, Inhibitor 9 and Inhibitor 12 have a cysteine-reactive group (sulphonamide and acrylamide) that allows covalent binding of i9 and i12 to a cysteine residue in proteins. We further tested two analogue compounds, ZT-1 and ZT-2, that are not capable of forming the covalent binding. ZT-1 is derived from inhibitor 9, and ZT-2 is derived from inhibitor 12. In these two compounds, the cysteine-reactive group is replaced by an acetamide group that cannot react with cysteine. Specifically, OT-I splenocytes were activated with OVA₂₅₇₋₂₆₄ peptide (500 nM) for 3 days in the presence of 60 IU/mL IL-2 and cultured for another 7 days. Inhibitor 9 (i9) or Inhibitor 12 (i12) was added on Day0 (D0) upon activation at 5 uM. ZT-1 and ZT-2 were added on Day0 (D0) upon activation at Sum or 10 μm and the concentration was kept till day 7 for flow cytometry analysis. Cell number was recorded on day 5 and day 7. It was found that all compounds didn't affect cell proliferation. However, cells treated with ZT-1 and ZT-2 that don't contain electrophilic warheads showed a similar phenotype to untreated cells, demonstrating that the activity of the inhibitors requires a cysteine-reactive group. In other words, the inhibitors interact with their targets via a covalent bond.

### Example 15: Activities of KRAS (G12C) Inhibitors and Derivatives on CAR-T Cells

In addition to T cells, we further examined whether the KRAS (G12C) inhibitors and derivative compounds have similar activities on CAR-T cells. In this study, cryopreserved human PBMCs were thawed and activated the same day with Dynabeads™ Human T-Activator CD3/CD28 for T Cell Expansion and Activation (Gibco, Cat #1113ID) at 1:1 beads:cell ratio in T cell medium containing 150 IU/mL of IL-2. T cells were transduced with HA-28z retroviral vector at 24-36h after the activation and maintained at 0.5×10⁶-1×10⁶ cells/mL in T cell medium with 100 IU/mL of IL-2 for 4-5 days. CAR-T cells were stained with anti-mouse IgG F(ab′)₂ (FITC, Jackson ImmunoResearch, Cat #515-095-072) and sorted out for expansion with 100 IU/mL of IL-2 in the presence or absence of Inhibitor 9 at 2 or 3 uM or Inhibitor 12 at 1 or 2 uM. Phenotype of CD8+ CAR-T cells was analyzed on day 15 and cell number was recorded. In vitro and in vivo activity against target cell (Nalm6-GD2 expressing luciferase) of the CAR-T cells expanded with inhibitor 9/12 or vehicle (untreated) were then examined. Specifically, six-to-eight-week-old male mice were intravenously inoculated with 1×106 Nalm6-GD2 expressing luciferase leukaemia on day 0. 2×10⁶ Mock T cells or CAR-T cells expanded with vehicle, i9 (3 uM) or i12 (1 uM) were injected intravenously on day 3 after the tumor inoculation. In vivo leukaemia progression was measured by bioluminescent imaging using the IVIS imaging system.

Results from the studies indicate that Inhibitor 9 or 12 treatment increased progenitor features and decreased the expression of inhibitory receptors (PD-1, LAG3, Tim-3 and CD39) of anti-GD2 CAR-T cells (FIG. 19A). Inhibitor 9 also facilitated the proliferation of CAR-T cells (FIG. 19B). Compared to vehicle treated CAR-T cells, anti-GD2 CAR-T cells treated with i9 (3 uM) or i12 (1 uM) exhibited higher anti-tumor activity against the target cell (FIG. 19C). The treatment also led to better in vivo tumor control and a prolonged survival of tumor bearing mice (FIGS. 20A and 20B).

What is claimed is:

1. A method of expanding activated T cells to produce T cells with enhanced in vivo persistence after adoptive transfer, comprising contacting a cell population comprising activated T cells with an effective amount of a compound that uncouples T cell expansion from differentiation, thereby expanding the activated T cells to produce T cells with enhanced in vivo persistence after adoptive transfer.

2. The method of claim 1, wherein the activated T cells are stem memory T cells (Tscm), central memory T cells (Tcm), effector memory T cells, effector T cells, progenitor exhausted T cells (Tpex), or terminally exhausted T cells (Ttex).

3. The method of claim 1, wherein the produced T cells are less differentiated relative to the activated T cells not treated with the compound.

4. The method of claim 1, wherein the compound is a sugar or sugar derivative.

5. The method of claim 4, wherein the sugar is trehalose, sucrose, lactose, fructose or neuraminic acid.

6. The method of claim 4, wherein the sugar derivative is N-Acetylglucosamine (GlcNAc) or N-acetylneuraminic acid (Neu5Ac).

7. The method of claim 1, wherein the compound is an inhibitor of Enhancer of zeste homolog 2 (EZH2).

8. The method of claim 7, wherein the inhibitor is Tazemetostat.

9. The method of claim 1, wherein the compound is a small molecule inhibitor of KRAS (G12C) mutant.

10. The method of claim 9, wherein the inhibitor is KRAS (G12C) Inhibitor 9 or KRAS (G12C) Inhibitor 12.

11. The method of claim 1, wherein the compound is guanosine, 5-Aminoimidazole-4-carboxamide ribonucleotide (AICAR), or Glucose-6-phosphate Dehydrogenase Inhibitor 1 (G6PDi-1).

12. The method of claim 1, wherein the T cell is a tumor infiltrating lymphocyte (TIL) or a genetically engineered T cell.

13. The method of claim 12, wherein the TIL is a CD8+ T cell or a CD4+ T cell.

14. The method of claim 12, wherein the genetically engineered T cell is a TCR-modified T cell or a CAR-T cell.

15. A method for converting a TCF-1 negative T cell into partially TCF-1 positive, comprising contacting a population of TCF-1 negative T cells with an effective amount of a compound that uncouples T cell expansion from differentiation, thereby converting the TCF-1 negative T cell into partially TCF-1 positive.

16. The method of claim 15, wherein the compound is N-acetylneuraminic acid (Neu5Ac) or N-Acetylglucosamine (GlcNAc).

17. A kit for in vitro expansion of an activated T cell for adoptive cell therapy, comprising (1) an effective amount of a compound that uncouples T cell expansion from differentiation, and (2) an instruction of co-culturing the compound with a population of cells comprising the activated T cell.

18. The kit of claim 17, wherein the activated T cell is stem memory T cell (Tscm), central memory T cell (Tcm), effector memory T cell, effector T cell, progenitor exhausted T cell (Tpex), or terminally exhausted T cell (Ttex).

19. A method of screening for compounds that promote proliferation of activated T cells and/or maintain expanded T cells in a less differentiated state, comprising:
(a) providing T cells from a T cell-containing tissue from a subject;
(b) culturing activated T cells from the T cell-containing tissue in the presence of a compound; maintaining said culturing for a time period sufficient to permit proliferation of T cells, wherein T cells from the T cell-containing tissue have undergone stimulating by an activating agent, wherein stimulating occurs prior to or concurrently with culturing; and
(c) measuring: (i) an amount of T cells expressing TCF-1; and/or (ii) an amount of TCF-1 expression, wherein an increase in both (i) and (ii) relative to T cells after expansion not treated with the compound identifies the compound as promoting proliferation of progenitor exhausted T cells and maintaining expanded T cells in a less differentiated state, and wherein an increase in (ii) and not (i) relative to T cells after expansion not treated with the compound identifies the compound as promoting maintaining expanded T cells in a less differentiated state.

20. The method of claim 19, further comprising re-stimulating and expanding the T cells in the culture following (c), and maintaining said culturing for a time period sufficient to permit expansion of T cells, wherein T cells are assessed by measuring: (i) an amount of T cells expressing TCF-1; and/or (ii) an amount of TCF-1 expression, wherein an increase in both (i) and (ii) relative to T cells after expansion not treated with the compound identifies the compound as promoting proliferation of progenitor exhausted T cells and maintaining expanded T cells in a less differentiated state, and wherein an increase in (ii) and not (i) relative to T cells after expansion not treated with the compound identifies the compound as promoting maintaining expanded T cells in a less differentiated state.
